(19) United States
(12) Patent Application Publication
(10) Pub. No.: US 2003/0148264 A1
(43) Pub. Date: Aug. 7, 2003

(54) Phage displayed PDZ domain ligands

(75) Inventors: Heike A. Held, Oakland, CA (US); Laurence A. Lasky, Sausalito, CA (US); Richard P. Laura, San Bruno, CA (US); Sachdev S. Sidhu, San Francisco, CA (US); Wai Lee Tan Wong, Los Altos, CA (US); Yan Wu, Foster City, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/190,082

(22) Filed: Jul. 3, 2002

Related U.S. Application Data
(60) Provisional application No. 60/303,634, filed on Jul. 6, 2001.

(51) Int. Cl. C12Q 1/70; G01N 33/53; C07H 21/04; C12N 7/00; C07K 14/005

(52) U.S. Cl. 435/5; 435/7.1; 435/235.1; 536/23.72; 530/350

(57) ABSTRACT

The invention pertains to a method of identifying PDZ interacting polypeptides, said polypeptides, and uses of said polypeptides.

### RELATED APPLICATIONS

This application claims the priority benefit of U.S. Provisional Application Serial No. 60/303,634 filed Jul. 6, 2001, which is incorporated herein by reference in its entirety.

### FIELD OF THE INVENTION

The invention relates to a method to identify protein-protein interactions mediated by PDZ domains, using phage display. The invention also relates to the polypeptides identified as those that interact with and bind PDZ domains.

### BACKGROUND

The normal functioning of a cell depends on the subcellular localization and compartmentalization of its components and processes. A consequence of aberrant cellular organization, which may be caused by pathological agents, genetic mutations, or environmental traumas, is the lack of proper function. Sequence-specific interactions between proteins provide the basis for structural and functional organization within cells. Structurally conserved protein domains that recognize variations on a short peptide motif, such as PDZ domains, mediate some of these interactions.

PDZ (PSD-95/Discs large/ZO-I) domains, originally described as conserved structural elements in the 95-kDa post-synaptic density protein (PSD-95), the Drosophila tumor suppressor discs-large, and the tight junction protein zonula occludens-1 (ZO-1), are contained in a large and diverse set of proteins (Craven and Bredt, 1998; Fanning and Anderson, 1999; Tsunoda et al., 1998). In general, PDZ domain-containing proteins appear to assemble various functional entities, including ion channels and other transmembrane receptors, at specialized subcellular sites such as epithelial cell tight junctions, neuromuscular junctions, and post-synaptic densities of neurons. These clustering and localization effects have important biological implications. For example, the membrane-associated guanylate kinase, PSD-95, segregates the N-methyl D-aspartate (NMDA) receptor and the Shaker potassium channel to the post-synaptic density of neurons (Tejedor et al., 1997). In another illustration, the aggregation of various components of the fruit fly visual system by the multi-PDZ protein INAD greatly enhances the efficiency of this signaling cascade (Tsunoda et al., 1997). Another compelling case is the use of several PDZ domain-containing proteinsin the appropriate basolateral localization of the LET-23 receptor tyrosine kinase of Caenorhabditis elegans (Kaech et al., 1998). This kinase is required for vulval development, and mutations in these PDZ domain-containing proteins result in the subcellular mislocalization of the LET-23 protein and a lack of vulval differentiation. Together with many other examples, these studies indicate that PDZ domains are important intracellular assembly and localization cofactors in diverse signaling pathways.

PDZ domains recognize three different types of ligands, with two of these interactions showing specificity for peptides at the extreme carboxyl termini of proteins (Cowburn and Riddihough, 1997; Harrison, 1996; Oschkinat, 1999). Type I and type II PDZ domains recognize carboxyl-terminal peptides with the consensus sequence Thr/Ser-X-Phe/Val/Ala-COOH or Phe/Tyr-X-Phe/Val/Ala-COOH, respectively. Interestingly, a third type of PDZ domain-ligand interaction involves the recognition of an internal peptide sequence. Structural analyses of these three types of PDZ interactions haveilluminated the mechanisms of ligand recognition. For example, the crystal structure of a type I PDZ domain from PSD95 showed that a 4-residue carboxyl-terminal peptide interacts with the protein via an antiparallel main chain association with a β strand, and the terminal carboxylate is inserted into a conserved “carboxylate binding loop” (Doyle et al., 1996; Morais Cabral et al., 1996) The crystal structure of a PDZ domain from human CASK revealed the nature of interactions mediated by type II motifs (Daniels et al., 1998). In both domain types, the peptide formed a new antiparallel β strand in the PDZ domain structure, and the overall conformations of the two interactions were similar. However, there were significant differences in side chain contacts that could account for the different ligand specificities of the two domain types. Finally, the interaction between a PDZ domain of syntrophin and a PDZ domain of the neuronal nitric oxide synthase has been examined by x-ray and NMR analyses (Hillier et al., 1999; Tochio et al., 1999). In this case, an extended loop of the neuronal nitric oxide synthase PDZdomain forms a β finger that binds to a β strand of the syntrophin PDZ domain, in a manner that mimics the carboxyl-terminal ligands of types I and II domains. Together, these data suggest that these three types of PDZ domains use similar but highly specialized regions to recognize diverse carboxyl-terminal and internal peptide ligands.

Initial forays into PDZ domain ligand specificities were performed using combinatorial libraries consisting of either free peptides (Songyang et al., 1997) or peptides fused to the carboxyl terminus of the Escherichia coli Lac repressor (Stricker et al., 1997). Although phage display is the most commonly used method for displaying combinatorial peptide libraries, phage-displayed peptide libraries reported to date have been displayed as fusions to the amino terminus of either the major coat protein (protein-8, P8) or the gene-3 minor coat protein, primarily because it is believed that neither coat protein can support carboxyl-terminal fusions (Palzkill et al., 1998; Stricker et al., 1997). Thus phage display has not been used for the display of peptides with free carboxyl termini, and the technology has not been amenable to the analysis of PDZ domain carboxyl-terminal binding specificities (Gee et al., 1998; Stricker et al., 1997).

Bacteriophage (phage) display is a technique by which variant polypeptides are displayed as fusion proteins to the coat protein on the surface of bacteriophage particles (Scott and Smith, 1990). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla et al., 1990) or protein (Clackson et al., 1991; Kang et al., 1991; Lowman et al., 1991; Marks et al., 1991a; Smith, 1991) libraries on phage have been used for screening millions of polypeptides for ones with specific binding properties (Smith, 1991) Sorting phage libraries of random mutants requires a strategy for constructing and propagating a large number of variants, a procedure for affinity purification using the target receptor, and a means of evaluating the results of binding enrichments (U.S. Pat. No. 5,223,409; U.S. Pat. No. 5,403,484; U.S. Pat. No. 5,571,689; U.S. Pat. No. 5,663,143).

Typically, variant polypeptides are fused to a gene-3 protein (P3), which is displayed at one end of the viron. Alternatively, the variant polypeptides may be fused to the major coat protein of the viron, gene-8 protein (P8). Such polyvalent display libraries are constructed by replacing the phage gene-3 with a cDNA encoding the foreign sequence fused to the amino terminus of the gene-3 protein. Such fusions can complicate efforts to sort high affinity variants from libraries because of the avidity effect; that is, phage can bind to the target through multiple point attachment. Moreover, because the gene-3 protein is required for attachment and propagation of phage in the host cell, e.g., E. coli, such fusion proteins can dramatically reduce infectivity of the progeny phage particles.

To overcome these difficulties, monovalent phage display was developed. In this approach, a protein or peptide sequence is fused to a portion of a gene-3 protein and expressed at low levels in the presence of wild-type gene-3 protein such that particles display mostly wild-type gene-3 protein and one or no copies of the fusion protein (Bass et al., 1990; Lowman and Wells, 1991). Significant advantages of monovalent over polyvalent phage display include (1) progeny phagemids retain full infectivity, (2) avidity effects are reduced, and consequently, sorting is mediated by intrinsic ligand affinity, and (3) phagemid vectors, which simplify DNA manipulations, are used. See also U.S. Pat. No. 5,750,373 and U.S. Pat. No. 5,780,279. Others have also used phagemids to display proteins, particularly antibodies (U.S. Pat. No. 5,667,988; U.S. Pat. No. 5,759,817; U.S. Pat. No. 5,770,356; and U.S. Pat. No. 5,658,727).

A two-step approach has been used to select high affinity ligands from peptide libraries displayed on M13 phage. Low affinity leads are first selected from naive, polyvalent libraries displayed on the major coat protein, P8. The low affinity selectants are subsequently transferred to the gene-3 minor coat protein and matured to high affinity in a monovalent format. Unfortunately, extension of this methodology from peptides to proteins has been difficult because display levels on P8 vary with fusion length and sequence: increasing fusion size generally decreases display. Thus, while monovalent phage display has been used to affinity many different proteins, polyvalent display on P8 has not been applicable to most protein scaffolds.

Although most phage display methods have used filamentous phage, lambdoid phage display systems (WO 95/34683; U.S. Pat. No. 5,627,024), T4 phage display systems (Efimov et al., 1995; Jiang, 1997; Ren and Black, 1998; Ren et al., 1996; Ren, 1997; Zhu, 1997) and T7 phage display systems (Smith and Scott, 1993); (U.S. Pat. No. 5,766,905) are also known.

Other improvements and variations of phage display have been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277), and phage display libraries have been used to analyze and control bimolecular interactions (WO 98/20169; WO 98/20159) and properties of constrained helical peptides (WO 98/20036). To selectively isolate binding ligands, for example, a method of isolating an affinity ligand in which a phage display library is contacted with one solution in which the ligand will bind to a target molecule, and a second solution in which the affinity ligand will not bind to the target molecule can be used (WO 97/35196). WO 97/46251 describes a method of panning a random phage display library with an affinity purified antibody and then isolating binding phage, followed by a panning process using microplate wells to isolate high affinity binding phage. The use of Staphlylococcus aureus protein A (“protein A”) as an affinity tag has also been reported (Li et al., 1998). WO 97/47314 describes the use of substrate subtraction libraries to distinguish enzyme specificities using a combinatorial library that may be a phage display library. A method for selecting enzymes suitable for use in detergents using phage display is described in WO 97/09446. Additional methods of selecting specific binding proteins are also described (U.S. Pat. No. 5,498,538; U.S. Pat. No. 5,432,018; and WO 98/15833).

Methods of generating peptide libraries and screening these libraries are also disclosed in U.S. Pat. No. 5,723,286; U.S. Pat. No. 5,432,018; U.S. Pat. No. 5,580,717; U.S. Pat. No. 5,427,908; and U.S. Pat. No. 5,498,530. See also U.S. Pat. No. 5,770,434; U.S. Pat. No. 5,734,018; U.S. Pat. No. 5,698,426; U.S. Pat. No. 5,763,192; and U.S. Pat. No. 5,723,323.

Methods that alter the infectivity of phage are also known. WO 95/34648 and U.S. Pat. No. 5,516,637 describe a method of displaying a target protein as a fusion protein with a pilin protein of a host cell, where the pilin protein is preferably a receptor for a display phage. U.S. Pat. No. 5,712,089 describes infecting a bacteria with a phagemid expressing a ligand and then superinfecting the bacteria with helper phage containing wild type P3 but not a gene encoding P3 followed by addition of a P3-second ligand where the second ligand binds to the first ligand displayed on the phage produced. See also WO 96/22393. A selectively infective phage system using non-infectious phage and an infectivity-mediating complex is also known (U.S. Pat. No. 5,514,548).

Phage systems displaying a ligand have also been used to detect the presence of a polypeptide binding to the ligand in a sample (WO/9744491), and in an animal (U.S. Pat. No. 5,622,699). Methods of gene therapy (WO 98/05344) and drug delivery (WO 97/12048) have also been proposed using phage which selectively bind to the surface of a mammalian cell.

Further improvements have enabled the phage display system to express antibodies and antibody fragments on a bacteriophage surface, allowing for selection of specific properties, i.e., binding with specific ligands (EP 844306; U.S. Pat. No. 5,702,892; U.S. Pat. No. 5,658,727) and recombination of antibody polypeptide chains (WO 97/09436). A method to generate antibodies recognizing specific peptide—MHC complexes has also been developed (WO 97/02342). See also U.S. Pat. No. 5,723,287; U.S. Pat. No. 5,565,332; and U.S. Pat. No. 5,733,743.

U.S. Pat. No. 5,534,257 describes an expression system in which foreign epitopes up to about 30 residues are incorporated into a capsid protein of a MS-2 phage. This phage is able to express the chimeric protein in a suitable bacterial host to yield empty phage particles free of phage RNA and other nucleic acid contaminants. The empty phage are useful as vaccines.

The expression of fusion proteins on the surface of bacteriophage particles is variable and depends, to some extent, on the size of the polypeptide. Conventional phage display systems use wild-type phage coat proteins and fuse the heterologous polypeptide to the amino terminus of the wild-type amino acid sequence or an amino terminus resulting from truncation of the wild-type coat protein sequence. Segments of linker amino acids have also been added to the amino terminus of the wild type coat protein sequence to improve selection and target binding.

All references cited herein, including patent applications and publications, are incorporated by reference in their entirety.

### SUMMARY

In one aspect, the invention provides methods of identifying peptides that bind to PDZ domains of intracellular proteins using a carboxyl-terminal phage display method. These peptides are useful to identify cognate protein ligands for the PDZ domains using the method of the invention. Structural analyses of such peptides are useful to understand PDZ domain structure and function, and also to identify intracellular biological functions for these motifs and the proteins that contain them. The peptides are further useful per se for example as PDZ domain inhibitors and are also useful as structural models in the design of small molecule inhibitors/agonists of the binding interaction between a PDZ domain containing protein and its cognate ligand.

Using methods of the invention, cognate ligands and synthetic peptides that bind to the PDZ domain of a number of proteins can be and have been discovered. These include peptides that bind to the PDZ domain of the proteins as listed below, with the corresponding cognate ligands for each PDZ domain/protein identified based on the peptide sequence(s):

(1) ERBIN: δ-catenin; Armadillo repeat gene deleted in velocardiofacial syndrome (ARVCF); p0071

(2) Densin: ARVCF; δ-catenin; p0071

(3) Scribble PDZ 1 & 3: Tight junction protein 2 (Z02); voltage-gated potassium channel (shaker-related subfamily 1) member 5 (Kvl 0.5); member of the rhodopsin family of G protein-coupled receptors (GPCR) (GPR87); actinin; beta-catenin; CD34

(4) Scribble PDZ2: δ-catenin; ARVCF; p0071

(5) MUPP PDZ7: 5-hydroxytryptamine 2B (seronin) receptor (HTR2B); platelet-derived growth factor receptor beta chain (PDGFRb); δ-catenin; serum glucocorticoid regulated kinase (SGK); somatostatin receptor 3 (SSTR3)

(6) Human INADL PDZ6: 5-hydroxytryptamine 2B (seronin) receptor (HTR2B); platelet-derived growth factor receptor beta chain (PDGFRb); δ-catenin; serum glucocorticoid regulated kinase (SGK); somatostatin receptor 3 (SSTR3)

(7) Human ZOI: claudin-17; claudin 1; claudin 3; claudin 7; claudin 9; claudin 18; PDGFRA; PDGFRB; δ-catenin; ARVCF; SGK

(8) AF6(MLLT4): FYCOI; BLTR2; TM7SF3; OR10C1; CNTNAP2 (contactin associated protein-like2); nectin3; SH3D5; utrophin

(9) MUPP PDZ3: drosophila NUMB homolog; TGFBRI; IGFBP7; CD3611

(10) MAGII PDZ3: SDOLF (olfactory receptor sdolf); PLEKHA1; PEPP2; MUC12; SLIT1; PARK2; HTR2A; PITPNB

(11) MAGI3 PDZ3: JAM1; JAM2; LLT1; PTTG3; CD83 antigen; delta-like homolog (drosophila) (also preadipocyte factor (fetal antigen 1); TNFRSF18; RGS20; TM4SF6; PARK2; GPR10; IL2RB

(12) INADL PDZ3: BLTR2; JAM1; JAM2; KV8.1; PTTG3; CNTNAP2; NRXN1; NRXN2; NRXN3; TNFRSF18; PTTGI; PARK2; GABRG2; CNTFR; CCR3; GABRG3; GABRP

(13) huINADL PDZ2: PIWI1 (Piwi (Drosophila)-like 1); likely ortholog of mouse piwi-like homolog; NRXN1; NRXN2; PPP2CA; PPP2CB

(14) huPARD3PDZ3: hara-kiri (HRK); downregulated in ovarian cancer 1 (DOC1); PIW1; PPP1R3D

(15) SNTAI PDZ: MRGX2; NLGN1; NLGN3; SEEK1; claudin 17; GPR56; SSTR5; SCTR; GRM1; GRM2; GRM3; GRM5

(16) MAG13 PDZ0: LANO; SSTR3; NRCAM; GPR19; GNG5; HTR2B

(17) MUPP PDZ13: NLGN3; NLGN1; claudin 16; GPR56; enigma; FZD9; SSTR5; VCAM1; GPRK6

(18) MAG13 PDZ2: PTEN/MMAC

In various aspects, the invention provides:

1. A fusion protein comprising at least a portion of a phage coat protein bonded through the carboxyl-terminus thereof, optionally through a peptide linker, to a PDZ domain binding peptide, where the peptide preferably contains 3-20, more preferably 4-12, more preferably 4-7 amino acid residues.

2. The fusion protein of aspect 1, where the phage is a filamentous phage.

3. The fusion protein of aspect 2, where the coat protein is a g3, g6 or g8 protein.

4. The fusion protein of aspect 1, where the PDZ domain binding peptide contains 3-20, preferably 4-12, more preferably 4-7 amino acid residues.

5. The fusion protein of any of aspects 1-4, where the phage coat protein comprises the mature phage coat protein.

6. A fusion gene encoding the fusion protein of any one of aspects 1-5.

7. A vector, preferably a phage or phagemid vector, comprising the fusion gene of aspect 6.

8. A virus particle comprising the vector of aspect 7.

9. A library of fusion proteins of any of aspects 1-5, where the fusion proteins in the library comprise a plurality of PDZ domain binding peptides.

10. A library of vectors of aspect 7, where the fusion genes encode fusion proteins comprising a plurality of PDZ domain binding peptides.

11. A library of virus particles of aspect 8, where the fusion genes encode fusion proteins comprising a plurality of PDZ domain binding peptides.

12. A method for producing a PDZ domain binding peptide library comprising: expressing in recombinant host cells a library of variant fusion proteins of aspect 9 to form a library of recombinant phage particles displaying the plurality of PDZ binding peptides on the surface thereof.

13. A method for selecting PDZ domain binding peptides comprising:(a) expressing in recombinant host cells a library of variant fusion proteins of aspect 9 to form a library of recombinant phage particles displaying the plurality of PDZ binding peptides on the surface thereof; (b) contacting the recombinant phage particles with a target containing a PDZ domain so that at least a portion of the phage particles bind to the target; and (c) separating phage particles that bind to the target from those that do not bind.

14. The method of aspect 13, where the phage particles contain fusion genes encoding the fusion proteins, further comprising sequencing at least a portion of the fusion gene of a selected phage particle to determine the amino acid sequence of a PDZ domain binding peptide, and optionally, synthesizing the PDZ domain binding peptide.

15. A method for identifying PDZ domain binding protein, comprising:(a) selecting PDZ domain binding peptides using the method of aspect 13 to obtain phage particles containing fusion genes encoding the selected PDZ domain binding peptides, and sequencing a portion of the fusion genes to identify the amino acid sequence of at least one of the selected PDZ domain binding peptides; (b) comparing the PDZ domain binding peptide sequence with the carboxyl-terminal amino acid sequence of a group of proteins, and selecting an intracellular protein having a carboxyl-terminal sequence which is identical to or similar to (preferably at least about 60%, 70%, 80%, 90% or 95% identical to) the PDZ domain binding peptide sequence.

16. The method of aspect 15, where the carboxyl-terminal sequence of the selected intracellular protein is identical to or differs at 1,2 or 3 positions from the PDZ domain binding peptide sequence.

17. The method of aspect 15, further comprising comparing the binding to a PDZ domain, of a selected PDZ domain binding peptide and of a selected intracellular protein or carboxyl-terminal sequence thereof.

18. An assay for a PDZ domain binding compound, comprising: contacting a PDZ domain containing polypeptide with a candidate PDZ domain binding compound, preferably in the presence of a PDZ domain binding peptide known to bind the PDZ domain, and detecting binding of the polypeptide and compound.

19. A host cell containing the vector of aspect 7.

20. An isolated polypeptide comprising a carboxy terminal amino acid sequence having the sequence of a member selected from the group consisting of SEQ ID NOs:14-181, 209 213 and 241-247. Preferably, said polypeptide does not comprise an amino acid sequence identical to any one of SEQ ID NOs:688-705. In some embodiments, the invention provides an isolated polypeptide comprising a carboxy terminal amino acid sequence having at least preferably 85%, preferably 80%, preferably 70%, preferably 60% identity to the sequence of a member selected from the group consisting of SEQ ID NOs: 14-181, 209-213 and 241-247.

21. The polypeptide of aspect 20, consisting essentially of a member selected from the group consisting of SEQ ID NOs: 14-181, 209-213 and 241-247.

22. The polypeptide of aspect 20, consisting of a member selected from the group consisting of SEQ ID NOs:14-181, 209-213 and 241-247.

23. An isolated polypeptide comprising a carboxy terminal amino acid sequence having the sequence of a member selected from the group consisting of SEQ ID NOs:1-12. Preferably, said polypeptide does not comprise an amino acid sequence identical to any one of SEQ ID NOs:797. In some embodiments, the invention provides an isolated polypeptide comprising a carboxy terminal amino acid sequence having at least preferably 85%, preferably 80%, preferably 70%, preferably 60% identity to the sequence of a member selected from the group consisting of SEQ ID NOs:1-12.

24. The polypeptide of aspect 20, consisting essentially of a member selected from the group consisting of SEQ ID NOs: I-12.

25. The polypeptide of aspect 20, consisting of a member selected from the group consisting of SEQ ID NOs:1-12.

26. An isolated polypeptide comprising a carboxy terminal amino acid sequence of a member selected from the group consisting of SEQ ID NOs: 13 and 512-575. Preferably, said polypeptide does not comprise an amino acid sequence identical to any one of SEQ ID NOs:744 and 747-757.

27. The polypeptide of aspect 20, consisting essentially of a member selected from the group consisting of SEQ ID NOs:13 and 512-575.

28. The polypeptide of aspect 20, consisting of a member selected from the group consisting of SEQ ID NOs:13 and 512-575.

29. An isolated polypeptide comprising a carboxy terminal amino acid sequence of a member selected from the group consisting of SEQ ID NOs:248-284. Preferably, said polypeptide does not comprise an amino acid sequence identical to any one of SEQ ID NOs:706-708.

30. The polypeptide of aspect 20, consisting essentially of a member selected from the group consisting of SEQ ID NOs:248-284.

31. The polypeptide of aspect 20, consisting of a member selected from the group consisting of SEQ ID NOs:248-284.

32. An isolated polypeptide comprising a carboxy terminal amino acid sequence of a member selected from the group consisting of SEQ ID NOs:285-292. Preferably, said polypeptide does not comprise an amino acid sequence identical to any one of SEQ ID NOs:688-705.

33. The polypeptide of aspect 20, consisting essentially of a member selected from the group consisting of SEQ ID NOs:285-292.

34. The polypeptide of aspect 20, consisting of a member selected from the group consisting of SEQ ID NOs:285-292.

35. An isolated polypeptide comprising a carboxy terminal amino acid sequence of a member selected from the group consisting of SEQ ID NOs:293-303. Preferably, said polypeptide does not comprise an amino acid sequence identical to any one of SEQ ID NOs:707 and 715-718.

36. The polypeptide of aspect 20, consisting essentially of a member selected from the group consisting of SEQ ID NOs:293-303.

37. The polypeptide of aspect 20, consisting of a member selected from the group consisting of SEQ ID NOs:293-303.

38. An isolated polypeptide comprising a carboxy terminal amino acid sequence of a member selected from the group consisting of SEQ ID NOs:304-315. Preferably, said polypeptide does not comprise an amino acid sequence identical to any one of SEQ ID NOs:707 and 715-718.

39. The polypeptide of aspect 20, consisting essentially of a member selected from the group consisting of SEQ ID NOs:304-315.

40. The polypeptide of aspect 20, consisting of a member selected from the group consisting of SEQ ID NOs:304-315.

41. An isolated polypeptide comprising a carboxy terminal amino acid sequence of a member selected from the group consisting of SEQ ID NOs:316-336. Preferably, said polypeptide does not comprise an amino acid sequence identical to any one of SEQ ID NOs:706-707, 717 and 719-726.

42. The polypeptide of aspect 20, consisting essentially of a member selected from the group consisting of SEQ ID NOs:316-336.

43. The polypeptide of aspect 20, consisting of a member selected from the group consisting of SEQ ID NOs:316-336.

44. An isolated polypeptide comprising a carboxy terminal amino acid sequence of a member selected from the group consisting of SEQ ID NOs:337-374.

45. The polypeptide of aspect 20, consisting essentially of a member selected from the group consisting of SEQ ID NOs:337-374.

46. The polypeptide of aspect 20, consisting of a member selected from the group consisting of SEQ ID NOs:337-374.

47. An isolated polypeptide comprising a carboxy terminal amino acid sequence of a member selected from the group consisting of SEQ ID NOs:375-391. Preferably, said polypeptide does not comprise an amino acid sequence identical to any one of SEQ ID NOs:709-714.

48. The polypeptide of aspect 20, consisting essentially of a member selected from the group consisting of SEQ ID NOs:375-391.

49. The polypeptide of aspect 20, consisting of a member selected from the group consisting of SEQ ID NOs:375-391.

50. An isolated polypeptide comprising a carboxy terminal amino acid sequence of a member selected from the group consisting of SEQ ID NOs:392-401. Preferably, said polypeptide does not comprise an amino acid sequence identical to any one of SEQ ID NOs:709-714.

51. The polypeptide of aspect 20, consisting essentially of a member selected from the group consisting of SEQ ID NOs:392-401.

52. The polypeptide of aspect 20, consisting of a member selected from the group consisting of SEQ IDNOs:392-401.

53. An isolated polypeptide comprising a carboxy terminal amino acid sequence of a member selected from the group consisting of SEQ ID NOs:402-413. Preferably, said polypeptide does not comprise an amino acid sequence identical to any one of SEQ ID NOs:776-777, 779 and 791-796.

54. The polypeptide of aspect 20, consisting essentially of a member selected from the group consisting of SEQ ID NOs:402-413.

55. The polypeptide of aspect 20, consisting of a member selected from the group consisting of SEQ ID NOs:402-413.

56. An isolated polypeptide comprising a carboxy terminal amino acid sequence of a member selected from the group consisting of SEQ ID NOs:414-419. Preferably, said polypeptide does not comprise an amino acid sequence identical to any one of SEQ ID NOs:719 and 775-785.

57. The polypeptide of aspect 20, consisting essentially of a member selected from the group consisting of SEQ ID NOs:414-419.

58. The polypeptide of aspect 20, consisting of a memberselected from the group consisting of SEQ ID NOs:414-419.

59. An isolated polypeptide comprising a carboxy terminal amino acid sequence of a member selected from the group consisting of SEQ ID NOs:420-426. Preferably, said polypeptide does not comprise an amino acid sequence identical to any one of SEQ ID NOs:768 and 772-774.

60. The polypeptide of aspect 20, consisting essentially of a member selected from the group consisting of SEQ ID NOs:420-426.

61. The polypeptide of aspect 20, consisting of a member selected from the group consisting of SEQ ID NOs:420-426.

62. An isolated polypeptide comprising a carboxy terminal amino acid sequence of a member selected from the group consisting of SEQ ID NOs:427-432. Preferably, said polypeptide does not comprise an amino acid sequence identical to any one of SEQ ID NOs:759-760 and 768-771.

63. The polypeptide of aspect 20, consisting essentially of a member selected from the group consisting of SEQ ID NOs:427-432.

64. The polypeptide of aspect 20, consisting of a member selected from the group consisting of SEQ ID NOs:427-432.

65. An isolated polypeptide comprising a carboxy terminal amino acid sequence of a member selected from the group consisting of SEQ ID NOs:433-463. Preferably, said polypeptide does not comprise an amino acid sequence identical to any one of SEQ ID NOs:728, 731, 744, 747-748, 750, 753 and 758-767.

66. The polypeptide of aspect 20, consisting essentially of a member selected from the group consisting of SEQ ID NOs:433-463.

67. The polypeptide of aspect 20, consisting of a member selected from the group consisting of SEQ ID NOs:433-463.

68. An isolated polypeptide comprising a carboxy terminal amino acid sequence of a member selected from the group consisting of SEQ ID NOs:464-511. Preferably, said polypeptide does not comprise an amino acid sequence identical to any one of SEQ ID NOs:739-746.

69. The polypeptide of aspect 20, consisting essentially of a member selected from the group consisting of SEQ ID NOs:464-511.

70. The polypeptide of aspect 20, consisting of a member selected from the group consisting of SEQ ID NOs:464-511.

71. An isolated polypeptide comprising a carboxy terminal amino acid sequence of a member selected from the group consisting of SEQ ID NOs:576-582. Preferably, said polypeptide does not comprise an amino acid sequence identical to any one of SEQ ID NOs:735-738.

72. The polypeptide of aspect 20, consisting essentially of a member selected from the group consisting of SEQ ID NOs:576-582.

73. The polypeptide of aspect 20, consisting of a member selected from the group consisting of SEQ ID NOs:576-582.

74. An isolated polypeptide comprising a carboxy terminal amino acid sequence of a member selected from the group consisting of SEQ ID NOs:583-601. Preferably, said polypeptide does not comprise an amino acid sequence identical to any one of SEQ ID NOs:727-734.

75. The polypeptide of aspect 20, consisting essentially of a member selected from the group consisting of SEQ ID NOs:583-601.

76. The polypeptide of aspect 20, consisting of a member selected from the group consisting of SEQ ID NOs:583-601.

77. A polypeptide that binds to the same epitope as a polypeptide of the invention. Preferably, a polypeptide that binds to the same epitope as a polypeptide of the invention is a peptide that is from about 3 to about 20, from about 4 to about 12, or from about 4 to about 7 amino acids in length.

78. A polypeptide that competes for binding to a PDZ domain with a polypeptide of the invention. Preferably, a polypeptide that competes for binding to a PDZ domain with a polypeptide of the invention is a peptide that is from about 3 to about 20, from about 4 to about 12, or from about 4 to about 7 amino acids in length. In some embodiments, the invention provides polypeptides that compete for binding to a PDZ domain with a polypeptide known to bind said PDZ domain. In some embodiments, the polypeptide known to bind said PDZ domain comprises, consists essentially of, or consists of GGWRWTTWL, GGERIWWV, GGWFLDV or GGWETWV. For example, a polypeptide that competes for binding to a PDZ domain with GGWRWTTWL is WRWTTWL, YRWTTWL, WRHTTWL, WGWTTWL or WRWTTWV, wherein the N-terminal residue of said polypeptide may be (but is not necessarily) acetylated.

79. In another aspect, the invention provides a polynucleotide (including a recominant vector and expression vector) encoding any of the polypeptides of the invention.

80. A method of inhibiting a polypeptide-polypeptide interaction, comprising: contacting a mixture comprising a first and a second polypeptide with an inhibitor of interaction between a PDZ domain and its ligand, wherein the first polypeptide comprises said PDZ domain and the second polypeptide comprises said ligand.

81. The method of aspect 80, wherein the first polypeptide is a fusion polypeptide which comprises a PDZ domain and the second polypeptide comprises a ligand of said PDZ domain, and the first polypeptide is attached to a substrate (such as a solid support).

82. The method of aspect 80, wherein the first polypeptide is a fusion polypeptide which comprises a PDZ domain and the second polypeptide comprises a ligand of said PDZ domain, and the second polypeptide is attached to the substrate.

83. A method of screening for a substance that modulates interaction (preferably binding) between a PDZ domain polypeptide and a molecule known to bind to the PDZ domain of said polypeptide (for example, a cognate ligand) comprising:

(a) contacting a sample containing said polypeptide and molecule with a candidate substance;

(b) determining amount of binding of said molecule to said polypeptide in the presence of said candidate substance;

(c) comparing the amount of binding of step (b) with amount of binding of said molecule to said polypeptide under similar conditions in the absence of said candidate substance; whereby a difference in amount of binding as determined in (c) indicates that said candidate substance is a substance that modulates said interaction.

84. A method of screening for a substance that inhibits binding of a PDZ domain polypeptide to a molecule known to bind to the PDZ domain of said polypeptide comprising:

(a) contacting a sample containing said polypeptide and molecule with a candidate substance;

(b) determining amount of binding said molecule to said polypeptide in the presence of the candidate substance;

(c) comparing the amount of binding of step (b) with amount of binding of said molecule to said polypeptide under similar conditions in the absence of the candidate substance; whereby a decrease in amount of binding of the polypeptide and said molecule in the presence of the candidate substance compared to the amount of binding in the absence of said candidate substance as determined in (c) indicates that said candidate substance is a substance that inhibits binding of the PDZ domain polypeptide to the molecule known to bind to the PDZ domain of said polypeptide.

85. A method of screening for a substance that increases binding of a PDZ domain polypeptide to a molecule known to bind to the PDZ domain of said polypeptide comprising:

(a) contacting a sample containing said polypeptide and molecule with a candidate substance;

(b) determining amount of binding said molecule to said polypeptide in the presence of the candidate substance;

(c) comparing the amount of binding of step (b) with amount of binding of said molecule to said polypeptide under similar conditions in the absence of the candidate substance; whereby an increase in amount of binding of the polypeptide and said molecule in the presence of the candidate substance compared to the amount of binding in the absence of said candidate substance as determined in (c) indicates that said candidate substance is a substance that increases binding of the PDZ domain polypeptide to the molecule known to bind to the PDZ domain of said polypeptide.

86. A method comprising administering a substance to a subject with a condition associated with abnormal binding interaction of a PDZ domain polypeptide and a ligand, wherein said substance is a modulator of said binding interaction. Preferably, the modulator is a substance known to affect affinity of binding interaction of the ligand to the PDZ domain. In some embodiments, the modulator inhibits (for example, as indicated by a decrease in the amount of PDZ domain polypeptide-ligand complex in a cell) said interaction. In some embodiments, the modulator enhances (for example, as indicated by an increase in the amount of PDZ domain polypeptide-ligand complex in a cell) said interaction. Conditions associated with abnormal interaction between a PDZ domain polypeptide and its ligand would be evident to one skilled in the art in view of the biological functions, roles and/or activities of the PDZ domain polypeptide and the ligand. For example, ARVCF, which is shown herein as a ligand for the PDZ domain of DENSIN-180 and ERBIN, is a gene whose deletion is shown to be associated with velocardiofacial syndrome, and whose gene product has binding affinity for cadherins and thus likely plays a role in cell adhesion at the adherens junction. Abnormal interaction between DENSIN or ERBIN and ARVCF is therefore associated with a known condition, i.e., velocardiofacial syndrom, and any condition associated with a change in cadherin-related cell adhesion function. Other examples of conditions associated with abnormal interaction of a PDZ domain polypeptide and its ligand would include, but are not limited to, Parkinson diseases (for example, related to PARK2); tumorigenesis (for example, related to PTEN/MMAC, PTTG3, DOCI); conditions associated with abnormalities in cytoskeletal function/regulation (for example, those related to actinin, catenins, utrophin); signal transduction (for example, those related to membrane-associated guanylate kinase signaling, serum glucocorticoid regulated kinase (SGK), FYCO1, TM7SF3, SH3D5, drosophila NUMB homolog, PLEKHA1, PEPP2, PITPNB, JAM1, JAM2, LLTI, RGS20, IL2RB, PPP2CA, PPP2CB, PPPIR3D, SSTR5, SCTR, GRMI, GRM2, GRM3, GRM5); receptor functions (such as those related to G protein-coupled receptors (e.g., GPR10), ion channels (e.g., KV8.1, KV1.5), CD34, serotonin receptor, PDGF receptor, somatostatin receptor 3 (SSTR3), BLTR2, ORIOCI, CNTNAP2, nectin3, TGFBR1, CD3611, SDOLF, HTR2A, NRXN1-3, GABRG2, CNTFR, CCR3, GABRG3, GABRP, MRGX2, GPRI9, GNG5, GPRK6); cell-cell junction/cell adhesion (such as tight junctions) (such as those related to claudins, JAM1, JAM2, TM4SF6, NRCAM, VCAM1); cell proliferation/survival/development (such as those related to IGFBP7, MUC12, CD83 antigen, delta-like homolog (drosophila), TNFRSF18, TM4SF6, PIWI1, likely ortholog of mouse PIWI like homolog 1, HARAKIRI, LANO, ENIGMA); neural function/development (such as those related to NLGN1, NLGN3, NRCAM); psoriasis (such as those related to to SEEK1); hypomagnesemia hypercalciuria syndrome (such as that related to claudin 16 (paracellin-1)); Williams Beuren Syndrome (such as that related to FZD9).

87. Any of the methods described herein, wherein the PDZ domain polypeptide comprises PDZ domain of ERBIN and the molecule known to bind to the polypeptide (for example, a ligand) is δ-catenin, ARVCF or p0071.

88. Any of the methods described herein, wherein the PDZ domain polypeptide comprises PDZ domain of DENSIN and the molecule known to bind to the polypeptide (for example, a ligand) is ARVCF, p0071 or δ-CATENIN.

89. Any of the methods described herein, wherein the PDZ domain polypeptide comprises PDZ1 and/or 3 of SCRIBBLE and the molecule known to bind to the polypeptide (for example, a ligand) is ZO2 (tight junction protein 2), KV1.5, GPR87, ACTININ, β-CATENIN or CD34.

90. Any of the methods described herein, wherein the PDZ domain polypeptide comprises PDZ2 domain of SCRIBBLE and the molecule known to bind to the polypeptide (for example, a ligand) is δ-catENIN, ARVCF or pO071.

91. Any of the methods described herein, wherein the PDZ domain polypeptide comprises PDZ7 domain of MUPP and the molecule known to bind to the polypeptide (for example, a ligand) is HTR2B, PDGFRb, δ-catenin, SGK or SSTR3.

92. Any of the methods described herein, wherein the PDZ domain polypeptide comprises PDZ6 domain of human INADL and the molecule known to bind to the polypeptide (for example, a ligand) is HTR2B, PDGFRb, δ-CATENIN, SGK or SSTR3.

93. Any of the methods described herein, wherein the PDZ domain polypeptide comprises PDZ domain of human ZO1 and the molecule known to bind to the polypeptide (for example, a ligand) is CLAUDIN-17, CLAUDIN-1, CLAUDIN-3, CLAUDIN-7, CLAUDIN-9, CLAUDIN-18, PDGFRA, PDGFRB, δ-catENIN, ARVCF or SGK.

94. Any of the methods described herein, wherein the PDZ domain polypeptide comprises PDZ domain of AF6 (MLLT4) and the molecule known to bind to the polypeptide (for example, a ligand) is FYCO1, BLTR2, TM7SF3, OR10C1, CNTNAP2, NECTIN3, SH3D5 or UTROPHIN.

95. Any of the methods described herein, wherein the PDZ domain comprises PDZ3 domain of MUPP and the molecule known to bind to the polypeptide (for example, a ligand) is drosqphila NUMB homolog, TGFBRI, IGFBP7 or CD3611.

96. Any of the methods described herein, wherein the PDZ domain polypeptide comprises PDZ3 domain of MAG11 and the molecule known to bind to the polypeptide (for example, a ligand) is SDOLF, PLEKHAI, PEPP2, MUC12, SLIT1, PARK2, HTR2A or PITPNB.

97. Any of the methods described herein, wherein the PDZ domain polypeptide comprises PDZ3 domain of MAG13 and the molecule known to bind to the polypeptide (for example, a ligand) is JAM1, JAM2, LLTI, PTTG3, CD83 antigen, DELTA-LIKE homolog (Drosophila), TNFRSF18, RGS20, TM4SF6, PARK2, GPRI0 or IL2RB.

98. Any of the methods described herein, wherein the PDZ domain polypeptide comprises PDZ3 domain of INADL and the molecule known to bind to the polypeptide (for example, a ligand) is BLTR2, JAM1, JAM2, KV8.1, PTTG3, CNTNAP2, NRXNI, NRXN2, NRXN3, TNFRSF18, PTTG1, PARK2, GABRG2, CNTFR, CCR2, GABRG3 or GABRP.

99. Any of the methods described herein, wherein the PDZ domain polypeptide comprises PDZ2 of huINADL and the molecule known to bind to the polypeptide (for example, a ligand) is PIW11, ortholog of mouse PIWI-LIKE HOMOLOG 1, NRXN1, NRXN2, PPP2CA or PPP2CB.

100. Any of the methods described herein, wherein the PDZ domain polypeptide comprises PDZ3 domain of huPARD3 and the molecule known to bind to the polypeptide (for example, a ligand) is HRK, DOC1, PIW1 or PPPIR3D.

101. Any of the methods described herein, wherein the PDZ domain polypeptide comprises PDZ domain of SNTA1 and the molecule known to bind to the polypeptide (for example, a ligand) is MRGX2, NLGN1, NLGN3, SEEK1, CLAUDIN-17, GPR56, SSTR5, SCTR, GRM I, GRM2, GRM3 or GRM5.

102. Any of the methods described herein, wherein is the PDZ domain polypeptide comprises PDZ0 of MAG13 and the molecule known to bind to the polypeptide (for example, a ligand) is LAN0, SSTR3, NRCAM, GPR19, GNG5 or HTR2B.

103. Any of the methods described herein, wherein the PDZ domain polypeptide comprises PDZ13 domain of MUPP and the molecule known to bind to the polypeptide (for example, a ligand) is NLGN3, NLGN1, CLAUDIN-16, GPR56, ENIGMA, FZD9, SSTR5, VCAMI or GPRK6.

104. Any of the methods described herein, wherein the PDZ domain polypeptide comprises PDZ2 domain of MAG13 and the molecule known to bind to the polypeptide (for example, a ligand) is PTEN/MMAC.

Although methods and materials similar or equivalent to those described herein can be used in the practice or testing of the present invention, suitable methods and materials are described below. In the case of conflict, the present specification, including definitions, will control. In addition, the materials, methods, and examples are illustrative only and not intended to be limiting.

### BRIEF DESCRIPTION OF THE DRAWINGS

FIG. 1. Phage display of a penta-His FLAG peptide fused to the carboxyl terminus of P8. The FLAG was connected to P8 with intervening polyglycine linkers of varying length. Phage solutions (1.3×10¹² phage/ml) were incubated in wells coated with an anti-tetra-His antibody to capture phage displaying the penta-His FLAG (circles) or in wells coated with BSA as a negative control (squares). Bound phage were detected in a Phage ELISA. The optical density is proportional to the amount of phage bound and thus measures peptide display levels.

FIG. 2. Homology modeling of PDZ2 in complex with the high affinity peptide ligand GVTWV (SEQ ID NO:240). A, sequence alignment of PDZ2 with the third PDZ domains of PSD-95 (Protein Data Bank code 1BE9) and the human homologue of discs large protein (Protein Data Bank code 1PDR), and the PDZ domains of Syntrophin (Protein Data Bank code 2PDZ), and neuronal nitric oxide synthase (Protein Data Bank code 1B8Q). Numbering corresponds to the PDZ2 modeled structure. Secondary structure elements are indicated at the bottom of the alignment as arrows (β strand) and rectangles (α helix). B, the homology model. Top left, ribbon representation of the modeled PDZ2/GVTWV (SEQ ID NO:240) complex. The secondary structural elements are labeled. The dashed ellipse shows the area zoomed in. Right, zoom in of β2, β3, α2 and the peptide ligand. The peptide side chains are shown in a ball and stick representation. For comparison at P(−1), the Ser side chain of the ligand in the PSD-95-3/KQTSV crystal structure is shown Hydrogen bonds are shown as white dashed lines. Some protein side chains have been omitted for clarity. Bottom left, schematic view of the PDZ domain binding sites for each of the four residues in a tetrapeptide ligand. In addition to previously described interactions with the residues at P(0) and P(−2), the schematic also depicts proposed interactions between the peptide side chains at P(−1) and P(−3) and PDZ side chains in the β3 strand.

FIG. 3. Molecular surface of the modeled PDZ2-GVTWV (SEQ ID NO:240) complex. Protein residues conferring binding affinity and/or specificity are shown .

FIG. 4. Peptides phage-selected against PDZ 2 or PDZ 3 of MAGI-3 bind specifically to the PDZ domain they were phage-selected against and not to other PDZ domains

FIG. 5. Phage-selected peptides against MAGI-3 PDZ2 are targeted to the tight junctions in live Caco-2 cells

FIG. 6. δ-catenin binds to the ERBIN PDZ domain and an important component of the interaction is mediated by its C-terminus.

FIG. 7. The ERBIN PDZ domain associates with δ-catenin in vivo

FIG. 8. A single amino acid change at the (−3) position of a PDZ peptide ligand alters its binding specificity

FIG. 9. Amino acid sequence of MAGI-3 (SEQ ID NO:200).

FIG. 10. Amino acid sequence of ERBIN (SEQ ID NO:201).

FIG. 11. Illustration of database search parameters using consensus and expanded sequences based on phage-selected peptide sequences.

FIG. 12. IC50 values indicating binding affinities of various peptides to PDZ domains

### DETAILED DESCRIPTION

I. Method of Identifying PDZ Binding Phage Peptides

A. Summary

The invention provides a method of identifying peptides that bind to PDZ domains of intracellular proteins using a carboxyl-terminal phage display method. The invention provides fusion genes, each fusion gene comprising a candidate PDZ binding peptide gene and a gene encoding at least a portion of a phage coat protein, where the fusion genes each encode a candidate PDZ binding peptide fused, optionally through a peptide linker, to a carboxyl-terminal amino acid residue of a phage coat protein. In phage display, the fusion proteins are incorporated into phage particles such that the particles display the candidate PDZ binding peptide on the surface of the phage particle. In a preferred embodiment, a library of carboxyl-terminal fusion proteins comprising a candidate PDZ-binding peptideis displayed on phage particles and the library isthen panned against a PDZ domain target to identify the candidate peptides that bind to specific PDZ domains. Phage displaying PDZ domain binding peptidesare then isolated, and the sequence of the displayed peptide is determined, for example, by sequencing the fusion gene. The sequence of one or more binding peptides can then be compared to the carboxyl-terminal sequences of known proteins to determine which known intracellular proteins have a carboxyl-terminal sequence identical to or similar to the PDZ domain binding peptide(s) to identify cognate protein ligands for the PDZ domain containing proteins.

In a preferred aspect, the P8 protein of a filamentous bacteriophage is used to form the carboxyl-terminal fusion proteins, and the preferred method of the invention for the analysis of PDZ domain binding specificities utilizes this display format. For example, it has been shown below that two different PDZ domains from a membrane-associated guanylate kinase selected consensus sequences from highly diverse peptide libraries fused to the carboxyl terminus of P8. Synthetic peptides corresponding to the selected sequences bound the PDZ domains with high affinity and specificity, and synthetic peptides were used to determine the binding contributions of individual peptide side chains (See Examples). In another example, a PDZ domain from the ERBIN protein was applied to the methods of the invention, and phage peptide and cognate protein ligands were discovered that had higheraffinity than previously described ligands.

B. Definitions

1. Protein, Polypeptides and Peptides

The terms protein, peptide and polypeptide are well known in the art. A protein has an amino acid sequence that is longer than a peptide. A peptide contains 2 to about 50 amino acid residues. The term polypeptide includes proteins and peptides. Examples of proteins include antibodies, enzymes, lectins and receptors; lipoproteins and lipopolypeptides; and glycoproteins and glycopolypeptides. Examples of polypeptides include neuropeptides, functional domains (e.g. PDZ domains) of proteins, peptides having 3-20 residues obtained from phage display libraries, etc.

2. PDZ Domain (PDZD)

PDZ domains (also known as DHR (DLG homology region) or, the GLGF repeat), originally described as conserved structural elements in the 95 kDa post-synaptic density protein (PSD-95), the Drosophila tumor suppressor discs-large, and the tight junction protein zonula occludens-1 (ZO-1), are contained in a large and diverse set of proteins. In general, PDZ domain-containing proteins appear to assemble various functional entities, including ion channels and other transmembrane receptors, at specialized subcellular sites such as epithelial cell tight junctions, neuromuscularjunctions, and post-synaptic densities of neurons.

PDZ domains generally bind to short carboxyl-terminal peptide sequences located on the carboxyl-terminal end of interacting proteins. Usually, PDZ domains comprise two a helixes and six β sheets. An example of a PDZD is residues 1217-1371 of SEQ ID NO:201, an ERBIN PDZ domain.

PDZDs can be encoded by a PDZD nucleic acid (PDZD).

3. PDZ Protein (PDZP)

A PDZ protein contains at least one PDZ domain. A PDZP may be a naturally occuring protein, or a protein modified to contain at least one PDZ domain. PDZPs can be encoded by a PDZP nucleic acid (PDZP). Examples of PDZs include MAGI 3 and ERBIN. Also see Table B.

4. PDZ Domain Ligand (PDL)

A ligand refers to a molecule or moiety that binds a specific site on a protein or other molecule; a PDZ domain ligand is a molecule or moiety that binds at least one PDZ domain. Proteins, peptides, small organic and inorganic molecules, and nucleic acids are examples of PDLs.

5. PDZ Domain Binding Peptide (PDBP)

A peptide, such as natural or phage display-derived peptides, that physically, but non-covalently, interacts with (“binds” to) a PDZ domain. The PDZ domain with which a PDBP may interact may be isolated or contained within a PDZ protein, or fragment or derivative thereof. A PDBP may contain only those amino acid residues necessary to bind with a PDZ domain, or contain up to a total of about 50 amino acid residues. Peptides (proteins) larger than 50 amino acids that interact with PDZ domains are PIPs (see below). PDBPs may be encoded by a PDBP nucleic acid (PDBP). Examples of PDBPs include those peptides that bind to the ERBIN PDZ domain, SEQ ID NOs:14-181, 209-213 and 241-247.

6. PDZ Interacting Protein (PIP)

A protein, comprising at least one PDBP, that physically, butnon-covalently, interacts with (“binds” to) a PDZ protein via a PDZ domain. PIPs include those proteins that are found in nature, variants thereof, as well as those proteins that have been modified to contain at least one PDBP. PIPs may be encoded by a PlPnucleic acid (PIP). An example of a PIP includes δ-catenin, which contains a PDBP that binds ERBIN PDZ domains.

7. Affinity Purification

Affinity purification means the isolation of a molecule based on a specific attraction or binding of the molecule to a chemical or binding partner to form a combination or complex which allows the molecule to be separated from impurities while remaining bound or attracted to the partner moiety.

8. Cell, Cell Line, Cell Culture

Cell, cell line, and cell culture are used interchangeably, and such designations include all progeny of a cell or cell line. Progeny may not be precisely identical in DNA content, due to deliberate or inadvertent mutations. Mutant progeny that have the same function or biological activity as screened for in the originally transformed cell are included.

9. Coat Protein (in Context of Phage)

A phage coat protein comprises at least a portion of the surface of the phage virus particle. Functionally, a coat protein is any protein that associates with a virus particleduring the viral assembly process in a host cell and remains associated with the assembled virus until infection. A major coat protein is that which principally comprises the coat and is present in 10 copies or more copies/particle; a minor coat protein is less abundant.

10. Fusion Protein

A fusion protein is a polypeptide having two portions covalently linked together, where each of the portions is derived from different proteins. The two portions may be linked directly by a single peptide bond or through a peptide linker containing one or more amino acid residues. Generally, the two portions and the linker will be in reading frame with each other and are produced using recombinant techniques.

11. Heterologous DNA

Heterologous DNA is any DNA that is introduced into a host cell. The DNA may be derived from a variety of sources including genomic DNA, cDNA, synthetic DNA and fusions.

12. Phage Display

Phage display is a technique by which variant polypeptides are displayed as fusion proteins to a coat protein on the surface of phage, such as filamentous phage, particles. Polyvalent phage display methods have been used for displaying small random peptides and small proteins through fusions to a coat protein, gnerally protein 3 or protein 8, of filamentous phage (Wells and Lowman, 1992). In monovalent phage display, a gene encoding a protein or peptide library is fused to a phage coat protein gene or a portion thereof and the corresponding protein fusion is expressed at low levels in the presence of wild type coat protein so that no more than a minor amount of phage particles display more than one copy of the fusion protein. Avidity effects are reduced relative to polyvalent phage so that sorting is on the basis of intrinsic ligand affinity. When phagemid vectors are used, DNA manipulations are simplified (Lowman and Wells, 1991).

13. Phagemid Vector

A phagemid is a plasmid vector having a phage origin of replication, a bacterial origin of replication, e.g., ColE1, and a copy of an intergenic region of a bacteriophage. The phagemid may be based on any known bacteriophage, including filamentous and lambdoid bacteriophage. The plasmid may also contain a selectable marker. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids that contain a phage coat protein gene or fragment thereof linked to a heterologous polypeptide gene as a gene fusion such that the heterologous polypeptide is displayed on the surface of the phage particle (Sambrook, 1989).

14. Phage Vector

A phage vector is a double stranded nucleic acid replicative form of a bacteriophage DNA containing a heterologous gene and capable of replication. The phage vector has a phage origin of replication allowing phage replication and phage particle formation. The phage is preferably a filamentous bacteriophage, such as an M 13, f1, fd, Pf3 phage or a derivative thereof, or a lambdoid phage, such as lambda, 21, phi80, phi81, 82, 424, 434, etc., or a derivative thereof.

15. Polymerase Chain Reaction (PCR)

PCR refers the technique in which minute amounts of a specific piece of nucleic acid, RNA and/or DNA, are amplified as described in U.S. Pat. No. 4,683,195. PCR can be used to amplify specific RNA sequences, specific DNA sequences from total genomic DNA, and cDNA transcribed from total cellular RNA, bacteriophage or plasmid sequences, etc. (Ehrlich, 1992; Mullis et al., U.S. Pat. No. 4,683,195, 1987)

16. Wild Type

A wild-type sequence or the sequence of a wild-type protein, such as a coat protein, is the reference sequence from which variant polypeptides are derived through the introduction of mutations. In general, the wild-type sequence for a given protein is the sequence that is most common in nature. Similarly, a wild-type gene sequence is the sequence for that gene which is most commonly found in nature. Mutations introduced into wild4ype sequences create “variant” or “mutant” forms of the original wild-type protein or gene.

C. Carboxyl-Terminal Phase Display

In the first step of identifying a PDZ phage peptide, carboxyl-terminal (C-terminal) display libraries of heterologous peptides on the surface of a phage, preferably a filamentous phage using protein fusions with protein 3 or 8, are prepared. C-terminal display has been reported on protein 6 of M13 (Jespers et al., 1995); methods of C-terminal display of peptides and proteins generally are disclosed in WO 00/06717. These methods may be used to prepare the fusion genes, fusion proteins, vectors, recombinant phage paticles, host cells and libraries thereof of the invention. The C-terminal display of a heterologous peptide or library of peptides may be accomplished in a manner similar to display at the N-terminus (N-terminal display) of a phage coat protein. C-terminal display may be accomplished using a wild type coat protein or a mutant coat proteinas set forth in WO 00/06717.

Any of the well known laboratory methods of phage or phagemid display, creating coat protein variants and protein fusions with a heterologous peptide, libraries of such variants and fusion proteins, expression vectors encoding the variants and proteinfusions, libraries of the vectors, a library of host cells containing the vectors, methods for preparing and panning the same to obtain binding peptides may also be used in this aspect of the invention for C-terminal display. References describing these methods are noted above.

The variant protein fusion proteins will contain one or more alterations including substitutions, additions or deletions relative to the wild type coat protein sequence. A large number of alterations are possible and are tolerated by the phage while retaining the ability to display peptides on the phage surface. The chemical nature of the residue may be changed, i.e. a hydrophobic residue may be altered to a hydrophilic residue or vice versa. Variants containing 2-50, preferably 5-40, more preferably 7-20, altered residues are possible. Fusion proteins containing any mature coat protein sequence or portion thereof that varies from the wild type sequence of the coat protein or portion thereof is within the scope of the invention. Coat protein variants containing 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 variant residues are contemplated, although most preferably 4-10 variant residues. Variants that do not enable surface display of the heterologous peptide are selected against during the phage display, panning and selection process.

As with N-terminal display, libraries, in which amino acids residues within desired segments of the coat protein are varied, can be made to obtain a library of coat protein variants having amino acid additions, substitutions or deletions within defined regions of the coat protein. As an example, the coatprotein may be divided into an arbitrary number of zones, generally 2-10 zones, and a library constructed of variants within one or more of the zones. The mature coat proteins of M13, fl and fd phage, for example, contain 50 amino acids and might be divided into 10 zones of 5 amino acid residues each or into zones with unequal numbers of residues in each zone, e.g. zones containing 15, 10, 9, and 8 residues. Zones corresponding to the cytoplasmic, transmembrane and periplasmic regions of the coat protein may be used. A separate library may be constructed for each of the zones in which amino acid alterations are desired. If fusion proteins are desired in which the coat protein variant has an amino acid alteration in zone 1, for example, a single library may be constructed in which one or more of the amino acid residues within zone 1 is varied. Alternatively, one may wish to produce fusion proteins in which 2 zones contain amino acid alterations. Two libraries, each library containing alterations within one of the 2 zones, can be prepared.

Preferably, the heterologous peptide is attached to the coat protein or variant thereof through a linker peptide. The linker may contain any number of residues that allow C-terminal display, and will generally contain about 4 to about 30, preferably about 8 to about 20, amino acid residues. The linker may contain any of the naturally occurring residues, although linkers containing predominantly (greater than 50%) glycine and/or serine are preferred. The optimum linker composition and length for display of a particular peptide may be selected using phage display as described above and demonstrated in the examples. For example, phage libraries each containing a different linker length may be constructed and phage selection and panning used to isolate the amino acid composition of the linker of any length the optimizes expression and display of the heterologous peptide. See the Examples for an example of effective linkers.

If a variant coat protein that improves display of a heterologous peptide on the surface of phage particles contains multiple mutations relative to wild type, it is also possible to obtain variants which display the heterologous peptide at levels intermediate between the levels obtained with the new variant and wild type coat protein. This can be accomplished by separately back mutating each mutated amino acid of the variant back to the wild type sequence or to another altered residue. These back mutations will generally reduce display levels of the heterologous peptide to levels varying between display levels obtained with the variant and wild type coat protein. By combining the back mutations, display may be tailored to a desired level that is between that obtained with the variant and wild type coat protein.

A similar process may be use to make variants that display at a level below the level of the wild type coat protein. For example, mutations may be made in one or more zones and the libraries produced panned for phage that bind only weakly (weaker than phage displaying wild type fusions). The weaker binding phage will be displaced by phage displaying wild type coat protein fusions and can be isolated and sequenced using known methods.

Mutant coat proteins can also be obtained that are hypofunctional (less functional than wild-type) for incorporation into the viral coat and thus reduce fusion protein display relative to wild type coat protein. In this case, mutations are made in residues that tend to be conserved as wild type. Variants obtained through mutations at these sites can then be screened for their ability to display a given fusion protein relative to the wild type coat protein display levels. Hypofunctional variants displaying the fusion at the desired reduced levels relative to wild type can then be used for the construction of libraries of the fusion protein for the purposes of phage display. Although the preferred residues for the production of hypofunctional variants are those that are conserved, any residue of the coat protein can be mutated and the resulting variant tested for its ability to allow display of a fusion protein. A lower display level than wild type is achieved by using the appropriate hypofunctional mutant. While the selection of hypofunctional variants requires a screen rather than a selection, the method is relatively simple since most mutations in proteins cause reductions in activity rather increases and suitable screening procedures are known.

C-terminal display, as described above, is useful to display peptides encoded by DNA libraries (containing nucleic acid encoding candidate PDZ binding peptides) on the surface of phage particles. A phagemid or phage vector containing an open reading frame is constructed recombinantly, and the DNAs are ligated into the vectors at the 3′ end of the coat protein gene. Host cells are then transformed with the library of vectors, and phage particles displaying heterologous peptides corresponding to the DNA library members are obtained (with superinfection of helper phage for phagemid vectors). The C-terminal phage display library obtained may be panned and analyzed using conventional phage display techniques.

C-terminal display is especially useful for PDZ binding peptide identification, in particular since most PDZ domains recognize and bind to the C-terminal portion of PDZ domain binding ligands.

Preferably, the C-terminal phage display library is prepared using a phagemid vector to construct a library of vectors containing a plurality of fusion genes using recombinant techniques. The fusion genes are preferably prepared as 3′ fusions of peptide library genes with gene 8 of a filamentous phage or a variant thereof, so that the protein fusions encoded thereby are expressed as phage protein 8 having a carboxy-terminal candidate binding peptide fusioned thereto. Further, the fusion gene may also contain a nucleic acid portion that codes for a peptide linker between the phage coat protein and the candidate binding peptide. The sequence of the peptide linker may be optimized using known phage display methods as described above. The linker may vary in length in order to provide the optimum display of the candidate binding peptides, but is generally from 2 to 50 residues, preferably 4 to 25 residues, more preferably 5 to 20 residues. Consequently, a different linker, both in length and amino acid residues may allow more efficient display of different length display peptides. The peptide library genes generally code for random peptides having 4-20, preferably 4-10 amino acid residues. At each library position, a degenerate codon that encodes all 20 naturally occuring amino acids is preferably used, although one or more positions may be fixed as a single amino acid residue or a degenerate codon encoding a limited set of residues may used if desired. The library may also code for stop codons, such as amber, ochre or umber stop codons, if display of shorter peptides is desired. Once prepared, the library is then cycled through one, two or several rounds of binding selection with prepared PDZ domains.

D. Preparation of PDZ Domains

I. General Approach

PDZ domains may be produced conveniently as protein fragments containing the domain or as fusion polypeptides using conventional synthetic or recombinant techniques. Fusion polypeptides are useful in expression studies, cell-localization, bioassays, and PDZ domain purification. A PDZ domain “chimeric protein” or “fusion protein” comprises a PDZ domain fused to a non-PDZ domain polypeptide. A non-PDZ domain polypeptide is not substantially homologous (homology is later defined below) to the PDZ domain. A PDZ domain fusion protein may include any portion to the entire PDZ domain, including any number of the biologically active portions. The fusion protein can then be purified according to known methods using affinity chromatography and a capture reagent that binds to the non-PDZ domain polypeptide. A PDZ domain may be fused to the C-terminus of the GST (glutathione S-transferase) sequences, for example. Such fusion proteins facilitate the purification of the recombinant PDZ domain using glutathione bound to a solid support. Additional exemplary fusions are presented in Table A, including some common uses for such fusions.

Fusion proteins can be easily created using recombinant methods. A nucleic acid encoding PDZ domain can be fused in-frame with a non-PDZ domain encoding nucleic acid, to the PDZ domain N -terminus, C-terminus or internally; preferably, PDZ fusions are fused at the N-terminus. Fusion genes may also be synthesized by conventional techniques, including automated DNA synthesizers. PCR amplification using anchor primers that give rise to complementary overhangs between two consecutive gene fragments that can subsequently be annealed and reamplified to generate a chimeric gene sequence (Ausubel et al., 1987) is also useful. Many vectors are commercially available that facilitate sub-cloning a PDZ domain in-frame to a fusion protein.

TABLE A
Useful non-PDZ domain fusion polypeptides

| Fusion partner | in vitro | in vivo | Reference |
|---|---|---|---|
| Human growth | Radioimmuno-assay | none | (Selden et al., |
| hormone (hGH) | | | 1986) |
| β-glucuronidase | Colorimetric, | colorimetric (histo- | (Gallagher, |
| (GUS) | fluorescent, or | chemical staining | 1992) |
| | chemi-luminescent | with X-gluc) | |
| Green fluorescent | Fluorescent | fluorescent | (Chalfie et |
| protein (GFP) and | | | al., 1994) |
| related molecules | | | |
| (RFP, BFP, YFP | | | |
| domain, etc.) | | | |
| Luciferase (firefly) | bioluminsecent | Bioluminescent | (de Wet et |
| | | | al., 1987) |
| Chloramphenicoal | Chromatography, | none | (Gorman et |
| acetyltransferase | differential | | al., 1982) |
| (CAT) | extraction, | | |
| | fluorescent, or | | |
| | immunoassay | | |
| β-galacto-sidase | colorimetric, | colorimetric | (Alam and |
| | fluorescence, chemi- | (histochemical | Cook, 1990) |
| | luminscence | staining with X- | |
| | | gal), bio- | |
| | | luminescent in live | |
| | | cells | |
| Secrete alkaline | colorimetric, | none | (Berger et al., |
| phosphatase (SEAP) | bioluminescent, | | 1988) |
| | chemi-luminescent | | |
| Tat from HIV | Mediates delivery | Mediates delivery | (Frankel et |
| | into cytoplasm and | into cytoplasm and | al., US Pat. |
| | nuclei | nuclei | No. |
| | | | 5,804,604, |
| | | | 1998) |

As an example of a PDZ domain fusion, GST-PDZ fusion may be prepared from a gene of interest. With the full-length gene of interest as the template, the PCR is used to amplify DNA fragments encoding the PDZ domain using primers that introduce convenient restriction endonuclease sites to facilitate sub-cloning. Each amplified fragment is digested with the appropriate restriction enzymes and cloned into a similarly digested plasmid, such as pGEX-4T-3, that contains GST and designed such that the sub-cloned fragments will be in-frame with the GST and operably linked to a promoter, resulting in plasmids encoding GST-PDZ fusion proteins.

To produce the fusion protein, E. coli cultures harboring the appropriate expression plasmids are generally grown to mid-log phase (A600=1.0) in LB broth, preferablyat about 37° C., and may be induced with IPTG. The bacteria are pelleted by centrifugation, resuspended in PBS and lysed by sonication. The suspension is centrifuged, and GST-PDZ fusion proteins are purified from the supernatant by affinity chromatography on 0.5 ml of glutathione-Sepharose.

However, it will be apparent to one of skill in the art that many variations will achieve the goal of isolated PDZ domain protein and may be used in this invention. For example, fusions of the PDZ domain and an epitope tag may be constructed as described above and the tags used to affinity purify the PDZ domain. Epitope tags are described more fully below. PDZ domain proteins/peptides may also be prepared without any fusions; in addition, instead of using the microbial vectors to produce the protein, in vitro chemical synthesis may instead be used. Other cells may be used to produce PDZ domain proteins/peptides, such as other bacteria, mammalian cells (such as COS), or baculoviral systems. A wide variety of polynucleotide vectors to produce a variety of fusions are also available. The final purification of a PDZ domain fusion protein will obviously depend on the fusion partner; for example, a poly-histidine tag fusion can be purified on nickel columns.

2. PDZ Domains

PDZ domains have a characteristic of assembling protein complexes, usually at cell plasma membranes. Many PDZ domain -containing proteins are currently known. Any PDZ domain and any PDZ domain containing protein may be used in the method of the invention. Table B lists a subset of known PDZ domain-containing human proteins. These and other PDZ domains are contemplated as targets for the method of the invention, as well as the non-human homologs thereof.

TABLE B
Human PDZ domain-containing proteins

| | Protein | Nucleotide | |
|---|---|---|---|
| Protein (all Homo sapiens) | accession | accession | Reference |
| multiple PDZ domain protein | NP_003820.1 | NM_003829 | (Ullmer et al., 1998) |
| PDZ domain protein (Drosophila inaD-like) | NP_005790.1 | NM_005799 | (Lennon et al., 1996) |
| The KIAA0147 gene product is related | BAA09768.1 | D63481 | direct submission |
| to adenylyl cyclase | | | |
| protein tyrosine phosphatase, | NP_006255.1 | NM_006264 | (Maekawa et al., 1994) |
| non-receptor type 13 (APO- | | | |
| 1/CD95 (Fas)-associated | | | |
| phosphatase); protein tyrosine | | | |
| phosphatase, nonreceptor type 13 | | | |
| discs, large (Drosophila) homolog 4 | NP_001356.1 | NM_001365 | (Kim et al., 1996b; Stathakis et al., |
| | | | 1997) |
| discs, large (Drosophila) homolog 2; | NP_001355.1 | NM_001364 | (Kim et al., 1996b; Kim et al., 1995; |
| chapsyn-110 | | | Stathakis et al., 1998) |
| discs, large (Drosophila) homolog 1 | NP_004078.1 | NM_004087 | (Azim et al., 1995; Lue et al., 1994) |
| neuroendocrine-dlg | AAB61453.1 | U49089 | (Makino et al., 1997) |
| BAI1-associated protein 1 | BAA32002.1 | AB010894 | (Shiratsuchi et al., 1998) |
| tight junction protein 1 | NP_003248.1 | NM_003257 | (Mohandas et al., 1995; Willott et al., |
| (zona occludens 1) | | | 1993; Willott et al., 1992) |
| KIAA0705 protein | BAA31680.1 | AB014605 | (Ishikawa et al., 1998) |
| KIAA1634 protein | BAB13460.1 | AB046854 | (Nagase et al., 2000) |
| GRIP1 protein | CAB39895.1 | AJ133439 | (Bruckner et al., 1999) |
| tight junction protein 2 (zona occludens 2); | NP_004808.1 | NM_004817 | (Beatch et al., 1996; Duclos et al., |
| Friedreich ataxia | | | 1994) |
| region gene X104 (tight junction protein ZO-2) | | | |
| ZO-3 | AAC72274.1 | AC005954 | direct submission |
| PDZ domain containing 1 | NP_002605.1 | NM_002614 | (Kocher et al., 1998; White et al., |
| | | | 1998) |
| amyloid β (A4) precursor protein-binding, | NP_001154.1 | NM_001163 | (Borg et al., 1998; Duclos et al., |
| family A, member 1 (X11); amyloid β (A4) | | | 1993; Duclos and Koenig, 1995; |
| precursor protein-binding, family A, | | | Okamoto and Sudhof, 1997) |
| member 1 (XII); Munc18-1-interacting protein 1; | | | |
| Amyloid β A4 precursor protein-binding, | | | |
| family A, member 1 | | | |
| protease-activated receptor 3 | NP_062565.1 | NM_019619 | (Joberty et al., 2000) |
| amyloid β (A4) precursor protein-binding, | NP_004877.1 | NM_004886 | (Tanahashi and Tabira, 1999b) |
| family A, member 3 (XII-like 2); XIIL2 protein, | | | |
| interacts with Alzheimer's β- | | | |
| amyloid | | | |
| amyloid β (A4) precursor protein-binding, | NP_005494.1 | NM_005503 | (Borg et al., 1998; McLoughlin and |
| family A, member 2 | | | Miller, 1996; Okamoto and Sudhof, |
| (XII-like); Amyloid β A4 precursor | | | 1997; Tomita et al., 1999) |
| protein-binding, family A, | | | |
| member 2; Munc18-1-interacting protein 2 | | | |
| PDZ-73 protein | NP_005700.1 | NM_005709 | (Kobayashi et al., 1999; Scanlan |
| | | | et al., 1998) |
| KIAA1095 protein | BAA83047.1 | AB029018 | (Kikuno et al., 1999) |
| solute carrier family 9 (sodium/hydrogen | NP_004243.1 | NM_004252 | (Murthy et al., 1998; Reczek et al., |
| exchanger), isoform 3 regulatory factor 1 | | | 1997) |
| regulatory factor 1 | | | |
| palmitoylated membrane protein 1; erythrocyte membrane | NP_002427.1 | NM_002436 | (Bryant and Woods, 1992; Kim et al., |
| protein p55 | | | 1996a; Marfatia et al., 1995; |
| | | | Metzenberg and Gitschier, 1992; Ruff |
| | | | et al., 1999) |
| solute carrier family 9 (sodium/hydrogen exchanger), | NP_004776.1 | NM_004785 | (Hall et al., 1998; Imai et al., 1998; |
| isoform 3 regulatory factor 2 | | | Poulat et al., 1997; Reczek et al., |
| | | | 1997; Yun et al., 1997) |
| protein tyrosine phosphatase, non-receptor type 3 | NP_002820.1 | NM_002829 | (Arimura et al., 1992; Itoh et al., |
| | | | 1993; Yang and Tonks, 1991) |
| protein tyrosine phosphatase, non-receptor type 4 | NP_002821.1 | NM_002830 | (Gu et al., 1991) |
| (megakaryocyte) | | | |
| dishevelled 1 | NP_004412.1 | NM_004421 | (Pizzuti et al., 1996a; Pizzuti et al., |
| | | | 1996b; Semenov and Snyder, 1997) |
| myeloid/lyrnphoid or mixed-lineage leukemia (trithorax | NP_005927.1 | NM_005936 | (Prasad et al., 1993; Saha et al., |
| (Drosophila) homolog); translocated to, 4; Myeloid/lymphoid or | | | 1995; Saito et at., 1998) |
| mixed-lineage leukemia, translocated to, 4 | | | |
| KIAA0300 | BAA20760.1 | AB002298 | (Nagase et al., 1997) |
| hypothetical protein FLJ11271 | NP_060843.1 | NM_018373 | direct submission |
| dishevelled 2 | NP_004413.1 | NM_004422 | (Greco et al., 1996; Pizzuti et al., |
| | | | 1996a; Semenov and Snyder, 1997) |
| interleukin 16; lymphocyte chemoattractant factor | NP_004504.1 | NM_004513 | (Baier et al., 1997; Bannert et al., |
| | | | 1996; Kim, 1999a) |
| discs, large (Drosophila) homolog 5 | NP_004738.1 | NM_004747 | (Nagase et al., 1998a; Nakamura et |
| | | | al., 1998) |
| Tax interaction protein 1 | NP_055419.1 | NM_014604 | (Andersson et al., 1996; Reynaud et |
| | | | al., 2000; Rousset et al., 1998; |
| | | | Touchman et al., 2000) |
| nitric oxide synthase | BAA03895.1 | D16408 | (Fujisawa et al., 1994) |
| calcium/calmodulin-dependent serine protein | NP_003679.1 | NM_003688 | (Cohen et al., 1998; Dimitratos et al., |
| kinase (MAGUK family) | | | 1998) |
| Vertebrate LIN7 homolog 1, Tax interaction protein 33; | NP_004655.1 | NM_004664 | (Butz et al., 1998; Jo et al., 1999; |
| vertebrate LIN7 homolog 1 | | | Rousset et al., 1998) |
| LIN-7 protein 3 | NP_060832.1 | NM_018362 | direct submission |
| LIM domain protein | NP_003678.1 | NM_003687 | (Bashirova et al., 1998) |
| syndecan binding protein (syntenin) | NP_005616.1 | NM_005625 | (Lin et al., 1998) |
| LIM domain kinase 2 isoform 2b | NP_057952.1 | NM_016733 | (Nomoto et al., 1999; Okano et al., |
| | | | 1995; Osada et al., 1996) |
| KIAA0613 protein | BAA31588.1 | AB014513 | (Ishikawa et al., 1998) |
| syntrophin 5 | CAB92969.1 | AJ003029 | direct submission |
| LIM domain kinase I isoform 1; | NP_002305.1 | NM_002314 | (Edwards and Gill, 1999; |
| LIM motif-containing protein | | | Frangiskakis et al., 1996; |
| kinase | | | Mizuno et al., 1994; Okano |
| | | | et al., 1995; Osborne et |
| | | | al., 1996; Tassabehji |
| | | | et al., 1996) |
| hypothetical protein | CAB53685.1 | AL110228 | direct submission |
| PDZ domain-containing guanine nucleotide exchange factor I | NP_057424.1 | NM_016340 | direct submission |
| β2-syntrophin. | AAC50449.1 | U40572 | (Ahn et al., 1996) |
| LIM protein (similar to rat protein kinase C-binding enigma) | NP_006448.1 | NM_006457 | (Ueki et al., 1999) |
| hypothetical protein | NP_057568.1 | NM_016484 | direct submission |
| Tax interaction protein 43 | AAB84253.1 | AF028828 | (Rousset et al., 1998) |
| hypothetical protein | CAB82311.1 | AL161971 | direct submission |
| erbb2-interacting protein ERBIN | NP_061165.1 | NM_018695 | (Borg et al., 2000; Nagase et al., |
| | | | 1999a) |
| component); syntrophin, α (dystrophin-associated protein A1, | NP_003089.1 | NM_003098 | (Ahn et al., 1996; Castello et al., |
| 59 kD, acidic component) | | | 1996) |
| regulator of G-protein signalling 12 | NP_002917.1 | NM_002926 | (Snow et al., 1998) |
| GTPase-activating protein | BAA22197.1 | AB005666 | direct submission |
| PDZ-LIM protein mystique | NP_067643.1 | NM_021630 | direct submission |
| PIST | NP_065132.1 | NM_020399 | direct submission |
| apical protein, Xenopus laevis-like | NP_001640.1 | NM_001649 | (Schiaffino et al., 1995) |
| pleckstrin homology, Sec7, and coiled-coil domains protein- | NP_004279.1 | NM_004288 | (Dixon et al., 1993; Kim, 1999b) |
| binding protein | | | |
| hypothetical protein FLJ10324 | NP_060529.1 | NM_018059 | direct submission |
| protease, serine, 11 (IGF binding) | NP_002766.1 | NM_002775 | (Hu et al., 1998; Zumbrunn and |
| | | | Trueb, 1996; Zumbrunn and Trueb, |
| | | | 1997) |
| KIAA0380 protein | BAA20834.1 | AB002378 | (Fukuhara et al., 1999) |
| palmitoylated membrane protein 3; discs, large (Drosophila) | NP_001923.1 | NM_001932 | (Smith et al., 1996) |
| homolog 3; MACUK p55 subfamily member 3 | | | |
| MACUK protein p55T; Protein Associated with Lins 2; | NP_057531.1 | NM_016447 | (Kamberov et al., 2000) |
| MACUK protein p55T | | | |
| Tax interaction protein 40 | AAB84252.1 | AF028827 | (Rousset et al., 1998) |
| KIAA0973 protein | BAA76817.1 | AB023190 | (Nagase et al., 1999b) |
| KIAA0316 | BAA20774.1 | AB002314 | (Nagase et al., 1997) |
| somatostatin receptor interacting protein splice variant a | AAD45121.1 | AF163302 | direct submission |
| KIAA0967 protein | BAA76811.1 | AB023184 | (Nagase et al., 1999b) |
| hypothetical protein FLJ20075 | NP_060125.1 | NM_017655 | direct submission |
| KIAA0561 protein | BAA25487.1 | AB011133 | (Nagase et al., 1998a) |
| T-cell lymphoma invasion and metastasis 2 | NP_036586.1 | NM_012454 | (Chiu et al., 1999) |
| palmitoylated membrane protein 2; MACUK p55 subfamily | NP_005365.1 | NM_005374 | (Mazoyer et al., 1995) |
| member 2; discs large, homolog 2 | | | |
| KIAA0807 protein | BAA34527.1 | AB018350 | (Nagase et al., 1998c) |
| T-celI lymphoma invasion and metastasis 1; human T-lymphoma | NP_003244.1 | NM_003253 | (Chen and Antonarakis, 1995; Habets |
| invasion and metastasis inducing TIAM1 protein | | | et al., 1995a; Habets et al., 1995b; |
| | | | Hattori et al., 2000; Michiels et at., |
| | | | 1995) |
| KIAA0902 protein | BAA74925.1 | AB020709 | (Nagase et al., 1998b) |
| KIAA0751 protein | BAA34471.1 | AB018294 | (Nagase et al., 1998c) |
| GLUT1 C-terminal binding protein | NP_005707.1 | NM_005716 | (De Vries et at., 1998; Von Kap-Herr |
| | | | et al., 2000) |
| KIAA0545 protein | BAA25471.1 | AB011117 | (Nagase et al., 1998a) |
| proteasome (prosome, macropain) 26S subunit, non-ATPase, 9; | NP_002804.1 | NM_002813 | (Watanabe et al., 1998) |
| Proteasome 26S subunit, non-ATPase, 9 | | | |
| connector enhancer of KSR-like (Drosophila kinase | NP_006305.1 | NM_006314 | (Therrien et al., 1999; Therrien et at., |
| suppressor of ras) | | | 1998) |
| KIAA1284 protein | BAA86598.1 | AB033110 | (Nagase et al., 1999a) |
| signal transducer and activator of transcription 6, | NP_003144.1 | NM_003153 | (Hou et al., 1994; Leek et al., 1997; |
| interleukin-4 induced; Signal transducer and | | | Patel et al., 1998) |
| activator of transcription-6, interleukin-4 | | | Patel et al., 1998) |
| ATP-binding cassette, subfamily B, member 4, isoform A; P- | NP_000434.1 | NM_000443 | (Lincke et al., 1991; |
| glycoprotein-3/multiple drug resistance-3; P glycoprotein | | | Smit et al., 1995; |
| 3/multiple drug resistance 3; multiple drug resistance 3 | | | Van der Bliek et al., |
| | | | 1987; van der Bliek |
| | | | et al., 1988) |

E. Isolation of High-Affinity Binding Phase to the PDZ Domains of Interest

The phage display library with the carboxyl-terminal-displayed candidate PDZ binding peptides are then contacted with the PDZ domain proteins or PDZ domain fusion proteins in vitro to determine those members of the library that bind to the PDZ domain target. Any method, known to the skilled artisan, may be used to assay for in vitro protein binding.

For example, 1, 2, 3 or 4 rounds or more of binding selection may be performed, after which individual phage are isolated and, optionally, analyzed in a phage ELISA. Binding affinities of peptide-displaying phage particles to immobilized PDZ target proteins may be determined using a phage ELISA (Barrett et al., 1992).

F. Determining the Sequence of the Displayed Peptide

Phage that bind to the target PDZ or PDZ fusion, and optionally, not to unrelated PDZ domains, are subjected to sequence analysis. The phage particles displaying the candidate PDZ binding peptides are amplified in host cells, the DNA isolated, and the appropriate portion (fusion gene) of the genome sequenced using any appropriate known sequencing technique.

G. Determining the PDZ Binding Peptide Consensus Sequence(s)

A PDZ binding peptide consensus sequence(s) for a PDZ domain of interest may then be determined from the sequences of individual binding peptides. A consensus sequence is a derived amino acid sequence that represents a family of similar sequences. Each residue in the consensus sequence corresponds to the residue most frequently occuring at that position. A consensus sequence can be determined manually from a family of sequences by inspection.

Alternatively, amino acid sequences can be aligned using comercially available computer software, for example, the Eyeball Sequence Editor software (Cabot and Beckenbach, 1989). Gaps are manually introduced to maximize homology. Amino acid consensus sequences are manually derived from the alignments: a consensus residue occurs most frequently at a given position. Residues identified as invariant are present in all full-length sequences. Positions that exhibit no clear consensus may be represented as an “X” in consensus sequences, while positions that were not present in at least 50 percent of the sequences are usually not included in a consensus sequence.

H. Identifying Proteins that Contain a PDZ Binding Peptide Consensus Sequence(s) or a Specific Binding Sequence at the Carboxy Terminus

To identify potential binding partners of a PDZ domain of interest, those proteins that contain a PDZ binding consensus sequence(s) or a specific PDZ binding sequence at the C-terminus are identified. This identification may be performed in silico, querying public sequence databases, such as Swiss Prot, Dayhoff or Genbank. The sequences may be searched by amino acid sequence only, or nucleic acid sequences may be searched by creating an appropriate series of nucleic acid sequences that would encode a PDZ binding consensus sequence(s), taking into account the degeneracy of the genetic code.

For example, proteins with C-terminal residues that resemble the phage-selected peptides against a PDZ domain of interest can be identified using any available motif-searching algorithm or by inspection. Preferably, a plurality, for example, 10-20 or 10-50 or even greater than 100 phage peptides selected against the PDZ domain of interest may be aligned to establish a consensus sequence for tight binding to the PDZ domain of interest. The consensus sequence is then used to search available protein databases to identify similar C-terminal sequences, restricting the search criteria to the C-terminal amino acids of proteins within the database. The number of C-terminal amino acids in the criteria may vary as necessary to obtain a suitable or desired number of matching database proteins, but is preferably about 4 to about 10 residues.

Obvious to one of skill, various criteria may be adjusted, such as the number of phage to be aligned, the motif-searching algorithm, the databases to be queried, and the number of C-terminal residues to query in the database.

I. Eliminating Unlikely Candidates

To determine candidate proteins that bind to/interact with the PDZ domain of interest, a protein database is queried (as described above), to identify a list of proteins having a C-terminal sequence similar to the consensus or specific binding sequence determined by phage display. If desired, proteins that are not intracellular proteins (PDZ domains are found on cytoplasmic proteins) are removed from the list. Redundant database entries and orthologs may also be eliminated to simplify the list as desired. The list may be further culled if desired to remove proteins not associated with the organism from which the PDZ domain was obtained.

For example, orthologs or simply separate database entries of the same gene product may be found and can be reduced to one exemplary entry. In the case where the subcellular localization of a protein is unknown and/or can not be predicted by sequence homologies (especially for homologies for known sub-cellular targeting domains), such proteins may be maintained as candidate proteins of interest.

J. Assaying the Biology of the Candidate Proteins to Interact with the PDZ-Domains and PDZ-Domain-Containing Proteins in Vitro and in Vivo

Once a list of candidate PDZ domain binding proteins is identified, the candidates can be screened for interaction with the PDZ domain of interest, in vitro and/or in vivo. Suitable screening assays may use the prepared PDZ domain (see above) or the entire protein containing the PDZ domain of interest. For example, the assay may comprise contacting a PDZ domain or PDZ domain containing protein with the candidate binding peptide determined by phage display (or a longer peptide containing this sequence) and determining the binding, if any. Standard assay formats, such as for example, ELISA assaysmay be used.

One of skill in the arts of cell biology and biochemistry can readily select appropriate assays. Common assays include co-immunoprecipitation experiments, wherein the PDZ-containing protein is extracted from a cell, usually under non-denaturing conditions, and precipited using a specific antibody. Co-precipitating proteins specific to the PDZ-containing protein (and not, for example, precipitated non-specifically with the agents used to perform immunoprecipitations) are visualized and may be analyzed. Additional analyses include assays such as Western blotting (see below) and antibodies that recognize a PDZ binding peptide, micro-sequencing of co-precipitated peptides, mass-spectrophotometric sequencing, etc.

Western Blotting

Methods of Western blotting are well known to those of skill in the art. Generally, a protein sample, such as a cell or tissue extract, is subjected to SDS-PAGE at such conditions as to yield an appropriate separation of proteins within the sample. The proteins are then transferred to a membrane (e.g., nitrocellulose, nylon, etc.) in such a way as to maintain the relative positions of the proteins to each other.

Visibly labeled proteins of known molecular weight are included within a lane of the gel. These proteins serve as a method of insuring that adequate transfer of the proteins to the membrane has occurred and as molecular weight markers for determining the relative molecular weight of other proteins on the blot.

Subsequent to transfer of the proteins to the membrane, the membrane is submersed in a blocking solution to prevent nonspecific binding of the primary antibody.

The primary antibody, recognizing a PDZP, PDZD, PIP or PDBP may be labeled and the presence and molecular weight of the antigen may be determined by detection of the label at a specific location on the membrane. However, the primary antibody may not be labeled, and the blot is further reacted with a labeled secondary antibody. This secondary antibody is immunoreactive with the primary antibody; for example, the secondary antibody may be one to rabbit imunoglobulins and labeled with alkaline phosphatase. An apparatus for and methods of performing Western blots are described in U.S. Pat. No. 5,567,595.

Immunoprecipitation

Protein expression can be determined, and quantitated, by isolation of antigens by immunoprecipitation. Methods of immunoprecipitations are described in U.S. Pat. No. 5,629,197. Immunopreciptitation involves the separation of the target antigen component from a complex mixture, and is used to discriminate or isolate minute amounts of protein. For the isolation of cell-surface localized proteins, nonionic salts are preferred, since other agents such as bile salts, precipitate at acid pH or in the presence of bivalent cations.

Immunofluorescence/Immunohistochemical

Protein expression by cells or tissue can be ascertained by immunolocalization of an antigen. Generally, cells or tissue are preserved by fixation, exposed to an antibody that recognizes the epitope of interest, such as a PDZP, PDZD, PIP or PDBP, and the bound antibody visualized. Co-localization experiments are suggestive of protein interactions; in this approach, the two antigens of interest are labeled with two different markers, such as rhodamine and fluorescein. When rhodamine (red) and fluorescein (green) are co-localized, a yellow signal is produced. Ultrastructurally, labels may be different size of gold particles, and actual distances between the different sized particles can be assessed for the likelihood of a protein-protein interaction.

Any cell, cell line, tissue, or even an entire organism is appropriate for fixation. Cells may be cultured in vitro as primary cultures, cell lines, or harvested from tissue and separate mechanically or enzymatically. Tissue may be from any organ, plant or animal, and may be harvested after, or preferably prior to fixation. An entire organism may also be examined. Fixation may be by any known means in known in the art; the requirements are that the protein to be detected be not rendered unrecognizable by the binding agent, most often an antibody. Appropriate fixatives include paraformaldehyde-lysine-periodate, formalin, paraformaldehyde, methanol, acetic acid-methanol, glutareldehyde, acetone and the like; one of skill in the art will know the appropriate concentrations and will determine empirically the proper fixative, which depends on variables such as the protein of interest, the properties of a particular detecting reagent (such as an antibody), and the method of detection (fluorescence, enzymatic) and the method of observation (epi-fluorescence, confocal microscopy, light microscopy, ultrastructural analysis, etc.). Preferably, the sample is washed, most often with a biological buffer, prior to fixation. Fixatives are prepared in aqueous solutions or in biological buffers; many fixatives are prepared preferably to applying to the sample. Suitable biological buffers include salines (e.g., phosphate buffered saline), N-(carbamoylmethyl)-2-aminoethanesulfonic acid (ACES), N-2-acetamido-2-iminodiacetic acid (ADA), bicine, bis-tris, 3-cyclohexylamino-2-hydroxy-1-propanesulfonic acid (CAPSO), ethanolamines, glyccine, N-2-hydroxyethylpiperazine-N′-2-ethanesulfonic acid (HEPES), 2-N-morpholinoethanesulfonic acid (MES), 3-N-morpholinopropanesulfonic acid (MOPS), 3-N-morpholino-2-hyrdoxy-propanesulfonic acid (MOPSO), piperazine-N,N′-bis(2-ethanesulfonic acid) (PIPES), tricine, triethanolamine, etc. One of skill in the art will select an appropriate buffer according to the sample being analyzed, appropriate pH, and the requirements of the detection method. Preferably, the buffer is PBS.

After fixation from 5 minutes to 1 week, depending on the sample size, sample thickness, and viscosity of the fixative, the sample is washed in buffer. If the sample is thick or sections are desired, the sample may be embedded in a suitable matrix. For cryosectioning, sucrose is infused, and embedded in a matrix, such as OCT Tissue Tek (Andwin Scientific; Canoga Park, Calif.) or gelatin. Samples may also be embedded in paraffin wax, or resins suitable for electron microscopy, such as epoxy-based (Araldite, Polybed 812, Durcupan ACM, Quetol, Spurr's, or mixtures thereof; Polysciences, Warrington, Pa.), acrylates (London Resins (LR White, LR gold), Lowicryls, Unicryl; Polysciences), methylacrylates (JB-4, OsteoBed; Polysciences), melamine (Nanoplast; Polysciences) and other media, such as DGD, Immuno-Bed (Polysciences) and then polymerized. When embedded in wax or resin, samples are dehydrated by passing them through a concentration series of ethanol or methanol; in some cases, other solvents may be used, such as polypropylene oxide. Preferred resins are hydrophilic since these are less likely to denature the protein of interest during polymerization and will not repel antibody solutions (such as Lowicryls, London Resins, water-soluble Durcupan, etc.). Embedding may occur after the sample has been reacted with the detecting reagents, or samples may be first embedded, sectioned (via microtome, cyrotome, or ultramicrotome), and then the sections reacted with the detecting reagents.

Especially in the cases of immunofluorescent or enzymatic product-based detection, background signal due to residual fixative, protein cross-linking, protein preciptiation or endogenous enzymes may be quenched, using, e.g., ammonium hydroxide or sodium borohydride or a substance to deactivate or deplete confounding endogenous enzymes, such as hydrogen peroxide which acts on peroxidases. To detect intracellular proteins in samples that are not to be sectioned, samples may be permeabilized. Permabilizing agents include detergents, such as t-octylphenoxypolyethoxyethanols, polyoxyethylenesorbitans, and other agents, such as lysins, proteases, etc.

Non-specific binding sites are blocked by applying a protein solution, such as bovine serum albumin (BSA; denatured or native), milk proteins, or preferably in the cases wherein the detecting reagent is an antibody, normal serum or IgG from a non-immunized host animal whose species is the same as that of the detecting antibody's. For example, a procedure using a secondary antibody made in goats would employ normal goat serum.

The protein is then reacted with the detecting agent, preferably an antibody. If an antibody is used, it may be applied in any form, such as Fab fragments and derivatives thereof, purified antibody (affinity, precipitation, etc.), supernatant from hybridoma cultures, ascites and serum. The antibody may be diluted in buffer or media, preferably with a protein carrier, such as the solution used to block non-specific binding sites. The antibody may be diluted, usually determined empirically. In general, polyclonal sera, purified antibodies and ascites may be diluted 1:50 to 1:200,000, more often, 1:200 to 1:500. Hybridoma supernatants may be diluted 1:0 to 1:10, or may be concentrated by dialysis or ammonium sulfate precipitation and diluted if necessary. Incubation with the antibodies may be carried out for as little as 20 minutes at 37° C., 2 to 6 hours at room temperature (approximately 22° C.), or 8 hours or more at 4° C. Incubation times can easily be empirically determined by one of skill in the art.

To detect the binding of the antibody to the protein of interest, such as one that binds a globin, a label is used. The label may be coupled to the binding antibody, or to a second antibody that recognizes the first antibody, and is incubated with the sample after the primary antibody incubation and thorough washing. Suitable labels include fluorescent moieties, such as fluorescein isothiocyanate, fluorescein dichlorotriazine (and fluorinated analogs of fluorescein), naphthofluorescein carboxylic acid and its succinimidyl ester, carboxyrhodamine 6G, pyridyloxazole derivatives, Cy2, 3 and 5, phycoerythrin, succinimidyl esters, carboxylic acids, isothiocyanates, sulfonyl chlorides, dansyl chlorides, tetramethylrhodamine, lissamine rhodamine B, tetramethylrhodamine, tetramethylrhodamine isothiocyanate, succinimidyl esters of carboxytetramethylrhodamine, rhodamine Red-X succinimidyl ester, Texas Red sulfonyl chloride, Texas Red-X succinimidyl ester, Texas Red-X sodium tetrafluorophenol ester, Red-X, Texas Red dyes, naphthofluoresceins, coumarin derivatives, pyrenes, pyridyloxazole derivatives, dapoxyl dyes, Cascade Blue and Yellow dyes, benzofuran isothiocyanates, propionic acid succinimidyl esters, pentanoic acid succinimidyl esters, sodium tetrafluorophenols, 4,4-difluoro-4-bora-3a,4a-diaza-s-indacene; enzymatic, such as alkaline phosphatase or horseradish peroxidase; radioactive, including ³⁵S and ³⁵I-labels, avidin (or streptavidin)-biotin-based detection systems (often coupled with enzymatic or gold signal systems), and gold particles. In the case of enzymatic-based detection systems, the enzyme is reacted with an appropriate substrate, such as 3, 3′-diaminobenzidine (DAB) for horseradish peroxidase; preferably, the reaction products are insoluble. Gold-labeled samples, if not prepared for ultrastructural analyses, may be chemically reacted to enhance the gold signal; this approach is especially desirable for light microscopy. The choice of the label depends on the application, the desired resolution and the desired observation methods. For fluorescent labels, the fluor is excited with the appropriate wavelength, and the sample observed with a microscope, confocal microscope, or FACS machine. In the case of radioactive labeling, the samples are contacted with autoradiography film, and the film developed; alternatively, autoradiography may also be accomplished using ultrastructural approaches. For co-localization experiments, one of skill in the art will select appropriate visualization techniques that are compatible and informative.

Other experiments to determine protein-protein interactions will be known to one of skill. For example, in vitro binding assays under cellular physiological conditions can be performed with purified PDZ domain-containing proteins and a candidate binding peptide. Alternatively, a genetic approach can be used in an appropriate organism (C. elegans, E. coli, A. thaliana, Mus musculus, S. cerevisae, S. pombe, etc.), most often with suppressor analyses.

II. Uses for PDZ-Domain Ligands

The elucidation of the peptides that bind a particular PDZ domain and the further elucidation of those polypeptides that contain those PDZ domain ligands in their carboxy termini enable one to manipulate the interaction to advantage. Such manipulation may include inhibition of the association between a PDZ domain and its cognate PDZ-ligand-containing protein. Other uses include diagnostic assays for diseases related to PDZ-domain containing proteins and their associating partners, the use of the PDZ domains and ligands in fusion proteins as purification handles and anchors to substrates.

A. PDZ-Domain-Ligand-Interaction Inhibitor

One way to modulate the interaction between a PDZ-domain ligand and a PDZ protein is to inhibit the interaction between a PDZ ligand and its cognate PDZ domain. “PDZ-domain-ligand-interaction inhibitor” includes any molecule that partially or fully blocks, inhibits, or neutralizes the interaction between a PDZ domain and its ligand. Molecules that may act as such inhibitors include peptides that bind a specific PDZ domain, such as those that bind the MAGI 3 or ERBIN PDZ domains (SEQ ID NOs:1-181, 209-213, 241-247 & 512-575) and others as described herein, antibodies (Ab's) or antibody fragments, fragments or variants of endogenous PDZ-domain ligands, PDZ-domain ligands, cognate PDZ-containing proteins, peptides, antisense oligonucleotides, and small organic molecules.

1. Examples of Inhibitors of the PDZ Domain Ligand Interaction

Any molecule that disrupts PDZ-domain ligand binding to its cognate PDZ domain is an inhibitor. Screening techniques well known to those skilled in the art can identify these molecules. Examples of inhibitors include: (1) small organic and inorganic compounds, (2) small peptides, (3) antibodies and derivatives, (4) peptides closely related to PDZ-domain ligand (5) nucleic acid aptamers.

Small molecules that bind to a PDZ domain or to a PDZ domain ligand and inhibit the binding of the PDZ-domain ligand to the cognate PDZ domain are useful inhibitors. Examples of small molecule inhibitors include small peptides, peptide-like molecules, preferably soluble, and synthetic, non-peptidyl organic or inorganic compounds.

(a) Small Molecules

A “small molecule” refers to a composition that has a molecular weight of less than about 5 kD and more preferably less than about 4 kD, and most preferably less than 0.6 kD. Small molecules can be, nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays. Examples of methods for the synthesis of molecular libraries have been described (Carell et al., 1994a; Carell et al., 1994b; Cho et al., 1993; DeWitt et al., 1993; Gallop et al., 1994; Zuckermann et al., 1994).

Libraries of compounds may be presented in solution (Houghten et al., 1992) or on beads (Lam et al., 1991), on chips (Fodor et al., 1993), bacteria, spores (Ladner et al., U.S. Pat. No. 5,223,409, 1993), plasmids (Cull et al., 1992) or on phage (Cwirla et al., 1990; Devlin et al., 1990; Felici et al., 1991; Ladner et al., U.S. Pat. No. 5,223,409, 1993; Scott and Smith, 1990). A cell-free assay comprises contacting a PDZP, PDZD, PIP or PDBP or biologically-active fragment with a known compound that binds a PDZP, PDZD, PIP or PDBP to form an assay mixture, contacting the assay mixture with a test compound, and determining the ability of the test compound to interact with a PDZP, PDZD, PIP or PDBP, where determining the ability of the test compound to interact with a PDZP, PDZD, PIP or PDBP comprises determining the ability of a PDZP, PDZD, PIP or PDBP to preferentially bind to or modulate the activity of a PDZP, PDZD, PIP or PDBP target molecule.

B. Identifying Inhibitors of PDZ-Domain Ligand Binding

One approach to identify inhibitors of PDZ-domain ligand binding is to incorporate rational drug design; that is, to understand and exploit the biology of the PDZ interaction. In this approach, the critical residues in a PDZ ligand are determined, as is, optionally, the optimal peptide length. Then, small molecules are designed with this information in hand; for example, if a tyrosine is found to be a critical residue for binding to a PDZ domain, then small molecules that contain a tyrosine residue will be prepared and tested as inhibitors. Generally 2, 3, 4 or 5 amino acid residues will be determined to be critical for binding and candidate small molecule inhibitors will be prepared containing these residues or the residue sidechains. The test compounds are then screened for their ability to inhibit PDZ domain-ligand interactions using protocols well-known in the art, for example, a competitive inhibition assay.

Compounds, that inhibit PDZ-domain ligand binding interactions are useful to treat diseases and conditions that are mediated by binding interactions of PDZ proteins. Diseases and conditions that are mediated, or may be mediated, by PDZ proteins include, as examples, rickettsial diseases, murine typhus, tsutsugamushi disease (Kim and Hahn, 2000), Facioscapulohumeral muscular dystrophy (Bouju et al., 1999; Kameya et al., 1999), chronic myeloid leukemia (Nagase et al., 1995; Ruff et al., 1999), Alzheimer's disease (Deguchi et al., 2000; Lau et al., 2000; McLoughlin et al., 2001; Tanahashi and Tabira, 1999a; Tomita et al., 2000; Tomita et al., 1999), neurological disorders such as Parkinson's disease and schizophrenia (Smith et al., 1999), X-linked autoimmune enteropathy (AIE) (Kobayashi et al., 1999), late onset demyelinating disease (Gillespie et al., 2000), Usher syndrome type 1 (USHI) (DeAngelis et al., 2001), nitric oxide-mediated tissue damage (Kameya et al., 1999; McLoughlin et al., 2001), tumors (Inazawa et al., 1996) and cystic fibrosis (Raghuram et al., 2001).

1. Determining Critical Residues in a PDZ Binding Polypeptide

(a) Alanine Scanning

Alanine scanning a PDZ-domain binding peptide sequence can be used to determine the relative contribution of each residue in the ligand to PDZ binding. To determine the critical residues in a PDZ ligand, residues are substituted with a single amino acid, typically an alanine residue, and the effect on PDZ domain binding is assessed. See U.S. Pat. No. 5,580,723; U.S. Pat. No. 5,834,250.

(b) Truncations (Deletion Series)

Truncation of a PDZ-domain binding peptide can elucidate not only binding critical residues, but also determine the minimal length of peptide to achieve binding. In some cases, truncation will reveal a ligand that binds more tightly than the native ligand; such a peptide is useful to inhibit PDZ domain:PDZ ligand interactions.

Preferably, a series of PDZ-domain binding peptide truncations are prepared. One series will truncate the amino terminal amino acids sequentially; in another series, the truncations will begin at the carboxy terminus. As in the case for alanine scanning, the peptides may be synthesized in vitro or prepared by recombinant methods.

(c) Rational Inhibitor Design

Based on the information obtained from alanine scanning and truncation analysis, the skilled artisan can design and synthesize small molecules, or select small molecule libraries that are enriched in inhibitors that are likely to inhibit binding.

(d) Binding Assays

Forming a complex of a PDZ binding peptide and its cognate PDZ domain facilitates separation of the complexed from the uncomplexed forms thereof and from impurities. PDZ domain:binding ligand complexes can be formed in solution or where one of the binding partners is bound to an insoluble support. The complex can be separated from a solution, for example using column chromatography, and can be separated while bound to a solid support by filtration, centrifuagation, etc. using well-known techniques. Binding the PDZ domain containing polypeptide or the ligand therefor to a solid support facilitates high throughput assays.

Test compounds can be screened for the ability to inhibit the interaction of a PDZ binding polypeptide with a PDZ domain in the presence and absence of a candidate binding compound, and screening can be accomplished in any suitable vessel, such as microtiter plates, test tubes, and microcentrifuge tubes. Fusion proteins can also be prepared to facilitate testing or separation, where the fusion protein contains an additional domain that allows one or both of the proteins to be bound to a matrix. For example, GST-PDZ-binding peptide fusion proteins or GST-PDZ domain fusion proteins can be adsorbed onto glutathione sepharose beads (SIGMA Chemical, St. Louis, Mo.) or glutathione derivatized microtiter plates that are then combined with the test compound or the test compound and either the nonadsorbed PDZ domain protein or PDZ-binding-peptide, and the mixture is incubated under conditions allowing complex formation (e.g., at physiological conditions of salt and pH). Following incubation, the beads or microtiter plate wells are washed to remove any unbound components, the matrix immobilized in the case of beads, and the complex determined either directly or indirectly. Alternatively, the complexes can be dissociated from the matrix, and the level of PDBP binding or activity determined using standard techniques.

Other fusion polypeptide techniques for immobilizing proteins on matrices can also be used in screening assays. Either a PDZ binding peptide or its target PDZ domain can be immobilized using biotin-avidin or biotin-streptavidin systems. Biotinylation can be accomplished using many reagents, such as biotin-N-hydroxy-succinimide (NHS; PIERCE Chemicals, Rockford, Ill.), and immobilized in wells of streptavidin coated 96 well plates (PIERCE Chemical). Alternatively, antibodies reactive with PDZ binding peptides or target PDZ domains but do not interfere with binding of a PDZ binding peptide to its target molecule can be derivatized to the wells of the plate, and unbound target or PDBP trapped in the wells by antibody conjugation. Methods for detecting such complexes, in addition to those described for the GST-immobilized complexes, include immunodetection of complexes using antibodies reactive with PDZ-binding peptides or target PDZ domain.

(e) Assay for Binding: Competition ELISA

To assess the binding affinities of a peptide, proteins or other PDZ ligands, competition binding assays may be used, where the ability of the ligand to bind the corresponding PDZ domain (and the binding affinity, if desired) is assessed and compared to that of a compound known to bind the PDZ domain, for example, a consensus peptide sequence determined by phage display or the cognate protein ligand determined as described above, preferably in parallel.

Many methods are known and can be used to identify the binding affinities of PDZ domain binding ligands (e.g. peptides, proteins, small mollecules, etc.); for example, binding affinities can be determined as IC₅₀ values using competition ELISAs. The IC₅₀ value is defined as the concentration of ligand which blocks 50% of PDZ domain binding to a ligand. For example, in solid phase assays, assay plates may be prepared by coating microwell plates (preferably treated to efficiently absorb protein) with neutravidin, avidin or streptavidin. Non-specific binding sites are then blocked through addition of a solution of bovine serum albumin (BSA) or other proteins (for example, nonfat milk) and then washed, preferably with a buffer containing a detergent, such as Tween-20. A biotinylated known PDZ-domain ligand (for example, the phage peptides or cognate protein as fusions with GST or other such molecule to facilitate purification and detection) is prepared and bound to the plate. Serial delutions of the ligand to be tested with a PDZ domain polypeptide arc prepared and contacted with the bound ligand. The plate coated with the immobilized ligandis washed before adding each binding reaction to the wells and briefly incubated. After further washing, the binding reactions are detected, often with an antibody recognizing the non-PDZ fusion partner and a labeled (such as horseradish peroxidase (HRP), alkaline phosphatase (AP), or a fluorescent tag such as fluorescein) secondary antibody recognizing the primary antibody. The plates are then developed with the appropriate substrate (depending on the label) and the signal quantified, such as using a spectrophotometric plate reader. The absorption signal may be fit to a binding curve using a least squares fit. Thus the ability of the various ligands to inhibit PDZ domain from binding a known PDZ-domain ligand can be measured.

Apparent to one of skill are the many variations of the above assay. For example, instead of avidin-biotin based systems, PDZ-domain ligands may be chemically-linked to a substrate, or simply absorbed. An example of such a screen is found in the Examples.

2. PDZ-Domain Peptide Ligands found During Phage Display

PDZ domain peptide ligands, even those that bind with lower affinity than a consensus sequence, are potential useful inhibitors of the PDZ-domain ligand:PDZ domain interaction, including those found in the screens for MAGI 3 and ERBIN PDZ-domain ligands; densin; scribble PDZ1 and 3; scribble PDZ2; MUPP PDZ7; human INADL PDZ6; human ZO1; AF6(MLLT4); MUPP PDZ3; MAGI1 PDZ3; MAGI3 PDZ3; INADL PDZ3; huINADL PDZ2; huPARD3PDZ3; SNTA1 PDZ; MAGI3 PDZ0; MUPP PDZ13; and MAGI3 PDZ2. Thus a method to find such an inhibitor is that of carboxy-terminal phage display.

The competitive binding ELISA is a useful means to determine the efficacy of each phage-displayed PDZ-domain binding peptide.

3. Aptamers

Aptamers are short oligonucleotide sequences that can be used to recognize and specifically bind almost any molecule. The systematic evolution of ligands by exponential enrichment (SELEX) process (Ausubel et al., 1987; Ellington and Szostak, 1990; Tuerk and Gold, 1990) can be used to find such aptamers. Aptamers have many diagnostic and clinical uses; almost any use in which an antibody has been used clinically or diagnostically, aptamers too may be used. In addition, aptamers are less expensive to manufacture once they have been identified and can be easily applied in a variety of formats, including administration in pharmaceutical compositions, bioassays and diagnostic tests (Jayasena, 1999)

In the competitive ELISA binding assay described above, the screen for candidate aptamers includes incorporating the aptamers into the assay and determining their ability to inhibit PDZ domain:PDZ-domain ligand binding.

4. Antibodies (Abs)

Any antibody that inhibits PDZ-domain ligand:PDZ domain binding is an inhibitor of the PDZ domain-ligand interaction. Examples of antibody inhibitors include polyclonal, monoclonal, single-chain, anti-idiotypic, chimeric Abs, or humanized versions of such antibodies or fragments thereof. Antibodies may be from any species in which an immune response can be raised. The different types of antibodies are discussed more fully below.

C. Utility of the PDZ Domain:PDZ-Domain Ligand Interaction

1. Affinity Purification

Affinity purification means the isolation of a molecule based on a specific attraction or binding of the molecule to a chemical or binding partner to form a combination or complex which allows the molecule to be separated from impurities while remaining bound or attracted to the partner moiety. The interaction between a PDZ ligand and the corresponding PDZ domain can be exploited to purify any protein that contains or has been modified to contain a PDZ domain and/or ligand therefor. The advantages of such a system include the ability to modulate specificity, control binding, and the manipulation of the small size of most PDZ-domain ligands.

A PDZ “fusion protein” comprises a PDZ domain or PDZ-domain ligand fused to a non-PDZ domain or ligand protein partner, or a protein partner in which the particular PDZ domain or ligand is not present. The PDZ domain or ligand may be fused to the N-terminus or the C-terminus of the partner protein.

Such fusion proteins can be easily created using known recombinant methods. A nucleic acid encoding a PDZ domain or ligand can be fused in-frame with a non-PDZ domain or ligand encoding nucleic acid. Fusion genes may also be synthesized by conventional techniques, including automated DNA synthesizers. PCR amplification using anchor primers that give rise to complementary overhangs between two consecutive gene fragments that can subsequently be annealed and reamplified to generate a chimeric gene sequence (Ausubel et al., 1987) is also useful. Many vectors are commercially available that facilitate sub-cloning PDZP, PDZD, PIP or PDBP in-frame to a fusion moiety. If desired the proteins can be expressed in a host, such as a bacterium (such as E. coli) or eukaryotic cell (such as COS cells or a baculovirus-based system using insect cells), and purified.

Alternatively, proteins may be synthesized in vitro, using standard amino acid synthesizers.

To purify a PDZ ligand, for example, a PDZ domain containing polypeptide may be anchored to a solid support, such as sepharose, using for example, chemical cross-linking, such as cyanogens bromide, loaded into a column and used to separate a ligand from a mixture containing the same. A mixture comprising the ligand is passed over the support under conditions that allow for specific binding between the bound PDZ domain and the ligand. After washing, the PDZ ligand is eluted from the column, using methods well known in the art for disrupting non-covalent interactions, such as an increasing salt gradient. Obvious to one of skill in the art are the many permutations of the above method. For example, the fusion protein may comprise the PDZ domain, and the solid support may be prepared with the cognate PDZ ligand. The solid support may be used in a “batch” approach instead of loaded into a column. Elution conditions may also be varied; for example, changes in pH may be exploited or chaotropes used, or any phage-displayed peptide that was found to bind the specific PDZ domain may be used to release the bound fusion protein.

2. Anchor System

The binding between a PDZ domain and its ligand can be exploited to anchor a protein or other substance (such as nucleic acids, organic and inorganic small molecules, etc.) to a substrate, in a manner similar to avidin-biotin binding. The advantages of such a system include those enumerated for affinity purification, as well as the ability, for example, to array the molecules on a substrate as patterned by the specific placement of various PDZ domains (or PDZ-domain ligands) and the cognate PDZ domain-ligands (or PDZ domains).

Such anchoring systems have uses in high-throughput assays that utilize arrays.

D. Target Validation

As noted above, PDZ domains are responsible for protein-protein interactions associated with signaling, localization and transport of intracellular proteins. Disruption of these processes often leads to disease. The PDZ-domain binding peptides, cognate protein ligands and inhibitors found using the assays described above, can be used to verified the causual relationship between these protein-protein interactions and specific disease states or conditions in vitro or in vivo by monitoring thephenotypic or biologic response to disruption of the endogenous PDZ domain:PDZ-domain ligand interaction.

In this approach, the PDZ-domain ligands are allowed to compete for the endogenous ligand in a cell. The peptides can be introduced into the cell by any method known in the art, such as liposomes, microinjecttion, lipid transfection, antenapedic peptide transfection etc. Alternatively, the PDZ-domain ligand peptides may be expressed from a suitable vector (see vectors discussion, below).

Because PDZ domains target their proteins and cognate ligands to specific cellular sites, the ability of the PDZ-domain ligand candidates to disrupt this interaction is monitored, preferably by immunolocalization protocols, such as indirect immunofluorescence or immunoelectron microscopy.

E. Testing for Disease

Both PDZ-domain ligand peptides/polypeptides and polynucleotides can be used in clinical screens to test for disease etiology or to assess the level of risk for these disorders. Tissue samples of a patient can be examined for the amount of PDZ-domain cognate protein ligand or mRNA therefor. When amounts significantly smaller or larger than normal are found, they are indicative of disease or risk of disease associated with improper or abnormal protein-protein interaction. Mutation of PDZ-domain ligand nucleic acid can yield altered activity, and a patient with such a mutation may have a disease or be at risk for a disease. Finally, determining the amount of expression of PDZ-domain ligand in a mammal, in a tissue sample, or in a tissue culture, can be used to discover inducers or repressors of the gene.

Determination of PDZ-domain ligand mRNA, proteins or activity levels in clinical samples may have predictive value for tracking progression of disorders, or in cases in which therapeutic modalities are applied to correct disorders.

III. Methods of the invention provide a novel means to identify ligands that are the biological binding partners of PDZ domain-containing proteins. Identification of these novel interactions serves as a basis for novel diagnostic and therapeutic approaches in treating or ameliorating conditions and diseases associated with disruptions of the known biological functions of the newly-identified PDZ domain ligands. Thus, for example, as described herein, inhibitors of these interactions may be used, for example in diagnostic applications, wherein amounts of a ligand, or the amount and/or extent of interaction between a PDZ protein and a ligand of interest can be determined using quantitative binding assays, which are known in the art and described herein. For conditions associated with an abnormally low amount of interaction between a PDZ domain protein and a cognate ligand which may be due to, for example a mutation in either protein that decreases the binding interaction, a therapeutic approach/agent may be based on, for example, administering exogenous cognate ligand and/or PDZ domain protein, or nucleic acids that express said ligand or protein. The exogenous ligand and/or PDZ domain protein may be a version of the ligand or PDZ domain protein that has enhanced binding interaction affinity, which can be designed based on peptide sequence information described herein and/or determined based on methods herein described. As another example, importance of particular residues for the binding interaction can be determined based on information obtained from structure-activity analysis of PDZ domain sequence and/or selected peptide sequences as described herein. Such information can be used, using routine methods known in the art, to design better binding sequences. Such information can in turn be used to design potent and specific targeted therapeutic interventions, including those based on gene therapy. Examples of optimization of binding sequences are described herein.

As stated above, identification of cognate ligands for PDZ domain proteins of interest provides information critical in efforts to treat or diagnose conditions and diseases associated with these proteins and/or their interactions with each other. Methods of the invention can be used to obtain such information. The following describes a partial list of PDZ domain proteins and their respective cognate ligands as identified using these methods. A brief description of the known biological functions of the cognate ligands is also provided, along with the database accession number for references that further describe these ligands and the PDZ domain proteins that interact with them. References identified by these and other database accession numbers described herein are herein incorporated in their entirety by reference.

(1) Magi3 PDZ2

Membrane-associated guanylate kinase with inverted orientation 3 (MAGI-3), a member of the MAGUK family, contains guanylate kinase, WW and PDZ domains, associates with PTEN, may localize PTEN to the plasma membrane and enhance PTEN inhibition of Akt (AKT1). AF7238

Using the method of the invention described above, the feasibility of the method to identify a PDZ cognate ligand was shown by confirming the identity of PTEN/MMAC (SEQ ID NO:797) as a cognate ligand for PDZ2 of MAGI 3. See the Examples 1-6.

(2) ERBIN

Using the method of the invention described above, three gene products were identified by selecting phage peptides against the PDZ domain of ERBIN and then searching in the Dayhoff database using a consensus sequence [DE][ST]WV-COOH derived from alignment of the ERBIN PDZ domain selected phage peptides. See Examples 7-13. All three genes products, (a) δ-catenin (neural plakophilin-related arm-repeat protein [NPRAP], presenilin-1 interacting protein GT24 and δ 2-catenin), (b) armadillo repeat protein deleted in Velo-cardio-facial syndrome (ARVCF), and (c) p0071 are members of the Armadillo family of proteins. Importantly, all three of these proteins fall within the p120(ctn) subfamily of the larger Armadillo protein family indicating that the conserved DSWV PDZ binding motif reflects a shared characteristic of how these proteins function within the cell. Both p0071 and ARVCF are widely expressed (Hatzfeld and Nachtsheim, 1996 Journal of Cell Science; Sirotkin-H et al. 1997a Genomics) whereas δ-catenin expression is restricted to neurons, being found at high levels in proliferating neuronal progenitor cells and at lower levels in post-mitotic neurons (Carole-H et al. 2000 The Journal of Comparative Neurobiology). δ-catenin (NP_{—}001322.1) is a member of the catenin family of cadherin-binding proteins, is a cytoskeletal regulator that link cadherins to the cytoskeleton, and it plays a role in cell migration; loss of expression correlates with advanced bladder and colorectal cancer. It is know that all three may interact similarly with type I and II cadherens at adherens junctions and that the binding site on cadherens is distinct from that used by beta-catenin. Beta-catenin is the most well understood member of the armadillo protein family having roles in both cell-adhesion and transcription. It has been well established that mutations which disrupt ubiquitin-mediated proteolysis of beta-catenin in the cytoplasm lead to abnormally high nuclear levels of this protein. Such mutations are responsible for the majority of colon cancers. Similar to beta catenin, all three proteins are localized to adherens junction and both p0071 and ARVCF can also shuttle to the nucleus (Hatzfeld and Nachtsheim 1996 Journel of Cell Science; Mariner-D. J. et al 2000 Journal of Cell Science). The available data thus suggest that ARVCF, p0071 and δ catenin will have cellular roles parallel to beta-catenin both in the morphogenesis of cellular junctions and transcription. The physiological importance of these three proteins is also based on other traits which have been reported in the literature. p0071 and δ-catenin have both been shown to interact with presenilan-1, mutations in which have been linked to early-onset Alzheimer's disease. In addition data suggests that δ-catenin is important for the migration of neuronal precursor cells, a function which would invariably lead to increased metastasis of neuronal cancers if this process were to become disregulated such as occurs with beta catenin and colon cancer. Thus, disruption of the interaction of ERBIN with any or all of ARVCF, p0071 or δ catenin is useful to treat or modify one of these disease states.

(3) Densin

Densin (or Densin-180) (NP 476483.1) is a founding member of the LAP (leucine-rich repeat (LRR) and PDZ) family and may be involved in signal transduction and in synaptic adhesion. It forms a complex in vitro with CaM kinase II (Camk2a) and alpha actinin (human ACTN4).

Using methods described herein (for example, for ERBIN), the following gene products were identified as ligands for densin: (1) ARVCF (NP 001661.1) (SEQ ID NO.: 706)—Armadillo repeat gene deleted in velocardiofacial syndrome, binds cadherins and may play a role in cell adhesion at the adherens junction; hemizygosity of the corresponding gene is associated with velocardiofacial syndrome; (2) delta-catenin (SEQ ID NO.: 707); and (3) pO071 (SEQ ID NO.:708).

(4) Scribble PDZ1 and 3

Scribble is a protein containing PDZ (DHR, GLGF) domains, which targets signaling proteins to membranes, contains leucine rich repeats and which mediates protein-protein interactions. NP 056171.1.

Using methods described herein (for example, for ERBIN), the following gene products were identified as ligands for Scribble PDZ 1 and PDZ3:

1. ZO2: Tight junction protein 2, a member of the membrane-associated guanylate kinase-containing family, involved in the establishment and maintenance of tight junctions; deregulation may be associated with the development of ductal carcinomas. NP 004808.1 (SEQ ID NO.: 709)

2. Kv1.5: Voltage-gated potassium channel (shaker-related subfamily 1) member 5, a rapidly activating, slowly inactivating delayed rectifier K+ channel, contributes to membrane repolarization and regulation of action potential duration in the heart. 002225.1 (SEQ ID NO.: 710)

3. GPR87: Member of the rhodopsin family of G protein-coupled receptors (GPCR), has moderate similarity to platelet ADP receptor (rat P2y12), which is a G protein (Gi)-coupled receptor that induces platelet aggregation during blood clotting. NP_{—}115775.1 (SEQ ID NO.: 711)

4. Actinin: Alpha actinins belong to the spectrin gene superfamily which represents a diverse group of cytoskeletal proteins, including the alpha and beta spectrins and dystrophins. Alpha actinin is an actin-binding protein with multiple roles in different cell types. In nonmuscle cells, the cytoskeletal isoform is found along microfilament bundles and adherens-type junctions, where it is involved in binding. (SEQ ID NO.: 712)

5. beta-catenin: Links cadherins to the cytoskeleton, also functions in the wnt signal transduction pathway by transmitting signals to the nucleus in complexes with transcription factors, also required for anteroposterior axis formation; mutations in the gene are associated with various cancers. NP_{—}001895.1 (SEQ ID NO.: 713)

6. CD34: CD34 antigen, a transmembrane sialomucin associated with hematopoietic stem cells and an L-selectin ligand on high endothelial venules, transduces signals that regulate cytoadhesion of hematopoietic cells, may play a role in early stages of hematopoiesis. NP 001764.1 (SEQ ID NO.: 714)

(5) Scribble PDZ2

Ligands for SCRIBBLE PDZ2 as identified according to methods of the invention are the same as for ERBIN.

(6) MUPP PDZ7

MUPP is a multiple PDZ domain protein, a member of the multi-PDZ domain protein family with 13 PDZ domains, interacts with the C termini of serotonin receptors (HTR2A, HTR2B, and HTR2C), and may act as a multivalent scaffolding protein to regulate signaling.

Using methods described herein (for example, for ERBIN), the following gene products were identified as ligands for Scribble PDZ 1 and PDZ3:

1. HTR2B: 5-hydroxytryptamine 2B (serotonin) receptor, a G protein-coupled receptor that activates phospholipase C, mediates the physiologic functions of serotonin including smooth muscle contraction in the GI tract and fibroblast mitogenesis. NP_{—}00858.1 (SEQ ID NO.: 715)

2. PDGFRb: Platelet-derived growth factor receptor beta chain, a tyrosine kinase receptor that activates the MAPK kinase pathway and regulates both cell proliferation and cell migration. The PDGFRb gene encodes a cell surface tyrosine kinase receptor for members of the platelet-derived growth factor family. These growth factors are mitogens for cells of mesenchymal origin. The identity of the growth factor bound to a receptor monomer determines whether the functional receptor is a homodimer or a heterodimer, composed of both platelet-derived growth factor receptor alpha and beta polype. J03278 (SEQ ID NO.: 716)

3. delta-catenin.

4. SGK: Serum glucocorticoid regulated kinase, a serine/threonine protein kinase that inhibits apoptosis and stimulates renal sodium transport. NP_{—}005618.1 (SEQ ID NO.: 717)

5. SSTR3: Somatostatin receptor 3, a G protein-coupled receptor that inhibits adenylyl cyclase activity and mediates the inhibitory effects of somatostatin on cell proliferation. The protein encoded by this gene is a GTPase which belongs to the RAS superfamily of small GTP-binding proteins. Members of this superfamily appear to regulate a diverse array of cellular events, including the control of cell growth, cytoskeletal reorganization, and the activation of protein kinases. Somatostatin acts at many sites to inhibit the release of many hormones and other secretory proteins. The biological effects of somatostatin are probably mediated by a family of G protein-coupled receptors that are expressed in a tissue-specific manner. SSTR3 is a member of the superfamily of receptors having seven transmembrane segments and is expressed in highest levels in brain and pancreas. NP_{—}001042.1 (SEQ ID NO.: 718)

(7) Human INADL PDZ6

Ligands for human INDL PDZ6 as identified according to methods of the invention are the same as for MUPP PDZ7.

(8) Human ZO1

Tight junction protein ZO-1 (Zonula occludens 1 protein) (Zona occludens 1 protein) (Tight junction protein 1). NM-003257.

Using methods described herein (for example, for ERBIN), the following gene products were identified as ligands for human ZO1:

1. Claudin-17, a member of the claudin family of integral membrane proteins, contains four transmembrane domains, localizes to tight junction strands. It may be involved in tight junction formation and maintenance, and play a role in cell adhesion. NP036263.1 (SEQ ID NO.: 719)

2. Claudin1: another member of the claudin family, and may be involved in maintaining cell polarity. NP_{—}066924.1 (SEQ ID NO.: 720)

3. Claudin 3, another member of the claudin family of integral membrane proteins, Clostridium perfringens enterotoxin receptor, may be associated with ovarian tumor formation; CLDN3 gene maps to region commonly deleted in Williams syndrome. NP_{—}001297.1 (SEQ ID NO.: 721)

4. Claudin 7, a putative integral membrane protein which may be involved in tight junction formation. NP_{—}001298.1 (SEQ ID NO.: 722)

5. Claudin 9; a transmembrane protein of the claudin family that is involved in the formation of tight junction strands. (SEQ ID NO.: 723)

6. Claudin 18 (SEQ ID NO.: 724)

7. PDGFRA (SEQ ID NO.: 725)

8. PDGFRB (SEQ ID NO.: 726)

9. δ-Catenin (SEQ ID NO.: 707)

10. ARVCF (SEQ ID NO.: 706)

11. SGK (SEQ ID NO.: 717)

(9) AF6 (MLLT4)

A gene associated with myeloid/lymphoid or mixed-lineage leukemia, translocated to chromosome 4, myeloid/lymphoid or mixed-lineage leukemia (trithorax (Drosophila) homolog); translocated to 4. NM_{—}005936. Using methods described herein (for example, for ERBIN), the following gene products were identified as ligands for AF6 (MLLT4):

1. FYCO 1: Protein containing a FYVE zinc finger domain and a RUN domain, which may be involved in Ras-like GTPase signaling pathways, has a region of receptors (GPCR), has moderate similarity to rat Rn. 10680, which is a C5a chemoattractant (anaphylatoxin) receptor. AAK1264.1 (SEQ ID NO.: 727)

2. BLTR2: a seven transmembrane receptor; leukotriene B4 receptor BLT2. A G protein-coupled receptor that binds leukotriene B4 with low affinity, mediates intracellular calcium flux and chemotaxis, also may play a role in humoral defense mechanisms. NP_{—}062813.1 (SEQ ID NO.: 728)

3. TM7SF3: Transmembrane 7 superfamily member 3, contains seven transmembrane domains, may be involved in transmission of external signals into the cell. NP 057635.1 (SEQ ID NO.: 729)

4. OR10C1: Protein with high similarity to spermatid chemoreceptors, and to olfactory receptors, member of the rhodopsin family of G protein-coupled receptors (GPCR) NP039229.1 (SEQ ID NO.: 730)

5. CNTNAP2 (contactin associated protein-like 2): Protein containing three extracellular laminin G domains, two epidermal growth factor (EGF)-like domains and an F5 or 8 type C (discoidin) domain, has moderate similarity to neurexin 4 (contactin associated protein 1, mouse Cntnap 1). NP 054860.1 (SEQ ID NO.: 731)

6. Nectin3: Poliovirus receptor-related I (nectin), immunoglobulin-related cell adhesion molecule, mediates cellular entry for many alpha herpes viruses; autosomal recessive mutation in the corresponding gene is associated with cleft lip/palate-ectodermal dysplasia. NP_{—}002846.2 (SEQ ID NO.: 732)

7. SH3D5: SH3 domain-containing protein that is associated with the formation of focal adhesions and actin stress fibers, also binds the product of the proto-oncogene c-Cbl (Cbl) and may regulate insulin receptor signaling. NP_{—}033192.1 (SEQ ID NO.: 733)

8. Utrophin: a membrane-associated protein that interacts with cytoskeletal proteins, associated with muscle and neuromuscular junction development and cell adhesion, may partially compensate for dystrophin (DMD) deficiency in Duchenne's muscular dystrophy. NP_{—}009055.1 (SEQ ID NO.: 734)

(10) MUPP PDZ3

Using methods described herein (for example, for ERBIN), the following gene products were identified as ligands for MUPP PDZ3:

1. Drosophila NUMB homolog: Numb-like (Numb-related), a putative protein-binding protein that contains a phosphotyrosine binding domain and may regulate neurodevelopment or neuroplasticity. NP_{—}004747.1 (SEQ ID NO.: 735)

2. TGFBR1: Transforming growth factor beta receptor I, a serine-threonine kinase that is a member of the activin-TGF superfamily, involved in signal transduction and cell growth; dysfunction is associated with atherosclerosis and restinosis. NP_{—}004603.1 (SEQ ID NO.: 736)

3. IGFBP7: Insulin-like growth factor binding protein 7, functions in the regulation of cell proliferation and cell adhesion, may act as a tumor suppressor, may play a role in angiogenesis and in senescence. NP_{—}001544.1 (SEQ ID NO.: 737)

4. CD3611: CD36 antigen (collagen type I receptor, thrombospondin receptor)-like 1. Scavenger receptor BI, a member of the CD36 superfamily and high affinity cell surface high density lipoprotein (HDL) receptor, mediates the selective uptake of cholesterol from high density lipoprotein, also binds apoptotic thymocytes. NP_{—}005496.1 (SEQ ID NO.: 738)

(11) Magil PDZ3

BAI 1-associated protein 1, contains a guanylate kinase domain, two WW domains, and several PDZ domains, interacts with the brain-specific angiogenesis inhibitor 1 (BAI1), may be involved in signal transduction and cell adhesion in the brain. The protein encoded by this gene is a member of the membrane-associated guanylate kinase homologue (MAGUK) family. Characterized by two WW domains, a guanylate kinase domain, and five PDZ domains, this protein interacts with the cytoplasmic region of BAI1. Together, these proteins may play a role in cell adhesion and signal transduction. NP_{—}004733.1

Using methods described herein (for example, for ERBIN), the following gene products were identified as ligands for Magi1 PDZ3:

1. SDOLF: olfactory receptor sdolf, a member of the rhodopsin family of G protein-coupled receptors (GPCR), has moderate similarity to odorant receptor 83 (mouse Or83), which is a receptor that is present in distinct regions of the olfactory epithelium. NP_{—}277054.1 (SEQ ID NO.: 739)

2. PLEKHA1: Pleckstrin homology (PH) domain-containing family A member 1 (tandem PH domain-containing protein 1), binds specifically to phosphatidylinositol 3,4-bisphosphate via PH domain, binds PDZ domains, and regulates phosphoinositide signaling pathways. NP_{—}067635.1 (SEQ ID NO.: 740)

3. PEPP2: Phosphoinositol 3-phosphate-binding protein-2, contains a pleckstrin homology domain with a putative phosphatidylinositol 3,4,5-trisphosphate-binding motif and two WW domains, a probable phospholipid binding protein which may act as an adaptor protein. NP_{—}061885.1 (SEQ ID NO.: 741)

4. MUC12: an EGF-like cell surface glycoprotein that may play a role in the regulation of epithelial cell growth. AAD55678.1 (SEQ ID NO.: 742)

5. SLITI: a secreted protein that has EGF-like motifs and leucine-rich motifs, expressed only in the brain, has strong similarity to rat Rn.30002, which may act to guide the direction of neuronal migration in the developing olfactory system. NP_{—}003052.1 (SEQ ID NO.: 743)

6. PARK2: Parkinson disease (autosomal recessive, juvenile) 2, a ubiquitin-protein ligase with a RING-finger motif, functions to ubiquinate alpha synuclein (SNCA), Synphilin-1 (SNCAIP) and CDCrel 1 (PNUTL1); mutations cause autosomal recessive juvenile parkinsonism. NP_{—}054642.1 (SEQ ID NO.: 744)

7. HTR2A; 5-hydroxytryptamine (serotonin) 2A receptor, a G protein-coupled receptor that modulates intracellular calcium levels and plays roles in perception, mood, and appetite; may play a role in the pathophysiology of depressive and eating disorders. NP_{—}000612.1 (SEQ ID NO.: 745)

8. PITPNB: Phosphatidylinositol transfer protein alpha, catalyzes the transfer of phosphatidylinositol and phosphatidylcholine between membranes, essential for phospholipase C signaling and for constitutive and regulated vesicular traffic. NP_{—}006215.1 (SEQ ID NO.: 746)

(12) MAGI3 PDZ3

Using methods described herein (for example, for ERBIN), the following gene products were identified as ligands for Magi3 PDZ3:

1. JAM1: Junctional adhesion molecule 1, participates in platelet adhesion and aggregation and may play roles in intracellular signaling, the assembly of tight junctions, and the inflammatory response, may be involved in the pathogenesis of immune thrombocytopenia. NP_{—}058642.1 (SEQ ID NO.: 747)

2. JAM2: Junctional adhesion molecule 2, a member of the immunoglobulin superfamily, expressed on high endothelial venules and may help in neutrophil and monocyte transendothelial migration. NP_{—}067042.1 (SEQ ID NO.: 748)

3. LLT1: The human lectin-like NK cell receptor is a new member of the NK cell receptors located in the human NK gene complex. The protein structure contains a transmembrane domain near the N-terminus and an extracellular domain with similarity to the C-type lectin-like domains shared with other NK cell receptors. This protein may be involved in mediating activation signals. NP_{—}037401.1 (SEQ ID NO.: 749)

4. PTTG3: Pituitary tumor-transforming 3, a protein that may be associated with tumorigenesis. NP_{—}066280.1 (SEQ ID NO.: 750)

5. CD83 antigen, (activated B lymphocytes, immunoglobulin superfamily), may play a role in antigen presentation and lymphocyte activation, expressed on dendritic cells at final stage of their maturation. NP_{—}004224.1 (SEQ IDNO.: 751)

6. Delta-like homolog (Drosophila), preadipocyte factor (fetal antigen 1), putative growth factor, may be involved in regulation of hematopoesis, may inhibit adipocyte differentiation, may play a role in neuroendocrine differentiation. NP_{—}003827.1 (SEQ ID NO.: 752)

7. TNFRSF 18: Tumor necrosis factor receptor superfamily member 18, associates with TRAF1, TRAF2, and TRAF3; regulates activity of the NF kappa B transcription factor and may play a role in FAS (TNFRSF6) and FasL (TNFSF6) mediated apoptosis. NP_{—}004186.1 (SEQ ID NO.: 753)

8. RGS20: Regulator of G protein-signaling 20, negatively regulates G protein-signaling by binding to the unphosphorylated form of the G protein alpha z subunit (GNAZ) and stimulating its intrinsic GTPase activity. NP_{—}003693.2 (SEQ ID NO.: 754)

9. TM4SF6: Transmembrane 4 superfamily member 6, a member of the tetraspanin family, may be involved in cell adhesion, migration, and proliferation. NP_{—}003261.1 (SEQ ID NO.: 755)

10. PARK2 (SEQ ID NO.: 744)

11. GPR10; G protein-coupled receptor 10, putative G protein-coupled receptor that binds a peptide which stimulates prolactin (PRL) secretion. NP_{—}004239.1 (SEQ ID NO.: 756)

12. IL2RB: Interleukin 2 receptor beta, binds and activates signal transducer molecules in MAP kinase, JAK-STAT, and phosphoinositide 3-kinase mediated signaling pathways, plays a role in T cell mediated immune response and tumor growth. NP_{—}000869.1 (SEQ ID NO.: 757)

(13) INADL PDZ3

PDZ domain protein (Drosopila inad-like), may play a role in assembly of multiprotein complexes. NP_{—}005790.1, INADL

Using methods described herein (for example, for ERBIN), the following gene products were identified as ligands for INADL PDZ3:

1. BLTR2 (SEQ ID NO.: 728)

2. JAMI (SEQ ID NO.: 747)

3. JAM2 (SEQ ID NO.: 748)

4. KV8. 1: Neuronal potassium channel alpha subunit, functions as an inhibitory subunit in subclasses of outward rectifier potassium channels of the Kv2 and Kv3 subfamilies. NP_{—}055194.1 (SEQ ID NO.: 758)

5. PTTG3: Pituitary tumor-transforming 3, a protein that may be associated with tumorigenesis. NP_{—}066280.1 (SEQ ID NO.: 750)

6. CNTNAP2 (SEQ ID NO.: 731)

7. NRXN1; Neurexin I-alpha, a transmembrane protein that binds alpha-latrotoxin, which is a neurotoxin from black widow spider venom. NP_{—}004792.1 (SEQ ID NO.: 759)

8. NRXN2: Neurexin 2, protein with very strong similarity to rat Nrxn2, which is a member of the neurexin family of synaptic cell surface proteins that may be involved in axon guidance. BAA76765.1, KIAA0921 (SEQ ID NO.: 760)

9. NRXN3: Neurexin 3, member of the neurexin family of synaptic cell surface proteins, a putative integral membrane protein which may have a role in axon guidance. NP_{—}004787.1 (SEQ ID NO.: 761)

10. TNFRSF18 (SEQ ID NO.: 753)

11. PTTG 1 (SEQ ID NO.: 762)

12. PARK2 (SEQ ID NO.: 744)

13. GABRG2: GABA-A receptor gamma 2 subunit, a chloride channel that is the major inhibitory neurotransmitter in the brain, subunit confers benzodiazepine binding to the receptor; variants are associated with epilepsy. NP_{—}000807.1 (SEQ ID NO.: 763)

14. CNTFR: Ciliary neurotrophic factor receptor, non-signaling alpha component of complex with gp130 (IL6ST) and leukemia inhibitory factor receptor (LIFR), regulates motor neuron survival in development and in patients with sporadic amyotrophic lateral sclerosis. NP_{—}001833.1 (SEQ ID NO.: 764)

15. CCR3: chemokine (C-C motif) receptor 3, Eotaxin receptor, G protein-coupled receptor that binds chemokines of the CC subfamily and mediates intracellular, calcium flux; target of human immunodeficiency virus. NP_{—}001828.1 (SEQ ID NO.: 765)

16. GABRG3: Alpha 3 subunit of the gamma-amino butyric acid A receptor, which is the major inhibitory neurotransmitter receptor in the brain and a chloride channel modulated by benzodiazepines; certain variants of GABRA3 are associated with multiple sclerosis. NP_{—}000799.1 (SEQ ID NO.: 766)

17. GABRP; Gamma-aminobutyric acid (GABA) type A receptor pi subunit, assembles with GABAA receptor subunits and alters sensitivity of receptors to modulatory agents, inhibits uterine contraction and maintains pregnancy. NP_{—}055026.1 (SEQ ID NO.: 767)

(14) huINADL PDZ2

Using methods described herein (for example, for ERBIN), the following gene products were identified as ligands for huINADL PDZ2:

1. PIWI1: Piwi (Drosophila)-like 1, a homolog of Drosophila piwi, plays a role in the control of cell proliferation and apoptosis, may be involved in hemopoiesis. AAK69348.1 (SEQ ID NO.: 768)

2. likely ortholog of mouse piwi like homolog 1: Protein with high similarity to PIWI (homolog of Drosophila piwi), which may be required for germ-line stem cell division, contains a Piwi domain. NP_{—}060538.1 (SEQ ID NO.: 769)

3. NRXN1 (SEQ ID NO.: 759)

4. NRXN2 (SEQ ID NO.: 760)

5. PPP2CA: Protein phosphatase 2 catalytic subunit alpha, a catalytic subunit of protein phosphatase 2A involved in regulating diverse cellular processes via protein phosphorylation. NP_{—}002706.1 (SEQ ID NO.: 770)

6. PPP2CB: Beta isoform of the catalytic subunit of protein phosphatase 2A, which is a major serine-threonine phosphatase thought to play a regulatory role in many cellular pathways. NP_{—}004147.1 (SEQ ID NO.: 771)

(15) huPARD3 PDZ3

Multi-PDZ protein that is essential for asymmetric cell division and polarized growth, may have a in the formation of tight junctions at epithelial cell-cell contacts. NP_{—}062565.1, PARD3

Using methods described herein (for example, for ERBIN), the following gene products were identified as ligands for huPARD3 PDZ3:

1. HRK: Harakiri, protein with a putative BH3 domain, interacts with and may inhibit the antiapoptotic activities of BCL2 and BCL-XL (BCL2L1), induces apoptosis; may play a role in apoptotic events in amyotrophic lateral sclerosis (ALS) patients. NP_{—}003797.1 (SEQ ID NO.: 772)

2. DOC1: Downregulated in ovarian cancer 1, a putative protein expressed by normal ovarian surface epithelial cells but not by ovarian cancer cell lines. NP_{—}055705.1 (SEQ ID NO.: 773)

3. PIWI (SEQ ID NO.: 768)

4. PPP1R3D: Phosphorylation of serine and threonine residues in proteins is a crucial step in the regulation of many cellular functions ranging from hormonal regulation to cell division and even short-term memory. The level of phosphorylation is controlled by the opposing actions of protein kinases and protein phosphatases. Protein phosphatase 1 (PP 1) is 1 of 4 major serine/threonine-specific protein phospha. NP_{—}006233.1 (SEQ ID NO.: 774)

(16) SNTA1 PDZ

Alpha 1 syntrophin, a member of the family of dystrophin associated proteins, interacts with components of the dystrophin-associated glycoprotein complex at the sarcolemma. NP_{—}003089.1

Using methods described herein (for example, for ERBIN), the following gene products were identified as ligands for SNTA1 PDZ:

1. MRGX2 MASI-related G protein-coupled receptor X2, a putative G protein-coupled receptor resembling MASI. NP_{—}473371.1 (SEQ ID NO.: 775)

2. NLGN1: Neuroligin 1, protein with very strong similarity to rat Nlgn1 (neuroligin I), which is a neuronal cell surface protein that acts as a ligand for specific splice forms of the neuronal cell surface receptor beta-neurexin. NP_{—}055747.1 (SEQ ID NO.: 776)

3. NLGN3; Neuroligin, member of a expressed outside the CNS. NP_{—}061850.1 (SEQ ID NO.: 777)

4. SEEK1: Protein possibly associated with psoriasis vulgaris. NP_{—}054787.1 (SEQ ID NO.: 778)

5. Claudin17 (SEQ ID NO.: 719)

6. GPR56: (SEQ ID NO.: 779)

7. SSTR5: Somatostatin receptor 5, a G protein-coupled receptor that suppresses adenylyl cyclase activity, mediates the inhibitory effects of somatostatin on cell proliferation and secretion of pituitary growth hormone and pancreatic insulin. NP 001044.1 (SEQ ID NO.: 780)

8. SCTR; Secretin receptor, a class II G protein-coupled receptor that can couple the cAMP and phosphatisylinositol intracellular signaling pathways and is involved in the control of water, bicarbonate and enzyme secretion in pancreas, gall bladder and stomach. NP 002971.1 (SEQ ID NO.: 781)

9. GRM 1; Metabotropic glutamate receptor 1 alpha, G protein coupled neurotransmitter receptor that promotes phosphoinositide hydrolysis and regulates intracellular calcium flux and membrane potential. NP_{—}000829.1 (SEQ ID NO.: 782)

10. GRM2; Metabotropic glutamate receptor 2, a neurotransmitter receptor that is coupled to an inhibitory G-protein. NP_{—}000830.1 (SEQ ID NO.: 783)

11. GRM3: Metabotropic glutamate receptor type 3, a neurotransmitter receptor that is coupled to an inhibitory G-protein, expressed in brain. NP_{—}000831.1 (SEQ ID NO.: 784)

12. GRM5; Metabotropic glutamate receptor 5, a G protein-coupled neurotransmitter receptor that activates phospholipase C and calcium-induced chloride channels, may regulate synaptic transmission and pain perception, possible association with schizophrenia. NP_{—}000833.1 (SEQ ID NO.: 785)

(17) Magi3 PDZ0

Using methods described herein (for example, for ERBIN), the following gene products were identified as ligands for Magi3 PDZ0:

1. LANO: LAP and no PDZ domain, a cell protein which binds to the PDZ domain of MAGUK proteins and indirectly binds Erbin (ERBB21P), may participate in epithelial tissue homeostasis. NP_{—}079444.1 (SEQ ID NO.: 786)

2. SSTR3; Somatostatin receptor 3, a G protein-coupled receptor that inhibits adenylyl cyclase activity and mediates the inhibitory effects of somatostatin on cell proliferation. The protein encoded by this gene is a GTPase which belongs to the RAS superfamily of small GTP-binding proteins. Members of this superfamily appear to regulate a diverse array of cellular events, including the control of cell growth, cytoskeletal reorganization, and the activation of protein kinases. Somatostatin acts at many sites to inhibit the release of many hormones and other secretory proteins. The biological effects of somatostatin are probably mediated by a family of G protein-coupled receptors that are expressed in a tissue-specific manner. SSTR3 is a member of the superfamily of receptors having seven transmembrane segments and is expressed in highest levels in brain and pancreatic. NP_{—}001042.1 (SEQ ID NO.: 787)

3. NRCAM: Neuronal cell adhesion molecule, a member of the immunoglobulin superfamily, predicted to have a role in neuronal cell adhesion. NP_{—}005001.1 (SEQ ID NO.: 788)

4. GPRI 9: Member of the G protein-coupled receptor family, expressed in brain and peripheral tissues. NP_{—}006134.1 (SEQ ID NO.: 789)

5. GNG5: G-protein gamma 5 subunit, plays a role in the trafficking of heterotrimeric G protein complexes to the cell membrane as a result of geranylgeranylation. NP_{—}005265.1 (SEQ ID NO.: 790)

6. HTR2B (SEQ ID NO.: 715)

(18) MUPP PDZ13

Using methods described herein (for example, for ERBIN), the following gene products were identified as ligands for MUPP PDZ 13:

1. NLGN3 (SEQ ID NO.: 777)

2. NLGN 1 (SEQ ID NO.: 776)

3. Claudin 16 (Paracellin-1), a renal tightjunction protein involved in paracellular Mg2+ and Ca2+resorption in thethick ascending limb of Henle; mutation of the corresponding gene is associated with hypomagnesemia hypercalciuria syndrome. NP_{—}006571.1 (SEQ ID NO.: 791)

4. GPR56 (SEQ ID NO.: 779)

5. Enigma: (LIM mineralization protein 1), a LIM domain-containing protein that binds to various receptor proteins including the insulin receptor (INSR), and plays a role in cell proliferation. NP_{—}005442.2 (SEQ ID NO.: 792)

6. FZD9: Frizzled 9, a seven-transmembrane receptor that binds Wnt1 proteins, implicated in tissue polarity, may be involved in neurogensis; corresponding gene is deleted in patients with Williams Beuren syndrome. NP_{—}003499.1 (SEQ ID NO.: 793)

7. SSTR5: Somatostatin receptor 5, a G protein-coupled receptor that suppresses adenylyl cyclase activity, mediates the inhibitory effects of somatostatin on cell proliferation and secretion of pituitary growth hormone and pancreatic insulin. Somatostatin acts at many sites to inhibit the release of many hormones and other secretory proteins. The biological effects of somatostatin are probably mediated by a family of G protein-coupled receptors that are expressed in a tissue-specific manner. SSTR5 is a member of the superfamily of receptors having seven transmembrane segments. NP_{—}001044.1 (SEQ ID NO.: 794)

8. VCAM1: Vascular cell adhesion molecule 1, an immunoglobulin superfamily member that mediates recruitment and adhesion of specific leukocytes to endothelial cells during the inflammatory response and may have a role in atherosclerosis. NP_{—}001069.1 (SEQ ID NO.: 795)

9. GPRK6; G protein-coupled receptor kinase 6, a protein kinase that regulates desensitization of G protein-coupled receptors by phosphorylating agonist-stimulated receptors. NP_{—}002073.1 (SEQ ID NO.: 796)

The utility of the peptides selected against the ERBIN PDZ domain and against other PDZ domains described above and herein is at least three fold. First they serve to identify the protein ligands for a given PDZ domain by the sequence information contained within them, e.g. identification of ARVCF, p0071 and δ catenin as ligands of the ERBIN PDZ domain. Identification of cognate ligands for individual PDZ domains (and thus the proteins containing these domains) using methods of the invention points to biologically important PDZ domain-cognate ligand interactions that are hitherto unknown. The biological functions of these interactions are evident from the known biology of the cognate ligands and PDZ domain proteins, as discussed above. Thus, identification of these novel interactions points to avenues of therapeutic and/or diagnostic applications and strategies that would not be possible in the absence of knowledge of such interactions. Secondly, peptides can be delivered into live cells, via microinjection, antenapedia peptide or lipid transfection reagents, to serve as PDZ domain specific competitive inhibitors in order to validate the physiological relevance of a PDZ ligand interaction. Suitable assays exist to monitor the PDZ ligand interaction. This does not require that the physiological ligand for a PDZ domain is discovered by phage display, only that the ligand is specific for that PDZ domain and of sufficient affinity to disrupt the interaction of said ligand with the PDZ domain. Finally, as with any protein linked with a disease process, one must establish how a drug should affect the protein to achieve therapeutic benefit. Pepties/ligands may be delivered into live cells or animal models which are models for a disease (i.e. mimic certain properties of a disease) to determine if disruption of a particular PDZ-ligand interaction provides an outcome consistent with expectations for therapeutic benefit.

Methods of detecting protein-protein (or peptide) interactions in vivo are known in the art. For example, the methods described by Michnick et al. in U.S. Pat. Nos. 6,270,964 B1 & 6,294,330 B1 can be used to analyze interactions of a PDZ domain-containing protein (including any described herein) and a cognate ligand or synthetic peptide (including any described herein). Furthermore, these methods can be used to assess the ability of a molecule, such as a synthetic peptide, to modulate the binding interaction of a PDZ-domain protein and its cognate ligand in vivo.

A. Definitions

Unless defined otherwise, all technical and scientific terms have the same meaning as is commonly understood by one of skill in the art to which this invention belongs. The definitions below are presented for clarity.

The recommendations of (Demerec et al., 1966) where these are relevant to genetics are adapted herein. To distinguish between genes (and related nucleic acids) and the proteins that they encode, the abbreviations for genes are indicated by italicized (or underlined) text while abbreviations for the proteins are not italicized. Thus, a PDBP is encoded by the nucleic acid sequence PDBP.

“Isolated,” when referred to a molecule, refers to a molecule that has been identified and separated and/or recovered from a component of its natural environment. Contaminant components of its natural environment are materials that interfere with diagnostic or therapeutic use.

1. Nucleic Acid-Related Definitions

(a) Control Sequences

Control sequences are DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

(b) Operably-Linked

Nucleic acid is operably-linked when it is placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous. Linking can be accomplished by conventional recombinant DNA methods.

(c) Isolated Nucleic Acids

An isolated nucleic acid molecule is purified from the setting in which it is found in nature and is separated from at least one contaminant nucleic acid molecule. Isolated PDZP, PDZD, PDBP or PIP molecules are distinguished from the specific PDZP, PDZD, PDBP or PIP molecules, as they exist in cells. However, an isolated PDZP, PDZD, PDBP or PIP molecule includes PDZP, PDZD, PDBP or PIP molecules contained in cells that ordinarily express PDZP, PDZD, PDBP or PIP, where, for example, the nucleic acid molecules are in a chromosomal location different from that of natural cells.

2. Protein-Related Definitions

(a) Purified Polypeptide

When the molecule is a purified polypeptide, the polypeptide will be purified (1) to obtain at least 3 residues of N-terminal or internal amino acid sequence using a sequenator, or (2) to homogeneity by SDS-PAGE under non-reducing or reducing conditions using Coomassie blue or silver stain. Isolated polypeptides include those expressed heterologously in genetically-engineered cells or expressed in vitro, since at least one component of a PDZP, PDZD, PDBP or PIP natural environment will not be present. Ordinarily, isolated polypeptides are prepared by at least one purification step.

(b) Active Polypeptide

An active PDZP, PDZD, PDBP or PIP, or fragments thereof, retains a biological and/or an immunological activity of native or naturally-occurring PDZP, PDZD, PDBP or PIP. Immunological activity refers to the ability to induce the production of an antibody against an antigenic epitope possessed by a native PDZP, PDZD, PDBP or PIP; biological activity refers to a function mediated by a native PDZP, PDZD, PDBP or PIP that excludes immunological activity. For example, a PIP binding to a cognate PDZP.

(c) Abs

Antibody may be single anti-PDZP, PDZD, PDBP or PIP monoclonal Abs (including agonist, antagonist, and neutralizing Abs), anti-PDZP, PDZD, PDBP or PIP antibody compositions with polyepitopic specificity, single chain anti-PDZP, PDZD, PDBP or PIP Abs, and fragments of anti-PDZP, PDZD, PDBP or PIP Abs. A “monoclonal antibody” refers to an antibody obtained from a population of substantially homogeneous Abs, i.e., the individual Abs comprising the population are identical except for naturally-occurring mutations that may be present in minor amounts

(d) Epitope Lags

An epitope tagged polypeptide refers to a chimeric polypeptide fused to a “tag polypeptide”. Such tags provide epitopes against which Abs can be made or are available, but do not interfere with polypeptide activity. To reduce anti-tag antibody reactivity with endogenous epitopes, the tag polypeptide is preferably unique. Suitable tag polypeptides generally have at least six amino acid residues, usually between about 8 and 50 amino acid residues, preferably between 8 and 20 amino acid residues. Examples of epitope tag sequences include HA from Influenza A virus, GD, and c-myc, poly-His and FLAG.

The PDBPs of the invention include the sequences provided in Tables 1 and 3. The invention also includes PDBP mutant or variant proteins, any of whose residues may be changed from the corresponding residue shown in Tables 1 and 3 while still encoding a protein that maintains its native activities and physiological functions, or a functional fragment.

PDZP, PDZD, PDBP or PIP Polynucleolides

One aspect of the invention pertains to isolated nucleic acid molecules that encode PDZPs, PDZDs, PDBPs or PIPs or biologically-active portions. A “nucleic acid molecule” includes DNA molecules (e.g., cDNA or genomic DNA), RNA molecules (e.g., mRNA), analogs of the DNA or RNA generated using nucleotide analogs, and derivatives, fragments and homologs. The nucleic acid molecule may be single-stranded or double-stranded, but preferably comprises double-stranded DNA.

A polynucleotide that encodes a PDZP, PDZD, PDBP or PIP can be deduced from the standard genetic code (Table C). Such sequences can be easily synthesized in vitro using standard techniques, or isolated from existing polynucleotides, such as those used in phage display.

TABLE C
Preferred Human DNA Codons

| | | | |
|---|---|---|---|
| | 3 letter | 1 letter | |
| Amino Acids | abbrev. | abbrev. | Codons |

| | | | | | | | | |
|---|---|---|---|---|---|---|---|---|
| Alanine | Ala | A | gcc | gct | gca | gcg | | |
| Cysteine | Cys | C | tgc | tgt | | | | |
| Aspartic acid | Asp | D | gac | gat | | | | |
| Glutamic acid | Glu | E | gag | gaa | | | | |
| Phenylalanine | Phe | F | ttc | ttt | | | | |
| Glycine | Gly | G | ggc | ggg | gga | ggt | | |
| Histidine | His | H | cac | cat | | | | |
| Isoleucine | Ile | I | atc | att | ata | | | |
| Lysine | Lys | K | aag | aaa | | | | |
| Leucine | Leu | L | ctg | ctc | ttg | ctt | cta | tta |
| Methionine | Met | M | atg | | | | | |
| Asparagine | Asn | N | aac | aat | | | | |
| Proline | Pro | P | ccc | cct | cca | ccg | | |
| Glutamine | Gln | Q | cag | caa | | | | |
| Arginine | Arg | R | cgc | agg | cgg | aga | cga | cgt |
| Serine | Ser | S | agc | tcc | tct | agt | tca | tcg |
| Threonine | Thr | T | acc | aca | act | acg | | |
| Valine | Val | V | gtg | gtc | gtt | gta | | |
| Tryptophan | Trp | W | tgg | | | | | |
| Tyrosine | Tyr | Y | tac | tat | | | | |

1. Isolated Nucleic Acid

An isolated nucleic acid molecule is separated from other nucleic acid molecules that are present in the natural source of the nucleic acid. An isolated nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule encoding PDZPs, PDZDs, PDBPs or PIPs, or a complement, can be isolated using standard molecular biology techniques and the provided sequence information or chemically synthesized (Ausubel et al., 1987; Sambrook, 1989).

PCR amplification techniques can be used to amplify PDZP, PDZD, PDBP or PIP using CDNA, mRNA or alternatively, genomic DNA, as a template and appropriate oligonucleotide primers. Such nucleic acids can be cloned into an appropriate vector and characterized by DNA sequence analysis. Furthermore, oligonucleotides corresponding to PDZP, PDZD, PIP or PDBP sequences can be prepared by standard synthetic techniques, e.g., an automated DNA synthesizer.

2. Oligonucleotide

An oligonucleotide comprises a series of linked nucleotide residues, which oligonucleotide has a sufficient number of nucleotide bases to be used in a PCR reaction or other application. A short oligonucleotide sequence may be based on, or designed from, a genomic or CDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, 100 or 150 nt in length, preferably about 15 nt to 30 nt in length. Oligonucleotides may be chemically synthesized and may also be used as probes.

3. Complementary Nucleic Acid Sequences; Binding

An isolated nucleic acid molecule of the invention comprises a nucleic acid molecule that is a complement of the nucleotide sequence encoding a PDZP, PDZD, PDBP or PIP, or a portion of this nucleotide sequence (e.g., a fragment that can be used as a probe or primer or a fragment encoding a biologically-active portion of a PIP or PDZP, such as a PDZD or PDBP). A nucleic acid molecule that is complementary to a PDZP, PDZD, PIP or PDBP-encoding nucleotide sequence is one that is sufficiently complementary to the nucleotide sequence to form hydrogen bonds with little or no mismatches to a PDZP, PDZD, PIP or PDBP-encoding nucleotide sequence, thereby forming a stable duplex.

“Complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

4. Conservative Mutations

Changes can be introduced by mutation into PDZP, PDZD, PIP or PDBP-encoding nucleic acids that incur alterations in the amino acid sequences of the encoded PDZP, PDZD, PIP or PDBP but that do not alter PDZP, PDZD, PIP or PDBP function. A “non-essential” amino acid residue is a residue that can be altered from the wild-type sequences of a PDZP, PDZD, PIP or PDBP without altering biological activity, whereas an “essential” amino acid residue is required for such biological activity. For example, amino acid residues that are conserved in a PDZP, PDZD, PIP or PDBP are predicted to be particularly non-amenable to alteration. Also see Examples. Amino acids for which conservative substitutions can be made are well known in the art.

Useful conservative substitutions are shown in Table D, “Preferred substitutions.” Conservative substitutions whereby an amino acid of one class is replaced with another amino acid of the same type fall within the scope of the invention so long as the substitution does not materially alter the biological activity of the compound. If such substitutions result in a change in biological activity, then more substantial changes, indicated in Table D as exemplary, are introduced and the products screened for PDZ domain binding.

TABLE D
Preferred substitutions

| Original | Exemplary | Preferred |
|---|---|---|
| residue | substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | Norleucine | |

Non-conservative substitutions that effect (1) the structure of the polypeptide backbone, such as a β-sheet or a-helical conformation, (2) the charge (3) hydrophobicity, or (4) the bulk of the side chain of the target site can modify PDZP, PDZD, PIP or PDBP function or immunological identity. Residues are divided into groups based on common side-chain properties as denoted in Table E. Non-conservative substitutions entail exchanging a member of one of these classes for another class. Substitutions may be introduced into conservative substitution sites or more preferably into non-conserved sites.

### Table E Amino Acid Classes

| Class | Amino acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant PDZPs, PDBPs, PIPs or PDZDs can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce a PDZP, PDZD, PIP orPDBP variant DNA (Ausubel et al., 1987; Sambrook, 1989).

5. Antisense Nucleic Acids

Antisense methods can be used to validate predicted interactions, i.e. antisense-induced loss of a predicted PDZ binding partner may alter the subcellular localization or activity of a protein.

Using antisense and sense PDZP, PDZD, PIP or PDBP oligonucleotides can prevent PDZP, PDZD, PIP or PDBP. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are single-stranded nucleic acids, either RNA or DNA, which can bind target PDZP, PDZD, PIP or PDBP mRNA (sense) or PDZP, PDZD, PIP or PDBP DNA (antisense) sequences. Antisense nucleic acids can be designed according to Watson and Crick or Hoogsteen base pairing rules. The antisense nucleic acid molecule can be complementary to the entire coding region of PDZP, PDZD, PIP or PDBP mRNA, but more preferably, to only a portion of the coding or noncoding region of PDZP, PDZD, PIP or PDBP mRNA. For example, the antisense oligonucleotide can be complementary to the region surrounding the translation start site of a PDZP, PDZD, PIP or PDBP mRNA. Antisense or sense oligonucleotides may comprise a fragment of a PDZP, PDZD, PIP or PDBP DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988b) describe methods to derive antisense or a sense oligonucleotides from a given CDNA sequence.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation such that the transcribed RNA will be complementary to a target nucleic acid of interest.

To introduce antisense or sense oligonucleotides into target cells (cells containing the target nucleic acid sequence), any gene transfer method may be used. Examples of gene transfer methods include (1) biological, such as gene transfer vectors like Epstein-Barr virus or conjugating the exogenous DNA to a ligand-binding molecule, (2) physical, such as electroporation and injection, and (3) chemical, such as CaPO₄ precipitation and oligonucleotide-lipid complexes.

An antisense or sense oligonucleotide can be inserted into a suitable gene transfer retroviral vector. A cell containing the target nucleic acid sequence is contacted with the recombinant retroviral vector, either in vivo or ex vivo. Examples of suitable retroviral vectors include those derived from the murine retrovirus M-MuLV, N2 (a retrovirus derived from M-MuLV), or the double copy vectors designated DCT5A, DCT5B and DCT5C (WO 90/13641, 1990). To achieve sufficient nucleic acid molecule transcription, vector constructs in which the transcription of the antisense nucleic acid molecule is controlled by a strong pol II or pol III promoter are preferred. Alternatively, inducible promoters may be preferred when the expression of the construct is desired to be controlled.

To specify target cells in a mixed population, cell surface receptors that are specific to the target cells can be exploited. Antisense and sense oligonucleotides are conjugated to a ligand-binding molecule, as described in (WO 91/04753, 1991). Ligands are chosen for receptors that are specific to the target cells. Examples of suitable ligand-binding molecules include cell surface receptors, growth factors, cytokines, or other ligands that bind to cell surface receptors or molecules. Preferably, conjugation of the ligand-binding molecule does not substantially interfere with the ability of the receptors or molecule to bind the ligand-binding molecule conjugate, or block entry of the sense or antisense oligonucleotide or its conjugated version into the cell.

Liposomes efficiently transfer sense or an antisense oligonucleotide to cells (WO 90/10448, 1990). The sense or antisense oligonucleotide-lipid complex is preferably dissociated within the cell by an endogenous lipase.

The antisense nucleic acid molecule may be an α-anomeric nucleic acid molecule. An α-anomeric nucleic acid molecule forms specific double-stranded hybrids with complementary RNA in which, contrary to the usual α-units, the strands run parallel to each other (Gautier et al., 1987). The antisense nucleic acid molecule can also comprise a 2′-o-methylribonucleotide (Inoue et al., 1987a) or a chimeric RNA-DNA analogue (Inoue et al., 1987b).

In one embodiment, an antisense nucleic acid is a ribozyme. Ribozymes are catalytic RNA molecules with ribonuclease activity that are capable of cleaving a single-stranded nucleic acid, such as an mRNA, to which they have a complementary region. Ribozymes, such as hammerhead ribozymes (Haseloff and Gerlach, 1988) can be used to catalytically cleave PDZP, PDZD, PIP or PDBP mRNA transcripts and thus inhibit translation. A ribozyme specific for aPDZP, PDZD, PIP or PDBP can be designed based on the nucleotide sequence of a PDZP, PDZD, PIP or PDBP cDNA. For example, a derivative of a Tetrahymena L-19 IVS RNA can be constructed in which the nucleotide sequence of the active site is complementary to the nucleotide sequence to be cleaved in a PDZP, PDZD, PIP or PDBP mRNA (Cech et al., U.S. Pat. No. 5,116,742, 1992; Cech et al., U.S. Pat. No. 4,987,071, 1991). PDZP, PDZD, PIP or PDBP mRNA can also be used to select a catalytic RNA having a specific ribonuclease activity from a pool of RNA molecules (Bartel and Szostak, 1993).

Alternatively, PDZP, PDZD, PIP or PDBP expression can be inhibited by targeting nucleotide sequences complementary to the regulatory region of a PDZP, PIP or PDBP (e.g., a PDZP, PIP or PDBPpromoter and/or enhancers) to form triple helical structures that prevent transcription of a PDZP, PDZD, PIP or PDBP in target cells (Helene, 1991; Helene et al., 1992; Maher, 1992).

Modifications of antisense and sense oligonucleotides can augment their effectiveness. Modified sugar-phosphodiester bonds or other sugar linkages (WO 91/06629, 1991), increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents.

For example, the deoxyribose phosphate backbone of the nucleic acids can be modified to generate peptide nucleic acids (Hyrup and Nielsen, 1996). “Peptide nucleic acids” or “PNAs” refer to nucleic acid mimics (e.g., DNA mimics) in that the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols (Hyrup and Nielsen, 1996; Perry-O'Keefe et al., 1996).

PNAs of PDZP, PDZD, PIP or PDBP can be used in therapeutic and diagnostic applications. For example, PNAs can be used as antisense or antigene agents for sequence-specific modulation of gene expression by inducing transcription or translation arrest or inhibiting replication. PDZP, PDZD, PIP or PDBP PNAs may also be used in the analysis of single base pair mutations (e.g., PNA directed PCR clamping; as artificial restriction enzymes when used in combination with other enzymes, e.g., S₁ nucleases (Hyrup and Nielsen, 1996); or as probes or primers for DNA sequence and hybridization (Hyrup and Nielsen, 1996; Perry-O'Keefe et al., 1996).

PNAs of PDZP, PDZD, PIP or PDBP can be modified to enhance their stability or cellular uptake. Lipophilic or other helper groups may be attached to PNAs, PNA-DNA dimers formed, or the use of liposomes or other drug delivery techniques. For example, PNA-DNA chimeras can be generated that may combine the advantageous properties of PNA and DNA. Such chimeras allow DNA recognition enzymes (e.g., RNase H and DNA polymerases) to interact with the DNA portion while the PNA portion provides high binding affinity and specificity. PNA-DNA chimeras can be linked using linkers of appropriate lengths selected in terms of base stacking, number of bonds between the nucleobases, and orientation (Hyrup and Nielsen, 1996). The synthesis of PNA-DNA chimeras can be performed (Finn et al., 1996; Hyrup and Nielsen, 1996). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Finn et al., 1996; Hyrup and Nielsen, 1996). PNA monomers are then coupled in a stepwise manner to produce a chimeric molecule with a 5′ PNA segment and a 3′ DNA segment (Finn et al., 1996). Alternatively, chimeric molecules can be synthesized with a 5′ DNA segment and a 3′ PNA segment (Petersen et al., 1976).

The oligonucleotide may include other appended groups such as peptides (e.g., for targeting host cell receptors in vivo), or agents facilitating transport across the cell membrane (Lemaitre et al., 1987; Letsinger et al., 1989; Tullis, U.S. Pat. No. 4,904,582, 1988) or the blood-brain barrier (e.g., (Pardridge and Schimmel, WO89/10134, 1989)). In addition, oligonucleotides can be modified with hybridization-triggered cleavage agents (van der Krol et al., 1988a) or intercalating agents (Zon, 1988). The oligonucleotide may be conjugated to another molecule, e.g., a peptide, a hybridization triggered cross-linking agent, a transport agent, a hybridization-triggered cleavage agent, and the like.

PDZP, PDZD, PIP or PDBP Peptides/Polypeptides

One aspect of the invention pertains to isolated PDZP, PDZD, PIP or PDBP, and biologically active portions derivatives, fragments, analogs or homologs thereof. Also provided are polypeptide fragments suitable for use as immunogens to raise anti-PDZP, PDZD, PIP or PDBP Abs. In one embodiment, native PDZP or PIP can be isolated from cells or tissue sources by an appropriate purification scheme using standard protein purification techniques. In another embodiment, PDZPs, PDZDs, PIPs or PDBPs are produced by recombinant DNA techniques. Alternative to recombinant expression, a PDZP, PDZD, PIP or PDBP can be synthesized chemically using standard peptide synthesis techniques.

1. Peptides/Polypeptides

A PDBP or PIP peptide includes the amino acid sequence provided in SEQ ID NOs:1-163. The invention also includes a mutant or variant protein any of which residues may be changed from the corresponding residues shown in SEQ ID NOs:1-163, while still encoding a protein that maintains PDBP or PIP activities and physiological functions, or a functional fragment thereof.

2. Variant PDZP, PDZD, PIP or PDBP Peptides/Polypeptides

In general, a PDZP, PDZD, PIP or PDBP variant that preserves PDZP, PDZD, PIP or PDBP-like function and includes any variant in which residues at a particular position in the sequence have been substituted by other amino acids, and further includes the possibility of inserting an additional residue or residues between two residues of the parent protein as well as the possibility of deleting one or more residues from the parent sequence or adding one or more residues to the parent sequence. Any amino acid substitution, insertion, or deletion is encompassed by the invention. In favorable circumstances, the substitution is a conservative substitution as previously defined.

“Percent (%) amino acid sequence identity” is defined as the percentage of amino acid residues that are identical with amino acid residues in a candidate sequence in a disclosed PDZP, PDZD, PIP or PDBP polypeptide sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y·100

where

X is the number of amino acid residues scored as identical matches by the sequence alignment program's or algorithm's alignment of A and B and

Y is the total number of amino acid residues in B.

If the length of amino acid sequence A is not equal to the length of amino acid sequence B, the % amino acid sequence identity of A to B will not equal the % amino acid sequence identity of B to A.

3. Isolated/Purified Peptides and Polypeptides

An “isolated” or “purified” peptide, polypeptide, protein or biologically active fragment is separated and/or recovered from a component of its natural environment. Contaminant components include materials that would typically interfere with diagnostic or therapeutic uses for the polypeptide, and may include enzymes, hormones, and other proteinaceous or non-proteinaceous materials. To be substantially isolated, preparations having less than 30% by dry weight of non-PDZP, PDZD, PIP or PDBP contaminating material (contaminants), more preferably less than 20%, 10% and most preferably less than 5% contaminants. An isolated, recombinantly-produced PDZP, PDZD, PIP or PDBP or biologically active portion is preferably substantially free of culture medium, i.e., culture medium represents less than 20%, more preferably less than about 10%, and most preferably less than about 5% of the volume of a PDZP, PDZD, PIP or PDBP preparation. Examples of contaminants include cell debris, culture media, and substances used and produced during in vitro synthesis of PDZP, PDZD, PIP or PDBP.

4. Biologically Active

Biologically active portions of PDZP, PDZD, PIP or PDBP exhibit at least one activity of a PDZP, PDZD, PIP or PDBP, such as PDZ interactions.

Biologically active portions of a PDBP may have an amino acid sequence shown in SEQ ID NOs:1-163, or substantially homologous to SEQ ID NOs:1-163, and retains the functional activity of the protein of SEQ ID NOs:1-163, yet differs in amino acid sequence due to natural allelic variation or mutagenesis.

5. Chimeric and Fusion Proteins

Fusion polypeptides are useful in expression studies, cell-localization, bioassays, and PDZP, PDZD, PIP or PDBP purification. A PDZP, PDZD, PIP or PDBP “chimeric protein” or “fusion protein” comprises PDZP, PDZD, PIP or PDBP fused to a non-PDZP, PDZD, PIP or PDBP polypeptide. PDZP, PDZD, PIP or PDBP may be fused to the C-terminus of the GST (glutathione S-transferase) sequences. Such fusion proteins facilitate the purification of recombinant PDZP, PDZD, PIP or PDBP. Additional exemplary fusions are presented in Table A above.

Other fusion partners can adapt PDZPs, PDZDs, PIPs or PDBPs therapeutically. Fusions with members of the immunoglobulin (Ig) protein family are useful in therapies that inhibit PDZ interactions, consequently suppressing PDZ-mediated signal transduction in vivo. PDZP, PDZD, PIP or PDBP-Ig fusion polypeptides can also be used as immunogens to produce anti-PDZP, PDZD, PIP or PDBP Abs in a subject and to screen for molecules that inhibit PDZ binding interactions.

Fusion proteins can be easily created using recombinant methods. A nucleic acid encoding PDZP, PDZD, PIP or PDBP can be fused in-frame with a non-PDZP, PDZD, PIP or PDBP-encoding nucleic acid, to a PDZP, PDZD, PIP or PDBP NH₂— or COO— -terminus, or internally. Fusion genes may also be synthesized by conventional techniques, including automated DNA synthesizers. PCR amplification using anchor primers that give rise to complementary overhangs between two consecutive gene fragments that can subsequently be annealed and reamplified to generate a chimeric gene sequence (Ausubel et al., 1987) is also useful. Many vectors are commercially available that facilitate sub-cloning PDZP, PDZD, PIP or PDBP in-frame to a fusion moiety.

Therapeutic applications ofPDZPs, PDZDs, PIPs and PDBPs

Altering the expression of PDZP, PDZD, PIP or PDBP in a mammal, such as a human, through gene therapy may be effective to combat diseases.

Compounds that have the property of increasing or decreasing PDZP, PDZD, PIP or PDBP activity are useful. This increase in activity may come about in a variety of ways, for example: (1) by increasing or decreasing the copies of the gene in the cell (amplifiers and deamplifiers); (2) by increasing or decreasing transcription of a PDZP, PDZD, PIP or PDBP-containing gene (transcription up-regulators and down-regulators); (3) by increasing or decreasing the translation of PDZP, PDZD, PIP or PDBP-containing mRNA into protein (translation up-regulators and down-regulators); or (4) by increasing or decreasing the activity of PDZP, PDZD, PIP or PDBP itself (agonists and antagonists).

Contacting cells or organisms with the compound may identify compounds that are amplifiers and deamplifiers, and then measuring the amount of DNA present that encodes a PDZP, PDZD, PIP or PDBP (Ausubel et al., 1987). Contacting cells or organisms with the compound may identify compounds that are transcription up-regulators and down-regulators, and then measuring the amount of MRNA produced that encodes PDZP, PDZD, PIP or PDBP (Ausubel et al., 1987). Compounds that are translation up-regulators and down-regulators may be identified by contacting cells or organisms with the compound, and then measuring the amount of PDZP, PDZD, PIP or PDBP polypeptide produced (Ausubel et al., 1987).

Compounds that are amplifiers, transcription up-regulators, translation up-regulators or agonists, are effective to combat diseases that can be ameliorated by decreasing PDZP, PDZD, PIP or PDBP activity. Conversely, compounds that are deamplifiers, transcription down-regulators, translation down-regulators or antagonists, are effective to combat diseases that can be ameliorated by increasing PDZP, PDZD, PIP or PDBP activity. Gene therapy is another way to up-regulate or down-regulate transcription and/or translation.

Both PDZP, PDZD, PIP or PDBP peptides/polypeptides and polynucleotides can be used in clinical screens to test for disease etiology or to assess the level of risk for these disorders. Tissue samples of a patient can be examined for the amount of PDZP, PDZD, PIP or PDBP protein or mRNA. When amounts significantly smaller or larger than normal are found, they are indicative of disease or risk of disease. Mutation of PDZP, specifically a PDZD or a PIP, specifically a PDBP, can yield altered activity, and a patient with such a mutation may have a disease or be at risk for a disease. Finally, determining the amount of expression of PDZP, PDZD, PIP or PDBP in a mammal, in a tissue sample, or in a tissue culture, can be used to discover inducers or repressors of the gene.

Determination of PDZP, PDZD, PIP or PDBP mRNA, proteins or activity levels in clinical samples may have predictive value for tracking progression of disorders, or in cases in which therapeutic modalities are applied to correct disorders.

1. Agonists and Antagonists

“Antagonist” includes any molecule that partially or fully blocks, inhibits, or neutralizes a biological activity of endogenous PDZP, PDZD, PIP or PDBP, such as binding a PDZ domain. Similarly, “agonist” includes any molecule that mimics or enhances a biological activity of endogenous PDZPs or PIPs. Molecules that can act as agonists or antagonists include Abs or antibody fragments, fragments or variants of endogenous PDZPs or PIPs, or PDBPs, PDZDs, peptides, antisense oligonucleotides, small organic molecules, and other PDLs.

2. Identifying Antagonists and Agonists

(a) Specific Examples of Potential Antagonists and Agonist

Any molecule that alters PDZP or PIP cellular effects is a candidate antagonist or agonist. Screening techniques well known to those skilled in the art can identify these molecules. Examples of antagonists and agonists include: (1) small organic and inorganic compounds, (2) small peptides, (3) Abs and derivatives, (4) polypeptides closely related to PDZP, PDZD, PIP or PDBP, (5) antisense DNA and RNA, (6) ribozymes, (7) triple DNA helices and (8) nucleic acid aptamers.

Small molecules that bind to a PDZP or PIP active site (e.g., the PDZD of a PDZP) and inhibit the biological activity of a PDZP, are antagonists. Examples of small molecule antagonists include small peptides, peptide-like molecules, preferably soluble, synthetic non-peptidyl organic or inorganic compounds and other PDLs. These same molecules, if they enhance a PDZP or PIP activity, are examples of agonists.

Almost any antibody that affects PDZP, PDZD, PIP or PDBP function is a candidate antagonist, and occasionally, agonist. Examples of antibody antagonists include polyclonal, monoclonal, single-chain, anti-idiotypic, chimeric Abs, or humanized versions of such Abs or fragments. Abs may be from any species in which an immune response can be raised. Humanized Abs are also contemplated.

Alternatively, a potential antagonist or agonist may be a closely related protein, for example, a PDZD or PDBP. Alternatively, a mutated PDZP, PDZD, PIP or PDBP may result in an interaction that is non-reversible and may act as angonist.

Antisense RNA or DNA constructs can be effective antagonists. Antisense RNA or DNA molecules block function by inhibiting translation by hybridizing to targeted mRNA. Antisense technology can be used to control gene expression through triple-helix formation or antisense DNA or RNA, both of which depend on polynucleotide binding to DNA or RNA. For example, the 5′ coding portion of a PDZP, PDZD, PIP or PDBP sequence is used to design an antisense RNA oligonucleotide of from about 10 to 40 base pairs in length. A DNA oligonucleotide is designed to be complementary to a region of the gene involved in transcription (triple helix) (Beal and Dervan, 1991; Cooney et al., 1988; Lee et al., 1979), thereby preventing transcription and the production of a PDZP, PDZD, PIP or PDBP. The antisense RNA oligonucleotide hybridizes to the mRNA in vivo and blocks translation of the mRNA molecule into a PDZP, PDZD, PIP or PDBP (antisense) (Cohen, 1989; Okano et al., 1991). These oligonucleotides can also be delivered to cells such that the antisense RNA or DNA may be expressed in vivo to inhibit production of a PDZP, PDZD, PIP or PDBP. When antisense DNA is used, oligodeoxyribonucleotides derived from the translation-initiation site, e.g., between about −10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques (WO 97/33551, 1997; Rossi, 1994).

To inhibit transcription, triple-helix nucleic acids that are single-stranded and comprise deoxynucleotides are useful antagonists. These oligonucleotides are designed such that triple-helix formation via Hoogsteen base-pairing rules is promoted, generally requiring stretches of purines or pyrimidines (WO 97/33551, 1997).

Aptamers are short oligonucleotide sequences that can be used to recognize and specifically bind almost any molecule. The systematic evolution of ligands by exponential enrichment (SELEX) process (Ausubel et al., 1987; Ellington and Szostak, 1990; Tuerk and Gold, 1990) can be used to find such aptamers. Aptamers have many diagnostic and clinical uses; almost any use in which an antibody has been used clinically or diagnostically, aptamers too may be used. In addition, aptamers are less expensive to manufacture once they have been identified and can be easily applied in a variety of formats, including administration in pharmaceutical compositions, bioassays and diagnostic tests (Jayasena, 1999).

Anti-PDZP, PDZD, PIP or PDBP Abs

The invention encompasses Abs and antibody fragments, such as Fab or (Fab)2, that bind immunospecifically to any PDZP, PDZD, PIP or PDBP epitopes.

“Antibody” (Ab) comprises single Abs directed against PDZP, PDZD, PIP or PDBP (anti-PDZP, PDZD, PIP or PDBP Ab; including agonist, antagonist, and neutralizing Abs), anti-PDZP, PDZD, PIP or PDBP Ab compositions with poly-epitope specificity, single chain anti-PDZP, PDZD, PIP or PDBP Abs, and fragments of anti-PDZP, PDZD, PIP or PDBPAbs. A “monoclonal antibody” is obtained from a population of substantially homogeneous Abs, i.e., the individual Abs comprising the population are identical except for possible naturally-occurring mutations that may be present in minor amounts. Exemplary Abs include polyclonal (pAb), monoclonal (mAb), humanized, bi-specific (bsAb), and heteroconjugate Abs.

1. Polyclonal Abs (pAbs)

Polyclonal Abs can be raised in a mammalian host, for example, by one or more injections of an immunogen and, if desired, an adjuvant. Typically, the immunogen and/or adjuvant are injected in the mammal by multiple subcutaneous or intraperitoneal injections. The immunogen may include PDZP, PDZD, PIP or PDBP or a fusion protein. Examples of adjuvants include Freund's complete and monophosphoryl Lipid A synthetic-trehalose dicorynomycolate (MPL-TDM). To improve the immune response, an immunogen may be conjugated to a protein that is immunogenic in the host, such as keyhole limpet hemocyanin (KLH), serum albumin, bovine thyroglobulin, and soybean trypsin inhibitor. Protocols for antibody production are described (Ausubel et al., 1987; Harlow and Lane, 1988). Alternatively, pAbs may be made in chickens, producing IgY molecules (Schade et al., 1996).

2. Monoclonal Abs (mA bs)

Anti-PDZP, PDZD, PIP or PDBP mAbs may be prepared using hybridoma methods (Milstein and Cuello, 1983). Hybridoma methods comprise at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-PDZP, PDZD, PIP or PDBP) mAb.

A mouse, rat, guinea pig, hamster, or other appropriate host is immunized to elicit lymphocytes that produce or are capable of producing Abs that will specifically bind to the immunogen. Alternatively, the lymphocytes may be immunized in vitro. If human cells are desired, peripheral blood lymphocytes (PBLs) are generally used; however, spleen cells or lymphocytes from other mammalian sources are preferred. The immunogen typically includes PDZP, PDZD, PIP or PDBP or a fusion protein thereof.

The lymphocytes are then fused with an immortalized cell line to form hybridoma cells, facilitated by a fusing agent such as polyethylene glycol (Goding, 1996). Rodent, bovine, or human myeloma cells immortalized by transformation may be used, or rat or mouse myeloma cell lines. Because pure populations of hybridoma cells and not unfused immortalized cells are preferred, the cells after fusion are grown in a suitable medium that contains one or more substances that inhibit the growth or survival of unfused, immortalized cells. A common technique uses parental cells that lack the enzyme hypoxanthine guanine phosphoribosyl transferase (HGPRT or HPRT). In this case, hypoxanthine, aminopterin and thymidine are added to the medium (HAT medium) to prevent the growth of HGPRT-deficient cells while permitting hybridomas to grow.

Preferred immortalized cells fuse efficiently; can be isolated from mixed populations by selecting in a medium such as HAT; and support stable and high-level expression of antibody after fusion. Preferred immortalized cell lines are murine myeloma lines, available from the American Type Culture Collection (Manassas, Va.). Human myeloma and mouse-human heteromyeloma cell lines also have been described for the production of human mAbs (Kozbor et al., 1984; Schook, 1987).

Because hybridoma cells secrete antibody extracellularly, the culture media can be assayed for the presence of mAbs directed against PDZP, PDZD, PIP or PDBP (anti-PDZP, PDZD, PIP or PDBP mAbs). Immunoprecipitation or in vitro binding assays, such as radio immunoassay (RIA) or enzyme-linked immunoabsorbent assay (ELISA), measure the binding specificity of mAbs (Harlow and Lane, 1988; Harlow and Lane, 1999), including Scatchard analysis (Munson and Rodbard, 1980).

Anti-PDZP, PDZD, PIP or PDBP mAb secreting hybridoma cells may be isolated as single clones by limiting dilution procedures and sub-cultured (Goding, 1996). Suitable culture media include Dulbecco's Modified Eagle's Medium, RPMI-1640, or if desired, a protein-free or -reduced or serum-free medium (e.g., Ultra DOMA PF or HL-1; Biowhittaker; Walkersville, Md.). The hybridoma cells may also be grown in vivo as ascites.

The mAbs may be isolated or purified from the culture medium or ascites fluid by conventional Ig purification procedures such as protein A-Sepharose, hydroxylapatite chromatography, gel electrophoresis, dialysis, ammonium sulfate precipitation or affinity chromatography (Harlow and Lane, 1988; Harlow and Lane, 1999).

The mAbs may also'be made by recombinant methods (U.S. Pat. No. 4,166,452, 1979). DNA encoding anti-PDZP, PDZD, PIP or PDBP mAbs can be readily isolated and sequenced using conventional procedures, e.g., using oligonucleotide probes that specifically bind to murine heavy and light antibody chain genes, to probe preferably DNA isolated from anti-PDZP, PDZD, PIP or PDBP-secreting mAb hybridoma cell lines. Once isolated, the isolated DNA fragments are sub-cloned into expression vectors that are then transfected into host cells such as simian COS-7 cells, Chinese hamster ovary (CHO) cells, or myeloma cells that do not otherwise produce Ig protein, to express mAbs. The isolated DNA fragments can be modified, for example, by substituting the coding sequence for human heavy and light chain constant domains in place of the homologous murine sequences (U.S. Pat. No. 4,816,567, 1989; Morrison et al., 1987), or by fusing the Ig coding sequence to all or part of the coding sequence for a non-Ig polypeptide. Such a non-Ig polypeptide can be substituted for the constant domains of an antibody, or can be substituted for the variable domains of one antigen-combining site to create a chimeric bivalent antibody.

3. Monovalent Abs

The Abs may be monovalent Abs that consequently do not cross-link with each other. For example, one method involves recombinant expression of Ig light chain and modified heavy chain. Heavy chain truncations at any point in the F_{c} region will prevent heavy chain cross-linking. Alternatively, the relevant cysteine residues are substituted with another amino acid residue or are deleted, preventing crosslinking. In vitro methods are also suitable for preparing monovalent Abs. Abs can be digested to produce fragments, such as Fab fragments (Harlow and Lane, 1988; Harlow and Lane, 1999) that will not cross-link.

4. Humanized and Human Abs

Anti-PDZP, PDZD, PIP or PDBP Abs may further comprise humanized or human Abs. Humanized forms of non-human Abs are chimeric Igs, Ig chains or fragments (such as Fᵥ, F_{ab}, F_{ab′}, F_{(ab′)2} or other antigen-binding subsequences of Abs) that contain minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., 1986; Riechmann et al., 1988; Verhoeyen et al., 1988). Such “humanized” Abs are chimeric Abs (U.S. Pat. No. 4,816,567, 1989), wherein substantially less than an intact human variable domain has been substituted by the corresponding sequence from a non-human species. In practice, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues of the human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (F_{c}), typically that of a human Ig (Jones et al., 1986; Presta, 1992; Riechmann et al., 1988).

Human Abs can also be produced using various techniques, including phage display libraries (Hoogenboom et al., 1991; Marks et al., 1991b) and the preparation of human mAbs (Boerner et al., 1991; Reisfeld and Sell, 1985). Similarly, introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire (U.S. Pat. No. 5,545,807, 1996; U.S. Pat. No. 5,545,806, 1996; U.S. Pat. No. 5,569,825, 1996; U.S. Pat. No. 5,633,425, 1997; U.S. Pat. No. 5,661,016, 1997; U.S. Pat. No. 5,625,126, 1997; Fishwild et al., 1996; Lonberg and Huszar, 1995; Lonberg et al., 1994; Marks et al., 1992).

5. Bi-Specific Mabs

Bi-specific Abs are monoclonal, preferably human or humanized, that have binding specificities for at least two different antigens. For example, a binding specificity is PDZP, PDZD, PIP or PDBP; the other is for any antigen of choice, preferably a cell-surface protein or receptor or receptor subunit.

Traditionally, the recombinant production of bi-specific Abs is based on the co-expression of two Ig heavy-chain/light-chain pairs, where the two heavy chains have different specificities (Milstein and Cuello, 1983). Because of the random assortment of Ig heavy and light chains, the resulting hybridomas (quadromas) produce a potential mixture of ten different antibody molecules, of which only one has the desired bi-specific structure. The desired antibody can be purified using affinity chromatography or other techniques (WO 93/08829, 1993; Traunecker et al., 1991).

To manufacture a bi-specific antibody (Suresh et al., 1986), variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. Nucleotide sequences encoding the Ig heavy-chain fusions and, if desired, the Ig light chain, are inserted into separate expression vectors and are co-transfected into a suitable host organism.

The interface between a pair of antibody molecules can be engineered to maximize the percentage of heterodimers that are recovered from recombinant cell culture (WO 96/27011, 1996). The preferred interface comprises at least part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This mechanism increases the yield of the heterodimer over unwanted end products such as homodimers.

Bi-specific Abs can be prepared as full length Abs or antibody fragments (e.g. F_{(ab′)2} bi-specific Abs). One technique to generate bi-specific Abs exploits chemical linkage. Intact Abs can be proteolytically cleaved to generate F_{(ab′)2} fragments (Brennan et al., 1985). Fragments are reduced with a dithiol complexing agent, such as sodium arsenite, to stabilize vicinal dithiols and prevent intermolecular disulfide formation. The generated F_{ab′} fragments are then converted to thionitrobenzoate (TNB) derivatives. One of the F_{ab′}-TNB derivatives is then reconverted to the F_{ab′}-thiol by reduction with mercaptoethylamine and is mixed with an equimolar amount of the other F_{ab′}-TNB derivative to form the bi-specific antibody. The produced bi-specific Abs can be used as agents for the selective immobilization of enzymes.

F_{ab′} fragments may be directly recovered from E. coli and chemically coupled to form bi-specific Abs. For example, fully humanized bi-specific F_{(ab′)2} Abs can be produced (Shalaby et al., 1992). Each F_{ab′} fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited (Kostelny et al., 1992). Peptides from the Fos and Jun proteins are linked to the F_{ab′} portions of two different Abs by gene fusion. The antibody homodimers are reduced at the hinge region to form monomers and then re-oxidized to form antibody heterodimers. This method can also produce antibody homodimers. The “diabody” technology (Holliger et al., 1993) provides an alternative method to generate bi-specific antibody fragments. The fragments comprise a heavy-chain variable domain (V_{H}) connected to a light-chain variable domain (V_{L}) by a linker that is too short to allow pairing between the two domains on the same chain. The V_{H} and V_{L} domains of one fragment are forced to pair with the complementary V_{L} and V_{H} domains of another fragment, forming two antigen-binding sites. Another strategy for making bi-specific antibody fragments is the use of single-chain Fᵥ (sFᵥ) dimers (Gruber et al., 1994). Abs with more than two valencies are also contemplated, such as tri-specific Abs (Tutt et al., 1991).

Exemplary bi-specific Abs may bind to two different epitopes on a given PDZP, PDZD, PIP or PDBP. Alternatively, cellular defense mechanisms can be restricted to a particular cell expressing the particular PDZP, PDZD, PIP or PDBP: an anti-PDZP, PDZD, PIP or PDBP arm may be combined with an arm that binds to a leukocyte triggering molecule, such as a T-cell receptor molecule (e.g. CD2, CD3, CD28, or B7), or to Fc receptors for IgG (F_{c}γR), such as F_{c}γRI (CD64), F_{c}γRII (CD32) and F_{c}γRIII (CD16). Bi-specific Abs may also be used to target cytotoxic agents to cells that express a particular PDZP, PDZD, PIP or PDBP. These Abs possess a PDZP, PDZD, PIP or PDBP-binding arm and an arm that binds a cytotoxic agent or a radionuclide chelator.

6. Heteroconjugate Abs

Heteroconjugate Abs, consisting of two covalently joined Abs, have been proposed to target immune system cells to unwanted cells (U.S. Pat. No. 4,676,980, 1987) and for treatment of human immunodeficiency virus (HIV) infection (WO 91/00360, 1991; WO 92/20373, 1992). Abs prepared in vitro using synthetic protein chemistry methods, including those involving cross-linking agents, are contemplated. For example, immunotoxins may be constructed using a disulfide exchange reaction or by forming a thioether bond. Examples of suitable reagents include iminothiolate and methyl-4-mercaptobutyrimidate (U.S. Pat. No. 4,676,980, 1987).

7. Immunoconjugates

Immunoconjugates may comprise an antibody conjugated to a cytotoxic agent such as a chemotherapeutic agent, toxin (e.g., an enzymatically active toxin or fragment of bacterial, fungal, plant, or animal origin), or a radioactive isotope (i.e., a radioconjugate).

Useful enzymatically-active toxins and fragments include Diphtheria A chain, non-binding active fragments of Diphtheria toxin, exotoxin A chain from Pseudomonas aeruginosa, ricin A chain, abrin A chain, modeccin A chain, α-sarcin, Aleurites fordii proteins, Dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated Abs, such as ²¹²Bi, ¹³¹I, ¹³¹In, ⁹⁰Y, and ¹⁸⁶Re.

Conjugates of the antibody and cytotoxic agent are made using a variety of bi-functional protein-coupling agents, such as N-succinimidyl-3-(2-pyridyldithiol) propionate (SPDP), iminothiolane (IT), bi-functional derivatives of imidoesters (such as dimethyl adipimidate HCI), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared (Vitetta et al., 1987). ¹⁴C-labeled 1-isothiocyanatobenzyl-3-methyldiethylene triaminepentaacetic acid (MX-DTPA) is an exemplary chelating agent for conjugating radionuclide to antibody (WO 94/11026, 1994).

In another embodiment, the antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a streptavidin “ligand” (e.g., biotin) that is conjugated to a cytotoxic agent (e.g., a radionuclide).

8. Effector Function Engineering

The antibody can be modified to enhance its effectiveness in treating a disease. For example, cysteine residue(s) may be introduced into the F_{c} region, thereby allowing interchain disulfide bond formation in this region. Such homodimeric Abs may have improved internalization capability and/or increased complement-mediated cell killing and antibody-dependent cellular cytotoxicity (ADCC) (Caron et al., 1992; Shopes, 1992). Homodimeric Abs with enhanced anti-tumor activity can be prepared using hetero-bifunctional cross-linkers (Wolff et al., 1993). Alternatively, an antibody engineered with dual F_{c} regions may have enhanced complement lysis (Stevenson et al., 1989).

9. Immunoliposomes

Liposomes containing the antibody may also be formulated (U.S. Pat. No. 4,485,045, 1984; U.S. Pat. No. 4,544,545, 1985; U.S. Pat. No. 5,013,556, 1991; Eppstein et al., 1985; Hwang et al., 1980). Useful liposomes can be generated by a reverse-phase evaporation method with a lipid composition comprising phosphatidylcholine, cholesterol, and PEG-derivatized phosphatidylethanolamine (PEG-PE). Such preparations are extruded through filters of defined pore size to yield liposomes with a desired diameter. Fab′ fragments of the antibody can be conjugated to the liposomes (Martin and Papahadjopoulos, 1982) via a disulfide-interchange reaction. A chemotherapeutic agent, such as Doxorubicin, may also be contained in the liposome (Gabizon et al., 1989). Other useful liposomes with different compositions are contemplated.

10. Diagnostic Applications of Abs Directed Against PDZP, PDZD, PIP or PDBP

Anti-PDZP, PDZD, PIP or PDBP Abs can be used to localize and/or quantitate PDZP, PDZD, PIP or PDBP (e.g., for use in measuring levels of PDZP, PDZD, PIP or PDBPwithin tissue samples or for use in diagnostic methods, etc.). Anti-PDZP, PDZD, ′PIP or PDBP epitope Abs can be utilized as pharmacologically active compounds.

Anti-PDZP, PDZD, PIP or PDBPAbs can be used to isolate PDZP, PDZD, PIP or PDBP by standard techniques, such as immunoaffinity chromatography or immunoprecipitation. These approaches facilitate purifying endogenous PDZP, P or PIP antigen-containing polypeptides from cells and tissues. These approaches, as well as others, can be used to detect PDZP, PDZD, PIP or PDBP in a sample to evaluate the abundance and pattern of expression of the antigenic protein. Anti-PDZP, PDZD, PIP or PDBP Abs can be used to monitor protein levels in tissues as part of a clinical testing procedure; for example, to determine the efficacy of a given treatment regimen. Coupling the antibody to a detectable substance (label) allows detection of Ab-antigen complexes. Classes of labels include fluorescent, luminescent, bioluminescent, and radioactive materials, enzymes and prosthetic groups. Useful labels include horseradish peroxidase, alkaline phosphatase, β-galactosidase, acetylcholinesterase, streptavidin/biotin, avidin/biotin, umbelliferone, fluorescein, fluorescein isothiocyanate, rhodamine, dichlorotriazinylamine fluorescein, dansyl chloride, phycoerythrin, luminol, luciferase, luciferin, aequorin, and ¹²⁵I, ¹³¹I, ³⁵S or ³H.

11. Antibody Therapeutics

Abs of the invention, including polyclonal, monoclonal, humanized and fully human Abs, can be used therapeutically. Such agents will generally be employed to treat or prevent a disease or pathology in a subject. An antibody preparation, preferably one having high antigen specificity and affinity generally mediates an effect by binding the target epitope(s). Generally, administration of such Abs may mediate one of two effects: (1) the antibody may prevent ligand binding, eliminating endogenous ligand binding and subsequent signal transduction, or (2) the antibody elicits a physiological result by binding an effector site on the target molecule, initiating signal transduction.

A therapeutically effective amount of an antibody relates generally to the amount needed to achieve a therapeutic objective, epitope binding affinity, administration rate, and depletion rate of the antibody from a subject. Common ranges for therapeutically effective doses may be, as a nonlimiting example, from about 0.1 mg/kg body weight to about 50 mg/kg body weight. Dosing frequencies may range, for example, from twice daily to once a week.

12. Pharmaceutical Compositions of Abs

Anti-PDZP, PDZD, PIP or PDBP Abs, as well as other PDZP, PDZD, PIP or PDBP interacting molecules (such as aptamers) identified in other assays, can be administered in pharmaceutical compositions to treat various disorders. Principles and considerations involved in preparing such compositions, as well as guidance in the choice of components can be found in (de Boer, 1994; Gennaro, 2000; Lee, 1990).

Abs that are internalized are preferred when whole Abs are used as inhibitors. Liposomes may also be used as a delivery vehicle for intracellular introduction. Where antibody fragments are used, the smallest inhibitory fragment that specifically binds to the epitope is preferred. For example, peptide molecules can be designed that bind a preferred epitope based on the variable-region sequences of a useful antibody. Such peptides can be synthesized chemically and/or produced by recombinant DNA technology (Marasco et al., 1993). Formulations may also contain more than one active compound for a particular treatment, preferably those with activities that do not adversely affect each other. The composition may comprise an agent that enhances function, such as a cytotoxic agent, cytokine, chemotherapeutic agent, or growth-inhibitory agent.

The active ingredients can also be entrapped in microcapsules prepared by coacervation techniques or by interfacial polymerization; for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration are highly preferred to be sterile. This is readily accomplished by filtration through sterile filtration membranes or any of a number of techniques.

Sustained-release preparations may also be prepared, such as semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (Boswell and Scribner, U.S. Pat. No. 3,773,919, 1973), copolymers of L-glutamic acid and y ethyl-L-glutamate, non-degradable ethylene-vinyl acetate, degradable lactic acid-glycolic acid copolymers such as injectable microspheres composed of lactic acid-glycolic acid copolymer, and poly-D-(−)-3-hydroxybutyric acid. While polymers such as ethylene-vinyl acetate and lactic acid-glycolic acid enable release of molecules for over 100 days, certain hydrogels release proteins for shorter time periods and may be preferred.

PDZP, PDZD, PIP or PDBP Recombinant Expression Vectors and Host Cells

Vectors are tools used to shuttle DNA between host cells or as a means to express a nucleotide sequence. Some vectors function only in prokaryotes, while others function in both prokaryotes and eukaryotes, enabling large-scale DNA preparation from prokaryotes for expression in eukaryotes. Inserting the DNA of interest, such as PDZP, PDZD, PIP or PDBP nucleotide sequence or a fragment, is accomplished by ligation techniques and/or mating protocols well known to the skilled artisan. Such DNA is inserted such that its integration does not disrupt any necessary components of the vector. In the case of vectors that are used to express the inserted DNA protein, the introduced DNA is operably-linked to the vector elements that govern its transcription and translation.

Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably-linked to elements, such as promoters, that signal to the host cell to transcribe the inserted DNA. Some promoters are exceptionally useful, such as inducible promoters that control gene transcription in response to specific factors. Operably-linking PDZP, PDZD, PIP or PDBP or antisense construct to an inducible promoter can control the expression of PDZP, PDZD, PIP or PDBP or fragments, or antisense constructs. Examples of classic inducible promoters include those that are responsive to α-interferon, heat-shock, heavy metal ions, and steroids such as glucocorticoids (Kaufman, 1990) and tetracycline. Other desirable inducible promoters include those that are not endogenous to the cells in which the construct is being introduced, but, however, is responsive in those cells when the induction agent is exogenously supplied.

Vectors have many difference manifestations. A “plasmid” is a circular double stranded DNA molecule into which additional DNA segments can be introduced. Viral vectors can accept additional DNA segments into the viral genome. Certain vectors are capable of autonomous replication in a host cell (e.g., episomal mammalian vectors or bacterial vectors having a bacterial origin of replication). Other vectors (e.g., non-episomal mammalian vectors) are integrated into the genome of a host cell upon introduction into the host cell, and thereby are replicated along with the host genome. In general, useful expression vectors are often plasmids. However, other forms of expression vectors, such as viral vectors (e.g., replication defective retroviruses, adenoviruses and adeno-associated viruses) are contemplated.

Recombinant expression vectors that comprise PDZP, PDZD, PIP or PDBP (or fragments) regulate PDZP, PDZD, PIP or PDBP transcription by exploiting one or more host cell-responsive (or that can be manipulated in vitro) regulatory sequences that is operably-linked to PDZP, PDZD, PIP or PDBP. “Operably-linked” indicates that a nucleotide sequence of interest is linked to regulatory sequences such that expression of the nucleotide sequence is achieved.

Vectors can be introduced in a variety of organisms and/or cells (Table F). Alternatively, the vectors can be transcribed and translated in vitro, for example using T7 promoter regulatory sequences and T7 polymerase.

TABLE F
Examples of hosts for cloning or expression

| | | Sources and |
|---|---|---|
| Organisms | Examples | References* |
| Prokaryotes | E. coli | |
| Enterobacteriaceae | K 12 strain MM294 | ATCC 31, 446 |
| | X1776 | ATCC 31, 537 |
| | W3110 | ATCC 27, 325 |
| | K5 772 | ATCC 53, 635 |
| | Enterobacter | |
| | Erwinia | |
| | Kiebsiella | |
| | Proteus | |
| | Salmonella | |
| | (S. tyhpimurium) | |
| | Serratia (S. marcescans) | |
| | Shi ge/la | |
| | Bacilli (B. subtilis and B. | |
| | licheniformis) | |
| | Pseudomonas (P. | |
| | aeruginosa) | |
| Streptomyces | | |
| Eukaryotes | Saccharomyees | |
| Yeasts | cerevisiae | |
| | Schizosaceharomyces | |
| | pombe | |
| | Kluyveromyces | (Fleer et al., 1991) |
| | K. lactis MW98-8C, | (de Louvencourt et al., |
| | CBS683, CBS4574 | 1983) |
| | K. fragilis | ATCC 12, 424 |
| | K. bulgaricus | ATCC 16, 045 |
| | K. wickeramii | ATCC 24, 178 |
| | K. waltii | ATCC 56, 500 |
| | K. drosophilarum | ATCC 36, 906 |
| | K. thermotolerans | |
| | K. marxianus; yarrowia | (EPO 402226, 1990) |
| | Pichia pasioris | (Sreekrishna et at., |
| | | 1988) |
| | Candida | |
| | Trichoderma reesia | |
| | Neurospora crassa | (Case et al., 1979) |
| | Torulopsis | |
| | Rhodotorula | |
| | Schwanniomyces (S. | |
| | occidentalis) | |
| Filamentous Fungi | Neurospora | |
| | Penicillium | |
| | Tolypocladium | (WO 91/00357, 1991) |
| | Aspergillus | (Kelly and Hynes, |
| | (A. nidulans and | 1985; Tilburn |
| | A. niger) | et al., 1983; |
| | | Yelton et al., 1984) |
| Invertebrate cells | Drosophila S2 | |
| | Spodoptera Sf9 | |
| Vertebrate cells | Chinese Hamster Ovary | |
| | (CHO) | |
| | simian COS | ATCC CRL 1651 |
| | COS-7 | |
| | HEK 293 | |

*Unreferenced cells are generally available from American Type Culture Collection (Manassas, VA).

Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used and are easily determined. Some of these elements may be conditional, such as an inducible or conditional promoter that is turned “on” when conditions are appropriate. Examples of inducible promoters include those that are tissue-specific, which relegate expression to certain cell types, steroid-responsive, or heat-shock reactive. Some bacterial repression systems, such as the lac operon, have been exploited in mammalian cells and transgenic animals (Fieck et al., 1992; Wyborski et al., 1996; Wyborski and Short, 1991). Vectors often use a selectable marker to facilitate identifying those cells that have incorporated the vector. Many selectable markers are well known in the art for the use with prokaryotes, usually antibiotic-resistance genes or the use of autotrophy and auxotrophy mutants.

Using antisense and sense PDZP, PDZD, PIP or PDBP oligonucleotides can prevent PDZP, PDZD, PIP or PDBP polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target PDZP, PDZD, PIP or PDBP mRNA (sense) or PDZP, PDZD, PIP or PDBP DNA (antisense) sequences. According to the present invention, antisense or sense oligonucleotides comprise a fragment of a PDZP, PDZD, PIP or PDBP DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Among others, (Stein and Cohen, 1988; van der Krol et al., 1988b) describe methods to derive antisense or a sense oligonucleotides from a given CDNA sequence.

Modifications of antisense and sense oligonucleotides can augment their effectiveness. Modified sugar-phosphodiester bonds or other sugar linkages (WO 91/06629, 1991), increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents.

To introduce antisense or sense oligonucleotides into target cells (cells containing the target nucleic acid sequence), any gene transfer method may be used and are well known to those of skill in the art. Examples of gene transfer methods include 1) biological, such as gene transfer vectors like Epstein-Barr virus or conjugating the exogenous DNA to a ligand-binding molecule (WO 91/04753, 1991), 2) physical, such as electroporation, and 3) chemical, such as CaPO₄ precipitation and oligonucleotide-lipid complexes (WO 90/10448, 1990).

The terms “host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Methods of eukaryotic cell transfection and prokaryotic cell transformation are well known in the art. The choice of host cell will dictate the preferred technique for introducing the nucleic acid of interest. Table G, which is not meant to be limiting, summarizes many of the known techniques in the art. Introduction of nucleic acids into an organism may also be done with ex vivo techniques that use an in vitro method of transfection, as well as established genetic techniques, if any, for that particular organism.

TABLE G
Methods to introduce nucleic acid into cells

| Cells | Methods | References | Notes |
|---|---|---|---|
| Prokaryotes | Calcium chloride | (Cohen et al., 1972; | |
| (bacteria) | | Hanahan, 1983; Mandel and | |
| | | Higa, 1970) | |
| | Electroporation | (Shigekawa and Dower, | |
| | | 1988) | |
| Eukaryotes | | | |
| Mammalian | Calcium phosphate | N-(2- | Cells may be |
| cells | transfection | Hydroxyethyl)piperazine-N′- | “shocked” with |
| | | (2-ethanesulfonic acid | glycerol or |
| | | (HEPES) buffered saline | dimethylsulfoxide |
| | | solution (Chen and | (DMSO) to increase |
| | | Okayama, 1988; Graham and | transfection |
| | | van der Eb, 1973; Wigler et | efficiency (Ausubel |
| | | al., 1978) | et al., 1987). |
| | | BES (N,N-bis(2- | |
| | | hydroxyethyl)-2- | |
| | | aminoethanesulfonic acid) | |
| | | buffered solution (Ishiura et | |
| | | al., 1982) | |
| | Diethylaminoethyl | (Fujita et al., 1986; Lopata et | Most useful for |
| | (DEAE)-Dextran | al., 1984; Selden et al., 1986) | transient, but not |
| | transfection | | stable, transfections. |
| | | | Chloroquine can be |
| | | | used to increase |
| | | | efficiency. |
| | Electroporation | (Neumann et al., 1982; | Especially useful for |
| | | Potter, 1988; Potter et al., | hard-to-transfect |
| | | 1984; Wong and Neumann, | lymphocytes. |
| | | 1982) | |
| | Cationic lipid | (Elroy-Stein and Moss, | Applicable to both |
| | reagent | 1990; Felgner et al., 1987; | in vivo and in vitro |
| | transfection | Rose et al., 1991; Whitt et | transfection. |
| | | al., 1990) | |
| | Retroviral | Production exemplified by | Lengthy process, |
| | | (Cepko et al., 1984; Miller | many packaging |
| | | and Buttimore, 1986; Pear et | lines available at |
| | | al., 1993) | ATCC. Applicable |
| | | Infection in vitro and in vivo: | to both in vivo and |
| | | (Austin and Cepko, 1990; | in vitro transfection. |
| | | Bodine et al., 1991; Fekete | |
| | | and Cepko, 1993; Lemischka | |
| | | et al., 1986; Turner et al., | |
| | | 1990; Williams et al., 1984) | |
| | Polybrene | (Chancy et al., 1986; Kawai | |
| | | and Nishizawa, 1984) | |
| | Microinjection | (Capecchi, 1980) | Can be used to |
| | | establish cell lines | |
| | | carrying integrated | |
| | | copies of PDZP, | |
| | | PDZD, PIP or | |
| | | PDBP DNA | |
| | | sequences. | |
| | Protoplast fusion | (Rassoulzadegan et al., 1982; | |
| | | Sandri-Goldin et al., 1981; | |
| | | Schaffner, 1980) | |
| Insect cells | Baculovirus | (Luckow, 1991; Miller, | Useful for in vitro |
| (in vitro) | systems | 1988; O'Reilly et al., 1992) | production of |
| | | | proteins with |
| | | | eukaryotic |
| | | | modifications. |
| Yeast | Electroporation | (Becker and Guarente, 1991) | |
| | Lithium acetate | (Gietz et al., 1998; Ito et al., | |
| | | 1983) | |
| | Splieroplast fusion | (Beggs, 1978; Hinnen et al., | Laborious, can |
| | | 1978) | produce aneuploids. |
| Plant cells | Agrobacterium | (Bechtold and Pelletier, | |
| (general | transformation | 1998; Escudero and Hohn, | |
| reference: | | 1997; Hansen and Chilton, | |
| (Hansen and | | 1999; Touraev and al., 1997) | |
| Wright, | Biolistics | (Finer et al., 1999; Hansen | |
| 1999)) | (microprojectiles) | and Chilton, 1999; Shillito, | |
| | | 1999) | |
| | Electroporation | (Fromm et al., 1985; Ou-Lee | |
| | (protoplasts) | et al., 1986; Rhodes et al., | |
| | | 1988; Saunders et al., 1989) | |
| | | May be combined with | |
| | | liposomes (Trick and al., | |
| | | 1997) | |
| | Polyethylene | (Shillito, 1999) | |
| | glycol (PEG) | | |
| | treatment | | |
| | Liposomes | May be combined with | |
| | | electroporation (Trick and | |
| | | al., 1997) | |
| | in planta | (Leduc and al., 1996; Zhou | |
| | microinjection | and al., 1983) | |
| | Seed imbibition | (Trick and al., 1997) | |
| | Laser beam | (Hoffman, 1996) | |
| | Silicon carbide | (Thompson and al., 1995) | |
| | whiskers | | |

Vectors often use a selectable marker to facilitate identifying those cells that have incorporated the vector. Many selectable markers are well known in the art for the use with prokaryotes, usually antibiotic-resistance genes or the use of autotrophy and auxotrophy mutants. Table H lists often-used selectable markers for mammalian cell transfection.

TABLE H
Useful selectable markers for eukaryote cell transfection

| Selectable Marker | Selection | Action | Reference |
|---|---|---|---|
| Adenosine | Media includes 9-β-D- | Conversion of Xyl-A | (Kaufman et |
| deaminase (ADA) | xylofuranosyl adenine | to Xyl-ATP, which | al., 1986) |
| | (Xyl-A) | incorporates into | |
| | | nucleic acids, killing | |
| | | cells. ADA detoxifies | |
| Dihydrofolate | Methotrexate (MTX) | MTX competitive | (Simonsen |
| reductase | and dialyzed serum | inhibitor of DHFR. In | and |
| (DHFR) | (purine-free media) | absence of exogenous | Levinson, |
| | | purines, cells require | 1983) |
| | | DHFR, a necessary | |
| | | enzyme in purine | |
| | | biosynthesis. | |
| Aminoglycoside | G418 | G418, an | (Southern |
| phosphotransferase | | aminoglycoside | and Berg, |
| (“APH”, “neo”, | | detoxified by APH, | 1982) |
| “G418”) | | interferes with | |
| | | ribosomal function | |
| | | and consequently, | |
| | | translation. | |
| Hygromycin-B- | hygromycin-B | Hygromycin-B, an | (Palmer et |
| phosphotransferase | | aminocyclitol | al., 1987) |
| (HPH) | | detoxified by HPH, | |
| | | disrupts protein | |
| | | translocation and | |
| | | promotes | |
| | | mistranslation. | |
| Thymidine kinase | Forward selection | Forward: Aminopterin | (Littlefield, |
| (TK) | (TK+): Media (HAT) | forces cells to | 1964) |
| | incorporates | synthesze dTTP from | |
| | aminopterin. | thymidine, a pathway | |
| | Reverse selection | requiring TK. | |
| | (TK−): Media | Reverse: TK | |
| | incorporates 5- | phosphorylates BrdU, | |
| | bromodeoxyuridine | which incorporates | |
| | (BrdU). | into nucleic acids, | |
| | | killing cells. | |

A host cell, such as a prokaryotic or eukaryotic host cell in culture, can be used to produce PDZP, PDZD, PIP or PDBP.

Pharmaceutical Compositions

PDZP, PDZD, PIP or PDBP-encoding nucleic acid molecules, PDZP, PDZD, PIP or pDBP peptides/polypeptides, and anti-PDZP, PDZD, PIP or PDBP Abs, PDLs, and derivatives, fragments, analogs and homologs thereof, can be incorporated into pharmaceutical compositions. Such compositions typically comprise the nucleic acid molecule, protein, or antibody and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (Gennaro, 2000). Preferred examples of such carriers or diluents include, but are not limited to, water, saline, Finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

1. General Considerations

A pharmaceutical composition is formulated to be compatible with its intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

2. Injectable Formulations

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents; for example, parabens, chlorobutanol, phenol, ascorbic acid, and thimerosal, can contain microorganism contamination. Isotonic agents; for example, sugars, polyalcohols such as manitol, sorbitol, and sodium chloride can be included in the composition. Compositions that can delay absorption include agents such as aluminum monostearate and gelatin.

Sterile injectable solutions can be prepared by incorporating the active compound (e.g., a PDZP, PDZD, PIP or PDBP or anti-PDZP, PDZD, PIP or PDBP antibody) in the required amount in an appropriate solvent with one or a combination of ingredients as required, followed by sterilization. Generally, dispersions are prepared by incorporating the active compound into a sterile vehicle that contains a basic dispersion medium, and the other required ingredients. Sterile powders for the preparation of sterile injectable solutions, methods of preparation include vacuum drying and freeze-drying that yield a powder containing the active ingredient and any desired ingredient from a sterile solutions.

3. Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

4. Compositions for Inhalation

For administration by inhalation, the compounds are delivered as an aerosol spray from a nebulizer or a pressurized container that contains a suitable propellant, e.g., a gas such as carbon dioxide.

5. Systemic Administration

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

6. Carriers

In one embodiment, the active compounds are prepared with carriers that protect the compound against rapid elimination from the body, such as a controlled release formulation, including implants and microencapsulated delivery systems. Biodegradable or biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art. Liposomal suspensions can also be used as pharmaceutically acceptable carriers. These can be prepared according to methods known to those skilled in the art, such as in (Eppstein et al., U.S. Pat. No. 4,522,811, 1985).

7. Unit Dosage

Oral formulations or parenteral compositions in unit dosage form can be created to facilitate administration and dosage uniformity. Unit dosage form refers to physically discrete units suited as single dosages for the subject to be treated, containing a therapeutically effective quantity of active compound in association with the required pharmaceutical carrier. The specification for the unit dosage forms are dictated by, and directly dependent on, the unique characteristics of the active compound and the particular desired therapeutic effect, and the inherent limitations of compounding the active compound.

8. Gene Therapy Compositions

The nucleic acid molecules can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

9. Dosage

The pharmaceutical composition and method may further comprise other therapeutically active compounds that are usually applied in the treatment of PDZP or PIP-related conditions.

In the treatment or prevention of conditions which require PDZP, PDZD, PIP or PDBP modulation an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

However, the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

10. Kits for Pharmaceutical Compositions

The pharmaceutical compositions can be included in a kit, container, pack, or dispenser together with instructions for administration. When supplied as a kit, the different components of the composition may be packaged in separate containers and admixed immediately before use. Such packaging of the components separately may permit long-term storage without losing the active components' functions.

Kits may also include reagents in separate containers that facilitate the execution of a specific test, such as diagnostic tests or tissue typing. For example, PDZP, PDZD, PIP or PDBP DNA templates and suitable primers may be supplied for internal controls.

(a) Containers or Vessels

The reagents included in kits can be supplied in containers of any sort such that the life of the different components are preserved and are not adsorbed or altered by the materials of the container. For example, sealed glass ampules may contain lyophilized PDZP, PDZD, PIP or PDBP or buffer that have been packaged under a neutral, non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, laserdisc, audio tape, etc. Detailed instructions may not be physically associated with the kit; instead, a user may be directed to an Internet web site specified by the manufacturer or distributor of the kit, or supplied as electronic mail.

B. Screening and Detection Methods

Isolated nucleic acid molecules encoding PDZPs, PDZDs, PIPs or PDBPs can be used to express PDZPs, PDZDs, PIPs or PDBPs (e.g., via a recombinant expression vector in a host cell in gene therapy applications), to detect PDZP, PDZD, PIP or PDBP mRNA (e.g., in a biological sample) or a genetic lesion in a PDZP, PDZD, PIP or PDBP, and to modulate a PDZP, PDZD, PIP or PDBP activity. In addition, PDZP, PDZD, PIP or PDBP peptides/polypeptides can be used to screen drugs or compounds that modulate a PDZP, PDZD, PIP or PDBP activity or expression as well as to treat disorders characterized by insufficient or excessive production of PDZP, PDZD, PIP or PDBP or production of PDZP, PDZD, PIP or PDBP forms that have decreased or aberrant activity compared to PDZP or PIP wild-type protein, or modulate biological function that involve PDZP, PDZD, PIP or PDBP. In addition, anti-PDZP, PDZD, PIP or PDBP Abs can be used to detect and isolate PDZP, PDZD, PIP or PDBP and modulate PDZP, PDZD, PIP or PDBP activity.

(e) screens to Identify Modulators

Modulators of PDZP, PDZD, PIP or PDBP expression can be identified in a method where a cell is contacted with a candidate compound and the expression of PDZP, PDZD, PIP or PDBP mRNA or protein in the cell is determined. The expression level of PDZP, PDZD, PIP or PDBP mRNA or protein in the presence of the candidate compound is compared to PDZP, PDZD, PIP or PDBP mRNA or protein levels in the absence of the candidate compound. The candidate compound can then be identified as a modulator of PDZP, PDZD, PIP or PDBP mRNA or protein expression based upon this comparison. For example, when expression of PDZP, PDZD, PIP or PDBP mRNA or protein is greater (i.e., statistically significant) in the presence of the candidate compound than in its absence, the candidate compound is identified as a stimulator of PDZP, PDZD, PIP or PDBP mRNA or protein expression. Alternatively, when expression of PDZP, PDZD, PIP or PDBP mRNA or protein is less (statistically significant) in the presence of the candidate compound than in its absence, the candidate compound is identified as an inhibitor of PDZP, PDZD, PIP or PDBP mRNA or protein expression. The level of PDZP, PDZD, PIP or PDBP mRNA or protein expression in the cells can be determined by methods described for detecting PDZP, PDZD, PIP or PDBP mRNA or protein.

In a preferred embodiment, molecules are assayed for their ability to prevent a PDZP or PDZD from interacting with a cognate PIP or PDBP. For example, IC₅₀ values using competition ELISAs can be used to ascertain the effectiveness of a candidate modulator. The IC₅₀ value is defined as the concentration of a candidate substance that blocks 50% of PDZ domain binding to an immobilized cognate PIP or PDBP or PIP. Assay plates are prepared by coating microwell plates (preferably treated to efficiently absorb protein) with neutravidin, avidin or streptavidin. Non-specific binding sites are then blocked through addition of a solution of bovine serum albumin (BSA) or other proteins (for example, nonfat milk) and then washed, preferably with a buffer containing Tween-20. An amino-terminally biotinylated peptide PDBP, PIP or fragment thereof is then added (preferably at a concentration of 100 nM), preferably with 0.5% BSA and 0.05% Tween-20. Simultaneously, binding reactions consisting of serial dilutions of the test molecules, preferably with 0.5% BSA and 0.05% Tween-20 containing PDZ domain fusion protein, PDZ domain peptide/protein. The plate coated with the immobilized PDBP, PIP or fragment thereof is preferably again extensively washed before adding each binding reaction to the wells and incubating briefly, preferably 15 minutes. The plates are again washed extensively before binding being visualized, such as development with a HRP conjugated secondary antibody and a primary antibody that recognizes the PDZ domain fusion protein, PDBP or PIP whose binding is being assayed. The signal is then appropriately read, such as by a spectrophotometer.

Apparent to one of skill are the many variations of the above assay. For example, instead of avidin-biotin based systems, PDZP, PDZD, PIP or PDBP may be chemically-linked to a substrate, or simply absorbed. A specific example of such a screen is found in the Examples.

2. Detection Assays

PDZP, PDZD, PIP or PDBP-encoding nucleic acids are useful in themselves. By way of non-limiting example, these sequences can be used to: (1) identify an individual from a minute biological sample (tissue typing); and (2) aid in forensic identification of a biological sample.

C. Predictive Medicine

The field of predictive medicine pertains to diagnostic assays, prognostic assays, pharmacogenomics, and monitoring clinical trials used for prognostic (predictive) purposes to treat an individual prophylactically. Accordingly, one aspect relates to diagnostic assays for determining PDZP, PDZD, PIP or PDBP and/or nucleic acid expression as well as PDZP, PDZD, PIP or PDBP activity, in the context of a biological sample (e.g., blood, serum, cells, tissue) to determine whether an individual is afflicted with a disease or disorder, or is at risk of developing a disorder, associated with aberrant PDZP, PDZD, PIP or PDBP expression or activity, including cancer. The invention also provides for prognostic (or predictive) assays for determining whether an individual is at risk of developing a disorder associated with PDZP, PDZD, PIP or PDBP, nucleic acid expression or activity. For example, mutations in PDZP, PDZD, PIP or PDBP can be assayed in a biological sample. Such assays can be used for prognostic or predictive purpose to prophylactically treat an individual prior to the onset of a disorder characterized by or associated with PDZP, PDZD, PIP or PDBP, nucleic acid expression, or biological activity.

Another aspect provides methods for determining PDZP, PDZD, PIP or PDBP activity, or nucleic acid expression, in an individual to select appropriate therapeutic or prophylactic agents for that individual (referred to herein as “pharmacogenomics”). Pharmacogenomics allows for the selection of modalities (e.g., drugs, foods) for therapeutic or prophylactic treatment of an individual based on the individual's genotype (e.g., the individual's genotype to determine the individual's ability to respond to a particular agent). Another aspect pertains to monitoring the influence of modalities (e.g., drugs, foods) on the expression or activity of PDZP, PDZD, PIP or PDBP in clinical trials.

1. Diagnostic Assays

An exemplary method for detecting the presence or absence of PDZP, PDZD, PIP or PDBP in a biological sample involves obtaining a biological sample from a subject and contacting the biological sample with a compound or an agent capable of detecting PDZP, PDZD, PIP or PDBP polypeptides or nucleic acids (e.g., mRNA, genomic DNA) such that the presence of PDZP, PDZD, PIP or PDBP is confirmed in the sample. An agent for detecting PDZP, PDZD, PIP or PDBP mRNA or genomic DNA is a labeled nucleic acid probe that can hybridize to PDZP, PDZD, PIP or PDBP mRNA or genomic DNA. The nucleic acid probe can be, for example, a PDZP, PDZD, PIP or PDBP encoding nucleic acid or a portion thereof, such as an oligonucleotide of at least 15, 30, 50, 100, 250 or 500 nucleotides in length and sufficient to specifically hybridize under stringent conditions to PDZP, PDZD, PIP or PDBP mRNA or genomic DNA.

An agent for detecting PDZP, PDZD, PIP or PDBP polypeptide is an antibody capable of binding to PDZP, PDZD, PIP or PDBP, preferably an antibody with a detectable label. A labeled probe or antibody is coupled (i.e., physically linking) to a detectable substance, as well as indirect detection of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently-labeled streptavidin. The term “biological sample” includes tissues, cells and biological fluids isolated from a subject, as well as tissues, cells and fluids present within a subject. The detection method can be used to detect PDZP, PDZD, PIP or PDBP mRNA, protein, or genomic DNA in a biological sample in vitro as well as in vivo. For example, in vitro techniques for detection of PDZP, PDZD, PIP or PDBP mRNA include Northern hybridizations and in situhybridizations. In vitro techniques for detection of PDZP, PDZD, PIP or PDBP polypeptide include enzyme linked immunosorbent assays (ELISAs), Western blots, immunoprecipitations, and immunofluorescence. In vitro techniques for detection of PDZP, PDZD, PIP or PDBP genomic DNA include Southern hybridizations and fluorescent in situhybridization (FISH). Furthermore, in vivo techniques for detecting PDZP, PDZD, PIP or PDBP include introducing into a subject a labeled anti-PDZP, PDZD, PIP or PDBP antibody. For example, the antibody can be labeled with a radioactive marker whose presence and location in a subject can be detected by standard imaging techniques.

The methods may further involve obtaining a biological sample from a subject to provide a control, contacting the sample with a compound or agent to detect PDZP, PDZD, PIP or PDBP; PDZP, PDZD, PIP or PDBP mRNA, or genomic DNA, and comparing the presence of PDZP, PDZD, PIP or PDBP; PDZP, PDZD, PIP or PDBP mRNA or genomic DNA in the control sample with the presence of PDZP, PDZD, PIP or PDBP; PDZP, PDZD, PIP or PDBP mRNA or genomic DNA in the test sample.

The invention also encompasses kits for detecting PDZP, PDZD, PIP or PDBP in a biological sample. For example, the kit can comprise: a labeled compound or agent capable of detecting PDZP, PDZD, PIP or PDBP mRNA, peptide or protein in a sample; reagent and/or equipment for determining the amount of PDZP, PDZD, PIP or PDBP in the sample; and reagent and/or equipment for comparing the amount of PDZP, PDZD, PIP or PDBP in the sample with a standard. The compound or agent can be packaged in a suitable container. The kit can further comprise instructions for using the kit to detect PDZP, PDZD, PIP or PDBP or nucleic acid.

2. Prognostic Assays

The diagnostic methods described herein can furthermore be utilized to identify subjects having or at risk of developing a disease or disorder associated with aberrant PDZP, PDZD, PIP or PDBP expression or activity. For example, the described assays can be used to identify a subject having or at risk of developing a disorder associated with PDZP, PDZD, PIP or PDBP, nucleic acid expression or activity. Alternatively, the prognostic assays can be used to identify a subject having or at risk for developing a disease or disorder. The invention provides a method for identifying a disease or disorder associated with aberrant PDZP, PDZD, PIP or PDBP expression or activity in which a test sample is obtained from a subject and PDZP, PDZD, PIP or PDBP or nucleic acid (e.g., mRNA, genomic DNA) is detected. A test sample is a biological sample obtained from a subject. For example, a test sample can be a biological fluid (e.g., serum), cell sample, or tissue.

Prognostic assays can be used to determine whether a subject can be administered a modality (e.g., an agonist, antagonist, peptidomimetic, protein, peptide, nucleic acid, small molecule, food, etc.) to treat a disease or disorder associated with aberrant PDZP, PDZD, PIP or PDBP expression or activity. Such methods can be used to determine whether a subject can be effectively treated with an agent for a disorder. Methods of determining whether a subject can be effectively treated with an agent for a disorder associated with aberrant PDZP, PDZD, PIP or PDBP expression or activity involve acquiring a test sample and PDZP, PDZD, PIP or PDBP or nucleic acid is detected (e.g., where the presence of PDZP, PDZD, PIP or PDBP or nucleic acid is diagnostic for a subject that can be administered the agent to treat a disorder associated with aberrant PDZP, PDZD, PIP or PDBP expression or activity).

The methods can also be used to detect genetic lesions in a PDZP, PDZD, PIP or PDBP to determine if a subject with the genetic lesion is at risk for a disorder. Methods include detecting, in a sample from the subject, the presence or absence of a genetic lesion characterized by at an alteration affecting the integrity of a gene encoding a PDZP, PDZD, PIP or PDBP protein, or the mis-expression of PDZP, PDZD, PIP or PDBP. Such genetic lesions can be detected by ascertaining: (1) a deletion of one or more nucleotides from PDZP, PDZD, PIP or PDBP; (2) an addition of one or more nucleotides to PDZP, PDZD, PIP or PDBP; (3) a substitution of one or more nucleotides in PDZP, PDZD, PIP or PDBP, (4) a chromosomal rearrangement of a PDZP, PDZD, PIP or PDBP gene; (5) an alteration in the level of a PDZP, PDZD, PIP or PDBP mRNA transcripts, (6) aberrant modification of a PDZP, PDZD, PIP or PDBP, such as a change genomic DNA methylation, (7) the presence of a non-wild-type splicing pattern of a PDZP, PDZD, PIP or PDBP mRNA transcript, (8) a non-wild-type level of PDZP, PDZD, PIP or PDBP, (9) allelic loss of PDZP, PDZD, PIP or PDBP, and/or (10) inappropriate post-translational modification of PDZP, PDZD, PIP or PDBP protein. There are a large number of known assay techniques that can be used to detect lesions in PDZP, PDZD, PIP or PDBP. Any biological sample containing nucleated cells may be used.

In certain embodiments, lesion detection may use a probe/primer in a polymerase chain reaction (PCR) (e.g., (Mullis, U.S. Pat. No. 4,683,202, 1987; Mullis et al., U.S. Pat. No. 4,683,195, 1987), such as anchor PCR or rapid amplification of cDNA ends (RACE) PCR, or, alternatively, in a ligation chain reaction (LCR) (e.g., (Landegren et al., 1988; Nakazawa et al., 1994), the latter is particularly useful for detecting point mutations in PDZP, PDZD, PIP or PDBP (Abravaya et al., 1995). This method may include collecting a sample from a patient, isolating nucleic acids from the sample, contacting the nucleic acids with one or more primers that specifically hybridize to PDZP, PDZD, PIP or PDBP under conditions such that hybridization and amplification of a PDZP, PDZD, PIP or PDBP (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990), transcriptional amplification system (Kwoh et al., 1989); Qβ Replicase (Lizardi et al., 1988), or any other nucleic acid amplification method, followed by the detection of the amplified molecules using techniques well known to those of skill in the art. These detection schemes are especially useful for the detection of nucleic acid molecules present in low abundance.

Mutations in PDZP, PDZD, PIP or PDBP from a sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site.

Hybridizing a sample and control nucleic acids, e.g., DNA or RNA, to high-density arrays containing hundreds or thousands of oligonucleotides probes, can identify genetic mutations in PDZPs, PDZDs, PIPs or PDBPs (Cronin et al., 1996; Kozal et al., 1996). For example, genetic mutations in PDZP, PDZD, PIP or PDBP can be identified in two-dimensional arrays containing light-generated DNA probes as described (Cronin et al., 1996). Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a PDZP, PDZD, PIP or PDBP and detect mutations by comparing the sequence of the sample PDZP, PDZD, PIP or PDBP-with the corresponding wild-type (control) sequence. Examples of sequencing reactions include those based on classic techniques (Maxam and Gilbert, 1977; Sanger et al., 1977). Any of a variety of automated sequencing procedures can be used when performing diagnostic assays (Naeve et al., 1995) including sequencing by mass spectrometry (Cohen et al., 1996; Griffin and Griffin, 1993; Koster, WO94/16101, 1994).

Other methods for detecting mutations in a PDZP, PDZD, PIP or PDBP include those in which protection from cleavage agents is used to detect mismatched bases in RNA/RNA or RNA/DNA heteroduplexes (Myers et al., 1985). In general, the technique of “mismatch cleavage” starts by providing heteroduplexes formed by hybridizing (labeled) RNA or DNA containing the wild-type PDZP, PDZD, PIP or PDBP sequence with potentially mutant RNA or DNA obtained from a sample. The double-stranded duplexes are treated with an agent that cleaves single-stranded regions of the duplex such as those that arise from base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. The digested material is then separated by size on denaturing polyacrylamide gels to determine the mutation site (Grompe et al., 1989; Saleeba and Cotton, 1993). The control DNA or RNA can be labeled for detection.

Mismatch cleavage reactions may employ one or more proteins that recognize mismatched base pairs in double-stranded DNA (DNA mismatch repair) in defined systems for detecting and mapping point mutations in PDZP, PDZD, PIP or PDBP cDNAs obtained from samples of cells. For example, the mutY enzyme of E. coli cleaves A at G/A mismatches and the thymidine DNA glycosylase from HeLa cells cleaves T at G/T mismatches (Hsu et al., 1994). According to an exemplary embodiment, a probe based on a wild-type PDZP, PDZD, PIP or PDBP sequence is hybridized to a cDNA or other DNA product from a test cell(s). The duplex is treated with a DNA mismatch repair enzyme, and the cleavage products, if any, can be detected from electrophoresis protocols or the like (Modrich et al., U.S. Pat. No. 5,459,039, 1995).

Electrophoretic mobility alterations can be used to identify mutations in PDZP, PDZD, PIP or PDBP. For example, single strand conformation polymorphism (SSCP) may be used to detect differences in electrophoretic mobility between mutant and wild type nucleic acids (Cotton, 1993; Hayashi, 1992; Orita et al., 1989). Single-stranded DNA fragments of sample and control PDZP, PDZD, PIP or PDBP nucleic acids are denatured and then renatured. The secondary structure of single-stranded nucleic acids varies according to sequence; the resulting alteration in electrophoretic mobility allows detection of even a single base change. The DNA fragments may be labeled or detected with labeled probes. The sensitivity of the assay may be enhanced by using RNA (rather than DNA), in which the secondary structure is more sensitive to a sequence changes.

The method may use heteroduplex analysis to separate double stranded heteroduplex molecules on the basis of changes in electrophoretic mobility (Keen et al., 1991). The migration of mutant or wild-type fragments can be assayed using denaturing gradient gel electrophoresis (DGGE; (Myers et al., 1985). In DGGE, DNA is modified to prevent complete denaturation, for example by adding a GC clamp of approximately 40 bp of high-melting GC-rich DNA by PCR. A temperature gradient may also be used in place of a denaturing gradient to identify differences in the mobility of control and sample DNA (Rossiter and Caskey, 1990).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al., 1986; Saiki et al., 1989). Such allele-specific oligonucleotides are hybridized to PCR-amplified target DNA or a number of different mutations when the oligonucleotides are attached to the hybridizing membrane and hybridized with labeled target DNA.

Alternatively, allele specific amplification technology that depends on selective PCR amplification may be used. Oligonucleotide primers for specific amplifications may carry the mutation of interest in the center of the molecule, so that amplification depends on differential hybridization (Gibbs et al., 1989) or at the extreme 3′-terminus of one primer where, under appropriate conditions, mismatch can prevent, or reduce polymerase extension (Prosser, 1993). Novel restriction site in the region of the mutation may be introduced to create cleavage-based detection (Gasparini et al., 1992). Certain amplification may also be performed using Taq ligase for amplification (Barany, 1991). In such cases, ligation occurs only if there is a perfect match at the 3′-terminus of the 5′sequence, allowing detection of a known mutation by scoring for amplification.

The described methods may be performed, for example, by using pre-packaged kits comprising at least one probe (nucleic acid or antibody) that may be conveniently used, for example, in clinical settings to diagnose patients exhibiting symptoms or family history of a disease or illness involving PDZP, PDZD, PIP or PDBP.

Furthermore, any cell type or tissue in which PDZP, PDZD, PIP or PDBP is expressed may be utilized in the prognostic assays described herein.

3. Pharmacogenomics

Agents, or modulators that have a stimulatory or inhibitory effect on PDZP, PDZD, PIP or PDBP activity or expression, as identified by a screening assay, can be administered to individuals to treat prophylactically or therapeutically disorders. In conjunction with such treatment, the pharmacogenomics (i.e., the study of the relationship between a subject's genotype and the subject's response to a foreign modality, such as a food, compound or drug) may be considered. Metabolic differences of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Pharmacogenomics can further be used to determine appropriate dosages and therapeutic regimens. Accordingly, the activity of PDZP, PDZD, PIP or PDBP, expression of PDZP, PDZD, PIP or PDBP, or PDZP, PDZD, PIP or PDBP mutation(s) in an individual can be determined to guide the selection of appropriate agent(s) for therapeutic or prophylactic treatment.

Pharmacogenomics deals with clinically significant hereditary variations in the response to modalities due to altered modality disposition and abnormal action in affected persons (Eichelbaum and Evert, 1996; Linder et al., 1997). In general, two pharmacogenetic conditions can be differentiated: (1) genetic conditions transmitted as a single factor altering the interaction of a modality with the body (altered drug action) or (2) genetic conditions transmitted as single factors altering the way the body acts on a modality (altered drug metabolism). These pharmacogenetic conditions can occur either as rare defects or as nucleic acid polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) explains the phenomena of some patients who show exaggerated drug response and/or serious toxicity after taking the standard and safe dose of a drug. These polymorphisms are expressed in two phenotypes in the population, the extensive metabolizer (EM) and poor metabolizer (PM). The prevalence of PM is different among different populations. For example, the CYP2D6 gene is highly polymorphic and several mutations have been identified in PM, which all lead to the absence of functional CYP2D6. Poor metabolizers due to mutant CYP2D6 and CYP2C19 frequently experience exaggerated drug responses and side effects when they receive standard doses. If a metabolite is the active therapeutic moiety, PM shows no therapeutic response, as demonstrated for the analgesic effect of codeine mediated by its CYP2D6-formed metabolite morphine. At the other extreme are the so-called ultra-rapid metabolizers who are unresponsive to standard doses. Recently, the molecular basis of ultra-rapid metabolism has been identified to be due to CYP2D6 gene amplification.

The activity of PDZP, PDZD, PIP or PDBP, expression of PDZP, PDZD, PIP or PDBP-encoding nucleic acids, or mutation content of PDZP, PDZD, PIP or PDBP in an individual can be determined to select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This information, when applied to dosing or drug selection, can avoid adverse reactions or therapeutic failure and thus enhance therapeutic or prophylactic efficiency when treating a subject with a PDZP, PDZD, PIP or PDBP modulator, such as a modulator identified by one of the described exemplary screening assays.

1. Monitoring Effects During Clinical Trials

Monitoring the influence of agents (e.g., drugs, compounds) on the expression or activity of PDZP, PDZD, PIP or PDBP can be applied not only in basic drug screening, but also in clinical trials. For example, the effectiveness of an agent determined by a screening assay to increase PDZP, PDZD, PIP or PDBP expression, protein levels, or up-regulate PDZP, PDZD, PIP or PDBP activity can be monitored in clinical trails of subjects exhibiting decreased PDZP, PDZD, PIP or PDBP expression, protein levels, or down-regulated PDZP, PDZD, PIP or PDBP activity. Alternatively, the effectiveness of an agent determined to decrease PDZP, PDZD, PIP or PDBP expression, protein levels, or down-regulate PDZP, PDZD, PIP or PDBP activity, can be monitored in clinical trails of subjects exhibiting increased PDZP, PDZD, PIP or PDBP expression, protein levels, or up-regulated PDZP, PDZD, PIP or PDBP activity. In such clinical trials, the expression or activity of PDZP, PDZD, PIP or PDBP and, preferably, other genes that have been implicated in, for example, cancer can be used as a “read out” or markers for a particular cell's responsiveness.

For example, genes, including PDZP, PDZD, PIP or PDBP, that are modulated in cells by treatment with a modality (e.g., food, compound, drug or small molecule) can be identified. To study the effect of agents on disorders or disorders in a clinical trial, cells can be isolated and RNA prepared and analyzed for the levels of expression of PDZP, PDZD, PIP or PDBP and other genes implicated in the disorder. The gene expression pattern can be quantified by Northern blot analysis, nuclear run-on or RT-PCR experiments, or by measuring the amount of protein, or by measuring the activity level of PDZP, PDZD, PIP or PDBP or other gene products. In this manner, the gene expression pattern itself can serve as a marker, indicative of the cellular physiological response to the agent. Accordingly, this response state may be determined before, and at various points during, treatment of the individual with the agent.

A method for monitoring the effectiveness of treatment of a subject with an agent (e.g., an agonist, antagonist, protein, peptide, peptidomimetic, nucleic acid, small molecule, food or other drug candidate identified by the screening assays described herein) comprises the steps of (1) obtaining a pre-administration sample from a subject; (2) detecting the level of expression of a PDZP, PDZD, PIP or PDBP protein, PDZP, PDZD, PIP or PDBP mRNA, or genomic DNA in the preadministration sample; (3) obtaining one or more post-administration samples from the subject; (4) detecting the level of expression or activity of a PDZP, PDZD, PIP or PDBP, PDZP, PDZD, PIP or PDBP mRNA, or genomic DNA in the post-administration samples; (5) comparing the level of expression or activity of a PDZP, PDZD, PIP or PDBP, PDZP, PDZD, PIP or PDBP mRNA, or genomic DNA in the pre-administration sample with a PDZP, PDZD, PIP or PDBP, PDZP, PDZD, PIP or PDBP mRNA, or genomic DNA in the post administration sample or samples; and (6) altering the administration of the agent to the subject accordingly. For example, increased administration of the agent may be desirable to increase the expression or activity of PDZP, PDZD, PIP or PDBP to higher levels than detected, i.e., to increase the effectiveness of the agent. Alternatively, decreased administration of the agent may be desirable to decrease expression or activity of PDZP, PDZD, PIP or PDBP to lower levels than detected, i.e., to decrease the effectiveness of the agent.

2. Methods of Treatment

The invention provides for both prophylactic and therapeutic methods of treating a subject at risk of (or susceptible to) a disorder or having a disorder associated with aberrant PDZP, PDZD, PIP or PDBP expression or activity.

3. Disease and Disorders

Diseases and disorders that are characterized by altered PDZP, PDZD, PIP or PDBP levels or biological activity, such as rickettsial diseases, murine typhus, tsutsugamushi disease (Kim and Hahn, 2000), Facioscapulohumeral muscular dystrophy (Bouju et al., 1999; Kameya et al., 1999), chronic myeloid leukemia (Nagase et al., 1995; Ruff et al., 1999), Alzheimer's disease (Deguchi et al., 2000; Lau et al., 2000; McLoughlin et al., 2001; Tanahashi and Tabira, 1999a; Tomita et al., 2000; Tomita et al., 1999), neurological disorders such as Parkinson's disease and schizophrenia (Smith et al., 1999), X-linked autoimmune enteropathy (AIE) (Kobayashi et al., 1999), late onset demyelinating disease (Gillespie et al., 2000), Usher syndrome type 1 (USH1) (DeAngelis et al., 2001), nitric oxide-mediated tissue damage (Kameya et al., 1999; McLoughlin et al., 2001), tumors (Inazawa et al., 1996) and cystic fibrosis (Raghuram et al., 2001), may be treated with therapeutics that antagonize (i.e., reduce or inhibit) activity. Antagonists may be administered in a therapeutic or prophylactic manner. Therapeutics that may be used include: (1) PDZP, PDZD, PIP or PDBP peptides, or analogs, derivatives, fragments or homologs thereof; (2) Abs to PDZP, PDZD, PIP or PDBP; (3) PDZP, PDZD, PIP or PDBP-encoding nucleic acids; (4) administration of antisense nucleic acid and nucleic acids that are “dysfunctional” (i.e., due to a heterologous insertion within the coding sequences) that are used to eliminate endogenous function of by homologous recombination (Capecchi, 1989); or (5) modulators (i.e., inhibitors, agonists and antagonists, including additional peptide mimetic or Abs specific to PDZP, PDZD, PIP or PDBP) that alter the PDZD-mediated interaction.

Diseases and disorders that are characterized by decreased PDZP, PDZD, PIP or PDBP levels or biological activity may be treated with therapeutics that increase (i.e., are agonists to) activity. Therapeutics that up regulate activity may be administered therapeutically or prophylactically. Therapeutics that may be used include peptides, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying in vitro for RNA or peptide levels, structure and/or activity of the expressed peptides (or PDZP, PDZD, PIP or PDBP mRNAs). Methods include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situhybridization, and the like).

4. Prophylactic Methods

The invention provides a method for preventing, in a subject, a disease or condition associated with an aberrant PDZP, PDZD, PIP or PDBP expression or activity, by administering an agent that modulates PDZP, PDZD, PIP or PDBP expression or at least one PDZP, PDZD, PIP or PDBP activity. Subjects at risk for a disease that is caused or contributed to by aberrant PDZP, PDZD, PIP or PDBP expression or activity can be identified by, for example, any or a combination of diagnostic or prognostic assays. Administration of a prophylactic agent can occur prior to the manifestation of symptoms characteristic of a PDZP, PDZD, PIP or PDBP aberrancy, such that a disease or disorder is prevented or, alternatively, delayed in its progression. Depending on the type of PDZP, PDZD, PIP or PDBP aberrancy, for example, a PDZP, PDZD, PIP or PDBP agonist or PDZP, PDZD, PIP or PDBP antagonist can be used to treat the subject. The appropriate agent can be determined based on screening assays.

5. Therapeutic Methods

Another aspect pertains to methods of modulating PDZP, PDZD, PIP or PDBP expression or activity for therapeutic purposes. The modulatory method involves contacting a cell with an agent that modulates one or more of the activities of PDZP, PDZD, PIP or PDBP activity associated with the cell. An agent that modulates PDZP, PDZD, PIP or PDBP activity can be a nucleic acid or a protein, a PDZP, PDZD, PIP or PDBP, a peptide, a PDZP, PDZD, PIP or PDBP peptidomimetic, or other small molecule. The agent may stimulate PDZP, PDZD, PIP or PDBP activity. Examples of such stimulatory agents include active PDZP, PDZD, PIP or PDBP and a PDZP, PDZD, PIP or PDBP that has been introduced into the cell. In another embodiment, the agent inhibits PDZP, PDZD, PIP or PDBP activity. Examples of inhibitory agents include antisense PDZP, PDZD, PIP or PDBP nucleic acids and anti-PDZP, PDZD, PIP or PDBP Abs. Modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a PDZP, PDZD, PIP or PDBP or nucleic acid molecule. In one embodiment, the method involves administering an agent (e.g., an agent identified by a screening assay), or combination of agents that modulates (e.g., up-regulates or down-regulates) PDZP, PDZD, PIP or PDBP expression or activity. In another embodiment, the method involves administering a PDZP, PDZD, PIP or PDBP or nucleic acid molecule as therapy to compensate for reduced or aberrant PDZP, PDZD, PIP or PDBP expression or activity.

Stimulation of PDZP, PDZD, PIP or PDBP activity is desirable in situations in which PDZP, PDZD, PIP or PDBP is abnormally down-regulated and/or in which increased PDZP, PDZD, PIP or PDBP activity is likely to have a beneficial effect. Conversely, diminished PDZP, PDZD, PIP or PDBP activity is desired in conditions in which PDZP, PDZD, PIP or PDBP activity is abnormally up-regulated and/or in which decreased PDZP, PDZD, PIP or PDBP activity is likely to to have a beneficial effect.

6. Determination of the Biological Effect of the Therapeutic

Suitable in vitro or in vivo assays can be performed to determine the effect of a specific therapeutic and whether its administration is indicated for treatment of the affected tissue.

In various specific embodiments, in vitro assays may be performed with representative cells of the type(s) involved in the patient's disorder, to determine if a given therapeutic exerts the desired effect upon the cell type(s). Modalities for use in therapy may be tested in suitable animal model systems including, but not limited to rats, mice, chicken, cows, monkeys, rabbits, dogs and the like, prior to testing in human subjects. Similarly, for in vivo testing, any of the animal model system known in the art may be used prior to administration to human subjects.

7. Prophylactic and Therapeutic Uses of the Compositions

PDZP, PDZD, PIP or PDBP nucleic acids and proteins are useful in potential prophylactic and therapeutic applications implicated in a disorder.

PDZP, PDZD, PIP or PDBP nucleic acids, or fragments thereof, may also be useful in diagnostic applications, wherein the presence or amount of the nucleic acid or the protein is to be assessed. A further use could be as an anti-bacterial molecule (i.e., some peptides have been found to possess anti-bacterial properties). These materials are further useful in the generation of Abs that immunospecifically bind to the novel substances for use in therapeutic or diagnostic methods.

### EXAMPLES

The following examples are included to demonstrate preferred embodiments of the present invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing form the spirit and scope of the invention.

### Example 1.0

### Materials and Methods

1.1 Materials

Reagents for dideoxynucleotide sequencing were from United States Biochemical Corp. Enzymes and plasmid pMal-p2 were from New England Biolabs. Maxisorp immunoplates were from NUNC (Roskilde, Denmark). E. coli XL1-Blue and M13-VCS were from Stratagene. Bovine serum albumin (BSA) and Tween 20 were from Sigma (St. Louis, Mo.). Streptavidin was from Pierce.(Rockford, Ill.). Horseradish peroxidase/anti-M13 antibody conjugate, pGEX-4T-3, and glutathione-Sepharose were from Amersham Pharmacia Biotech. Anti-tetra-His antibody was from Qiagen. Anti-GST antibody was from Zymed Laboratories Inc. Horseradish peroxidase rabbit anti-mouse IgG antibody conjugate was from Jackson ImmunoResearch Laboratories. 3,3′,5,5′-Tetramethyl-benzidine/H₂O₂ (TMB) peroxidase substrate was from Kirkegaard & Perry Laboratories Inc. Preloaded Fmoc-Val-Wang resin and 2-(1H-benzotriazole-1-yl)-1,1,3,3-tetramethyluronium hexafluorophosphate (HBTU) were purchased from NovaBiochem.

1.2 Peptide Synthesis

Peptides were synthesized using standard 9-fluroenylmethoxycarbonyl (Fmoc) protocols, beginning with preloaded Fmoc-Val-Wang resin. Couplings were performed with a fourfold excess of amino acid activated with HBTU in the presence of a sixfold excess of diisopropylethylamine (DIPEA). Completed peptides were cleaved from the resin using a mixture of 2.5% water and 2.5% triisopropylsilane in trifluoroacetic acid (TFA) for 1 hour, purified by reversed phase high pressure liquid chromatography, and their masses verified by electrospray mass spectroscopy.

1.3 PDZ Domain Purification

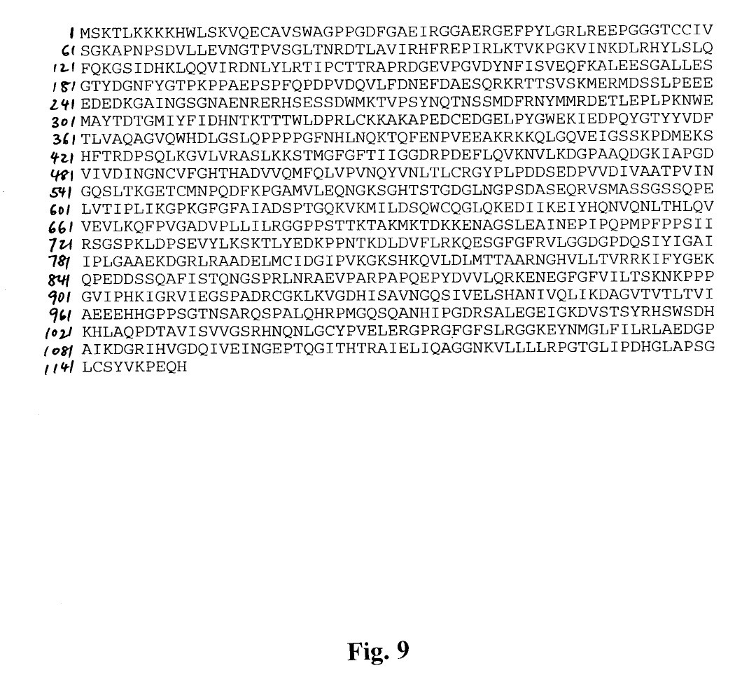

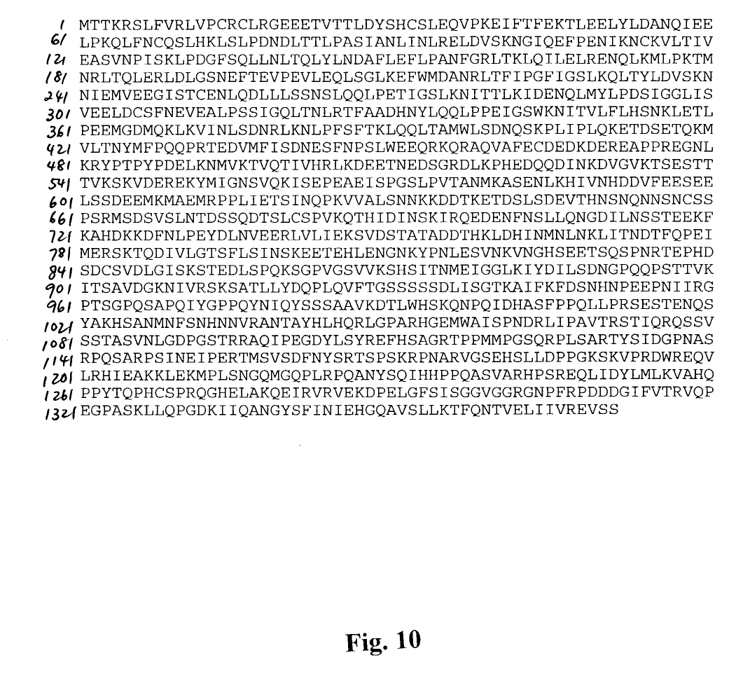

Mammalian: Expression constructs containing the six individual PDZ domains of MAGI-3 were constructed via PCR cloning using a full length cDNA of human MAGI-3 (Wu, Y. et al., 2000 J. Biol. Chem.) cloned into the pcDNA3.1/V5/His TOPO cloning vector (Invitrogen) as the template. PDZ 1 (aa. 417-535 of SEQ ID NO:200; FIG. 9), PDZ 2 (aa. 584-707 of SEQ ID NO:200), PDZ 3 (aa. 741-840 of SEQ ID NO:200) and PDZ 4 (aa. 870-976 of SEQ ID NO:200) were cloned into the BamHI/Not I sites of pEBG (Sanchez et al., 1994 Nature) creating in-frame fusions at the carboxy-terminus of GST. Regions of MAGI-3 containing PDZ 0 (aa. 1-406 of SEQ ID NO:200) and PDZ 5 (aa. 980-1151 of SEQ ID NO:200) were cloned into the Hind III/Sal I sites of pEGFP-N3 (Clontech) creating fusions onto the amino terminus of EGFP. The PDZ domain of ERBIN (aa. 1273-1371 of SEQ ID NO:201; FIG. 10) was amplified using PCR from EST AA992250 and cloned into the pcDNA 3.1-NT/GFP TOPO vector (Invitrogen) creating a fusion onto the C-terminus of GFP. The PDZ domain of ERBIN (aa. 1273-1371 of SEQ ID NO:201) was amplified using PCR from pGEX-6P-1 and cloned into pcDNA 3.1/V5/His to create a fusion protein with GST on the amino terminus, ERBIN PDZ in the middle and V5/His tags on the carboxy-terminus. Her 2 and Her 2 kinase dead (KD) constructs were cloned into pRK as described (Schaefer, G. et al, 1999 J. Biol. Chem.). Human δ-catenin and δ-catenin (Δ6 COOH aa.) were PCR cloned into pEGFP-C1 (Clontech) creating fusions onto the carboxy-terminus of EGFP.

Prokaryotic: The ERBIN PDZ domain (aa. 1217-1371 of SEQ ID NO:201) or MAGI-3 PDZ 2 (aa. 584-707 of SEQ ID NO:200) were cloned into the EcoR 1/Not 1 or BamH 1/Not 1 sites of pGEX 6P-1 and pGEX 4T-3 E. coli expression vectors (Pharmacia) respectively. Expression and affinity purification of E coli expressed GST-proteins was performed as recommended by the manufacturer (Pharmacia).

1.4 Vector Construction and Site-Directed Mutagenesis

A polymerase chain reaction was performed to amplify a 1.6-kilobase pair fragment of pMal-p2 containing the lacl^{q} gene and a gene fragment encoding the signal peptide from maltose-binding protein under the control of the P_{tac} promoter (forward primer, aaaagaattcccgacaccatcgaatggtgc (SEQ ID NO:202, and reverse primer, accagatgcataagccgaggcggaaaacatcatcg (SEQ ID NO:203; EcoRI site is in bold and NsiI site is in bold italics). The DNA fragment was digested with EcoRl and NsiI and ligated with the large fragment resulting from a similar digestion of a P8 display phagemid (Lowman et al., 1998). The method of Kunkel et al. (Kunkel et al., 1987) was used to insert eight codons (taataacatcaccatcaccatgcg; SEQ ID NO:204) immediately following the final codon of the P8 open reading frame. The resulting phagemid (designated pS1290a) contained the following DNA sequence downstream of the IPTG-inducible P_{tac} promoter: DNA encoding the maltose-binding protein signal peptide, mature P8, two stop codons (taataa; SEQ ID NO:205), apenta-His FLAG (HHHHHA; SEQ ID NO:206), and two more stop codons (tgataa; SEQ ID NO:207). Site-directed mutagenesis was used to delete the two stop codons between P8 and the penta-His FLAG or to replace them with varying numbers of Gly codons. The resulting phagemids secreted P8 moieties with carboxyl-terminal fusions consisting of various numbers of Gly residues followed by the penta-His FLAG.

1.5 Optimization of the Sequence Linking Peptides to the Carboxyl Terminus of P8

With phagemid pS 1290a as the template, a previously described method (Sidhu et al., 2000) was used to construct and sort linker libraries that replaced the two stop codons between P8 and the penta-His FLAG with 4, 5, 6, 8, or 10 degenerate codons. The libraries were pooled together to give a total diversity of 1.1×10¹¹. The pool was cycled through rounds of binding selection with an anti-tetra-His antibody as the capture target. After two rounds of binding selection, individual phage were isolated and analyzed in a phage ELISA by capturing the phage with the anti-tetra-His antibody and detecting bound phage (see below). Phage exhibiting strong signals in the phage ELISA were subjected to sequence analysis. The phagemid exhibiting the strongest ELISA signal was designated pS1403a.

1.6 Isolation of MAGI 3 PDZ Domain Binding Peptides (PDBPs)

Phagemid pS1403a was used as a template to construct a library (Sidhu et al., 2000) of P8 moieties with carboxyl-terminal fusions consisting of a 13-residue linker (AWEENIDSAPGGG; SEQ ID NO:199) followed by seven degenerate codons (NNS, where N=A/C/G/T and S=C/G). The diversity of the library was 2.0×10¹⁰. The library was cycled through rounds of binding selection with a GST-PDZ fusion protein coated on 96-well Maxisorp immunoplates as the capture target. Phage were propagated in E. coli SS320 (Sidhu et al., 2000) either with or without 10 μM IPTG induction. After three or four rounds of binding selection, individual phage were isolated and analyzed in a phage ELISA (see below). Phage that bound to the target GST-PDZ, but not to an unrelated GST-PDZ, were subjected to sequence analysis.

1.7 Library Synthesis

Compounds were synthesized beginning from the resin-attached dipeptide Fmoc-Trp-Val-Wang resin, prepared according to the peptide synthesis protocol. The Fmoc group was then removed through treatment of the resin with 20% piperidine in dimethylformamide (DMF) for 5 minutes, after which the liquids were filtered off and the resin washed 3 times with dichloromethane and 3 times with dimethylacetamide. When derivatizing the resin with isocyanates, the resin was suspended in N-methyl pyrrolidinone (NMP) and treated with 10 equivalents of reagent and agitated for 14 hours at room temperature. When derivatizing the resin with sulfonyl chlorides and chloroformates, the resin was suspended in NMP and treated with 10 equivalents of reagent and 30 equivalents of DIPEA and agitated for 14 hours at room temperature. When derivatizing the resin with acids, the resin was suspended in NMP and treated with a solution of 10 equivalents of acid, 10 equivalents of HBTU, and 30 equivalents of DIPEA and agitated for 14 hours at room temperature. Following the coupling reaction, the resin was washed 2 times with methanol, 2 times with dichloromethane, 2 times with NMP, 2 times with NMP containing 5% acetic acid and 2 times with dichloromethane. Finally, the compounds were cleaved from the resin through treatment with a mixture of 2.5% water and 2.5% triisopropylsilane in trifluoroacetic acid (TFA) for 1 hour, purified by reversed phase high pressure liquid chromatography, and their masses verified by electrospray mass spectroscopy.

1.8 Binding Assays

Binding of peptide-displaying phage particles to immobilized target proteins was detected using a phage ELISA. The assay was performed as described (Pearce et al., 1997), except that phage were produced in E. coli SS320, and assay plates were developed using a TMB peroxidase substrate system, read spectrophotometrically at 450 nm.

Binding affinities of the peptides for the ERBIN PDZ domain were determined as IC₅₀ values using competition ELISAs. The IC₅₀ value is defined as the concentration of peptide which blocks 50% of PDZ domain binding to an immobilized peptide. Assay plates were prepared by coating Maxisorp plates overnight at 4° C. with 65 μl of a 2 μg/ml solution of neutravidin in PBS. The plates were then blocked through addition of 65 μl of a 1% solution of bovine serum albumin (BSA) in PBS for 1 hour at room temperature, then washed 10 times with PBS containing 0.05% Tween-20. 65 μl of the amino-terminally biotinylated peptide PDZ 501 (TGWETWV; SEQ ID NO:222) was then added at a concentration of 100 nM in PBS with 0.5% BSA and 0.05% Tween-20 and incubated for 1 hour at room temperature. Simultaneously, binding reactions consisting of serial dilutions of the test compounds in PBS with 0.5% BSA and 0.05% Tween-20 containing 2 μg/ml ERBIN PDZ-GST fusion protein were incubated for 1 hour at room temperature. The plate coated with the immobilized peptide was again washed 10 times before 65 μl of each binding reaction was added to a well and incubated for 15 minutes at room temperature. The plates were again washed 10 times before being developed by incubating for 30 minutes with a 1:1000 dilution of anti-mouse HRP conjugated antibody and a 1:2000 dilution of a mouse anti-GST antibody in PBS with 0.5% BSA and 0.05% Tween-20. The plates were washed 10 times, then incubated with 100 μl HRP substrate for 5 minutes and the color developed through addition of 100 μl of 1 M H₃PO₄. The plates were read at 450 nm and the absorption fit to a binding curve using a least squares fit.

1.9 Peptide Concentration

Peptide concentrations were determined as described (Edelhoch, 1967). A concentrated stock of peptide was diluted into PBS and its absorbance measured at 267, 280 and 288 nm. The concentrations at each wavelength were calculated from their respective extinction coefficients and then averaged to give a final value.

1.10 Database Search and Determining Candidate PDZ Binding Partners

To determine candidate interacting proteins with the ERBIN PDZ domain, a three-step process was used. In the first step, a protein database was queried, examining only the C-termini, for the consensus binding sequence. In a second step, those proteins that were neither vertebrate nor intracellular (PDZ domains are found on cytoplasmic proteins) were removed. Finally, in a third step, redundant database entries and orthologs are eliminated.

Proteins with C-termini that resemble the phage-selected peptides against the ERBIN PDZ domain were identified using a motif-searching algorithm. Alignment of >100 phage selected peptides against the ERBIN PDZ established a clear consensus of D/E T/S W V (SEQ ID NO:208) as the preferred four C-terminal amino acids for tight binding to the ERBIN PDZ domain. This consensus was used to search the Dayhoff database (Dayhoff et al., 1978), restricting the search criteria to the C-terminal four amino acids of proteins within the database. Twenty-five proteins that ended with this C-terminal consensus were identified. Non-vertebrate proteins as well as one extracellular protein were manually filtered, leaving a total of 18 sequences that fit the criteria. Of these, several are orthologs or simply separate Genbank entries of the same gene product. Final examination of the 18 sequences suggests that at least three unique gene products are represented including, δ-catenin (not to be confused with δ-1 catenin which ′is another name for pp120ctn), armadillo protein deleted in velo-cardio-facial syndrome (ARVCF) (Sirotkin et al., 1997) and p0071 (plakophilin 4). These three proteins are all members of the Armadillo family of proteins which, based on their C-terminal four amino acids, were candidate ligands for the ERBIN PDZ domain in vivo. With this limited list, these Armadillio family members were selectively tested to determine if, in fact, they are ligands for the PDZ domain of ERBIN through subsequent in vitro and in vivo methods.

1.11 Co-Precipitation Assays

HEK 293 (293) cells grown in high glucose Dulbeco's Modified Eagle Medium (DMEM), 10% fetal calf serum, 1× non-essential amino acid supplement, 1×L-glutamine supplement, 10 mM HEPES (pH 7.4) and penicillin/streptomycin (all from Life Technologies) to ˜80% confluence were transfected with 2 μg DNA/35 mm diameter well (for example, DNAs encoding the sequences described in EXAMPLE 5.0) using Fugene reagent (Roche Biochemical). 24 hours post-transfection, cells were washed once with PBS and then scraped into 1 ml/well of 20 mM Tris (pH 7.5), 1% Triton X-100, 200 mM NaCl, 1 mM dithiothreitol, and protease inhibitor cocktail with EDTA (Roche Biochemical, catalog #1836145) and homogenized gently with three to five strokes in a dounce (Wheaton) using a loose glass pestle. Extracts were centrifuged at 12,000 rpm in a tabletop centrifuge at 4° C. for 10 minutes; the supernatant was combined with an equal volume of homogenization buffer without NaCl to achieve a final salt concentration of 100 mM and frozen at −70° C. until use. For peptide-pull-down experiments of MAGI-3 PDZ domains or the ERBIN PDZ domain, 100 μl of 293 cell extract was diluted to 400 μl in binding buffer (homogenization buffer modified to 100 mM NaCl) and incubated with 10 μM amino-terminally biotinylated peptide and 100 μl of strepavidin agarose (Sigma) for 2 hours on a rotator at 4° C. The beads were washed three times with 1 ml binding buffer and boiled in 60 μl of Laemmli's reducing sample buffer, of which 15 μl was loaded onto SDS-gels. PDZ domains co-precipitated with a given biotinylated peptide were visualized by immunoblot analysis using anti-GST (Genentech) or anti-GFP (Clontech) antibodies. For binding experiments with 293 cells expressing δ-catenin, δ-catenin (Δ6 COOH aa.) and Her 2, 400 μl of extract was diluted to 500 μl in binding buffer and incubated with 20 μg of E. coli-expressed GST-MAGI-3 PDZ 2 or GST-ERBIN PDZ and 50 μl of glutathione sepharose (Pharmacia) for 2 hours on a rotator at 4° C. Binding of 293 cell-expressed proteins was detected by immunoblot analysis using antibodies against GFP (Clontech) and Her 2 (Santa Cruz Biotechnology).

1.12 Peptide Targeting in Live Cells

Caco-2 cells were grown on polycarbonate transwell filters (12 mm diameter, 0.4 μm pore size; Costar) in same media as HEK 293 cells) until a fully polarized monolayer was obtained as determined by resistance measurements. The live cells were then incubated overnight with amino terminally, fluorescein (FAM) coupled peptides: (A) 2 μM of ATQITWV (SEQ ID NO:214), (B) 2 μM ATQITWA (SEQ ID NO:215) or (C) 5 μM ASKITWV (SEQ ID NO:216) added into the media of the lower transwell chamber. The cells were then washed with Hanks Balanced Salt Solution (HBSS) with 1.8 mM CaCl₂, fixed with ice cold methanol, permeabilized with 0.25% Triton X-100 in PBS, blocked with 5% donkey serum in PBS and stained with 1.5 μg monoclonal anti-γ-catenin antibody. The basolateral marker protein γ-catenin was visualized using Cy3-conjugated donkey anti-mouse antibodies (Jackson Immunolabs), diluted 1:1000. Processed filters were excised with a razor and mounted between a slide and coverslip with Vectashield mounting medium (Vector Labs; Burlingame, Calif.). Images were taken on a Leica confocal microscope using a 63X oil immersion objective.

1.13 Co-Localization of ERBIN PDZ and δ-Catenin

HEK 293 cells were grown to 70% confluence on collagen IV coated coverslips and then transfected with 1.4 μg of GST-ERBIN PDZ in pcDNA 3.1/V5/His and 1.1 μg of the indicated EGFP construct. 24 to 48 hours post-transfection, the cells were washed in PBS, fixed for 30 minutes in 2.5% formaldehyde, permeabilized with 0.25% Triton X-100 in PBS, and blocked with 5% donkey serum in PBS. The ERBIN PDZ domain was visualized by staining with monoclonal anti-V5 antibody (Invitrogen) and Cy3-conjugated secondary antibodies (Jackson Immunolabs) whereas GFP fusions were visualized directly. Images were taken on a standard fluorescence microscope using a 40× objective and digital CCD camera and SPOT imaging software (Diagnostic Instruments, Inc.; Sterling Heights, Mich.).

### Example 2.0

### Phage Display of Peptides Fused to the Carboxyl Terminus of P8

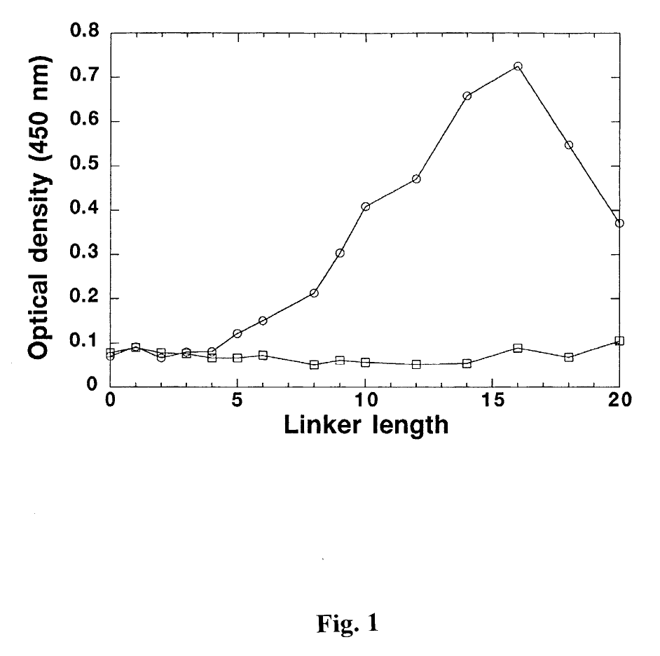

A series of phagemids were constructed, designed to ascertain whether peptides fused to the carboxyl terminus of P8 could be displayed on the surface of Ml 3 phage. Each phagemid was designed to secrete a P8 moiety with a penta-His FLAG epitope (HHHHHA; SEQ ID NO:217) fused to its carboxyl terminus. Co-infecting E. coli with the phagemid and a helper phage produced phage particles containing phagemid DNA. In such a system, the majority of the phage coat is composed of P8 molecules supplied by the helper phage, but the incorporation of some phagemid-encoded P8 molecules result in the display of the carboxyl-terminally fused penta-His FLAG. Penta-His FLAG display was detected with a phage ELISA using an anti-tetra-His antibody as the capture target. FIG. 1 shows that direct fusion of the FLAG to the carboxyl terminus of P8 did not result in display, but display was achieved by inserting five or more Gly residues between the P8 carboxyl terminus and the FLAG. Display levels increased steadily with increasing linker length, reaching a maximum with a 16-residue linker.

To optimize the linker sequence, libraries were constructed in which the linker connecting the penta-His FLAG to the P8 carboxyl terminus was designed to contain 4-6, 8, or 10 randomized residues. The libraries were pooled together and cycled through two rounds of binding selection on plates coated with the anti-tetra-His antibody. Many diverse sequences were selected, but all selectants contained either 8 or 10 residues. The best linker sequence (AWEENIDSAP, SEQ ID NO:218) increased display about 10-fold relative to polyglycine linkers of comparable length.

### Example 3.0

### Isolation of PDZ Domain Binding Peptides (PDBPs) for MAGI 3 (PDZ2 and PDZ3 Domains)

A library of random peptides fused to the carboxyl terminus of P8 with an optimized, intervening linker of 13 residues (AWEENIDSAPGGG, SEQ ID NO: 199) was constructed. At each library position, a degenerate codon that encoded all 20 natural amino acids and an amber (TAG) stop codon were used. The library contained seven degenerate codons and thus predominantly encoded heptapeptides, but the possible occurrence of amber stop codons also provided for the display of shorter peptides. The library contained 2.0×10¹⁰ unique members and thus exceeded the diversity of all possible natural heptapeptides (˜10⁹).

The library was used to investigate the binding specificities of PDZ domains 2 and 3 (PDZ2 and PDZ3, respectively) of MAGI 3, a membrane-associated guanylate kinase with inverted domain structure-3. PDZ2 interacts with the tumor suppressor PTEN/MMAC, whereas the binding specificity of PDZ3 is not known (Wu et al., 2000). PDZ2 and PDZ3 were purified as glutathione S-transferase (GST) fusions from E coli, and the phage-displayed peptide library was cycled through four rounds of binding selection against each domain. Transcription of the phagemid-encoded P8 gene is regulated by the Lac repressor, and display could thus be increased by the addition of IPTG. The PDZ2 sort was successful with or without IPTG, but the PDZ3 sort yielded binding clones only with IPTG induction.

The PDZ2 sort yielded a variety of sequences varying in length from seven to four residues (Table 1). The four carboxyl-terminal residues showed a strong consensus to the sequence Cys/Val-Ser/Thr-Trp-Val-COOH (SEQ ID NO:219), a type 1 PDZ binding consensus related to, but distinctly different from, the carboxyl-terminal sequence of PTEN/MMAC (Tables 1 and 2). Although many of the sequences were represented by unique clones, two carboxyl-terminal sequences appeared multiple times (CSWV and VTWV, SEQ ID NOs:2 and 4), both as tetrapeptides and also at the carboxyl termini of longer peptides. Thus, these two sequences represented minimal, high affinity ligands of PDZ2. The PDZ3 sort yielded only a single heptapeptide (TRWWFDI, SEQ ID NO: 13), a type II PDZ-binding motif that differs completely from the PDZ2 binding consensus.

TABLE 1
Phage-displayed selectants,
MAGI 3 PDZ2 and PDZ3 domains

| Peptide sequence | SEQ ID NO: |
|---|---|
| PDZ2 binders | |
| DGICSWV | 1 |
| CSWV | 2 |
| ASKVTWV | 3 |
| VTWV | 4 |
| EAQCTWV | 5 |
| LEVCSWV | 6 |
| WGPCTWV | 7 |
| PCSWV | 8 |
| IERTTWV | 9 |
| HEEWTWV | 10 |
| GGDCHWV | 11 |
| HKDCHWV | 12 |
| PDZ3 binders | |
| TRWWFDI | 13 |

Peptides corresponding to the selected sequences were synthesized and assayed for binding (Table 2). The selected peptides bound their cognate PDZ domains with high affinity while exhibiting no detectable binding to non-cognate PDZ domains. Amidation of the carboxyl terminus of the PDZ3-specific peptide resulted in a 300-fold reduction in binding affinity, demonstrating the importance of interactions between PDZ3 and the terminal carboxylate of its ligand. The data also confirmed that the minimal tetrapeptide selectants from the PDZ2 sort bind PDZ2 with high affinity. Surprisingly, the selectants bound PDZ2 much more tightly than a heptapeptide corresponding to the carboxyl-terminal sequence of PTEN/MMAC. It appears that this large difference in binding affinity is attributable to the residue at P(−1), which is a Trp in the selected peptide as opposed to a Lys in PTEN/MMAC (compare HTQITWV with HTQITKV (SEQ ID NO:220), Table 2; The IC₅₀ values are the concentrations of peptide that blocked 50% of PDZ domain binding to immobilized peptide in an ELISA).

TABLE 2
IC₅₀ values for MAGI 3 PDZ2 and PDZ3 domain-binding
synthetic peptides

| Position | | | | | | | IC₅₀ (μM) | | |
|---|---|---|---|---|---|---|---|---|---|
| −6 | −5 | −4 | −3 | −2 | −1 | 0 | PDZ2 | PDZ3 | SEQ ID NO: |
| H | T | Q | I | T | K | V | 200 | NDI | 182 |
| H | T | Q | I | T | W | V | 0.3 | | 183 |
| D | G | I | C | S | W | V | 0.3 | NDI | 184 |
| G | C | G | C | S | W | V | 2.0 | | 185 |
| | | | C | S | W | V | 1.4 | | 186 |
| | | | A | S | W | V | 35 | | 187 |
| | | | C | A | W | V | 7.3 | | 188 |
| | | | C | S | A | V | 200 | | 189 |
| | | | C | S | W | A | 400 | | 190 |
| A | S | K | V | T | W | V | 0.8 | NDI | 191 |
| | | | V | T | W | V | 4.0 | | 192 |
| T | R | W | W | F | D | I | NDI | 0.9 | 193 |
| T | R | W | W | F | D | I-NH₂ | | 300 | 194 |

^{a}The carboxy-terminal sequence of PTEN/MMAC.
^{b}NDI indicates no detectable inhibition at peptide concentrations greater than 1 mM.

To assess the contributions of individual ligand side chains to the binding interaction, the tetrapeptide exhibiting the highest affinity for PDZ2 (CSWV, SEQ ID NO:2) was subjected to an alanine scan. A peptide series was synthesized to convert individually each amino acid within the tetrapeptide to an Ala residue. The results indicate that all four side chains contribute favorably to the binding interaction (Table 2), but the magnitudes of the contributions vary. Ala substitution at P(0) or P(−1) reduced binding by more than 100-fold, whereas substitution of the serine residue at P(−2) caused only a 5-fold reduction. Ala substitution of the cysteine residue at P(−3) caused an intermediate 25-fold reduction in binding.

### Example 4.0

### Modeling the PDZ2-PDBP Interaction

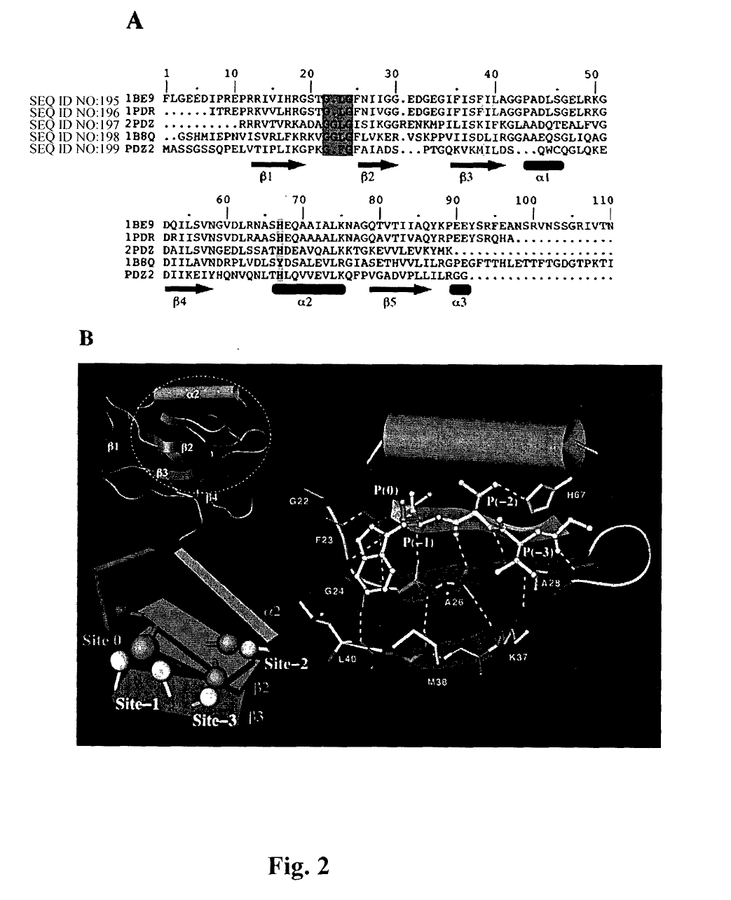

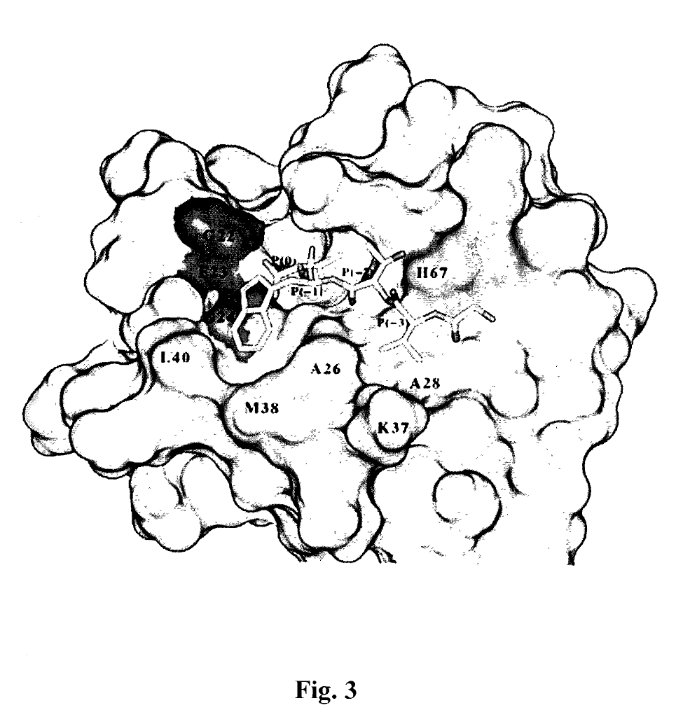

Homology modeling techniques were used to build a three-dimensional model of PDZ2 in complex with the high affinity pentapeptide ligand GVTWV (SEQ ID NO:240) (FIGS. 2 and 3). The model was based on the crystal structures of the third PDZ domain from the human homolog of discs-large protein (Morais Cabral et al., 1996) and the third PDZ domain of PSD-95 (PSD-95-3) in complex with a pentapeptide (KQTSV) (Doyle et al., 1996). The model and the peptide alanine scan data help to define the binding interactions between PDZ2 and peptide ligands. In both the crystal structure and the model, the peptide ligand forms a β strand that intercalates between β2 and β2 of the PDZ domain, extending the antiparallel β sheet formed by β2 and β3 of the protein (FIG. 2). The terminal carboxylate of the peptide interacts with the highly conserved carboxylate binding loop (main chain of residues Gly-22, Phe-23, and Gly-24), whereas the P(0) Val side chain resides in a well defined hydrophobic pocket. In the PSD-95-3/KQTSV crystal structure, the side chain of Ser at P(−1) is solvent-exposed, and it does not interact with the protein (FIG. 2). Thus, the P(−1) side chain in PDZ domain ligands has been considered unimportant for binding, and the type I consensus sequence X-Ser/Thr-X-Val-COOH has been proposed (Doyle et al., 1996). In contrast, the bulky Trp side chain at P(−1) of our high affinity ligands can be modeled to pack against the protein (FIG. 2), establishing favorable Van der Waals contacts with the side chains of Met-38 and Leu-40 in the β3 strand (FIG. 3). These interactions would bury a large hydrophobic area and greatly stabilize the complex. This prediction is supported by the dramatic reduction in binding upon substitution of Trp with Ala at P(−1) (Table 2). Met-38 and Leu-40 are not conserved in the PDZ family (FIG. 2), indicating that interactions between side chains at these positions and peptide side chains at P(−1) may contribute not only to binding affinity but also to specificity. At P(−2), the Thr side chain makes a hydrogen bond to the conserved His-67 residue in both the crystal structure and the model (FIG. 2). However, the interaction is solvent-exposed, and Ala substitution at this position has only a modest effect on affinity (Table 2). Thus, the side chain at P(−2) may determine specificity, but it makes only a minor contribution to affinity in the case of PDZ2 binding to the selected peptides. Finally, the binding contribution of the hydrophobic side chain at P(−3) can be rationalized in terms of favorable Van der Waals contacts with a hydrophobic patch on the protein formed by the side chains of residues Ala-26 and Ala-28 in the β2 strand and the side chain of Lys-37 in the β3 strand (FIGS. 2 and 3). These results confirm the importance of the previously described interactions between the carboxyl terminus of the peptide ligand and the carboxylate binding loop of the PDZ domain. In addition, these data highlight contributions to binding affinity and specificity attributable to interactions between hydrophobic side chains at P(−1) and P(−3) of the peptide ligand and side chains in the P2 and P3 strands of the PDZ domain.

### Example 5.0

### PDBPs for MAGI 3 PDZ 2 or PDZ 3 Bind Specifically

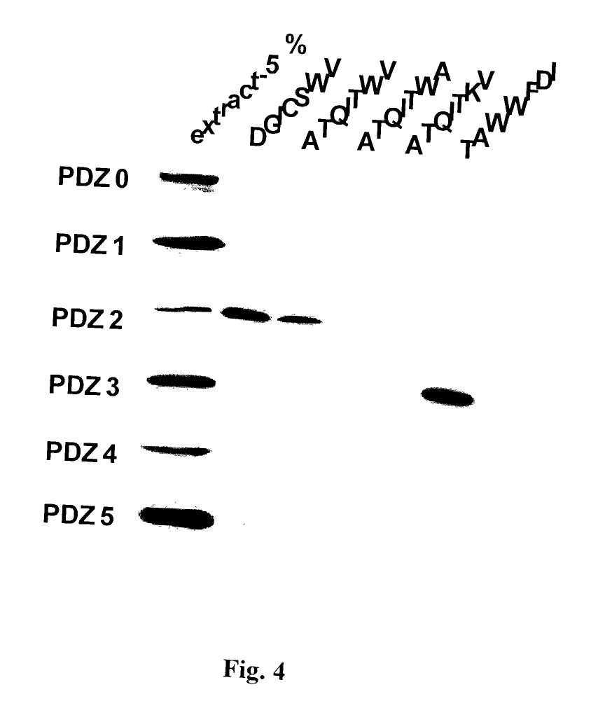

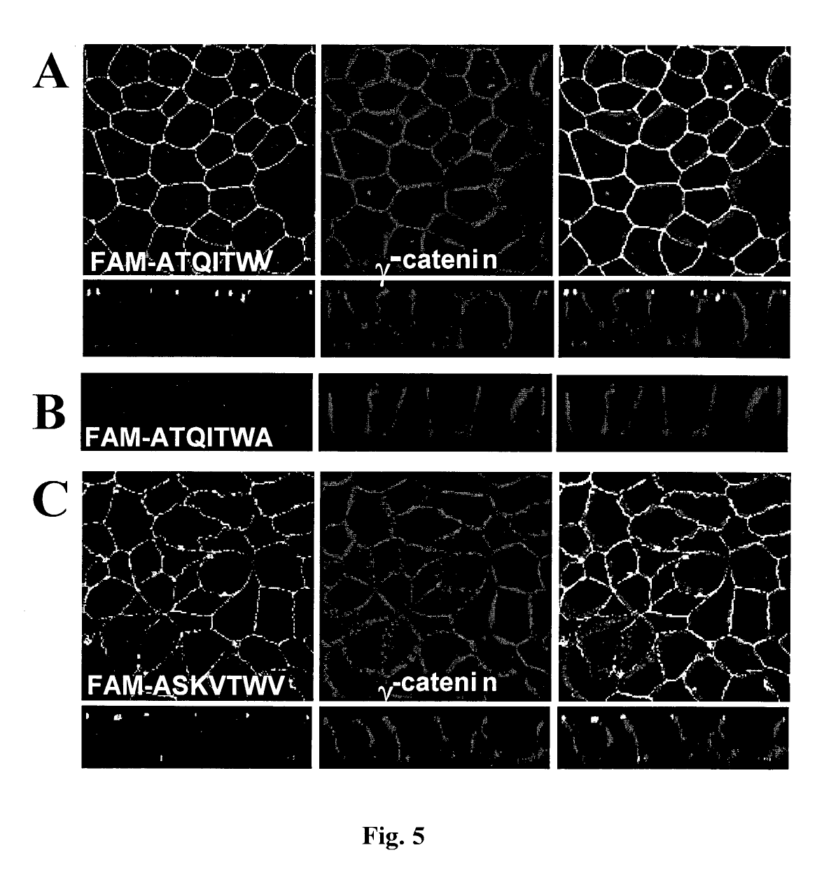

Each of the six PDZ domains of MAGI 3 was expressed in HEK 293 cells as GST fusions (PDZ 1; aa. 417-535, SEQ ID NO:200, PDZ 2; aa. 584-707, SEQ ID NO:200, PDZ 3; aa. 741-840, SEQ ID NO:200, and PDZ 4; aa. 870-976, SEQ ID NO:200) or EGFP (PDZ 0; aa. 1-406, SEQ ID NO:200, and PDZ 5; aa. 980-1151, SEQ ID NO:200) and tested for the ability to be precipitated from cell extracts by the indicated biotinylated peptide. Only PDZ 2 and 3 significantly bound to their cognate phage-selected peptides (FIG. 4). These same PDZ domains did not bind to the peptides ATQITWA (SEQ ID NO:215 or ATQITKV (SEQ ID NO:214) which contain V to A or W to K changes at the (0) and (−1) positions respectively. Note: ATQITWV (SEQ ID NO:214) was not obtained from the phage screen but is a derivative of the C-terminus of PTEN (HTQITKV; SEQ ID NO:220), a low affinity ligand of MAGI-3 PDZ 2. Examination of phage-selected peptides of PDZ 2 suggested that changing K to W at the (−1) position of the PTEN C-terminus would increase binding affinity. Comparison of the results in lanes 3 and 5 clearly show this to be true (FIG. 4).

### Example 6.0

### MAGI-3 PDZ2 PDBPs are Targeted to the Tight Junctions in Live Caco-2 Cells

Caco-2 cells were grown on polycarbonate transwell filters until a fully polarised monolayer was obtained. The live cells were then incubated overnight with the fluorescein (green) coupled peptides: (A) 2 mM of ATQITWV (SEQ ID NO:214), (B) 2 mM ATQITWA (SEQ ID NO:215) or (C) 5 mM ASKITW (SEQ ID NO:221) (FIG. 5). The cells were then fixed and counterstained with antibodies against the protein γ-catenin (FIG. 5). In contrast to the basolateral staining pattern observed for g-catenin, (A) ATQITWV (SEQ ID NO:214) and (C) ASKITWV (SEQ ID NO:216) localize apically on the lateral membrane to the tight junction. Substitution of A for V at the peptide carboxyl terminus should disrupt the interaction of a ligand with its cognate PDZ binding partner. Accordingly, the peptide ATQITWA (SEQ ID NO:215) in panel B (FIG. 5) does not target to the tight junction. Notably, MAGI-3 is found at the tight junction in these cells.

### Example 7.0

### Isolation of PDBPs for ERBIN PDZ Domain

A library of random peptides fused to the carboxyl terminus of P8 with an optimized, intervening linker of 13 residues (AWEENIDSAPGGG, SEQ ID NO: 199) was constructed. At each library position, a degenerate codon that encoded all 20 natural amino acids and an amber (TAG) stop codon were used. The library contained seven degenerate codons and thus predominantly encoded heptapeptides, but the possible occurrence of amber stop codons also provided for the display of shorter peptides. The library contained 2.0×10¹⁰ unique members and thus exceeded the diversity of all possible natural heptapeptides (˜10⁹).

ERBIN PDZ domain was purified as a glutathione S-transferase (GST) fusion from E. coli, and the phage-displayed peptide library was cycled through four rounds of binding selection against the ERBIN PDZ domain. The Lac repressor regulates transcription of the phagemid-encoded P8 gene, and display could thus be increased by the addition of IPTG.

The ERBIN PDZ sort yielded a variety of sequences varying in length from seven to four residues (Table 3). The four carboxyl-terminal residues showed a strong consensus to the sequence D(E)T(S)WV (SEQ ID NO:221).

TABLE 3
Phage-displayed selectants, ERBIN PDZ domain

| | | | |
|---|---|---|---|
| ERBIN PDBP | | ERBIN PDBP | |
| candidates | SEQ ID NO: | candidates | SEQ ID NO: |

| | | | |
|---|---|---|---|
| G Q D E T W V | 14 | V G S D T W V | 89 |
| D T W S T W V | 15 | R L W D S W V | 90 |
| N A W D E W V | 16 | C N I E S W V | 92 |
| W E T W V | 17 | A G G E S W V | 93 |
| S D W E S W V | 18 | C Y Q D T W V | 94 |
| L W V E T W V | 19 | E W G G T W V | 95 |
| R W Y D D W V | 20 | A G R D T W V | 96 |
| G G W E T W V | 21 | Y Q K E T W V | 97 |
| W G S D T W V | 22 | R F H D T W V | 98 |
| S Y F D S W V | 23 | T R F E T W V | 99 |
| P K W D T W V | 24 | R W R E S W V | 100 |
| Q H W D T W V | 25 | R S Y E T W V | 101 |
| R S R E T W V | 26 | T L L E T W V | 102 |
| V F H D T W V | 27 | S W D S W V | 103 |
| R H A D T W V | 28 | L T P E T W V | 104 |
| W T E G T W V | 29 | V Q D T W V | 105 |
| K F M D T W V | 30 | G A M D T W V | 106 |
| W P W D S W V | 31 | K G P E T W V | 107 |
| C E G D T W V | 32 | S V W E S W V | 108 |
| A W Y E T W V | 33 | G W Y D S W V | 109 |
| G Q F D S W V | 34 | C H K D T W V | 110 |
| S W W D T W V | 35 | T G I D T W V | 111 |
| F S D T W V | 36 | A S G E S W V | 112 |
| S P F E T W V | 37 | S H N E T W V | 113 |
| R W E T W V | 38 | W E T W V | 114 |
| W D E T W V | 39 | L G R E T W V | 115 |
| G E Y D T W V | 40 | D R E T W V | 116 |
| S C N D T W V | 41 | W D T W V | 117 |
| R W R D T W V | 42 | W K G D T W V | 118 |
| S V W E T W L | 43 | I H S D T W V | 119 |
| P C K D T W V | 44 | G Q W D S W V | 120 |
| R Y D D T W V | 45 | G A S D T W V | 121 |
| K G W D T W V | 46 | R Y D E T W I | 122 |
| S Y L E T W V | 47 | R G M E T W V | 123 |
| K P P E T W V | 48 | S S Y D S W V | 124 |
| S Q R D T W V | 49 | R D M D T W V | 125 |
| T R F E T W V | 50 | W H D T W V | 126 |
| L R R E T W V | 51 | R R E T W V | 127 |
| Q E W D S W V | 52 | V F F D T W V | 128 |
| R D I D T W L | 53 | H G W D T W V | 129 |
| Q D R E T W V | 54 | S A W D S W V | 130 |
| N F E T W V | 55 | S R V E T W V | 131 |
| R G L D T W V | 56 | R P E T W V | 132 |
| N G C D T W V | 57 | S D W D T W V | 133 |
| Y G D S W V | 58 | T R W D T W V | 134 |
| R Q L D T W V | 59 | G T L D T W V | 135 |
| K S L D S W V | 60 | L W H D T W V | 136 |
| V F W E S W V | 61 | W P R D T W V | 137 |
| S Y F D T W V | 62 | G P W E T W V | 138 |
| S W D S W V | 63 | H K E T W V | 139 |
| I E D S W V | 64 | Q D S W V | 140 |
| W W A D V W V | 65 | G R D T W V | 141 |
| R G T D T W V | 66 | R E D T W V | 142 |
| Q E Y D T W V | 67 | K G W E S W V | 143 |
| G W D G T W V | 68 | W L E S W V | 144 |
| D T W V | 69 | L W D E T W V | 145 |
| S Y D E S W L | 70 | G N V D T W V | 146 |
| R D M D T W V | 71 | C H R D T W V | 147 |
| Y D G D T W V | 73 | R G S D T W V | 148 |
| A F P D V W V | 74 | K D T W V | 149 |
| S W W D T W V | 75 | G W M D T W V | 150 |
| H W I E T W V | 76 | R D L D T W V | 151 |
| V R R E T W V | 77 | D T W V | 152 |
| W D G D S W V | 78 | A V R D T W V | 153 |
| A D T W V | 79 | M E W E T W V | 154 |
| V K R E T W V | 80 | K E Y D T W V | 155 |
| G F D D T W V | 81 | R G I D T W V | 156 |
| K G K D T W V | 82 | M S R D T W V | 157 |
| R F E S W V | 83 | R Q W D S W V | 158 |
| R G G D T W V | 84 | R G G D T W V | 159 |
| G V F D S W V | 85 | E T W V | 160 |
| R G W E T W L | 86 | R V W D T W V | 161 |
| S D W E S W V | 87 | R Y E E T W L | 162 |
| D W Y D T W V | 88 | W D I D V W V | 163 |

### Example 8.0

### Database Search for Proteins Whose Carboxyl Termini Resemble the ERBIN PDBPs

To determine candidate proteins that interact/bind with the ERBIN PDZ domain, the Dayhoff protein database was queried, examining only the C-terminal 4 amino acid residues, for the consensus binding sequence noted above. Those proteins that were neither vertebrate nor intracellular were removed. Finally, redundant database entries and orthologs were eliminated.

A total of 25 proteins were identified from the search. The search criteria consisted of the four amino acid consensus sequence D(E)T(S)WV (SEQ ID NO:221), with this motif being constrained to the carboxy-terminus of the protein. Extracellular proteins or those from non-vertebrate species have been removed from the list shown in Table 4. All 18 proteins are members of the Armadillo family of proteins.

TABLE 4
Vertebrate proteins with carboxy termini resembling ERBIN PDBPs

| | SEQ |
|---|---|
| Protein | ID NO: |
| DSWV T42209 neural plakophilin related arm-repeat protein | 688 |
| NPRAP - mouse, 135,000 Da | |
| DSWV ARVC_HUMAN Armadillo repeat protein deleted in | 689 |
| velo-eardio-facial syndrome, 104,642 Da | |
| DSWV P_AAW24559 Presenilin-interacting protein GT24 - | 690 |
| Homo sapiens., 112,826 Da | |
| DSWV P_AAW60664 Human ALARM protein - Homo | 691 |
| sapiens., 83,140 Da | |
| DSWV P_AAY23899 Human resenilin binding armadillo | 692 |
| protein p0071 - Homo sapiens., 131,868 Da | |
| DSWV P_AAY23 900 Human resenilin binding armadillo | 693 |
| protein GT24/hNPRAP - Homo, 117,435 Da | |
| DSWV P_AAB07973 A human neural plakophilin related | 694 |
| armidillo protein - Homo, 132,656 Da | |
| DSWV P_AAB07974 A murine neural plakophilin related | 695 |
| armidillo protein - Mus sp., 135,000 Da | |
| DSWV NM_001670_1 armadillo repeat | 696 |
| protein - Homo sapiens, 104,642 Da | |
| DSWV NM_008729_1 neural plakophilin-related arm-repeat | 697 |
| protein - Mus musculus, 135,000 Da | |
| DSWV AB013805_1 neural plakophilin-related arm-repeat | 698 |
| protein (NPRAP) - Homo, 132,656 Da | |
| DSWV AF287051_1 catenin arvcf-2ABC protein - Xenopus | 699 |
| laevis, 101,573 Da | |
| DSWV HSU52351_1 arm-repeat protein NPRAP/neurojungin - | 700 |
| Homo sapiens, 96,443 Da | |
| DSWV HSU52828_1 δ-catenin - Homo sapiens, 40,247 Da | 701 |
| DSWV HSU72665_1 GT24 - Homo sapiens, 34,417 Da | 702 |
| DSWV HSU81004_1 GT24 - Homo sapiens, 112,810 Da | 703 |
| DSWV HSU96136_1 δ-catenin - Homo sapiens, 132,665 Da | 704 |
| DSWV AF035302_1 similar to δ-catenin - | 705 |
| Homo sapiens, 36,108 Da | |

### Example 9.0

### δ-Catenin Binds to the ERBIN PDZ Domain and an Important Component of the Interaction is Mediated by its C-Terminus

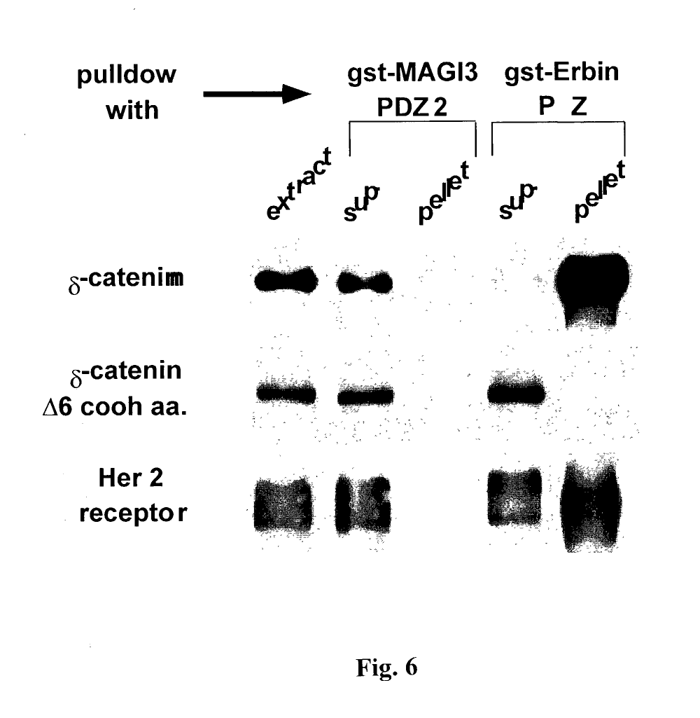

Amino acids 1217-1371 of ERBIN and 584-707 corresponding to PDZ 2 of MAGI-3 (Sidhu et al., 2000); were expressed in E. coli as GST fusions. The PDZ-fusions were then tested for their ability to precipitate (A) δ-catenin, (B) δ-catenin with the six C-terminal amino acids deleted or (C) the Her 2 receptor present in extracts from transfected HEK 293 cells (FIG. 6). Examination of the amino acid sequence of phage-selected peptides against the ERBIN PDZ domain suggested that δ-catenin was a potential ligand for this PDZ domain. The results in the top panelof FIG. 6 demonstrate that δ-catenin binds strongly to the ERBIN PDZ domain but not to PDZ 2 of MAGI-3. The middle panel of FIG. 6 demonstrates a common characteristic of PDZ ligands, that the C-terminus of δ-catenin is necessary for tight binding. The lower panel shows that Her 2, a previously reported ligand for the ERBIN PDZ, is specifically precipitated in this assay. However, much less Her 2 than δ-catenin is depleted from the cell extract, suggesting that the δ-catenin:ERBIN PDZ interaction is higher affinity. Equal volumes of extract and depleted extract (sup.) were analyzed.

### Example 10.0

### The Erbin pdz Domain Associates with δ-catenin in Vivo

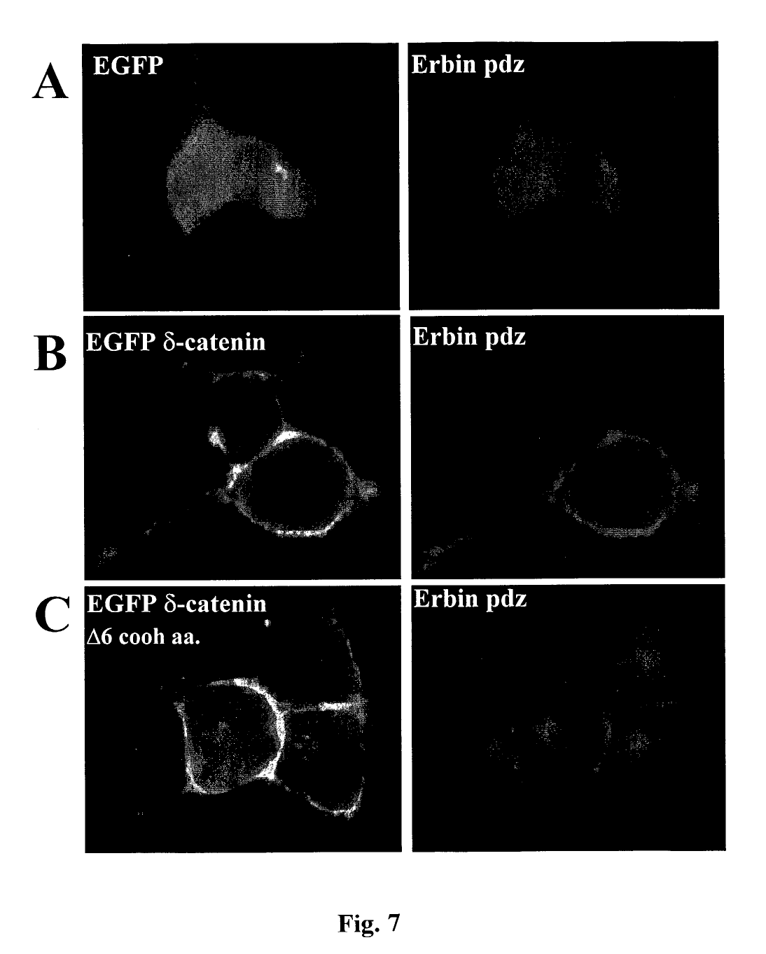

The ERBIN PDZ domain was co-transfected into HEK 293 E cells with EGFP, human δ-catenin or human δ-catenin missing the six C-terminal amino acids (FIG. 7). Panel A shows that in the absence of δ-catenin the ERBIN PDZ domain resides primarily in the cytoplasm or endoplasmic recticulum whereas complete recruitment of ERBIN PDZ to the cell junction is observed in the presence of δ-catenin (B). Deletion of the six carboxy-terminal amino acids of δ-catenin abrogates most, but not all, of the co-localization of ERBIN PDZ with δ-catenin. These data suggest that the C-terminus of 6-catenin is required for a high affinity interaction with the PDZ domain of ERBIN.

### Example 11.0

### A Single Amino Acid Change at the (−3) Position of a PDZ Peptide Ligand Alters its Binding Specificity (ERBIN and MAGI 3 PDZ Domains)

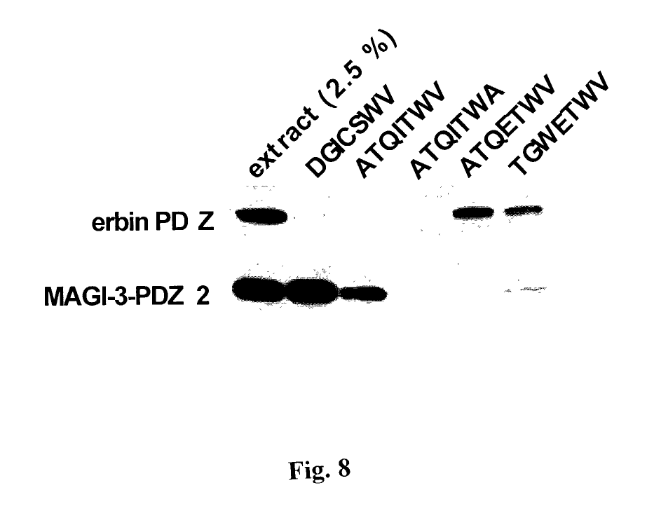

The ERBIN PDZ domain or the second PDZ domain of MAGI-3 was expressed in HEK 293 cells as fusions with GFP and GST, respectively. The indicated biotinylated peptides (FIG. 8) were then tested for their ability to bind to each PDZ domain in cell extracts. The results (FIG. 8) show that the peptides phage selected against MAGI-3 PDZ 2 and ERBIN PDZ, lanes 2 and 6 respectively, efficiently precipitate only the PDZ domain that they were phage-selected against. This is also true of the ATQITWV (SEQ ID NO:214) peptide (lane 3), a derivative of the PTEN protein C-terminus (a low affinity ligand for MAGI-3 PDZ 2), altered at the (−1) position from K to W to increase its affinity for PDZ 2. All phage-selected peptides against MAGI-3 PDZ 2 have an I, V or C at the (−3) position, whereas, a D or E appear exclusively in peptides phage selected against the ERBIN PDZ. Simply changing the I to an E in the PDZ 2 binding peptide ATQITWV (SEQ ID NO:214) at this position switches the binding specificity of the peptide from a MAGI-3 PDZ 2 binder to an ERBIN PDZ binder. These data suggest that amino acids with significantly different side chains at the (−3) position of PDZ protein ligands allows the ligand to discriminate between multiple potential PDZ binding partners, even if the C-termini PDZ-binding motifs are otherwise identical.

### Example 12.0

### The ERBIN PDZ Binding Peptides Found by Phage Display Bind with Higher Affinity to ERBIN Than Previously-Identified PDZ Protein ERBB2/Her2

ERBIN has been identified as a ligand for ERBB2/HER2 receptor. However, the database query did not identify ERBB2/Her2 receptor as having the consensus sequence for an ERBIN PDBP as identified by phage display.

The binding of ERBIN to the phage displayed-identified ligands (TGWETWV and TGWDTWV, SEQ ID NOs:222-223) was compared to that of the previously-identified ligand described in ERBB2/Her2, DVPV (SEQ ID NO:224) (Borg et al., 2000) in the in vitro assay (described above).

ERBIN bound to the phage display-identified PDBPs TGWETWV and TGWDTWV (SEQ ID NOs:222-223) with high affinity in the presence of competitor, PDZ 501 (TGWETWV; SEQ ID NO:222). The IC₅₀ for TGWETWV (SEQ ID NO:222) was 0.5 to 1 μM, and that for TGWDTWV (SEQ ID NO:223) was 4.5 to 5.0 μM. However, the previously identified ligand DVPV (SEQ ID NO:224) bound poorly, giving an IC50 of greater than 400 μM, while the DVPA ligand was greater than 100 μM.

When a similar experiment was carried out examining the MAGI 3 PDZ2 domain “naturally-selected” ligand, ITKV (SEQ ID NO:225) and compared to those identified by phage display (CSWV and VTWV, SEQ ID NOs:2 and 4), a similar difference in binding affinities was observed. Where as MAGI 3 bound to ITKV with an IC₅₀ of 200 μM, the phage displayed PDBPs bound with observed IC₅₀s of 1 μM (CSWV; SEQ ID NO:2) and 41M (VTWV; SEQ ID NO:4).

### Example 13.0

### Analysis ERBIN PDBP Consensus

Alanine scanning the of the ERBIN PDZ binding consensus peptide, WETWV (SEQ ID NO:225) was performed to determine the relative contribution to PDZ binding.

Binding affinities of the peptides for the ERBIN PDZ domain were determined as IC₅₀ values using competition ELISAs. The IC₅₀ value is defined as the concentration of peptide which blocks 50% of PDZ domain binding to an immobilized peptide. Assay plates were prepared by coating microwell plates overnight with neutravidin. The plates were then blocked through addition of BSA, and then amino-terminally biotinylated WETWV (SEQ ID NO:225) was then bound to the plates. Simultaneously, binding reactions consisting of serial dilutions of the test peptides with ERBIN PDZ-GST fusion proteins were performed. The plate coated with the immobilized WETWV (SEQ ID NO:225) was extensively washed before adding each binding reaction to the wells and briefly incubated. After further washing, anti-mouse HRP conjugated antibody and a mouse anti-GST antibody were added. The plates were then developed with HRP substrate and H₃PO₄, and then read at 450 nm. The absorption fit to a binding curve using a least squares fit. Thus the ability of the various peptides to inhibit ERBIN PDZ domain from binding its cognate was measured.

Alanine scanning an acylated WETWV (SEQ ID NO:225) peptide results in peptides that are less potent inhibitors of ERBIN PDZ-GST fusion binding to the immobilized PDBP (Table 5); reducing potency from 8.2 to 69.3 fold.

TABLE 5
Alanine scanning of WETWV (SEQ ID NO:225) peptide

| Peptide | SEQ ID | IC₅₀ | fold less potent than Ac-WETWV |
|---|---|---|---|
| sequence | NO: | (μM) | (SEQ ID NO:225) |
| Ac-WETWV | 225 | 0.5 | 1 |
| Ac-AETWV | 226 | 4.0 | 8.2 |
| Ac-WATWV | 227 | 14.8 | 30.3 |
| Ac-WEAWV | 228 | 12.4 | 25.3 |
| Ac-WETAV | 229 | 34.0 | 69.3 |

Substituting for tryptophan at the −1 position with alanine, phenylalanine or tyrosine also significantly reduces the potency of the peptide to act as an inhibitor (Table 6); however, 2-napthylalanine had almost no effect.

TABLE 6
Substitutions for tryptophan at the −1 position

| Peptide | SEQ ID | IC₅₀ | fold less potent |
|---|---|---|---|
| sequence | NO: | (μM) | than Ac-WETWV |
| Ac-WET WV | 225 | 0.5 | 1 |
| (SEQ ID | | | |
| NO:225) | | | |
| Ac-WETAV | 230 | 34.0 | 69.3 |
| Ac-WETFV | 231 | 14.5 | 295 |
| Ac-WETNapV | 232 | 0.6 | 1.1 |
| Ac-WETYV | 233 | 42.5 | 86.7 |

However, when the −3 (threonine) and 4 (glutamate) positions are substituted (with serine, valine, or threonine), potency is reduced, but not to the extent of most of the −1 position substitutions (Table 7).

TABLE 7
Substitutions for threonine at the −2 position and
glutamate at the −3 position

| Peptide | SEQ ID | IC₅₀ | fold less potent |
|---|---|---|---|
| sequence | NO: | (μM) | than Ac-WETWV |
| Ac-WETWV | 225 | 0.5 | 1 |
| Ac-WESWV | 234 | 2.5 | 5.2 |
| Ac-WEVWV | 235 | 4.8 | 9.8 |
| Ac-WDTWV | 236 | 1.7 | 3.4 |

Truncation analysis also revealed that most of the sequence is necessary for potent function. Interestingly, the deletion of the amino-terminal glycine results in a peptide that is more potent than wild-type, whether the peptide is acylated (Table 8) or not (Table 9).

TABLE 8
ERBIN peptide truncations with N-terminal acylation

| | SEQ ID | IC₅₀ | fold less potent |
|---|---|---|---|
| Peptide sequence | NO: | (μM) | than Ac-GWETWV |
| Ac-GWETWV | 237 | 0.9 | 1.0 |
| Ac-WETWV | 225 | 0.5 | 0.5 |
| Ac-ETWV | 238 | 4.9 | 5.1 |
| Ac-TWY | 239 | 77.4 | 81.5 |
| Ac-WV | | 77.8 | 78.7 |

TABLE 9
ERBIN peptide truncations without N-terminal acylation

| | SEQ ID | IC₅₀ | fold less potent |
|---|---|---|---|
| Peptide sequence | NO: | (μM) | than H₁N -GWETWV |
| H₁N-GWETWV | 237 | 1.4 | 1.0 |
| H₁N -WETWV | 225 | 0.2 | 0.2 |
| H₁N -ETWV | 238 | 16.5 | 11.5 |
| H₁N -TWV | 239 | 105.2 | 73.6 |
| H₁N -WV | | N/D | |

### Example 14.0

### PDZ Binding Peptides can be used to Discover Small Molecule Inhibitors

Using the same assay as Example 12.0, small molecules containing a W-V structural backbone were substituted for the peptide and assayed for their ability to inhibit the GST-PDZ domain to bind the immobilized WETWV (SEQ ID NO:225). The most effective compounds are presented in Table 10 and their structures illustrated below.

TABLE 10
Small molecules that inhibit ERBIN PDZ domain from binding PDZB

| Compound | IC₅₀ (μM) |
|---|---|
| WV 38 | 304 |
| WV 46 | 334 |
| WV 58 | 697 |
| WV 66 | 549 |

The corresponding structures are:

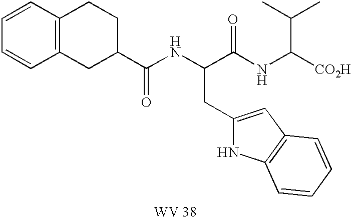

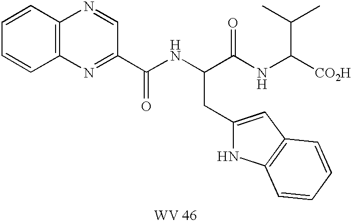

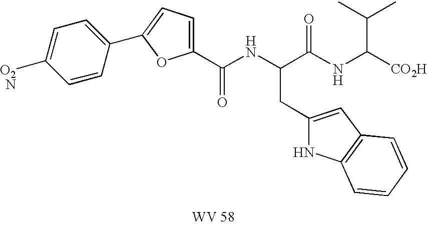

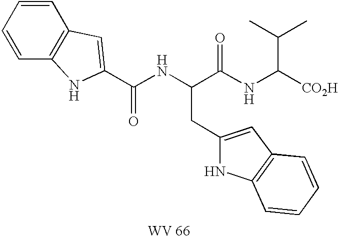

These data demonstrate the usefulness of PDZBs as pharmaceutical targets.

### Example 15.0

### Selection of PDBPs for a Variety of PDZ Domains

Phage display technology was further employed essentially as described above, with minor modifications, to select ligands of a variety of PDZ domains (including additional, independent rounds of selection for ERBIN PDZ and MAGI3 PDZ3). Briefly, peptide ligands were selected from pools of randomized peptides. The phage-displayed peptide pool comprised linear, hard-randomized hepta-, octa-, nona-, deca- and dodecamers in equal amounts and had a theoretical idversity of 3×10¹⁰. The peptides were fused to the M13 phage major coat proteins such that the C-termini of the randomized peptides were free and available for binding. PDZ domains were utilized as their GST-fusions (referred to in this Example simply as “PDZ domains”). The particular amino acids comprising each PDZ domain target are indicated in the heading of Tables 11-29.

Peptide ligands were selected and identified for 17 (18 including ERBIN) PDZ domains. Results are summarized in Tables 11-29 below. Each table shows a list of the peptides selected for a particular PDZ domain, with the occurrence of each amino acid residue in the position 0 to −7 (as indicated; in some cases, position −8 is also included; “−” indicates an undetermined residue, and thus can be any amino acid). At the bottom of each table, the occurrence of each amino acid residue is expressed as a percentage of the total number of residues in the relevant position. Siblings (peptides with identical DNA that appear as more than one copy) were counted as individuals. The numbers for occurrence were corrected for codon usage. The relative codon usage is indicated after each amino acid in the header of the bottom section of each table. “n” refers to the number of sequences (siblings counted as individuals) on which the occurrence value is based; this number is also shown as normalized with respect to codon usage.

TABLE 11
ERBIN (NP061165.1) PDZ domain

| occurence | | | | | −7 | −6 | −5 | −4 | −3 | −2 | −1 | 0 | ID No |
|---|---|---|---|---|---|---|---|---|---|---|---|---|---|
| 3 | | R | — | R | — | W | D | T | W | V | 164 | | |
| 1 | Q | R | E | S | P | W | D | T | W | V | 165 | | |
| 1 | R | A | A | E | R | W | D | T | W | V | 166 | | |
| 2 | S | T | G | K | F | F | D | T | W | V | 167 | | |
| 1 | | | | | | A | Y | F | D | T | W | V | 168 |
| 1 | | | | L | D | R | F | F | D | T | W | V | 169 |
| 2 | | | S | T | G | K | F | F | D | T | W | V | 170 |
| 1 | | | S | T | G | K | F | F | D | T | W | V | 171 |
| 1 | | | | | | R | L | F | D | T | W | V | 172 |
| 1 | | | T | T | A | S | W | Y | D | T | W | V | 173 |
| 1 | | | Q | S | S | F | W | Y | D | T | W | V | 174 |
| 2 | | | | | | L | S | G | 0 | T | W | V | 175 |
| 1 | | | R | D | R | C | S | L | D | T | W | V | 176 |
| 1 | H | A | A | R | S | V | D | V | D | T | W | V | 177 |
| 2 | | | R | L | S | L | F | D | D | T | W | V | 178 |
| 1 | | | | | | H | F | D | D | T | W | V | 179 |
| 1 | | | | G | S | T | F | H | D | T | W | V | 180 |
| 2 | P | V | G | R | G | R | W | M | D | T | W | V | 181 |
| 1 | | | | | | G | D | Q | D | T | W | V | 209 |
| 2 | E | S | Q | S | S | S | H | W | E | T | W | V | 210 |
| 2 | | | | | Q | S | W | I | E | T | W | V | 211 |
| 1 | | | | A | N | A | F | E | E | T | W | V | 212 |
| 1 | R | N | S | C | R | G | Y | W | D | S | W | V | 213 |
| 1 | | | | | E | S | W | H | D | 5 | W | V | 241 |
| 1 | E | S | — | Q | S | W | W | P | D | S | W | V | 242 |
| 1 | | | | R | V | Q | W | F | D | S | W | V | 243 |
| 1 | | | | K | Q | S | Q | W | D | S | W | V | 244 |
| 1 | | | E | R | K | G | V | F | E | S | W | V | 245 |
| 1 | | | R | E | Q | R | Y | F | D | T | W | L | 246 |
| 1 | E | R | A | R | N | P | F | W | D | V | W | V | 247 |

ERBIN peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 98 | 2 | | | | | | | | | | | | | | | | | | 19 |
| −1 | | | | | | | | 100 | | | | | | | | | | | | | 39 |
| −2 | | 3 | | | | | | | | 11 | 84 | | | | | | | | | | 19 |
| −3 | | | | | | | | | | | | | | 85 | 15 | | | | | | 39 |
| −4 | | 2 | 1 | 6 | 6 | 29 | 6 | 29 | 3 | | | | 3 | 9 | 3 | | | 6 | | 2 | 35 |
| −5 | | 2 | 1 | | | 34 | 9 | 31 | | 3 | | | 3 | 6 | | 1 | | 6 | | 2 | 32 |
| −6 | 5 | 2 | 5 | | | 5 | | 5 | 9 | 14 | 2 | | 5 | | 5 | 14 | 18 | 5 | 5 | 2 | 22 |
| −7 | 5 | 3 | | | | | | | 16 | 16 | | 11 | 21 | 5 | 11 | 5 | 5 | | | | 19 |

TABLE 11
DENSIN-180 (NP476483.1) PDZ4

| occurence | | | | | −7 | −6 | −5 | −4 | −3 | −2 | −1 | 0 | ID No |
|---|---|---|---|---|---|---|---|---|---|---|---|---|---|
| 13 | | | E | S | N | R | W | P | E | T | W | V | 248 |
| 7 | | | Q | V | G | F | W | P | E | T | W | V | 249 |
| 2 | S | R | R | R | T | Y | Y | P | E | T | W | V | 250 |
| 2 | | P | S | R | A | S | W | R | E | T | W | V | 251 |
| 1 | E | A | T | Q | R | A | F | R | E | T | W | V | 252 |
| 1 | | | | R | R | S | H | R | E | T | W | V | 253 |
| 1 | K | R | S | L | S | L | H | R | E | T | W | V | 254 |
| 5 | | | K | A | A | G | W | W | E | T | W | V | 255 |
| 1 | | | Q | R | R | W | P | W | E | T | W | V | 256 |
| 1 | | | | R | G | S | W | F | E | T | W | V | 257 |
| 1 | R | K | R | G | A | L | W | F | E | T | W | V | 258 |
| I | R | G | S | Q | T | R | Y | I | E | T | W | V | 259 |
| I | R | R | Q | Q | A | A | W | L | E | T | W | V | 260 |
| 1 | | | R | N | Q | G | W | D | E | T | W | V | 261 |
| 1 | | | — | — | — | — | W | — | E | T | W | V | 262 |
| 1 | — | — | K | — | K | G | W | — | E | T | W | V | 263 |
| 1 | | | | P | R | S | W | F | E | S | W | V | 264 |
| 1 | | | | | S | S | F | F | E | S | W | V | 265 |
| 3 | | | | | | R | W | F | D | T | W | V | 266 |
| 1 | | | | P | D | C | W | Y | D | T | W | V | 267 |
| 1 | | | T | T | A | S | W | Y | D | T | W | V | 268 |
| 1 | | | | | E | R | Y | H | D | T | W | V | 269 |
| 1 | | | | H | S | S | I | K | D | T | W | V | 270 |
| 1 | | | R | S | G | R | Y | L | D | T | W | V | 271 |
| 13 | H | P | K | H | K | G | W | F | E | T | W | L | 272 |
| 1 | S | R | K | A | R | T | W | W | E | T | W | L | 273 |
| 1 | | | | | Q | S | W | Y | E | T | W | L | 274 |
| 1 | | | | R | R | D | W | Y | E | T | W | L | 275 |
| 2 | | | R | L | S | R | F | K | E | T | W | L | 276 |
| 1 | C | R | G | G | I | S | W | K | E | T | W | L | 277 |
| 1 | | | R | K | R | L | W | V | E | T | W | L | 278 |
| 1 | | | | K | N | R | Y | L | E | T | W | L | 279 |
| 1 | | | | | | A | W | L | E | T | W | L | 280 |
| 1 | — | R | K | — | — | — | W | — | E | T | W | L | 281 |
| 1 | | | | R | — | V | Y | — | E | T | W | L | 282 |
| 1 | | | | G | S | W | Y | T | — | T | W | L | 283 |
| 1 | H | S | V | V | W | F | P | W | V | T | W | I | 284 |

DENSIN-180 PDZ4 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 74 | 24 | 3 | | | | | | | | | | | | | | | | | 34 |
| −1 | | | | | | | | 100 | | | | | | | | | | | | | 76 |
| −2 | | | | | | | | | | 2 | 97 | | | | | | | | | | 38 |
| −3 | | 1 | | | | | | | | | | | | 11 | 88 | | | | | | 75 |
| −4 | | 1 | 2 | 2 | | 37 | 7 | 15 | | | 1 | | | 2 | | 3 | 7 | 2 | | 20 | 54 |
| −5 | | | | 1 | | 5 | 11 | 79 | | | | | | | | | | 3 | | 1 | 75 |
| −6 | 4 | 1 | 3 | | | 21 | 5 | 5 | 26 | 9 | 1 | | | 3 | | 19 | | | 3 | | 38 |
| −7 | 10 | | | | | | | 2 | 9 | 4 | 4 | 29 | 4 | 2 | 2 | 5 | 29 | | | | 49 |

TABLE 14
Human Scribble (KIAA0147, NP_056171.1) PDZ2 (aa 788-913)

| | | | | | | | | | | | Seq |
|---|---|---|---|---|---|---|---|---|---|---|---|
| occurence | | | −7 | −6 | −5 | −4 | −3 | −2 | −1 | 0 | ID No |
| 21 | | | H | R | V | R | E | T | W | V | 285 |
| 4 | | | L | T | V | R | E | T | W | V | 286 |
| 2 | | | | A | W | F | E | T | W | V | 287 |
| 1 | R | K | S | R | T | F | E | T | W | V | 288 |
| 1 | E | S | V | R | G | F | D | T | W | V | 289 |
| 1 | S | T | G | K | F | F | D | T | W | V | 290 |
| 6 | | R | S | R | Y | R | F | T | D | V | 291 |
| 1 | | R | S | R | Y | — | E | T | D | V | 292 |

Human Scribble PDZ2 peptides: Percentage corrected for codon usage

| | | | | | | | | | | | | | | | | | | | | |
|---|---|---|---|---|---|---|---|---|---|---|---|---|---|---|---|---|---|---|---|---|
| A2 | V2 | L3 | I1 | M1 | F1 | Y1 | W1 | G2 | S3 | T2 | N1 | Q1 | D1 | E1 | R3 | K1 | H1 | C1 | P2 | n |

| | | | | | | | | | | | | | | | |
|---|---|---|---|---|---|---|---|---|---|---|---|---|---|---|---|
| 0 | | 100 | | | | | | | | | | | | | 19 |
| −1 | | | | | | 81 | | | | 37 | | | | | 37 |
| −2 | | | | | | | | | 100 | | | | | | 19 |
| −3 | | | | | | | | | | 5 | 95 | | | | 37 |
| −4 | | | | 33 | | | | | | | | 67 | | | 15 |
| −5 | | 54 | | 4 | 29 | 8 | 2 | | 2 | | | | | | 24 |
| −6 | 7 | | | | | | | | 14 | | | 71 | 7 | | 14 |
| −7 | | 2 | 4 | | | | 2 | 12 | | | | | | 81 | 26 |

TABLE 15
MUPP (MPDZ NM_003829) PDZ7

| occurence | | | | | −7 | −6 | −5 | −4 | −3 | −2 | −1 | 0 | ID No |
|---|---|---|---|---|---|---|---|---|---|---|---|---|---|
| 1 | | | | | | L | G | R | E | T | W | L | 293 |
| 1 | | | | R | S | S | G | R | E | T | W | L | 294 |
| 1 | | | V | R | F | L | G | R | E | T | W | L | 295 |
| 11 | W | L | R | L | G | A | Q | R | E | T | W | L | 296 |
| 1 | | | | | | P | D | Q | E | T | W | L | 297 |
| 4 | | | | | S | M | W | P | E | T | W | L | 298 |
| 1 | R | K | R | S | T | T | S | W | E | T | W | L | 299 |
| 1 | | | | | | | | | E | T | W | L | 300 |
| 12 | | | | | | L | F | K | I | T | W | L | 301 |
| 5 | | | G | W | L | R | G | R | V | T | W | L | 302 |
| 1 | V | L | A | I | V | G | G | W | Q | R | L | P | 303 |

MUPP PDZ7 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | | 93 | | | | | | | | | | | | | | | | | 4 | 14 |
| −1 | | | 0.8 | | | | | 100 | | | | | | | | | | | | | 38 |
| −2 | | | | | | | | | | | 100 | | | | | 1.5 | | | | | 19 |
| −3 | | 7 | | 32 | | | | | | | | | 3 | | 57 | | | | | | 37 |
| −4 | | | | | | | | 9 | | | | | 4 | | | 26 | 52 | | | 9 | 23 |
| −5 | | | | | | | | 12 | 14 | 0.9 | | | 33 | 3 | 36 | | | | | | 33 |
| −6 | 32 | | 26 | | 21 | | | | 3 | 2 | 3 | | | | | 11 | | | | 3 | 19 |
| −7 | | 5 | 15 | | | 9 | | | 50 | 15 | 5 | | | | | | | | | | 11 |

TABLE 16
Human INADL (NM_005799) PDZ6

| occurence | | | | | −7 | −6 | −5 | −4 | −3 | −2 | −1 | 0 | ID No |
|---|---|---|---|---|---|---|---|---|---|---|---|---|---|
| 1 | | | | | | | D | R | E | T | W | L | 304 |
| 1 | | | | | | | E | R | E | T | W | L | 305 |
| 1 | | | | V | K | G | L | R | E | T | W | L | 306 |
| 2 | E | W | T | A | L | L | G | R | E | T | W | L | 307 |
| 1 | | | | H | N | R | E | W | E | T | W | L | 308 |
| 11 | L | L | W | I | W | M | L | P | E | T | W | L | 309 |
| 1 | T | M | R | R | G | E | W | Y | E | T | W | L | 310 |
| 4 | | | | | W | L | G | H | S | T | W | L | 311 |
| 5 | F | M | L | F | L | G | E | K | S | T | W | L | 312 |
| 1 | — | W | R | — | — | — | — | R | E | S | W | L | 313 |
| 1 | A | S | W | F | K | D | S | P | S | S | W | V | 314 |
| 1 | | | | — | — | G | — | W | E | — | W | — | 315 |

Human INADL PDZ6 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 6 | 91 | | | | | | | | | | | | | | | | | 4 | 11 |
| −1 | | | | | | | | 100 | | | | | | | | | | | | | 32 |
| −2 | | | | | | | | | | 4 | 94 | | | | | | | | | | 16 |
| −3 | | | | | | | | | | 13 | | | | | 87 | | | | | | 23 |
| −4 | | | | | | | 5 | 10 | | | | | | | | 10 | 25 | 20 | | 30 | 20 |
| −5 | | | 24 | | | | | 6 | 18 | 2 | | | | 6 | 47 | | | | | | 17 |
| −6 | | | 11 | | 58 | | | | 18 | | | | | 5 | 5 | 2 | | | | | 19 |
| −7 | | | 10 | | | | | 73 | 2 | | | 5 | | | | | 9 | | | | 22 |

TABLE 17
Human ZO1 (NM_003257) PDZ1

| occurence | | | | −8 | −7 | −6 | −5 | −4 | −3 | −2 | −1 | 0 | ID No |
|---|---|---|---|---|---|---|---|---|---|---|---|---|---|
| 1 | | | | | T | H | R | I | K | T | W | L | 316 |
| 2 | | | | R | S | Y | Q | R | T | T | W | L | 317 |
| 1 | | | R | S | V | F | R | M | T | T | W | L | 318 |
| 1 | | | R | S | E | Y | R | L | R | T | W | L | 319 |
| 1 | | | Q | S | G | W | G | M | R | T | W | L | 320 |
| 1 | | | R | V | A | W | R | W | T | T | W | L | 321 |
| 1 | | | R | K | S | W | L | F | T | T | W | L | 322 |
| 1 | | | Q | R | L | W | R | T | S | T | W | L | 323 |
| 1 | | | R | S | E | G | I | F | K | T | W | L | 324 |
| 2 | | | L | K | A | W | K | W | S | T | W | L | 325 |
| 2 | V | R | S | R | N | F | R | L | E | T | W | L | 326 |
| 1 | Q | Q | L | R | R | W | R | E | T | T | W | L | 327 |
| 1 | H | S | Q | S | C | W | R | I | K | T | W | L | 328 |
| 1 | R | S | I | S | F | Y | K | W | S | S | W | L | 329 |
| 2 | R | R | H | T | Y | W | D | K | T | E | W | L | 330 |
| 4 | | | R | R | P | W | Q | H | T | T | Y | L | 331 |
| 1 | | | | L | P | Y | R | M | S | T | W | V | 332 |
| 1 | | | R | R | S | S | S | F | S | T | W | V | 333 |
| 1 | | | R | K | S | W | V | F | T | T | W | V | 334 |
| 1 | | | | S | T | R | P | F | R | S | W | V | 335 |
| 1 | | | G | K | G | W | R | I | S | T | Y | V | 336 |

Human ZO1 PDZ1 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 23 | 73 | | | | | | | | | | | | | | | | | | 11 |
| −1 | | | | | | | 18 | 82 | | | | | | | | | | | | | 28 |
| −2 | | | | | | | | | | 5 | 80 | | | | 13 | | | | | | 15 |
| −3 | | | | | | | | | | 13 | 47 | | | | 13 | 7 20 | | | | 15 | |
| −4 | | | 4 | 13 | 13 | 21 | | 17 | | | 2 | | | | 4 | 3 | 8 | 17 | | | 24 |
| −5 | | 3 | 2 | 6 | | | | | 3 | 2 | | | 33 | 11 | | 22 | 17 | | | 3 | 18 |
| −6 | | | | | | 12 | 19 | 62 | 2 | 1 | | | | | | 1 | | 4 | | | 26 |
| −7 | 11 | 3 | 2 | | | 6 | 11 | | 6 | 11 | 6 | 11 | | | 11 | 2 | | | 6 | 17 | 18 |
| −8 | | 4 | 2 | | | | | | | 15 | 8 | | | | | 31 | 38 | | | | 13 |

TABLE 18
Human PDZK1 (NM_002614) PDZ1

| occurence | | | | | −7 | −6 | −5 | −4 | −3 | −2 | −1 | 0 | ID No |
|---|---|---|---|---|---|---|---|---|---|---|---|---|---|
| 1 | | | R | P | V | V | R | W | S | T | W | L | 337 |
| 1 | | | R | K | V | Y | L | W | S | T | W | L | 338 |
| 1 | | | R | E | R | V | V | W | S | T | W | L | 339 |
| 1 | | | | | S | T | V | W | S | T | W | L | 340 |
| 1 | | | | | | I | R | F | S | T | W | L | 341 |
| 1 | P | G | K | K | A | T | S | F | S | T | W | L | 342 |
| 1 | | | H | K | K | W | Y | F | S | T | W | L | 343 |
| 1 | | | | | V | V | R | K | S | T | W | L | 344 |
| 1 | | | | K | K | R | E | E | S | T | W | L | 345 |
| 1 | | | D | R | R | V | V | L | S | T | W | L | 346 |
| 2 | | | | | R | I | V | K | Q | T | W | L | 347 |
| 1 | | | Q | R | G | I | V | H | Q | T | W | L | 348 |
| 1 | E | I | V | S | W | D | T | R | G | T | W | L | 349 |
| 1 | | | | | L | F | I | Y | S | S | W | L | 350 |
| 5 | P | A | R | K | Q | S | E | W | S | T | F | L | 351 |
| 1 | | | R | Q | K | T | L | W | S | T | F | L | 352 |
| 1 | P | P | R | S | S | W | F | Y | S | T | F | L | 353 |
| 1 | | | | | R | V | I | K | S | T | F | L | 354 |
| 2 | | | | | | V | L | H | S | T | F | L | 355 |
| 1 | | | | S | V | V | L | F | E | T | F | L | 356 |
| 1 | | | K | A | K | T | V | F | E | T | F | L | 357 |
| 1 | | | R | G | G | D | I | W | S | T | Y | L | 358 |
| 1 | | | Q | K | A | W | L | W | S | I | Y | L | 359 |
| 1 | | | R | M | S | V | L | F | S | I | Y | L | 360 |
| 1 | | | | | Q | I | L | R | S | I | Y | L | 361 |
| 1 | | | | R | H | F | V | L | S | I | Y | L | 362 |
| 1 | | | G | K | R | V | V | S | S | I | Y | L | 363 |
| 1 | | | R | R | R | S | F | W | E | I | Y | L | 364 |
| 1 | | | | | V | V | V | R | S | I | L | L | 365 |
| 1 | | | A | K | S | W | I | W | S | T | L | L | 366 |
| 2 | | | | R | V | T | L | F | E | T | L | L | 367 |
| 1 | | | | | L | V | V | F | S | T | R | L | 368 |
| 1 | | | | S | P | I | V | K | S | T | R | L | 369 |
| 1 | | | | | T | W | I | F | S | S | R | L | 370 |
| 1 | A | Q | V | S | R | I | L | Y | S | S | R | L | 371 |
| 1 | | | | | V | I | I | Y | S | I | R | M | 372 |
| 1 | | | E | V | P | W | L | W | S | S | R | M | 373 |
| 1 | | | | | V | R | E | F | S | I | W | M | 374 |

Human PDZK1 PDZ1 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | | 82 | | 18 | | | | | | | | | | | | | | | | 17 |
| −1 | | | 3 | | | 32 | 18 | 42 | | | | | | | | 5 | | | | | 38 |
| −2 | | | | | | | | | | 6 | 95 | | | | | | | | | | 22 |
| −3 | | | | | | | | | 2 | 57 | | | 14 | | 24 | | | | | | 21 |
| −4 | | | 2 | | | 27 | 10 | 37 | | 1 | | | | | 2 | 2 | 12 | 7 | | | 41 |
| −5 | | 21 | 14 | 21 | | 7 | 4 | | | 1 | 2 | | | | 25 | 4 | | | | | 28 |
| −6 | | 20 | | 23 | | 7 | 3 | 20 | | 7 | 12 | | | 7 | | 2 | | | | | 30 |
| −7 | 4 | 18 | 3 | | | | | 4 | 4 | 5 | 2 | | 24 | | | 11 | 16 | 4 | | 4 | 25 |

TABLE 19
Human Scribble (KIAA0147, NP_056171.1) PDZ1 (aa 650-760)

| occurence | | | | −8 | −7 | −6 | −5 | −4 | −3 | −2 | −1 | 0 | ID No |
|---|---|---|---|---|---|---|---|---|---|---|---|---|---|
| 1 | | | | | P | R | Y | L | E | T | D | L | 375 |
| 3 | | | | N | R | V | W | R | E | T | D | L | 376 |
| 2 | | | | S | R | L | W | R | E | T | D | L | 377 |
| 2 | | | | P | R | R | W | M | E | T | D | L | 378 |
| 1 | | | | R | R | I | F | L | E | T | D | L | 379 |
| 3 | | | R | S | S | R | F | L | E | T | D | L | 380 |
| 2 | | | H | R | P | K | W | S | E | T | D | L | 381 |
| 5 | | | K | S | R | S | Y | F | E | T | D | L | 382 |
| 6 | | | R | G | R | C | W | F | E | T | D | L | 383 |
| 1 | G | K | R | R | V | G | L | L | E | T | D | L | 384 |
| 3 | | | Q | K | K | P | F | F | W | T | D | L | 385 |
| 2 | S | N | G | Q | R | R | S | F | W | T | D | L | 386 |
| 1 | T | G | P | R | K | R | Y | L | E | S | D | L | 387 |
| 1 | P | G | P | T | R | S | W | R | E | T | E | L | 388 |
| 1 | L | G | S | K | R | S | Y | E | E | T | H | L | 389 |
| 2 | | | | | T | Y | R | E | G | D | W | L | 390 |
| 1 | | | | | Q | Y | K | P | G | D | W | L | 391 |

Human Scribble PDZ1 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | | 100 | | | | | | | | | | | | | | | | | | 12 |
| −1 | | | | | | | | 8 | | | | | | 86 | 3 | | | 3 | | | 37 |
| −2 | | | | | | | | | | 1.5 | 85 | | | 15 | | | | | | | 20 |
| −3 | | | | | | | | 14 | 4 | | | | | | 81 | | | | | | 36 |
| −4 | | | 8 | | 8 | 62 | | | | 3 | | | | | 12 | 8 | | | | 2 | 26 |
| −5 | | | 0.9 | | | 21 | 24 | 47 | | 2 | | | | | | 2 | 3 | | | | 34 |
| −6 | | 7 | 3 | | | | 14 | | 2 | 10 | 2 | | | | | 14 | 10 | | 29 | 7 | 21 |
| −7 | | 3 | | | | | | | | 6 | 6 | | 6 | | | 47 | 24 | | | 9 | 17 |
| −8 | | | | | | | | | 16 | 16 | 3 | 16 | 11 | | | 11 | 21 | | | 5 | 19 |

TABLE 20
hScribble (KIAA0147, NP_056171.1) PDZ3 (aa 913-1030)

| | | | | | | | | | | | Seq |
|---|---|---|---|---|---|---|---|---|---|---|---|
| occurence | | | −7 | −6 | −5 | −4 | −3 | −2 | −1 | 0 | ID No |
| 3 | R | G | R | C | W | F | E | T | D | L | 392 |
| 1 | | | C | R | I | R | E | T | D | L | 393 |
| 1 | L | Q | Q | A | W | R | Q | T | D | L | 394 |
| 2 | | R | R | P | W | K | E | T | W | L | 395 |
| 1 | K | S | C | S | S | R | E | T | W | L | 396 |
| 1 | | | | S | W | K | E | T | W | L | 397 |
| 1 | R | R | R | L | W | R | E | T | W | L | 398 |
| 1 | | | R | F | G | K | E | T | H | L | 399 |
| 1 | K | Q | A | S | W | F | E | T | H | L | 400 |
| 1 | | R | R | W | W | R | E | T | S | L | 401 |

Human Scribble PDZ3 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | | 100 | | | | | | | | | | | | | | | | | | 33 |
| −1 | | | | | | | | 10 | | 0.6 | | | | 86 | | | | 4 | | | 51 |
| −2 | | | | | | | | | | | 100 | | | | | | | | | | 50 |
| −3 | | | | | | | | | | | | | 8 | | 92 | | | | | | 53 |
| −4 | | | | | | 85 | | | | | | | | | | 4 | 11 | | | | 46 |
| −5 | | | | 2 | | | 2 | 94 | 1 | 1 | | | | | | | | | | | 52 |
| −6 | 2 | | 1 | | | 2 | | 2 | 1 | 2 | | | | | | 2 | | | 90 | 2 | 45 |
| −7 | 3 | | 2 | | | | | | | | | | 10 | | | 75 | | | 10 | | 20 |

TABLE 21
Human MUPP (MPDZ, NM_003829) PDZ13

| | | | | | | | | | | | | | Seq |
|---|---|---|---|---|---|---|---|---|---|---|---|---|---|
| occurence | | | | −8 | −7 | −6 | −5 | −4 | −3 | −2 | −1 | 0 | ID No |
| 7 | L | P | W | F | W | L | L | K | A | T | R | V | 402 |
| 1 | L | M | L | S | W | W | D | R | E | T | R | V | 403 |
| 1 | A | D | W | W | W | V | M | T | E | T | R | V | 404 |
| 1 | G | S | W | W | W | V | M | R | S | T | R | V | 405 |
| 1 | A | W | V | W | W | T | L | T | E | S | R | V | 406 |
| 2 | P | F | W | W | H | L | L | R | S | S | R | V | 407 |
| 1 | | | | P | X | Y | V | A | Q | S | N | V | 408 |
| 4 | | | E | S | N | R | W | P | E | T | W | V | 409 |
| 1 | G | I | W | F | W | L | A | K | S | V | R | L | 410 |
| 1 | | | | | F | A | T | L | I | L | C | S | 411 |
| 1 | | | Q | W | V | L | F | C | T | Y | C | S | 412 |
| 1 | | | | | | H | S | S | V | I | C | G | 413 |

Human MUPP PDZ13 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 82 | 3 | | | | | | 5 | 6 | | | | | | | | | | | 11 |
| −1 | | | | | | | | 31 | | | | 8 | | | | 36 | | | 23 | | 13 |
| −2 | | 5 | 3 | 9 | | | 9 | | | 12 | 64 | | | | | | | | | | 11 |
| −3 | 23 | 3 | | 7 | | | | | | 9 | 3 | | 7 | | 47 | | | | | | 15 |
| −4 | 4 | | 2 | | | | | | | 2 | 7 | | | | | 9 | 57 | | 7 | 14 | 14 |
| −5 | 4 | 4 | 23 | | 15 | 8 | | 31 | | 2 | 4 | | | 8 | | | | | | | 13 |
| −6 | 5 | 10 | 40 | | | | 10 | 10 | | | 5 | | | | | 10 | | 10 | | | 10 |
| −7 | | 3 | | | | 5 | | 60 | | | | 20 | | | | | | 10 | | | 20 |
| −8 | | | | | | 47 | | 35 | | 12 | | | | | | | | | | 3 | 17 |

TABLE 22
Human SNTA1 (NM_003098) PDZ

| | | | | | | | | | | | | | Seq |
|---|---|---|---|---|---|---|---|---|---|---|---|---|---|
| occurence | | | | −8 | −7 | −6 | −5 | −4 | −3 | −2 | −1 | 0 | ID No |
| 1 | | | E | W | I | S | L | F | S | T | R | L | 414 |
| 11 | W | L | S | Y | M | F | S | R | S | T | R | L | 415 |
| 5 | | | W | W | V | F | M | R | S | T | R | L | 416 |
| 4 | R | L | Q | W | L | F | G | R | S | T | S | L | 417 |
| 1 | — | P | Q | W | — | F | G | R | — | T | W | L | 418 |
| 1 | F | M | L | F | L | W | L | R | S | S | V | V | 419 |

Human SNTA1 PDZ peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 6 | 91 | | | | | | | | | | | | | | | | | | 8 |
| −1 | | 6 | | | | | | 11 | | 14 | | | | | | 63 | | | | | 9 |
| −2 | | | | | | | | | | 3 | 100 | | | | | | | | | | 11 |
| −3 | | | | | | | | | | 100 | | | | | | | | | | | 7 |
| −4 | | | | | | 13 | | | | | | | | | | 91 | | | | | 8 |
| −5 | | | 8 | | 38 | | | | 23 | 31 | | | | | | | | | | | 13 |
| −6 | | | | | | 95 | | 4 | | 1 | | | | | | | | | | | 22 |
| −7 | | 18 | 12 | 6 | 65 | | | | | | | | | | | | | | | | 17 |
| −8 | | | | | | 4 | 48 | 48 | | | | | | | | | | | | | 23 |

TABLE 23
Human PARD3 (NP_062565.1) PDZ3

| | | | | | | | | | | | Seq |
|---|---|---|---|---|---|---|---|---|---|---|---|
| occurence | | | −7 | −6 | −5 | −4 | −3 | −2 | −1 | 0 | ID No |
| | | | | | | | | | | | 392 |
| 21 | N | V | I | E | Y | F | L | G | W | L | 420 |
| 1 | N | V | — | E | Y | F | V | G | W | L | 421 |
| 1 | H | T | E | W | T | F | L | G | W | L | 422 |
| 4 | | | | D | E | D | V | W | W | L | 423 |
| 11 | R | T | V | W | Y | D | L | G | E | L | 424 |
| 1 | | | L | D | G | G | C | M | W | I | 425 |
| 2 | A | H | A | W | Y | D | L | G | N | I | 426 |

Human PARD3 PDZ3 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | | 81 | 19 | | | | | | | | | | | | | | | | | 16 |
| −1 | | | | | | | | 67 | | | | 7 | | | 26 | | | | | | 42 |
| −2 | | | | | 4 | | | 17 | 77 | | | | | | | | | | | | 24 |
| −3 | | 16 | 75 | | | | | | | | | | | | | | | | 6 | | 16 |
| −4 | | | | | | 55 | | | 1 | | | | | 43 | | | | | | | 42 |
| −5 | | | | | | | 88 | | 1 | | 1 | | | | 10 | | | | | | 41 |
| −6 | | | | | | | | 36 | | | | | | 12 | 52 | | | | | | 42 |
| −7 | 5 | 19 | 1 | 72 | | | | | | | | | | | 3 | | | | | | 29 |

TABLE 24
Human INADL (NM_005799) PDZ2

| | | | | | | | | | | | | | Seq |
|---|---|---|---|---|---|---|---|---|---|---|---|---|---|
| occurence | | | | | −7 | −6 | −5 | −4 | −3 | −2 | −1 | 0 | ID No |
| 1 | | | | | A | D | E | E | I | W | W | V | 427 |
| 1 | R | R | L | R | C | E | E | R | I | W | W | V | 428 |
| 3 | | | A | K | E | S | L | P | I | Y | W | V | 429 |
| 1 | | | | | | K | E | K | I | F | W | V | 430 |
| 4 | | | | | D | S | E | R | E | W | F | V | 431 |
| 1 | | | | | | R | D | R | E | W | F | V | 432 |

Human INADL PDZ2 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 100 | | | | | | | | | | | | | | | | | | | 6 |
| −1 | | | | | | 45 | | 55 | | | | | | | | | | | | | 11 |
| −2 | | | | | | 9 | 27 | 64 | | | | | | | | | | | | | 11 |
| −3 | | | | 55 | | | | | | | | | | | 45 | | | | | | 11 |
| −4 | | | | | | | | | | | | | | | 17 | 33 | 17 | | | 25 | 6 |
| −5 | | | 11 | | | | | | | | | | | 11 | 78 | | | | | | 9 |
| −6 | | | | | | | | | | 40 | | | | 20 | 20 | 6 | 20 | | | | 5 |
| −7 | 6 | | | | | | | | | | | | | 44 | 33 | | | | 11 | | 9 |

TABLE 25
Human INADL (NM_005799) PDZ3

| | | | | | | | | | | | | | Seq |
|---|---|---|---|---|---|---|---|---|---|---|---|---|---|
| occurence | | | | | −7 | −6 | −5 | −4 | −3 | −2 | −1 | 0 | ID No |
| 2 | | | | | | S | C | W | F | L | D | I | 433 |
| 1 | | | | | | R | S | W | F | L | D | I | 434 |
| 1 | | | | | | H | V | W | F | L | D | I | 435 |
| 1 | | | | | | S | V | W | F | L | D | I | 436 |
| 1 | | | | | A | T | P | W | Y | L | D | I | 437 |
| 1 | | | | | R | S | V | W | Y | L | D | I | 438 |
| 1 | | | R | R | E | S | P | W | Y | L | D | I | 439 |
| 1 | | | Q | S | R | S | W | W | Y | L | D | I | 440 |
| 2 | | | Q | D | T | G | C | W | W | L | D | I | 441 |
| 1 | | | S | K | L | R | T | W | W | L | D | I | 442 |
| 1 | | | | | | S | P | W | F | M | D | I | 443 |
| 1 | | | | | R | S | V | W | F | L | L | I | 444 |
| 3 | | | K | K | N | S | V | W | E | L | L | I | 445 |
| 1 | | | Q | R | N | S | I | W | E | L | L | I | 446 |
| 1 | P | R | K | P | L | D | W | W | E | L | L | I | 447 |
| 24 | | | T | R | S | P | D | W | S | L | W | I | 448 |
| 1 | V | D | G | S | F | S | L | W | S | L | W | I | 449 |
| 1 | | | S | C | P | G | W | W | S | L | W | I | 450 |
| 1 | | | | R | S | G | C | W | T | L | W | I | 451 |
| 1 | R | E | T | G | S | V | W | L | D | I | W | I | 452 |
| 1 | | | | | | P | V | W | Y | L | D | L | 453 |
| 1 | | | E | R | S | A | C | W | F | L | D | L | 454 |
| 1 | | | | | Q | A | R | W | F | Y | D | L | 455 |
| 1 | | | R | R | P | S | C | W | F | M | D | L | 456 |
| 1 | | | | | R | S | S | W | S | L | W | L | 457 |
| 1 | R | S | H | G | R | V | W | L | D | M | V | L | 458 |
| 1 | | | R | C | K | E | S | W | S | L | W | V | 459 |
| 1 | | | | | | R | C | W | F | F | D | W | 460 |
| 1 | | | | | R | P | D | W | S | F | W | W | 461 |
| 1 | | | G | W | G | S | T | W | T | Y | W | W | 462 |
| 1 | | | | P | S | R | L | Q | E | W | Y | F | 463 |

Human INADL PDZ3 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 1 | 4 | 86 | | 2 | | 7 | | | | | | | | | | | | | 57 |
| −1 | | 1 | 4 | | | | 2 | 58 | | | | | | 35 | | | | | | | 57 |
| −2 | | | 62 | 4 | 12 | 12 | 8 | 4 | | | | | | | | | | | | | 26 |
| −3 | | | | | | 28 | 13 | 10 | | 25 | 3 | | | 5 | 18 | | | | | | 40 |
| −4 | | | 1 | | | | | 97 | | | | | 2 | | | | | | | | 60 |
| −5 | | 8 | 2 | 2 | | | 2 | 10 | | 2 | 2 | | | 49 | | 1 | 2 | | 18 | 4 | 51 |
| −6 | 4 | 4 | | | | | | | 11 | 18 | 2 | | | 4 | 4 | 4 | | 4 | | 46 | 28 |
| −7 | 2 | | 4 | | | 4 | | | 2 | 36 | 4 | 16 | 8 | | 4 | 8 | 8 | | | 4 | 25 |

TABLE 26
Human MAGI1 PDZ3 (Bai1 PDZ4) (NP_004733.1)

| | | | | | | | | | | | | | Seq |
|---|---|---|---|---|---|---|---|---|---|---|---|---|---|
| occurence | | | | | −7 | −6 | −5 | −4 | −3 | −2 | −1 | 0 | ID No |
| 1 | | | | | | R | G | W | F | L | D | V | 464 |
| 1 | | | | | | R | V | W | F | L | D | V | 465 |
| 1 | | | | | H | S | G | W | F | L | D | V | 466 |
| 1 | | | | | R | S | A | W | F | L | D | V | 467 |
| 1 | | | | | T | R | G | W | F | L | D | V | 468 |
| 1 | | | | | P | K | A | W | F | L | D | V | 469 |
| 1 | | | | R | R | S | G | W | F | L | D | V | 470 |
| 1 | | | | S | S | K | A | W | F | L | D | V | 471 |
| 1 | | | R | P | A | G | G | W | F | L | D | V | 472 |
| 1 | | | | | | D | S | W | F | L | D | V | 473 |
| 1 | | | | K | S | G | S | W | F | L | D | V | 474 |
| 1 | | | | | | P | R | W | F | L | D | V | 475 |
| 1 | | | | | | S | H | W | F | L | D | V | 476 |
| 1 | | | | | E | R | R | W | F | L | D | V | 477 |
| 1 | | | | R | S | R | K | W | F | L | D | V | 478 |
| 1 | | | S | V | K | K | K | W | F | L | D | V | 479 |
| 1 | | | P | N | P | P | R | W | F | L | D | V | 480 |
| 1 | | | | | | T | R | W | F | L | D | V | 481 |
| 1 | | | | | R | R | N | W | F | L | D | V | 482 |
| 1 | | | | | R | N | F | W | F | L | D | V | 483 |
| 1 | | | R | G | R | Q | D | W | F | L | D | V | 484 |
| 1 | Q | A | R | S | G | G | M | W | F | L | D | V | 485 |
| 1 | | | | | Q | T | P | W | F | L | D | V | 486 |
| 1 | | | | | | Q | G | W | W | L | D | V | 487 |
| 1 | | | | | | P | V | W | W | L | D | V | 488 |
| 1 | | | | | S | A | G | W | W | L | D | V | 489 |
| 1 | | | | | S | P | V | W | W | L | D | V | 490 |
| 1 | R | Q | R | P | R | D | G | W | W | L | D | V | 491 |
| 1 | A | V | R | S | R | Q | G | W | W | L | D | V | 492 |
| 1 | | | G | E | S | L | P | W | W | L | D | V | 493 |
| 1 | | | K | E | R | S | F | W | W | L | D | V | 494 |
| 1 | | | P | S | K | S | A | W | Y | L | D | V | 495 |
| 1 | | | | | P | R | S | W | Y | L | D | V | 496 |
| 1 | | | | R | S | S | S | W | Y | L | D | V | 497 |
| 1 | K | E | K | C | R | P | S | W | Y | L | D | V | 498 |
| 1 | | | | | T | S | T | W | Y | L | D | V | 499 |
| 1 | | | | S | N | G | K | W | Y | L | D | V | 500 |
| 1 | | | | | L | S | A | W | F | I | D | V | 501 |
| 1 | | | | | R | S | V | W | W | F | D | V | 502 |
| 1 | | | | | P | R | G | W | W | F | D | A | 503 |
| 1 | | | | | S | S | G | W | W | Y | D | A | 504 |
| 1 | | | K | K | S | R | F | W | F | F | D | A | 505 |
| 1 | | | K | A | A | S | S | W | W | M | D | V | 506 |
| 1 | | | | | N | S | C | R | V | A | D | A | 507 |
| 1 | | | L | R | M | S | Y | D | M | S | T | A | 508 |
| 1 | Q | R | W | L | A | G | R | T | Y | S | D | W | 509 |
| 1 | | | | T | T | S | R | W | F | Y | D | A | 510 |
| 1 | | | | | | Q | W | C | A | I | C | R | 511 |

Human MAGI1 PDZ3 (Bai1 PDZ4) peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | 13 | 83 | | | | | | 4 | | | | | | | | 1 | | | | | 24 |
| −1 | | | | | | | | | | | 1 | | | 98 | | | | | 2 | | 47 |
| −2 | 2 | | 57 | 10 | 5 | 14 | 10 | | | 3 | | | | | | | | | | | 21 |
| −3 | 1 | 1 | | | 2 | 55 | 15 | 26 | | | | | | | | | | | | | 47 |
| −4 | | | | | | | | 94 | | | 1 | | | 2 | | 1 | | | 2 | | 47 |
| −5 | 8 | 7 | | | 3 | 7 | 3 | 3 | 18 | 10 | 2 | 3 | | 3 | 3 | 7 | 10 | 3 | 3 | 3 | 30 |
| −6 | 2 | | 1 | | | | | | 10 | 20 | 4 | 4 | 16 | 8 | | 12 | 12 | | | 10 | 25 |
| −7 | 10 | | 1 | | 5 | | | | 2 | 14 | 10 | 10 | 5 | | 5 | 14 | 10 | 5 | | 10 | 21 |

TABLE 27
MAGI3 PDZ3 (AF7238)

| | | | | | | | | | | | | | Seq |
|---|---|---|---|---|---|---|---|---|---|---|---|---|---|
| occurence | | | | | −7 | −6 | −5 | −4 | −3 | −2 | −1 | 0 | ID No |
| 1 | | | | | | D | R | W | W | F | D | I | 512 |
| 1 | | | | H | A | H | A | W | W | F | D | I | 513 |
| 1 | | | | K | S | N | T | W | W | F | D | I | 514 |
| 1 | | | | R | S | R | Q | W | W | F | D | I | 515 |
| 1 | | | Q | H | H | N | A | W | W | F | D | I | 516 |
| 1 | | | R | Y | S | E | R | W | W | F | D | I | 517 |
| 1 | | | Q | V | K | P | Y | W | W | F | D | I | 518 |
| 1 | R | S | L | S | R | S | V | W | W | F | D | I | 519 |
| 1 | C | S | R | P | A | S | S | W | S | F | W | I | 520 |
| 1 | | | | | | S | Y | W | W | F | D | A | 521 |
| 1 | | | | | | G | G | W | W | F | D | A | 522 |
| 1 | | | | | R | G | R | W | W | F | D | A | 523 |
| 1 | | | | | N | G | S | W | W | F | D | A | 524 |
| 1 | | | | | T | D | H | W | W | F | D | A | 525 |
| 1 | | | | H | T | A | R | W | W | F | D | A | 526 |
| 1 | | | | P | R | S | D | W | W | F | D | A | 527 |
| 1 | | | | V | E | R | K | W | W | F | D | A | 528 |
| 1 | | | | E | E | G | G | W | W | F | D | A | 529 |
| 1 | | | | S | G | S | W | W | W | F | D | A | 530 |
| 1 | | | P | R | R | V | T | W | W | F | D | A | 531 |
| 1 | | | R | G | T | F | T | W | W | F | D | A | 532 |
| 1 | | | N | R | V | E | I | W | W | F | D | A | 533 |
| 1 | | | G | T | K | R | E | W | W | F | D | A | 534 |
| 1 | | | R | R | R | G | G | W | W | F | D | A | 535 |
| 1 | | | K | Q | S | C | R | W | W | F | D | A | 536 |
| 1 | | | R | R | T | C | R | W | W | F | D | A | 537 |
| 1 | V | A | K | S | R | L | C | W | W | F | D | A | 538 |
| 1 | D | G | R | D | S | V | G | W | W | F | D | A | 539 |
| 1 | | | | R | K | I | F | W | F | F | D | A | 540 |
| 1 | | | H | R | G | I | I | W | F | F | D | A | 541 |
| 1 | | | | | T | S | G | W | S | F | L | A | 542 |
| 1 | | | | | | R | R | W | W | F | D | V | 543 |
| 2 | | | | | R | S | G | W | W | F | D | V | 544 |
| 2(unique | | | | | G | R | N | W | W | F | D | V | 545 |
| DNAs) | | | | | | | | | | | | | |
| 1 | | | | | K | S | Y | W | W | F | D | V | 546 |
| 1 | | | | | R | R | S | W | W | F | D | V | 547 |
| 1 | | | | R | S | R | V | W | W | F | D | V | 548 |
| 1 | | | P | Q | A | G | R | W | W | F | D | V | 549 |
| 1 | | | H | S | S | S | M | W | W | F | D | V | 550 |
| 1 | | | Q | L | R | K | S | W | W | F | D | V | 551 |
| 1 | | | R | P | S | R | W | W | W | F | D | V | 552 |
| 1 | | | S | E | Q | K | W | W | W | F | D | V | 553 |
| 1 | | | S | G | P | R | F | W | W | F | D | V | 554 |
| 1 | — | S | — | R | T | G | — | W | W | F | D | V | 555 |
| 1 | G | K | E | G | C | R | S | W | W | F | D | V | 556 |
| 1 | | | Q | R | R | G | F | W | F | F | D | V | 557 |
| 1 | | | K | D | H | V | S | W | W | L | D | V | 558 |
| 1 | | | R | T | R | S | C | W | W | L | D | V | 559 |
| 1 | H | K | R | N | A | S | C | W | F | L | D | V | 560 |
| 1 | | | R | E | T | K | V | W | F | L | D | V | 561 |
| 1 | | | R | S | K | G | K | W | Y | L | D | V | 562 |
| 1 | | | | K | S | S | G | W | Y | L | D | V | 563 |
| 1 | | | G | K | S | T | H | W | W | I | D | V | 564 |
| 1 | | | R | S | G | E | H | W | W | I | D | V | 565 |
| 1 | G | C | E | S | G | R | G | W | W | I | D | V | 566 |
| 1 | | | | | | R | C | W | F | I | D | V | 567 |
| 1 | R | N | T | G | W | G | G | W | F | I | D | V | 568 |
| 1 | | | G | V | S | S | S | W | W | I | D | F | 569 |
| 1 | | | | R | S | T | A | W | Y | E | D | F | 570 |
| 1 | | | R | V | K | G | G | W | F | H | D | F | 571 |
| 1 | | | | | Q | T | W | W | E | E | E | F | 572 |
| 1 | | | | K | V | R | G | W | S | E | L | F | 573 |
| 1 | L | T | G | S | S | R | Q | W | T | D | I | F | 574 |
| 1 | N | R | E | V | Q | T | F | W | D | V | L | F | 575 |

Human MAGI3 PDZ3 peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | 26 | 33 | | 24 | | 17 | | | | | | | | | | | | | | | 42 |
| −1 | | | 2 | 2 | | | | 2 | | | | | | 94 | 2 | | | | | | 64 |
| −2 | | 1 | 3 | 10 | | 77 | | | | | | | | 2 | 5 | | | 2 | | | 62 |
| −3 | | | | | | 12 | 5 | 77 | | 2 | 1 | | | 1 | 1 | | | | | | 65 |
| −4 | | | | | | | | 100 | | | | | | | | | | | | | 67 |
| −5 | 4 | 4 | | 2 | 2 | 9 | 7 | 9 | 13 | 4 | 4 | 4 | 4 | 2 | 2 | 7 | 4 | 7 | 9 | | 45 |
| −6 | 1 | 5 | 1 | 3 | | 3 | | | 16 | 14 | 8 | 5 | | 5 | 8 | 14 | 8 | 3 | 5 | 1 | 37 |
| −7 | 6 | 3 | | | | | | 3 | 9 | 13 | 10 | 3 | 9 | | 6 | 11 | 18 | 6 | 3 | 1 | 34 |

TABLE 28
MUPP (Human Multiple PDZ protein, MPDZ, NM_003829) PDZ3

| | | | | | | | | | | | | | Seq |
|---|---|---|---|---|---|---|---|---|---|---|---|---|---|
| occurence | | | | | −7 | −6 | −5 | −4 | −3 | −2 | −1 | 0 | ID No |
| 11 | | | | P | S | R | L | Q | E | W | Y | F | 576 |
| 1 | | | R | S | V | S | R | N | E | W | Y | F | 577 |
| 1 | K | S | S | S | D | G | W | N | T | W | Y | F | 578 |
| 2 | | | | | W | S | F | L | G | I | K | F | 579 |
| 3 | P | E | S | R | K | G | W | C | F | W | T | I | 580 |
| 1 | | | K | Q | E | G | W | T | F | W | E | L | 581 |
| 1 | | | C | P | R | D | W | I | C | A | R | M | 582 |

MUPP PDZ3 peptides: Corrected percentage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | | 2 | 16 | 5 | 79 | | | | | | | | | | | | | | | 19 |
| −1 | | | | | | | 72 | | | | 8 | | | | 6 | 2 | 11 | | | | 18 |
| −2 | 3 | | | 10 | | | | 85 | | | | | | | | | | | | | 20 |
| −3 | | | | | | 22 | | | 6 | | 3 | | | | 67 | | | | 6 | | 18 |
| −4 | | | 4 | 6 | | | | | | | 3 | 11 | 61 | | | | | | 17 | | 18 |
| −5 | | | 33 | | | 17 | | 50 | | | | | | | | 3 | | | | | 12 |
| −6 | | | | | | | | | 33 | 11 | | | | 11 | | 44 | | | | | 9 |
| −7 | | 5 | | | | | | 18 | | 36 | | | | 9 | 9 | 3 | 27 | | | | 11 |

TABLE 29
Human AF6 (NM_005936) PDZ (aa 967-1064)

| | | | | | | | | | | | | | Seq |
|---|---|---|---|---|---|---|---|---|---|---|---|---|---|
| occurence | | | | | −7 | −6 | −5 | −4 | −3 | −2 | −1 | 0 | ID No |
| 3 | | | | | F | I | S | K | P | W | F | W | 583 |
| 1 | | | | | F | E | S | E | P | W | F | W | 584 |
| 1 | | | | | R | I | S | K | E | W | F | W | 585 |
| 1 | | | | | R | V | Y | W | E | W | Y | W | 586 |
| 1 | P | S | V | P | W | M | S | S | T | W | Y | W | 587 |
| 2 | | | | | Y | V | S | R | E | W | W | W | 588 |
| 1 | | | | | F | V | — | K | P | W | L | W | 589 |
| 1 | R | T | T | G | W | I | G | K | P | W | L | W | 590 |
| 1 | | | | | W | V | S | V | E | W | L | W | 591 |
| 1 | T | H | H | G | I | I | F | W | E | M | L | W | 592 |
| 2 | | | | | F | I | S | D | P | W | E | W | 593 |
| 12 | | | | | Y | I | S | R | P | W | D | V | 594 |
| 1 | | | | | V | V | Y | W | T | M | D | V | 595 |
| 2 | | | S | G | V | I | L | W | F | M | D | V | 596 |
| 3 | | | | | R | V | F | W | E | L | D | I | 597 |
| 1 | Q | S | P | A | Q | V | L | W | W | M | L | I | 598 |
| 2 | R | N | G | L | S | I | F | W | E | M | L | V | 599 |
| 3 | | | | | V | F | Y | W | E | M | L | L | 600 |
| 1 | | | H | P | K | V | Y | W | V | L | W | L | 601 |

Human AF6 PDZ peptides: Percentage corrected for codon usage

| | A2 | V2 | L3 | I1 | M1 | F1 | Y1 | W1 | G2 | S3 | T2 | N1 | Q1 | D1 | E1 | R3 | K1 | H1 | C1 | P2 | n |
|---|---|---|---|---|---|---|---|---|---|---|---|---|---|---|---|---|---|---|---|---|---|
| 0 | | 27 | 4.2 | 13 | | | | 52 | | | | | | | 3.2 | | | | | | 31 |
| −1 | | | 8.9 | | | 16 | 5.4 | 11 | | | | | | 54 | 5.4 | | | | | | 37 |
| −2 | | | 3.3 | | 25 | | | 73 | | | | | | | | | | | | | 40 |
| −3 | | 1.6 | | | | 6.3 | | 3.1 | | | 3.1 | | | | 47 | | | | | 38 | 32 |
| −4 | | 1.5 | | | | | | 49 | | 0.9 | | | | 6.1 | 3.0 | 17 | 21 | | | | 33 |
| −5 | | | 4 | | | 26 | 26 | | 2 | 35 | | | | | | | | | 4 | | 23 |
| −6 | | 16 | | 70 | 3 | 8 | | | | | | | | | 3 | | | | | | 37 |
| −7 | | 9 | | 3 | | 20 | 43 | 9 | | 3 | | | 3 | | | 6 | 3 | | | 1 | 35 |

### Example 16.0

### Analysis of Sequence Database for Cognate Ligands of PDZ Domains

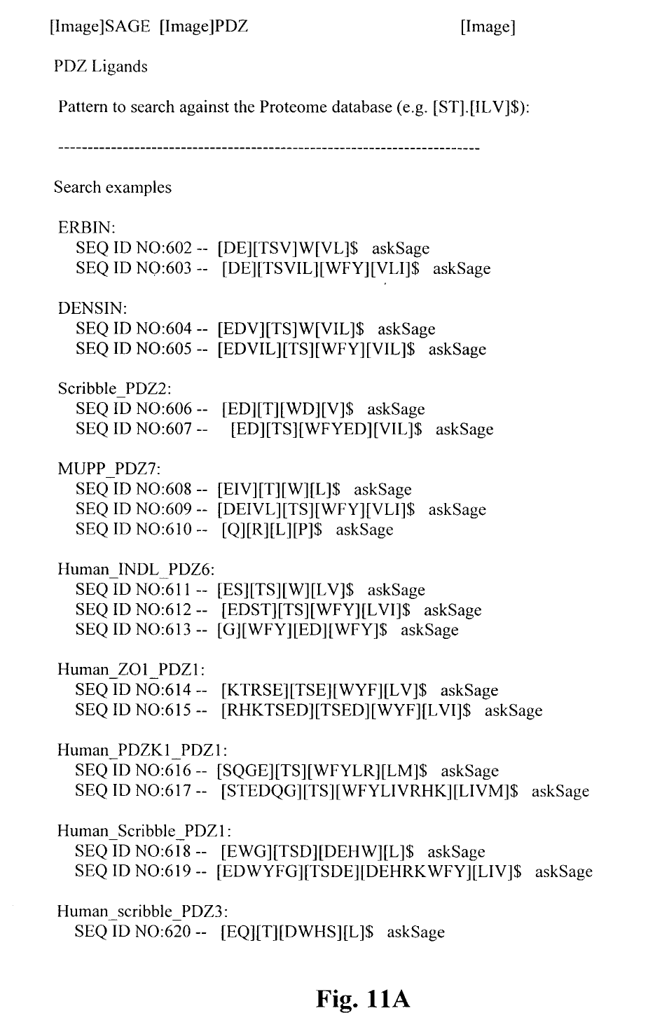

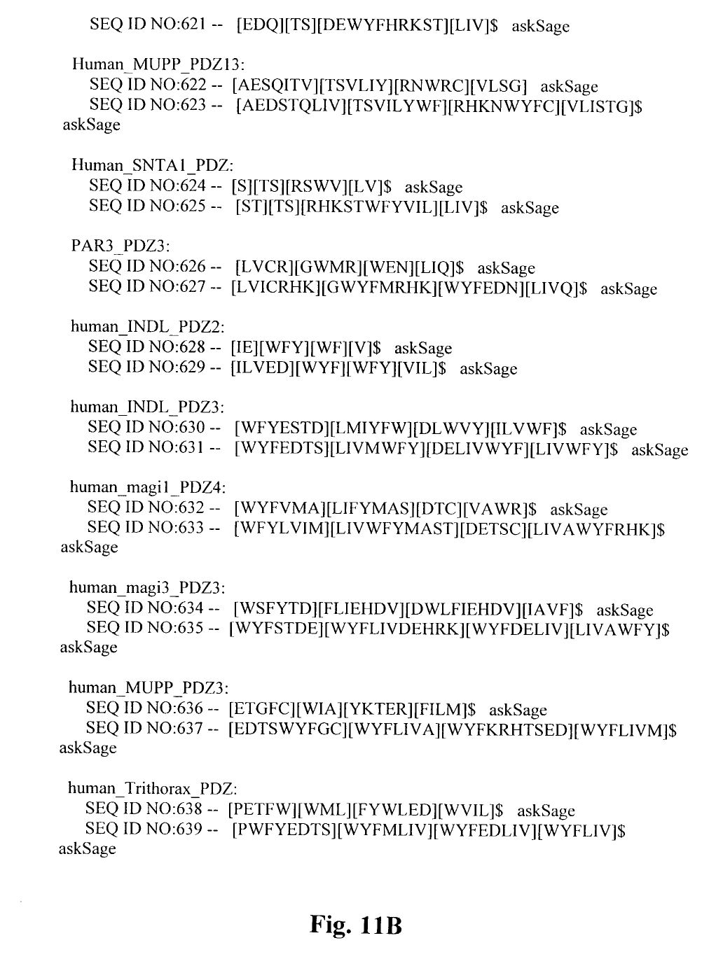

C-terminal consensus sequences were generated for each PDZ domain target based on the phage selected peptide sequences described in Example 15.0. A consensus sequence can be derived, for example, based on similarity of amino acid residues among commonly occurring residues in phage selected peptides. For example, for a sequence such as DETV$, a parameter sequence of [DE][DE][ST][VIL]$ can be used, because negative charged D and E are similar amino acids, alcoholic residues S and T are similar amino acids, aromatic residues W, Y and F are similar, and positively charged R, H and K are similar amino acids. Search results were then restricted to human sequences that contain the specific C-terminal sequences. Finally, ligands were picked based on similarity of function to the biological function(s) (including, for example, localization, tissue expression pattern) of the target protein containing the corresponding (as a phage display selection target) PDZ domain. These sequences were then searched against the Proteome motif database as exemplified in FIG. 11. In FIG. 11, the first line for each target PDZ domain refers to a sequence summary of the phage-selected peptide sequences, and the second/third lines refer to expanded sequences that were used for database searching. The expanded sequences were determined based on the criteria described above.

### Example 17.0

### Analysis of Binding Affinities of Peptides Based on Sequence of Selected PDBPs

Information derived from the sequences of the selected peptides as described above can be useful for a variety of purposes. For example, they can be used to determine the contribution of a particular residue in a peptide sequence to the binding affinity of the peptide to one or more PDZ domains. Structure-activity relationships can be determined in this manner. Design of binders with greater or lesser binding affinities to a particular PDZ domain can also be based on the sequences of the selected PDBPs as described above. Peptides with sequences that are of less than complete (100%) identity to the sequences of phage display-selected PDBPs can also be designed, and their binding capabilities to PDZ domains of interest determined as herein described.

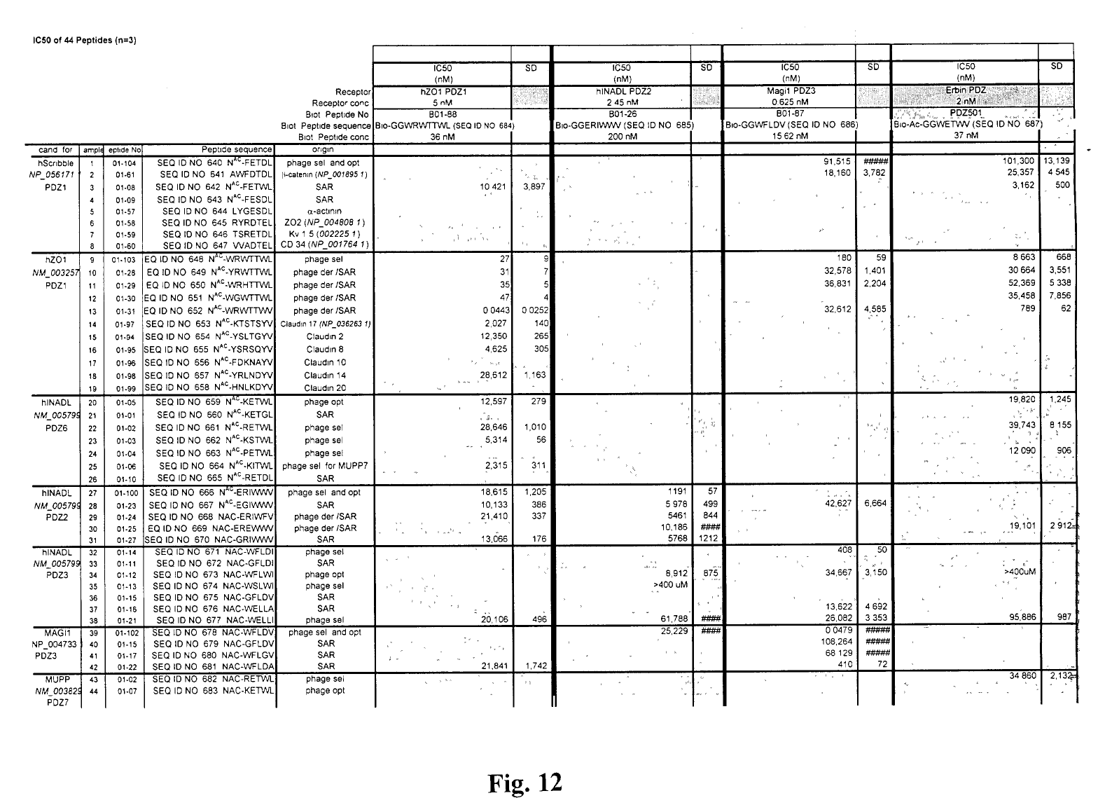

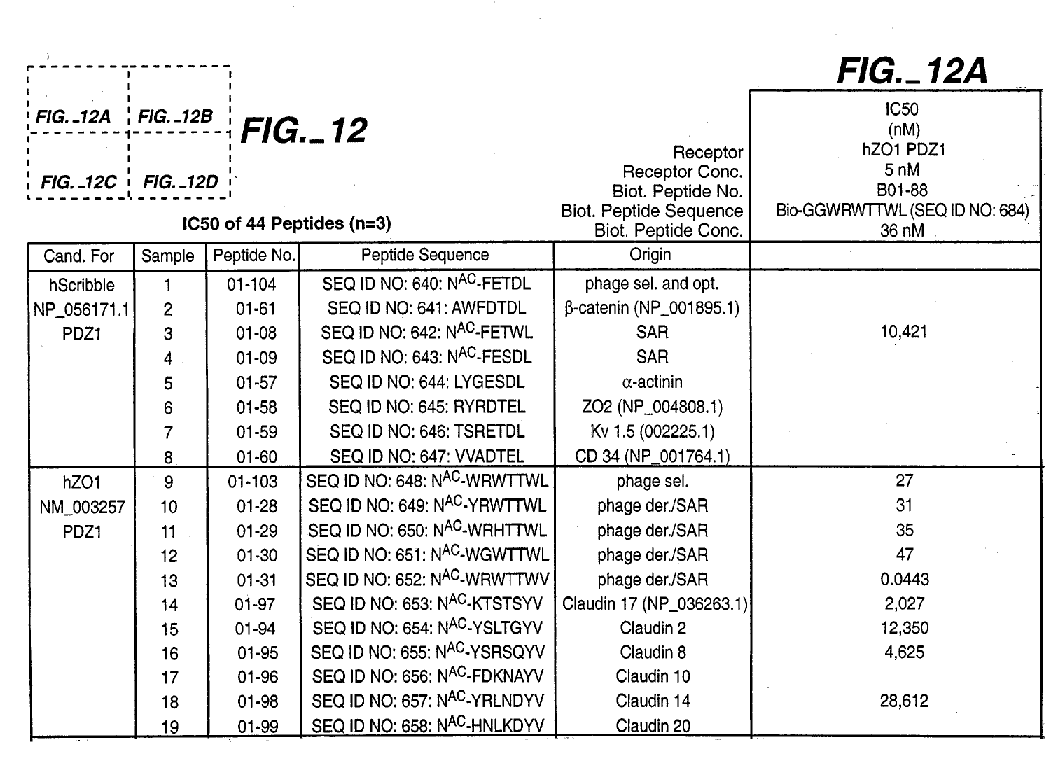

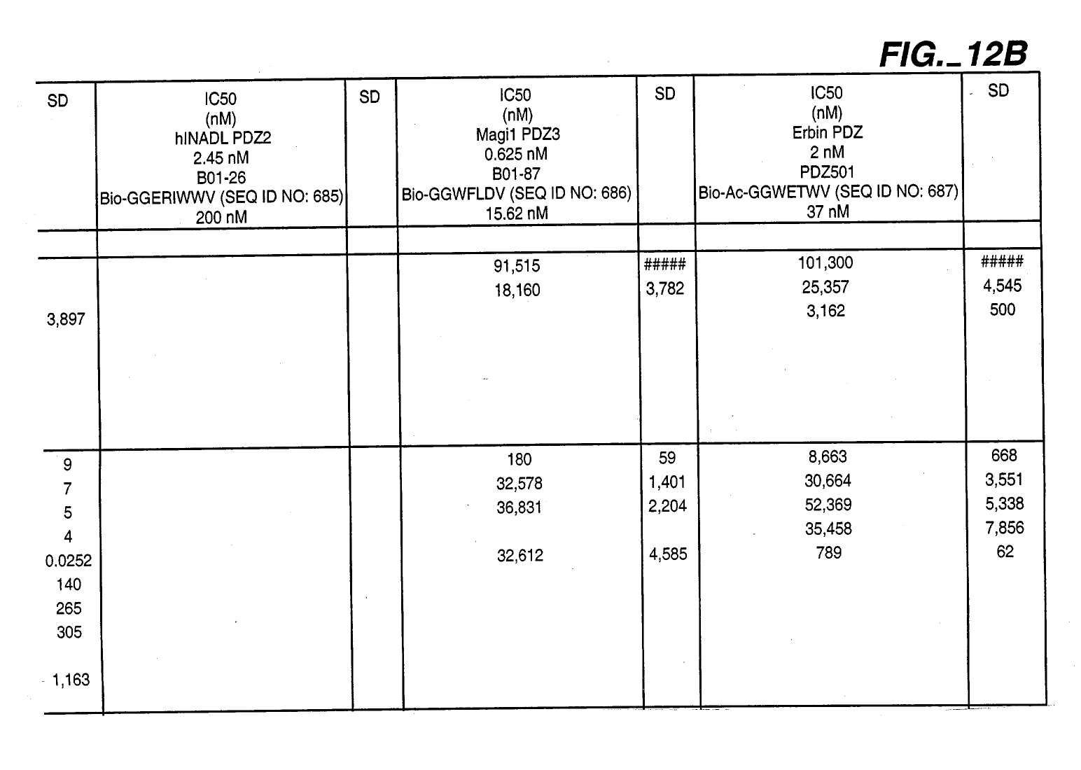

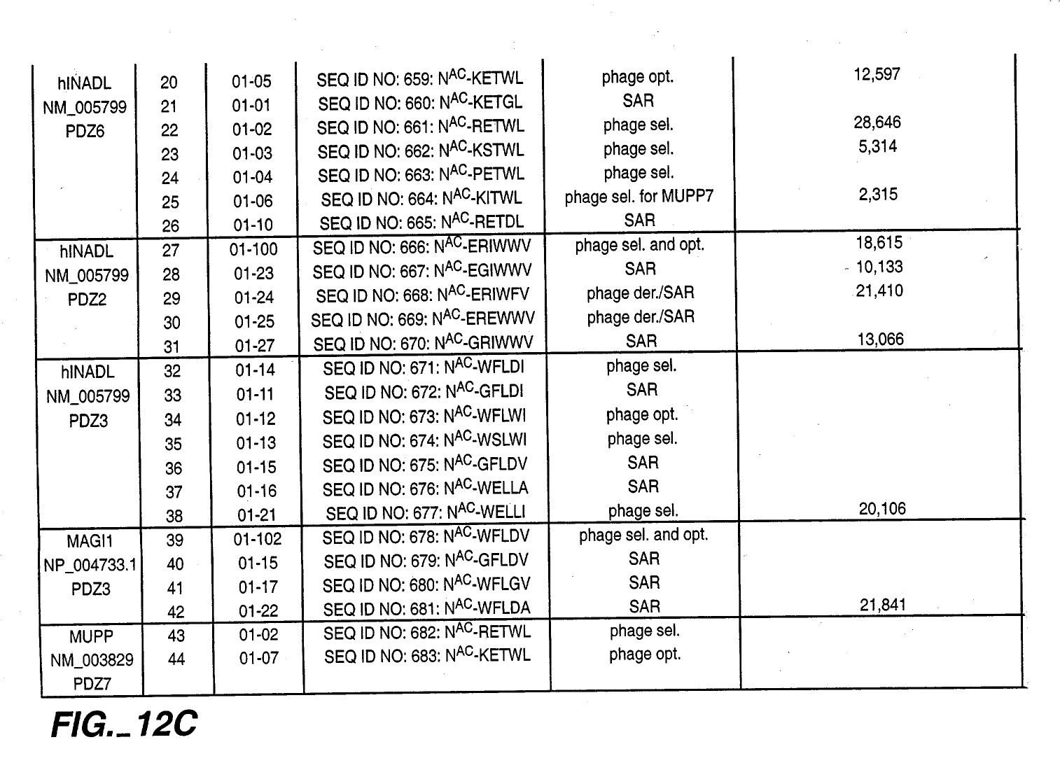

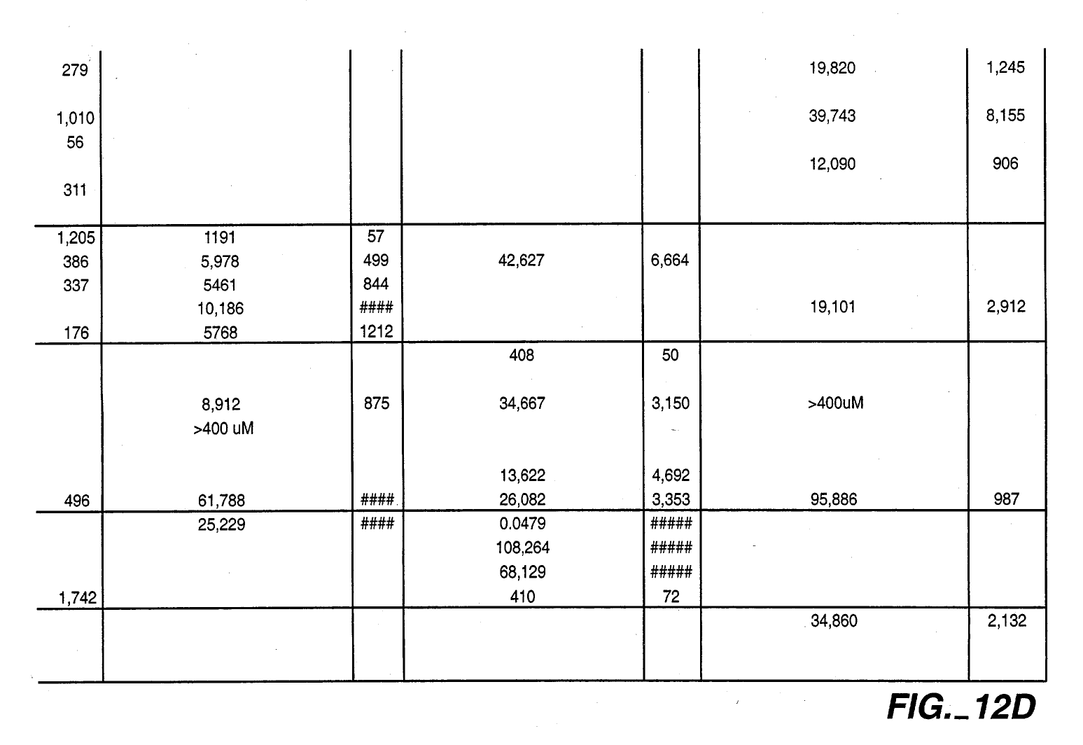

A variety of peptides with variations in sequence and/or modifications of the N-terminal residue (by acetylation) were tested against various PDZ domains. Binding affinity determinations were based on IC50 values, which are depicted in FIG. 12.

In FIG. 12, the sequences of tested peptides were designed based on (1) sequence of selected phage binder (“Phage sel.”); (2) sequence derived from selected phage binder or is based on selected phage binder sequence (“Phage der.”); (3) the sequence of a theoretical optimal binder, based on phaging results (“Phage opt.”); (4) a design appropriate to obtain information about structure-activity relationship (“SAR”); and/or (5) the sequence of a predicted cognate ligand. “N^{Ac}” refers to acetylation of the N-terminal residue. “Receptor” refers to the target PDZ domain for which a test peptide's binding affinity is determined. “Biot. Peptide” refers to a biotinylated peptide.

IC50 Assay

All test peptides were first tested at 400 uM for their ability to inhibit the binding of biotinylated peptides to a corresponding receptor. Peptides that showed >40% inhibition were then re-tested at varying concentrations for determination of IC50 values, which are depicted in FIG. 11. Values depicted are the average of 3 data points for each peptide/receptor.

Homogeneous binding assays were performed in either 384-well Optiplates from PerkinElmer Life Sciences (Meriden, Conn., USA) or 384-well NUNC™ white assay plates from Nalge Nunc International (Naperville, Ill., USA). Reaction mixtures containing reagent concentrations listed in Table 30 were prepared in assay buffer (phosphate buffer saline (PBS)) with 0.1% bovine gamma globulin; 0.05% Tween 20 and 10 ppm Proclin ph 7.4. 15 ul of this mixture was added to each well. Each sample was diluted to give 2 mM in 20% DMSO-assay buffer. 5 ul aliquots of diluted samples were added to each well containing 15 ul of reaction mixture. Reactions were allowed to proceed for 1 hour in the dark at room temperature with gentle agitation. 5 ul of donor beads (100 ug/ml) was added to each well and the incubation continued in the dark for 2 hours. The resulting plates were read on Packard AlphaQuest (PerkinElmer Life Sciences, Meriden, Conn., USA), which is a time resolved fluorescent plate reader at an excitation wavelength of 680 nm and emission wavelength of 520-620 nm.

Peptides showing >40% inhibition were initially prepared at a concentration of 1 mM in 20% DMSO-assay buffer. Additional 23 dilutions were made using 1:3 serial dilutions in 20% DMSO-assay buffer to give a total of 24 dilutions per peptide (sample). 5 ul of the each of these diluted samples was added to wells each containing 15 ul of reaction mixture. Assays were carried out as above.

TABLE 30
Concentration of reagents in the assay well

| | | | Acceptor | Donor |
|---|---|---|---|---|
| | Receptor | Biotin-peptide | beads* | beads* |
| Reagent | ERBIN PDZ-GST | Biotin-PDZ501 | Anti-GST | Strepavidin |
| Concentration | 2 nM | 37 nM | 20 ug/ml | 20 ug/ml |
| Reagent | hINADL PDZ2- | Biotin-B01-26 | Anti-GST | Strepavidin |
| | GST | | | |
| Concentration | 2.45 nM | 200 nM | 20 ug/ml | 20 ug/ml |
| Reagent | HZO1 PDZ1-GST | Biotin-B01-88 | Anti-GST | Strepavidin |
| Concentration | 5 nM | 36 nM | 20 ug/ml | 20 ug/ml |
| Reagent | hMagil PDZ3- | Biotin-B01-87 | Anti-GST | Strepavidin |
| | GST | | | |
| Concentration | 0.625 nM | 15.62 nM | 20 ug/ml | 20 ug/ml |

*Acceptor beads and donor beads were purchased from Packard Instrument (PerkinElmer Life Sciences, Meriden, CT, USA)

### PARTIAL LIST OF REFERENCES

U.S. Pat. No. 4,166,452. Apparatus for testing human responses to stimuli. 1979.

U.S. Pat. No. 4,485,045. Synthetic phosphatidyl cholines useful in forming liposomes. 1984.

U.S. Pat. No. 4,544,545. Liposomes containing modified cholesterol for organ targeting. 1985.

U.S. Pat. No. 4,676,980. Target specific cross-linked heteroantibodies. 1987.

U.S. Pat. No. 4,816,567. Recombinant immunoglobin preparations. 1989.

WO 90/10448. Covalent conjugates of lipid and oligonucleotide. 1990.

WO 90/13641. Stably transformed eucaryotic cells comprisng a foreign transcribable DNA under the control of a pol III promoter. 1990.

EPO 402226. Transformation vectors for yeast Yarrowia. 1990.

WO 91/00360. Bispecific reagents for AIDS therapy. 1991.

WO 91/04753. Conjugates of antisense oligonucleotides and therapeutic uses thereof. 1991.

U.S. Pat. No. 5,013,556. Liposomes with enhanced circulation time. 1991.

WO 91/00357. New strain with filamentous fungi mutants, process for the production of recombinant proteins using said strain, and strains and proteins. 1991.

WO 91/06629. Oligonucleotide analogs with novel linkages. 1991.

WO 92/20373. Heteroconjugate antibodies for treatment of HIV infection. 1992.

WO 93/08829. Compositions that mediate killing of HIV-infected cells. 1993.

WO 94/11026. Therapeutic application of chimeric and radiolabeled antibodies to human B lymphocyte restricted differentiation antigen for treatment of B cells. 1994.

WO 96/27011. A method for making heteromultimeric polypeptides. 1996.

U.S. Pat. No. 5,545,807. Production of antibodies from transgenic animals. 1996.

U.S. Pat. No. 5,545,806. Ransgenic <sic> non-human animals for producing heterologous antibodies. 1996.

U.S. Pat. No. 5,569,825. Transgenic non-human animals capable of producing heterologous antibodies of various isotypes. 1996.

WO 97/33551. Compositions and methods for the diagnosis, prevention, and treatment of neoplastic cell growth and proliferation. 1997.

U.S. Pat. No. 5,633,425. Transgenic non-human animals capable of producing heterologous antibodies. 1997.

U.S. Pat. No. 5,661,016. Transgenic non-human animals capable of producing heterologous antibodies of various isotypes. 1997.

U.S. Pat. No. 5,625,126. Transgenic non-human animals for producing heterologous antibodies. 1997.

U.S. Pat. No. 6,294,330 B1. Protein fragment complementation assays for the detection of biological or drug interactions. 2001.

U.S. Pat. No. 6,270,964 B1. Protein fragment complementation assays for the detection of biological or drug interactions. 2001.

Abravaya, K., J. J. Carrino, S. Muldoon, and H. H. Lee. 1995. Detection of point mutations with a modified ligase chain reaction (Gap-LCR). Nucleic Acids Res. 23:675-82.

Ahn, A. H., C. A. Freener, E. Gussoni, M. Yoshida, el al. 1996. The three human syntrophin genes are expressed in diverse tissues, have distinct chromosomal locations, and each bind to dystrophin and its relatives. J Biol Chem. 271:2724-30.

Alam, J., and J. L. Cook. 1990. Reporter genes: Application to the study of mammalian gene transcription. Anal. Biochem. 188:245-254.

Andersson, B., M. A. Wentland, J. Y. Ricafrente, W. Liu, et al. 1996. A “double adaptor” method for improved shotgun library construction. Anal Biochem. 236:107-13.

Arimura, Y., Y. Hinoda, F. Itoh, M. Takekawa, et al. 1992. cDNA cloning of new protein tyrosine phosphatases in the human colon. Tumour Biol. 13:180-6.

Austin, C. P., and C. L. Cepko. 1990. Cellular migration patterns in the developing mouse cerebral cortex. Development. 110:713-732.

Ausubel, F. M., R. Brent, R. E. Kingston, D. D. Moore, et al. 1987. Current protocols in molecular biology. John Wiley & Sons, New York.

Azim, A. C., J. H. Knoll, S. M. Marfatia, D. J. Peel, et al. 1995. DLG1: chromosome location of the closest human homologue of the Drosophila discs large tumor suppressor gene. Genomics. 30:613-6.

Baier, M., N. Bannert, A. Werner, K. Lang, et al. 1997. Molecular cloning, sequence, expression, and processing of the interleukin 16 precursor. Proc Natl Acad Sci USA. 94:5273-7.

Bannert, N., M. Baier, A. Werner, and R. Kurth. 1996. Interleukin-16 or not? Nature. 381:30.

Barany, F. 1991. Genetic disease detection and DNA amplification using cloned thermostable ligase. Proc Natl Acad Sci USA. 88:189-93.

Barrett, R. W., S. E. Cwirla, M. S. Ackerman, A. M. Olson, et al. 1992. Selective enrichment and characterization of high affinity ligands from collections of random peptides on filamentous phage. Anal Biochem. 204:357-64.

Bartel, D. P., and J. W. Szostak. 1993. Isolation of new ribozymes from a large pool of random sequences [see comment]. Science. 261:1411-8.

Bashirova, A. A., M. L. Markelov, T. V. Shlykova, E. V. Levshenkova, et al. 1998. The human RIL gene: mapping to human chromosome 5q31.1, genomic organization and alternative transcripts. Gene. 210:239-45.

Bass, S., R. Greene, and J. A. Wells. 1990. Hormone phage: an enrichment method for variant proteins with altered binding properties. Proteins. 8:309-14.

Beal, P. A., and P. B. Dervan. 1991. Second structural motif for recognition of DNA by oligonucleotide-directed triple-helix formation. Science. 251:1360-3.

Beatch, M., L. A. Jesaitis, W. J. Gallin, D. A. Goodenough, et al. 1996. The tight junction protein ZO-2 contains three PDZ (PSD-95/Discs-Large/ZO-1) domains and an alternatively spliced region. J Biol Chem. 271:25723-6.

Bechtold, N., and G. Pelletier. 1998. In planta Agrobacterium-mediated transformation of adult Arabidopsis thaliana plants by vacuum infiltration. Methods Mol Biol. 82:259-66.

Becker, D. M., and L. Guarente. 1991. High-efficiency transformation of yeast by electroporation. Methods Enzymol. 194:182-187.

Beggs, J. D. 1978. Transformation of yeast by a replicating hybrid plasmid. Nature. 275:104-109.

Berger, J., J. Hauber, R. Hauber, R. Geiger, et al. 1988. Secreted placental alkaline phosphatase: A powerful new qunatitative indicator of gene expression in eukaryotic cells. Gene. 66:1-10.

Bodine, D. M., K. T. McDonagh, N. E. Seidel, and A. W. Nienhuis. 1991. Survival and retrovirus infection of murine hematopoietic stem cells in vitro: effects of 5-FU and method of infection. Exp. Hematol. 19:206-212.

Boerner, P., R. Lafond, W. Z. Lu, P. Brams, et al. 1991. Production of antigen-specific human monoclonal antibodies from in vitro-primed human splenocytes. J Immunol. 147:86-95.

Borg, J. P., S. Marchetto, A. Le Bivic, V. Ollendorff, et al. 2000. ERBIN: a basolateral PDZ protein that interacts with the mammalian ERBB2/HER2 receptor. Nat Cell Biol. 2:407-14.

Borg, J. P., S. W. Straight, S. M. Kaech, M. de Taddeo-Borg, et al. 1998. Identification of an evolutionarily conserved heterotrimeric protein complex involved in protein targeting. J Biol Chem. 273:31633-6.

U.S. Pat. No. 3,773,919. Polylactide-drug mixtures. 1973.

Bouju, S., G. Pietu, M. Le Cunff, N. Cros, et al. 1999. Exclusion of muscle specific actinin-associated LIM protein (ALP) gene from 4q35 facioscapulohumeral muscular dystrophy (FSHD) candidate genes. Neuromuscul Disord. 9:3-10.

Brennan, M., P. F. Davison, and H. Paulus. 1985. Preparation of bispecific antibodies by chemical recombination of monoclonal immunoglobulin G1 fragments. Science. 229:81-3.

Bruckner, K., J. Pablo Labrador, P. Scheiffele, A. Herb, et al. 1999. EphrinB ligands recruit GRIP family PDZ adaptor proteins into raft membrane microdomains. Neuron. 22:511-24.

Bryant, P. J., and D. F. Woods. 1992. A major palmitoylated membrane protein of human erythrocytes shows homology to yeast guanylate kinase and to the product of a Drosophila tumor suppressor gene. Cell. 68:621-2.

Butz, S., M. Okamoto, and T. C. Sudhof. 1998. A tripartite protein complex with the potential to couple synaptic vesicle exocytosis to cell adhesion in brain. Cell. 94:773-82.

Cabot, E. L., and A. T. Beckenbach. 1989. Simultaneous editing of multiple nucleic acid and protein sequences with ESEE. Comput Appl Biosci. 5:233-4.

Capecchi, M. R. 1980. High efficiency transformation by direct microinjection of DNA into cultured mammalian cells. Cell. 22:479.

Capecchi, M. R. 1989. Altering the genome by homologous recombination. Science. 244:1288-92.

Carell, T., E. A. Wintner, and J. Rebek Jr. 1994a. A novel procedure for the synthesis of libraries containing small organic molecules. Angewandte Chemie International Edition. 33:2059-2061.

Carell, T., E. A. Wintner, and J. Rebek Jr. 1994b. A solution phase screening procedure for the isolation of active compounds from a molecular library. Angewandte Chemie International Edition. 33:2061-2064.

Caron, P. C., W. Laird, M. S. Co, N. M. Avdalovic, et al. 1992. Engineered humanized dimeric forms of IgG are more effective antibodies. J Exp Med. 176:1191-5.

Carter, P. 1986. Site-directed mutagenesis. Biochem J. 237:1-7.

Case, M. E., M. Schweizer, S. R. Kushner, and N. H. Giles. 1979. Efficient transformation of Neurospora crassa by utilizing hybrid plasmid DNA. Proc Natl Acad Sci USA. 76:5259-63.

Castello, A., V. Brocheriou, P. Chafey, A. Kahn, et al. 1996. Characterization of the dystrophin-syntrophin interaction using the two-hybrid system in yeast. FEBS Lett. 383:124-8.

U.S. Pat. No. 5,116,742. RNA ribozyme restriction endoribonucleases and methods. 1992.

U.S. Pat. No. 4,987,071. RNA ribozyme polymerases, dephosphorylases, restriction endoribonucleases and methods. 1991.

Cepko, C. L., B. E. Roberts, and R. E. Mulligan. 1984. Construction and applications of a highly transmissible murine retrovirus shuttle vector. Cell. 37:1053-1062.

Chalfie, M., Y. tu, G. Euskirchen, W. W. Ward, et al. 1994. Green fluorescent protein as a marker for gene expression. Science. 263:802-805.

Chaney, W. G., D. R. Howard, J. W. Pollard, S. Sallustio, et al. 1986. High-frequency transfection of CHO cells using Polybrene. Somatic Cell Mol. Genet. 12:237.

Chen, C., and H. Okayama. 1988. Calcium phosphate-mediated gene transfer: A highly efficient system for stably transforming cells with plasmid DNA. BioTechniques. 6:632-638.

Chen, H., and S. E. Antonarakis. 1995. Localization of a human homolog of the mouse Tiam-1 gene to chromosome 21q22.1. Genomics. 30:123-7.

Chen, S. H., H. D. Shine, J. C. Goodman, R. G. Grossman, et al. 1994. Gene therapy for brain tumors: regression of experimental gliomas by adenovirus-mediated gene transfer in vivo. Proc Natl Acad Sci U S A. 91:3054-7.

Chiu, C. Y., S. Leng, K. A. Martin, E. Kim, et al. 1999. Cloning and characterization of T-cell lymphoma invasion and metastasis 2 (TIAM2), a novel guanine nucleotide exchange factor related to TIAM1. Genomics. 61:66-73.

Cho, C. Y., E. J. Moran, S. R. Cherry, J. C. Stephans, et al. 1993. An unnatural biopolymer. Science. 261:1303-5.

Clackson, T., H. R. Hoogenboom, A. D. Griffiths, and G. Winter. 1991. Making antibody fragments using phage display libraries. Nature. 352:624-8.

Cohen, A. R., D. F. Woods, S. M. Marfatia, Z. Walther, et al. 1998. Human CASK/LIN-2 binds syndecan-2 and protein 4.1 and localizes to the basolateral membrane of epithelial cells. J Cell Biol. 142:129-38.

Cohen, A. S., D. L. Smisek, and B. H. Wang. 1996. Emerging technologies for sequencing antisense oligonucleotides: capillary electrophoresis and mass spectrometry. Adv Chromatogr. 36:127-62.

Cohen, J. S. 1989. Oligodeoxynucleotides: Antisense inhibitors of gene expression. CRC Press, Boca Raton, Fla. 255 pp.

Cohen, S. M. N., A. C. Y. Chang, and L. Hsu. 1972. Nonchromosomal antibiotic resistance in bacteria: Genetic transformation of Escherichia coli by R-factor DNA. Proc. Natl. Acad. Sci. USA. 69:2110.

Cooney, M., G. Czernuszewicz, E. H. Postel, S. J. Flint, et al. 1988. Site-specific oligonucleotide binding represses transcription of the human c-myc gene in vitro. Science. 241:456-9.

Cotton, R. G. 1993. Current methods of mutation detection. Mutat Res. 285:125-44.

Cowburn, D., and G. Riddihough. 1997. A never ending frontier. Nat Struct Biol. 4:761-2.

Craven, S. E., and D. S. Bredt. 1998. PDZ proteins organize synaptic signaling pathways. Cell. 93:495-8.

Cronin, M. T., R. V. Fucini, S. M. Kim, R. S. Masino, et al. 1996. Cystic fibrosis mutation detection by hybridization to light-generated DNA probe arrays. Hum Mutat. 7:244-55.

Cull, M. G., J. F. Miller, and P. J. Schatz. 1992. Screening for receptor ligands using large libraries of peptides linked to the C terminus of the lac repressor. Proc Natl Acad Sci USA. 89:1865-9.

Cwirla, S. E., E. A. Peters, R. W. Barrett, and W. J. Dower. 1990. Peptides on phage: a vast library of peptides for identifying ligands. Proc Natl Acad Sci U S A. 87:6378-82.

Daniels, D. L., A. R. Cohen, J. M. Anderson, and A. T. Brunger. 1998. Crystal structure of the hCASK PDZ domain reveals the structural basis of class II PDZ domain target recognition. Nat Struct Biol. 5:317-25.

Dayhoff, M. O., R. M. Schwartz, and B. C. Orcutt. 1978. A model of evolutionary change in proteins Matrices for detecting distant relationships. In Atlas of protein sequence and structure. Vol. 5. M. O. Dayhoff, editor. National biomedical research foundation, Washington, D.C. 345-362.

de Boer, A. G. 1994. Drug absorption enhancement: Concepts, possibilities, limitations and trends. Harwood Academic Publishers, Langhorne, Pa.

de Louvencourt, L., H. Fukuhara, H. Heslot, and M. Wesolowski. 1983. Transformation of Kluyveromyces lactis by killer plasmid DNA. J Bacteriol. 154:737-42.

De Vries, L., X. Lou, G. Zhao, B. Zheng, et al. 1998. GIPC, a PDZ domain containing protein, interacts specifically with the C terminus of RGS-GAIP. Proc Natl Acad Sci USA. 95:12340-5.

de Wet, J. R., K. V. Wood, M. DeLuca, D. R. Helinski, et al. 1987. Sturcture and expression in mammalian cells. Mol. Cell Biol. 7:725-737.

DeAngelis, M. M., T. L. McGee, B. J. Keats, R. Slim, et al. 2001. Two families from New England with usher syndrome type IC with distinct haplotypes. Am J Ophthalmol. 131:355-8.

Deguchi, M., T. Iizuka, Y. Hata, W. Nishimura, et al. 2000. PAPIN. A novel multiple PSD-95/Dlg-A/ZO-1 protein interacting with neural plakophilin-related armadillo repeat protein/delta-catenin and pO071. J Biol Chem. 275:29875-80.

Demerec, M., E. A. Adelberg, A. J. Clark, and P. E. Hartman. 1966. A proposal for a uniform nomenclature in bacterial genetics. Genetics. 54:61-76.

Devlin, J. J., L. C. Panganiban, and P. E. Devlin. 1990. Random peptide libraries: a source of specific protein binding molecules. Science. 249:404-6.

DeWitt, S. H., J. S. Kiely, C. J. Stankovic, M. C. Schroeder, et al. 1993. “Diversomers”: an approach to nonpeptide, nonoligomeric chemical diversity. Proc Natl Acad Sci USA. 90:6909-13.

Dimitratos, S. D., D. G. Stathakis, C. A. Nelson, D. F. Woods, et al. 1998. The location of human CASK at Xpl 1.4 identifies this gene as a candidate for X-linked optic atrophy. Genomics. 51:308-9.

Dixon, B., B. Sahely, L. Liu, and B. Pohajdak. 1993. Cloning a cDNA from human NK/T cells which codes for an unusual leucine zipper containing protein. Biochim Biophys Acta. 1216:321-4.

Doyle, D. A., A. Lee, J. Lewis, E. Kim, et al. 1996. Crystal structures of a complexed and peptide-free membrane protein-binding domain: molecular basis of peptide recognition by PDZ. Cell. 85:1067-76.

Duclos, F., U. Boschert, G. Sirugo, J. L. Mandel, et al. 1993. Gene in the region of the Friedreich ataxia locus encodes a putative transmembrane protein expressed in the nervous system. Proc Natl Acad Sci USA. 90:109-13.

Duclos, F., and M. Koenig. 1995. Comparison of primary structure of a neuron-specific protein, X11, between human and mouse. Mamm Genome. 6:57-8.

Duclos, F., F. Rodius, K. Wrogemann, J. L. Mandel, et al. 1994. The Friedreich ataxia region: characterization of two novel genes and reduction of the critical region to 300 kb. Hum Mol Genet. 3:909-14.

Edelhoch, H. 1967. Spectroscopic detemination of tryptophan and tyrosine in proteins. Biochemistry. 6:1948-54.

Edwards, D. C., and G. N. Gill. 1999. Structural features of LIM kinase that control effects on the actin cytoskeleton. J Biol Chem. 274:11352-61.

Efimov, V. P., I. V. Nepluev, and V. V. Mesyanzhinov. 1995. Bacteriophage T4 as a surface display vector. Virus Genes. 10:173-7.

Ehrlich, H. A. 1992. PCR technology: principles and applications for DAN application. W. H. Freeman & Co., New York, N.Y. 246.

Eichelbaum, M., and B. Evert. 1996. Influence of pharmacogenetics on drug disposition and response. Clin Exp Pharmacol Physiol. 23:983-5.

Ellington, A. D., and J. W. Szostak. 1990. In vitro selection of RNA molecules that bind specific ligands. Nature. 346:818-22.

Elroy-Stein, O., and B. Moss. 1990. Cytoplasmic expression system based on constitutive synthesis of bacteriqphage T7 RNA polymerase in mammalian cells. Proc. Natl. Acad. Sci. USA. 87:6743-6747.

U.S. Pat. No. 4,522,811. Serial injection of muramyldipeptides and liposomes enhances the anti-infective activity of muramyldipeptides Serial injection of muramyldipeptides and liposomes enhances the anti-infective activity of muramyldipeptides. 1985.

Eppstein, D. A., Y. V. Marsh, M. van der Pas, P. L. Feigner, et al. 1985. Biological activity of liposome-encapsulated murine interferon gamma is mediated by a cell membrane receptor. Proc Natl Acad Sci USA. 82:3688-92.

Escudero, J., and B. Hohn. 1997. Transfer and integration of T-DNA without cell injury in the host plant. Plant Cell. 9:2135-2142.

Fanning, A. S., and J. M. Anderson. 1999. PDZ domains: fundamental building blocks in the organization of protein complexes at the plasma membrane. J Clin Invest. 103:767-72.

Fekete, D. M., and C. L. Cepko. 1993. Retroviral infection coupled with tissue transplantation limits gene transfer in the chick embryo. Proc. Natl. Acad. Sci. USA. 90:2350-2354.

Felgner, P. L., T. R. Gadek, M. Holm, R. Roman, et al. 1987. Lipofectin: A highly efficient, lipid-mediated DNA/transfection procedure. Proc. Natl. Acad. Sci. USA. 84:7413-7417.

Felici, F., L. Castagnoli, A. Musacchio, R. Jappelli, el al. 1991. Selection of antibody ligands from a large library of oligopeptides expressed on a multivalent exposition vector. J Mol Biol. 222:301-10.

Fieck, A., D. L. Wyborski, and J. M. Short. 1992. Modifications of the E.coli Lac repressor for expression in eukaryotic cells: effects of nuclear signal sequences on protein activity and nuclear accumulation. Nucleic Acids Res. 20:1785-91.

Finer, J. J., K. R. Finer, and T. Ponappa. 1999. Particle bombardment-mediated transformation. Current Topics in microbiology and immunology. 240:59-80.

Finn, P. J., N. J. Gibson, R. Fallon, A. Hamilton, el al. 1996. Synthesis and properties of DNA-PNA chimeric oligomers. Nucleic Acids Res. 24:3357-63.

Fishwild, D. M., S. L. O'Donnell, T. Bengoechea, D. V. Hudson, et al. 1996. High-avidity human IgG kappa monoclonal antibodies from a novel strain of minilocus transgenic mice [see comments]. Nat Biotechnol. 14:845-51. nFleer, R., P. Yeh, N. Amellal, I. Maury, et al. 1991. Stable multicopy vectors for high-level secretion of recombinant human serum albumin by Kluyveromyces yeasts. Biotechnology (N Y). 9:968-75.

Fodor, S. P., R. P. Rava, X. C. Huang, A. C. Pease, et al. 1993. Multiplexed biochemical assays with biological chips. Nature. 364:555-6.

Frangiskakis, J. M., A. K. Ewart, C. A. Morris, C. B. Mervis, et al. 1996. LIM-kinasel hemizygosity implicated in impaired visuospatial constructive cognition. Cell. 86:59-69.

U.S. Pat. No. 5,804,604. Tat-derived transport polypeptides and fusion proteins. 1998.

Fromm, M., L. P. Taylor, and V. Walbot. 1985. Expression of genes transferred into monocot and dicot plant cells by electroporation. Proc. Natl. Acad. Sci. USA. 82:5824-5828.

Fujisawa, H., T. Ogura, Y. Kurashima, T. Yokoyama, et al. 1994. Expression of two types of nitric oxide synthase mRNA in human neuroblastoma cell lines. J Neurochem. 63:140-5.

Fujita, T., H. Shubiya, T. Ohashi, K. Yamanishi, et al. 1986. Regulation of human interleukin-2 gene: Functional DNA sequences in the 5′ flanking region for the gene expression in activated T lymphocytes. Cell. 46:401-407.

Fukuhara, S., C. Murga, M. Zohar, T. Igishi, et al. 1999. A novel PDZ domain containing guanine nucleotide exchange factor links heterotrimeric G proteins to Rho. J Biol Chem. 274:5868-79.

Gabizon, A., R. Shiota, and D. Papahadjopoulos. 1989. Pharmacokinetics and tissue distribution of doxorubicin encapsulated in stable liposomes with long circulation times. J Natl Cancer Inst. 81:1484-8.

Gallagher, S. R. 1992. GUS protocols: Using the GUS gene as a reporter of gene expression. Academic Press, San Diego, Calif.

Gallop, M. A., R. W. Barrett, W. J. Dower, S. P. Fodor, et al. 1994. Applications of combinatorial technologies to drug discovery. 1. Background and peptide combinatorial libraries. J Med Chem. 37:1233-51.

Gasparini, P., A. Bonizzato, M. Dognini, and P. F. Pignatti. 1992. Restriction site generating-polymerase chain reaction (RG-PCR) for the probeless detection of hidden genetic variation: application to the study of some common cystic fibrosis mutations. Mol Cell Probes. 6:1-7.

Gautier, C., F. Morvan, B. Rayner, T. Huynh-Dinh, et al. 1987. Alpha-DNA. IV: Alpha-anomeric and beta-anomeric tetrathymidylates covalently linked to intercalating oxazolopyridocarbazole. Synthesis, physicochemical properties and poly (rA) binding. Nucleic Acids Res. 15:6625-41.

Gee, S. H., S. A. Sekely, C. Lombardo, A. Kurakin, et al. 1998. Cyclic peptides as non-carboxyl-terminal ligands of syntrophin PDZ domains. J Biol Chem. 273:21980-7.

Gennaro, A. R. 2000. Remington: The science and practice of pharmacy. Lippincott, Williams & Wilkins, Philadelphia, Pa.

Gibbs, R. A., P. N. Nguyen, and C. T. Caskey. 1989. Detection of single DNA base differences by competitive oligonucleotide priming. Nucleic Acids Res. 17:2437-48.

Gietz, R. D., R. A. Woods, P. Manivasakam, and R. H. Schiestl. 1998. Growth and transformation of Saccharomyces cerevisiae. In Cells: A laboratory manual. Vol. I. D. Spector, R. Goldman, and L. Leinwand, editors. Cold Spring Harbor Press, Cold Spring Harbor, N.Y.

Gillespie, C. S., D. L. Sherman, S. M. Fleetwood-Walker, D. F. Cottrell, et al. 2000. Peripheral demyelination and neuropathic pain behavior in periaxin-deficient mice. Neuron. 26:523-31.

Goding, J. W. 1996. Monoclonal antibodies: Principles and Practice. Academic Press, San Diego. 492 pp.

Gorman, C. M., L. F. Moffat, and B. H. Howard. 1982. Recombinant genomes which express chloramphenicol acetyltransferase in mammalian cells. Mol. Cell. Biol. 2:1044-1051.

Graham, F. L., and A. J. van der Eb. 1973. A new technique for the assay of infectivity of human adenovirus 5 DNA. Virology. 52:456-.

Greco, T. L., D. J. Sussman, and S. A. Camper. 1996. Dishevelled-2 maps to human chromosome 17 and distal to Wnt3a and vestigial tail (vt) on mouse chromosome 11. Mamm Genome. 7:475-6.

Griffin, H. G., and A. M. Griffin. 1993. DNA sequencing. Recent innovations and future trends. Appl Biochem Biotechnol. 38:147-59.

Grompe, M., D. M. Muzny, and C. T. Caskey. 1989. Scanning detection of mutations in human ornithine transcarbamoylase by chemical mismatch cleavage. Proc Natl Acad Sci USA. 86:5888-92.

Gruber, M., B. A. Schodin, E. R. Wilson, and D. M. Kranz. 1994. Efficient tumor cell lysis mediated by a bispecific single chain antibody expressed in Escherichia coli. J Immunol. 152:5368-74.

Gu, M. X., J. D. York, I. Warshawsky, and P. W. Majerus. 1991. Identification, cloning, and expression of a cytosolic megakaryocyte protein-tyrosine-phosphatase with sequence homology to cytoskeletal protein 4.1. Proc Natl Acad Sci USA. 88:5867-71.

Guatelli, J. C., K. M. Whitfield, D. Y. Kwoh, K. J. Barringer, et al. 1990. Isothermal, in vitro amplification of nucleic acids by a multienzyme reaction modeled after retroviral replication. Proc Natl Acad Sci USA. 87:1874-8.

Habets, G. G., R. A. van der Kammen, N. A. Jenkins, D. J. Gilbert, et al. 1995a. The invasion-inducing TIAM1 gene maps to human chromosome band 21 q22 and mouse chromosome 16. Cytogenet Cell Genet. 70:48-51.

Habets, G. G., R. A. van der Kammen, J. C. Stam, F. Michiels, et al. 1995b. Sequence of the human invasion-inducing TIAM1 gene, its conservation in evolution and its expression in tumor cell lines of different tissue origin. Oncogene. 10:1371-6.

Hall, R. A., L. S. Ostedgaard, R. T. Premont, J. T. Blitzer, et al. 1998. A C-terminal motif found in the beta2-adrenergic receptor, P2Y1 receptor and cystic fibrosis transmembrane conductance regulator determines binding to the Na+/H+ exchanger regulatory factor family of PDZ proteins. Proc Natl Acad Sci USA. 95:8496-501.

Hanahan, D. 1983. Studies on transformation of Escherichia co/i with plasmids. J. Mol. Biol. 166:557-580.

Hansen, G., and M.-D. Chilton. 1999. Lessons in gene transfer to plants by a gifted microbe. Curr. Top. Microbiol. Immunol. 240:21-57.

Hansen, G., and M. S. Wright. 1999. Recent advances in the transformation of plants. Trends Plant Sci. 4:226-231.

Harlow, E., and D. Lane. 1988. Antibodies: A laboratory manual. Cold Spring Harbor Laboratory Press, Cold Spring Harbor. 726 pp.

Harlow, E., and D. Lane. 1999. Using antibodies: A laboratory manual. Cold Spring Harbor Laboratory PRess, Cold Spring Harbor, N.Y.

Harrison, S. C. 1996. Peptide-surface association: the case of PDZ and PTB domains. Cell. 86:341-3.

Haseloff, J., and W. L. Gerlach. 1988. Simple RNA enzymes with new and highly specific endoribonuclease activities. Nature. 334:585-91.

Hattori, M., A. Fujiyama, T. D. Taylor, H. Watanabe, et al. 2000. The DNA sequence of human chromosome 21. Nature. 405:311-9.

Hayashi, K. 1992. PCR-SSCP: A method for detection of mutations. Genetic and Analytical Techniques Applications. 9:73-79.

Helene, C. 1991. The anti-gene strategy: control of gene expression by triplex-forming-oligonucleotides. Anticancer Drug Des. 6:569-84.

Helene, C., N. T. Thuong, and A. Harel-Bellan. 1992. Control of gene expression by triple helix-forming oligonucleotides. The antigene strategy. Ann N Y Acad Sci. 660:27-36.

Hillier, B. J., K. S. Christopherson, K. E. Prehoda, D. S. Bredt, et al. 1999. Unexpected modes of PDZ domain scaffolding revealed by structure of nNOS-syntrophin complex. Science. 284:812-5.

Hinnen, A., J. B. Hicks, and G. R. Fink. 1978. Transformation of yeast. Proc. Natl. Acad. Sci. USA. 75:1929-1933.

Hoffman, F. 1996. Laser microbeams for the manipulation of plant cells and subcellular structures. Plant Sci. 113: 1-11.

Holliger, P., T. Prospero, and G. Winter. 1993. “Diabodies”: small bivalent and bispecific antibody fragments. Proc Natl Acad Sci USA. 90:6444-8.

Hoogenboom, H. R., A. D. Griffiths, K. S. Johnson, D. J. Chiswell, et al. 1991. Multi-subunit proteins on the surface of filamentous phage: methodologies for displaying antibody (Fab) heavy and light chains. Nucleic Acids Res. 19:4133-7.

Hou, J., U. Schindler, W. J. Henzel, T. C. Ho, et al. 1994. An interleukin-4-induced transcription factor: IL-4 Stat. Science. 265:1701-6.

Houghten, R. A., J. R. Appel, S. E. Blondelle, J. H. Cuervo, et al. 1992. The use of synthetic peptide combinatorial libraries for the identification of bioactive peptides. Biotechniques. 13:412-21.

Hsu, I. C., Q. Yang, M. W. Kahng, and J. F. Xu. 1994. Detection of DNA point mutations with DNA mismatch repair enzymes. Carcinogenesis. 15:1657-62.

Hu, S. I., M. Carozza, M. Klein, P. Nantermet, et al. 1998. Human HtrA, an evolutionarily conserved serine protease identified as a differentially expressed gene product in osteoarthritic cartilage. J Biol Chem. 273:34406-12.

Hwang, K. J., K. F. Luk, and P. L. Beaumier. 1980. Hepatic uptake and degradation of unilamellar sphingomyelin/cholesterol liposomes: a kinetic study. Proc Natl Acad Sci USA. 77:4030-4.

Hyrup, B., and P. E. Nielsen. 1996. Peptide nucleic acids (PNA): synthesis, properties and potential applications. Bioorg Med Chem. 4:5-23.

Imai, K., A. H. Sarker, K. Akiyama, S. Ikeda, et al. 1998. Genomic structure and sequence of a human homologue (NTHL1/NTH1) of Escherichia coli endonuclease III with those of the adjacent parts of TSC2 and SLC9A3R2 genes. Gene. 222:287-95.

Inazawa, J., T. Ariyama, T. Abe, T. Druck, et al. 1996. PTPN 13, a fas-associated protein tyrosine phosphatase, is located on the long arm of chromosome 4 at band q21.3. Genomics. 31:240-2.

Inoue, H., Y. Hayase, A. Imura, S. Iwai, et al. 1987a. Synthesis and hybridization studies on two complementary nona(2′-O-methyl)ribonucleotides. Nucleic Acids Res. 15:6131-48.

Inoue, H., Y. Hayase, S. Iwai, and E. Ohtsuka. 1987b. Sequence-dependent hydrolysis of RNA using modified oligonucleotide splints and RNase H. FEBS Lett. 215:327-30.

Ishikawa, K., T. Nagase, M. Suyama, N. Miyajima, et al. 1998. Prediction of the coding sequences of unidentified human genes. X. The complete sequences of 100 new CDNA clones from brain which can code for large proteins in vitro. DNA Res. 5:169-76.

Ishiura, M., S. Hirose, T. Uchida, Y. Hamada, et al. 1982. Phage particle-mediated gene transfer to cultured mammalian cells. Molecular and Cellular Biology. 2:607-616.

Ito, H., Y. Fukuda, K. Murata, and A. Kimura. 1983. Transformation of intact yeast cells treated with alkali cations. J. Bacteriol. 153:163-168.

Itoh, F., S. Ikuta, Y. Hinoda, Y. Arimura, et al. 1993. Expression and chromosomal assignment of PTPH I gene encoding a cytosolic protein tyrosine phosphatase homologous to cytoskeletal-associated proteins. Int J Cancer. 55:947-51.

Jayasena, S. D. 1999. Aptamers: an emerging class of molecules that rival antibodies in diagnostics. Clin Chem. 45:1628-50.

Jespers, L. S., J. H. Messens, A. De Keyser, D. Eeckhout, et al. 1995. Surface expression and ligand-based selection of cDNAs fused to filamentous phage gene VI. Biotechnology (NY). 13:378-82.

Jiang, J. e. a. 1997. CAN. 128:44380.

Jo, K., R. Derin, M. Li, and D. S. Bredt. 1999. Characterization of MALS/Velis-1, -2, and -3: a family of mammalian LIN-7 homologs enriched at brain synapses in association with the postsynaptic density-95/NMDA receptor postsynaptic complex. J Neurosci. 19:4189-99.

Joberty, G., C. Petersen, L. Gao, and I. G. Macara. 2000. The cell-polarity protein Par6 links Par3 and a typical protein kinase C to Cdc42. Nat Cell Biol. 2:531-9.

Jones, P. T., P. H. Dear, J. Foote, M. S. Neuberger, et al. 1986. Replacing the complementarity-determining regions in a human antibody with those from a mouse. Nature. 321:522-5.

Kaech, S. M., C. W. Whitfield, and S. K. Kim. 1998. The LIN-2/LIN-7/LIN-10 complex mediates basolateral membrane localization of the C. elegans EGF receptor LET-23 in vulval epithelial cells. Cell. 94:761-71.

Kamberov, E., O. Makarova, M. Roh, A. Liu, et al. 2000. Molecular cloning and characterization of Pals, proteins associated with mLin-7. J Biol Chem. 275:11425-31.

Kameya, S., Y. Miyagoe, I. Nonaka, T. Ikemoto, et al. 1999. alpha1-syntrophin gene disruption results in the absence of neuronal-type nitric-oxide synthase at the sarcolemma but does not induce muscle degeneration. J Biol Chem. 274:2193-200.

Kang, A. S., C. F. Barbas, K. D. Janda, S. J. Benkovic, et al. 1991. Linkage of recognition and replication functions by assembling combinatorial antibody Fab libraries along phage surfaces. Proc Natl Acad Sci USA. 88:4363-6.

Kaufman, R. J. 1990. Vectors used for expression in mammalian cells. Methods Enzymol. 185:487-511.

Kaufman, R. J., P. Murtha, D. E. Ingolia, C. -Y. Yeung, et al. 1986. Selection and amplification of heterologous genes encoding adenosine deaminase in mammalian cells. Proc. Natl Acad. Sci. USA. 83:3136-3140.

Kawai, S., and M. Nishizawa. 1984. New procedure for DNA transfection with polycation and dimethyl sulfoxide. Mol. Cell. Biol. 4:1172.

Keen, J., D. Lester, C. Inglehearn, A. Curtis, et al. 1991. Rapid detection of single base mismatches as heteroduplexes on Hydrolink gels. Trends Genet. 7:5.

Kelly, J. M., and M. J. Hynes. 1985. Transformation of Aspergillus niger by the amdS gene of Aspergillus nidulans. Embo J. 4:475-9.

Kikuno, R., T. Nagase, K. Ishikawa, M. Hirosawa, et al. 1999. Prediction of the coding sequences of unidentified human genes. XIV. The complete sequences of 100 new CDNA clones from brain which code for large proteins in vitro. DNA Res. 6:197-205.

Kim, A. C., A. B. Metzenberg, K. E. Sahr, S. M. Marfatia, et al. 1996a. Complete genomic organization of the human erythroid p55 gene (MPP 1), a membrane-associated guanylate kinase homologue. Genomics. 31:223-9.

Kim, E., K. O. Cho, A. Rothschild, and M. Sheng. 1996b. Heteromultimerization and NMDA receptor-clustering activity of Chapsyn-110, a member of the PSD-95 family of proteins. Neuron. 17:103-13.

Kim, E., M. Niethammer, A. Rothschild, Y. N. Jan, et al. 1995. Clustering of Shaker-type K+ channels by interaction with a family of membrane-associated guanylate kinases. Nature. 378:85-8.

Kim, H. S. 1999a. Assignment of human interleukin 16 (IL16) to chromosome 15q26.3 by radiation hybrid mapping. Cytogenet Cell Genet. 84:93.

Kim, H. S. 1999b. Assignment of the human B3-1 gene (PSCDBP) to chromosome 2 band q11.2 by radiation hybrid mapping. Cytogenet Cell Genet. 84:95.

Kim, J. H., and M. J. Hahn. 2000. Cloning and sequencing of the gene encoding the candidate HtrA of Rickettsia typhi. Microbiol Immunol. 44:275-8.

Kobayashi, I., K. Imamura, M. Kubota, S. Ishikawa, et al. 1999. Identification of an autoimmune enteropathy-related 75-kilodalton antigen. Gastroenterology. 117:823-30.

Kocher, O., N. Comella, K. Tognazzi, and L. F. Brown. 4998. Identification and partial characterization of PDZK1: a novel protein containing PDZ interaction domains. Lab Invest. 78:117-25.

Kostelny, S. A., M. S. Cole, and J. Y. Tso. 1992. Formation of a bispecific antibody by the use of leucine zippers. J Immunol. 148:1547-53.

WO94/16101. DNA SEQUENCING BY MASS SPECTROMETRY. 1994.

Kozal, M. J., N. Shah, N. Shen, R. Yang, et al. 1996. Extensive polymorphisms observed in HIV-1 clade B protease gene using high-density oligonucleotide arrays. Nat Med. 2:753-9.

Kozbor, D., P. Tripputi, J. C. Roder, and C. M. Croce. 1984. A human hybrid myeloma for production of human monoclonal antibodies. J Immunol. 133:3001-5.

Kunkel, T. A., J. D. Roberts, and R. A. Zakour. 1987. Rapid and efficient site-specific mutagenesis without phenotypic selection. Methods Enzymol. 154:367-82.

Kwoh, D. Y., G. R. Davis, K. M. Whitfield, H. L. Chappelle, et al. 1989. Transcription-based amplification system and detection of amplified human immunodeficiency virus type 1 with a bead-based sandwich hybridization format. Proc Natl Acad Sci USA. 86:1173-7.

U.S. Pat. No. 5,223,409. Directed evolution of novel binding proteins. 1993.

Lam, K. S., S. E. Salmon, E. M. Hersh, V. J. Hruby, et al. 1991. General method for rapid synthesis of multicomponent peptide mixtures. Nature. 354:82-84.

Landegren, U., R. Kaiser, J. Sanders, and L. Hood. 1988. A ligase-mediated gene detection technique. Science. 241:1077-80.

Lau, K. F., D. M. McLoughlin, C. Standen, and C. C. Miller. 2000. X11 alpha and x11 beta interact with presenilin-1 via their PDZ domains. Mol Cell Neurosci. 16:557-65.

Leduc, N., and e. al. 1996. Isolated maize zygotes mimic in vivo embryogenic development and express microinjected genes when cultured in vitro. Dev. Biol. 10:190-203.

Lee, J. S., D. A. Johnson, and A. R. Morgan. 1979. Complexes formed by (pyrimidine)n. (purine)n DNAs on lowering the pH are three-stranded. Nucleic Acids Res. 6:3073-91.

Lee, V. H. L. 1990. Peptide and protein drug delivery. Marcel Dekker, New York, N.Y.

Leek, J. P., P. J. Hamlin, S. M. Bell, and N. J. Lench. 1997. Assignment of the STAT6 gene (STAT6) to human chromosome band 12q13 by in situ hybridization. Cytogenet Cell Genet. 79:208-9.

Lemaitre, M., B. Bayard, and B. Lebleu. 1987. Specific antiviral activity of a poly(L-lysine)-conjugated oligodeoxyribonucleotide sequence complementary to vesicular stomatitis virus N protein mRNA initiation site. Proc Natl Acad Sci USA. 84:648-52.

Lemischka, I. R., D. H. Raulet, and R. C. Mulligan. 1986. Developmental potential and dynamic behavior of hematopoietic stem cells. Cell. 45:917-927.

Lennon, G., C. Auffray, M. Polymeropoulos, and M. B. Soares. 1996. The I.M.A.G.E. Consortium: an integrated molecular analysis of genomes and their expression. Genomics. 33:151-2.

Letsinger, R. L., G. R. Zhang, D. K. Sun, T. Ikeuchi, et al. 1989. Cholesteryl-conjugated oligonucleotides: synthesis, properties, and activity as inhibitors of replication of human immunodeficiency virus in cell culture. Proc Natl Acad Sci USA. 86:6553-6.

Li, Y., W. Cockburn, and G. C. Whitelam. 1998. Filamentous bacteriophage display of a bifunctional protein A::scFv fusion. Mol Biotechnol. 9:187-93.

Lin, J. J., H. Jiang, and P. B. Fisher. 1998. Melanoma differentiation associated gene-9, mda-9, is a human gamma interferon responsive gene. Gene. 207:105-10.

Lincke, C. R., J. J. Smit, T. van der Velde-Koerts, and P. Borst. 1991. Structure of the human MDR3 gene and physical mapping of the human MDR locus. J Biol Chem. 266:5303-10.

Linder, M. W., R. A. Prough, and R. Valdes. 1997. Pharmacogenetics: a laboratory tool for optimizing therapeutic efficiency. Clin Chem. 43:254-66.

Littlefield, J. W. 1964. Selection of hybrids from matings of fibroblasts in vitro and their presumed recombinants. Science. 145:709-710.

Lizardi, P. M., C. E. Guerra, H. Lomeli, I. Tussie-Luna, et al. 1988. Exponential amplification of recombinant-RNA hybridization probes. Biotechnology. 6:1197-1202.

Lonberg, N., and D. Huszar. 1995. Human antibodies from transgenic mice. Int Rev Immunol. 13:65-93.

Lonberg, N., L. D. Taylor, F. A. Harding, M. Trounstine, et al. 1994. Antigen-specific human antibodies from mice comprising four distinct genetic modifications [see comments]. Nature. 368:856-9.

Lopata, M. A., D. W. Cleveland, and B. Sollner-Webb. 1984. High-level expression of a chloramphenicol acetyltransferase gene by DEAEdextran-mediated DNA traansfection couled with a dimethylsulfoxide or glycerol shock treatment. Nucleic Acids Research. 12:5707.

Lowman, H., and J. Wells. 1991. Methods: A Companion to Methods in Enzymology. 3:205.

Lowman, H. B., S. H. Bass, N. Simpson, and J. A. Wells. 1991. Selecting high-affinity binding proteins by monovalent phage display. Biochemistry. 30:10832-8.

Lowman, H. B., Y. M. Chen, N. J. Skelton, D. L. Mortensen, et al. 1998. Molecular mimics of insulin-like growth factor 1 (IGF-1) for inhibiting IGF-1: IGF-binding protein interactions. Biochemistry. 37:8870-8.

Luckow, V. A. 1991. Cloning and expression of heterologous genes in insect cells with baculovirus vectors. In Recombinant DNA technology and applications. A. Prokop, R. K. Bajpai, and C. Ho, editors. McGraw-Hill, New York. 97-152.

Lue, R. A., S. M. Marfatia, D. Branton, and A. H. Chishti. 1994. Cloning and characterization of hdlg: the human homologue of the Drosophila discs large tumor suppressor binds to protein 4.1. Proc Natl Acad Sci U S A. 91:9818-22.

Maekawa, K., N. Imagawa, M. Nagamatsu, and S. Harada. 1994. Molecular cloning of a novel protein-tyrosine phosphatase containing a membrane-binding domain and GLGF repeats. FEBS Lett. 337:200-6.

Maher, L. J. 1992. DNA triple-helix formation: an approach to artificial gene repressors? Bioessays. 14:807-15.

Makino, K., H. Kuwahara, N. Masuko, Y. Nishiyama, et al. 1997. Cloning and characterization of NE-dlg: a novel human homolog of the Drosophila discs large (dlg) tumor suppressor protein interacts with the APC protein. Oncogene. 14:2425-33.

Mandel, M., and A. Higa. 1970. Calcium-dependent bacteriophage DNA infection. J. Mol. biol. 53:159-162.

Marasco, W. A., W. A. Haseltine, and S. Y. Chen. 1993. Design, intracellular expression, and activity of a human anti-human immunodeficiency virus type 1 gp 120 single-chain antibody. Proc Natl Acad Sci USA. 90:7889-93.

Marfatia, S. M., R. A. Leu, D. Branton, and A. H. Chishti. 1995. Identification of the protein 4.1 binding interface on glycophorin C and p55, a homologue of the Drosophila discs-large tumor suppressor protein. J Biol Chem. 270:715-9.

Marks, J. D., A. D. Griffiths, M. Malmqvist, T. P. Clackson, et al. 1992. By-passing immunization: building high affinity human antibodies by chain shuffling. Biotechnology (NY). 10:779-83.

Marks, J. D., H. R. Hoogenboom, T. P. Bonnert, J. McCafferty, et al. 1991a. By-passing immunization. Human antibodies from V-gene libraries displayed on phage. J Mol Biol. 222:581-97.

Marks, J. D., H. R. Hoogenboom, T. P. Bonnert, J. McCafferty, et al. 1991b. By-passing immunization. Human antibodies from V-gene libraries displayed on phage. J Mol Biol. 222:581-97.

Martin, F. J., and D. Papahadjopoulos. 1982. Irreversible coupling of immunoglobulin fragments to preformed vesicles. An improved method for liposome targeting. J Biol Chem. 257:286-8.

Maxam, A. M., and W. Gilbert. 1977. A new method for sequencing DNA. Proc Natl AcadSci USA. 74:560-4.

Mazoyer, S., S. A. Gayther, M. A. Nagai, S. A. Smith, et al. 1995. A gene (DLG2) located at 17q 12-q21 encodes a new homologue of the Drosophila tumor suppressor dIg-A. Genomics. 28:25-31.

McLoughlin, D. M., and C. C. Miller. 1996. The intracellular cytoplasmic domain of the Alzheimer's disease amyloid precursor protein interacts with phosphotyrosine-binding domain proteins in the yeast two-hybrid system. FEBS Lett. 397:197-200.

McLoughlin, D. M., C. L. Standen, K. F. Lau, S. Ackerley, et al. 2001. The neuronal adaptor protein X1 lalpha interacts with the copper chaperone for SODI and regulates SODI activity. J Biol Chem. 276:9303-7.

Metzenberg, A. B., and J. Gitschier. 1992. The gene encoding the palmitoylated erythrocyte membrane protein, p55, originates at the CpG island 3′ to the factor VIII gene. Hum Mol Genet. 1:97-101.

Michiels, F., G. G. Habets, J. C. Stam, R. A. van der Kammen, et al. 1995. A role for Rae in Tiam1-induced membrane ruffling and invasion. Nature. 375:338-40.

Miller, A. D., and C. Buttimore. 1986. Redesign of retrovirus packaging cell lines to avoid recombination leading to helper virus production. Mol. Cell biol. 6:2895-2902.

Miller, L. K. 1988. Baculoviruses as gene expression vectors. Annu. Rev. Microbiol. 42:177-199.

Milstein, C., and A. C. Cuello. 1983. Hybrid hybridomas and their use in immunohistochemistry. Nature. 305:537-40.

Mizuno, K., I. Okano, K. Ohashi, K. Nunoue, et al. 1994. Identification of a human cDNA encoding a novel protein kinase with two repeats of the LIM/double zinc finger motif. Oncogene. 9:1605-12.

U.S. Pat. No. 5,459,039. Methods for mapping genetic mutations. 1995.

Mohandas, T. K., X. N. Chen, L. B. Rowe, E. H. Birkenmeier, et al. 1995. Localization of the tight junction protein gene TJP1 to human chromosome 15q13, distal to the Prader-Willi/Angelman region, and to mouse chromosome 7. Genomics. 30:594-7.

Morais Cabral, J. H., C. Petosa, M. J. Sutcliffe, S. Raza, et al. 1996. Crystal structure of a PDZ domain. Nature. 382:649-52.

Morrison, S. L., L. Wims, S. Wallick, L. Tan, et al. 1987. Genetically engineered antibody molecules and their application. Ann N Y Acad Sci. 507:187-98.

U.S. Pat. No. 4,683,202. Process for amplifying nucleic acid sequences. 1987.

U.S. Pat. No. 4,683,195. Process for amplifying, detecting, and/or cloning nucleic acid sequences. 1987.

Munson, P. J., and D. Rodbard. 1980. Ligand: a versatile computerized approach for characterization of ligand-binding systems. Anal Biochem. 107:220-39.

Murthy, A., C. Gonzalez-Agosti, E. Cordero, D. Pinney, et al. 1998. NHE-RF, a regulatory cofactor for Na(+)−H+ exchange, is a common interactor for merlin and ERM (MERM) proteins. J Biol Chem. 273:1273-6.

Myers, R. M., Z. Larin, and T. Maniatis. 1985. Detection of single base substitutions by ribonuclease cleavage at mismatches in RNA:DNA duplexes. Science. 230:1242-6.

U.S. Pat. No. 5,328,470. Treatment of diseases by site-specific instillation of cells or site-specific transformation of cells and kits therefor. 1994.

Naeve, C. W., G. A. Buck, R. L. Niece, R. T. Pon, et al. 1995. Accuracy of automated DNA sequencing: a multi-laboratory comparison of sequencing results. Biotechniques. 19:448-53.

Nagase, T., K. Ishikawa, R. Kikuno, M. Hirosawa, et al. 1999a. Prediction of the coding sequences of unidentified human genes. XV. The complete sequences of 100 new cDNA clones from brain which code for large proteins in vitro. DNA Res. 6:337-45.

Nagase, T., K. Ishikawa, N. Miyajima, A. Tanaka, et al. 1998a. Prediction of the coding sequences of unidentified human genes. IX. The complete sequences of 100 new cDNA clones from brain which can code for large proteins in vitro. DNA Res. 5:31-9.

Nagase, T., K. Ishikawa, D. Nakajima, M. Ohira, et al. 1997. Prediction of the coding sequences of unidentified human genes. VII. The complete sequences of 100 new cDNA clones from brain which can code for large proteins in vitro. DNA Res. 4:141-50.

Nagase, T., K. Ishikawa, M. Suyama, R. Kikuno, et al. 1998b. Prediction of the coding sequences of unidentified human genes. XII. The complete sequences of 100 new CDNA clones from brain which code for large proteins in vitro. DNA Res. 5:355-64.

Nagase, T., K. Ishikawa, M. Suyama, R. Kikuno, et al. 1999b. Prediction of the coding sequences of unidentified human genes. XIII. The complete sequences of 100 new CDNA clones from brain which code for large proteins in vitro. DNA Res. 6:63-70.

Nagase, T., K. Ishikawa, M. Suyama, R. Kikuno, et al. 1998c. Prediction of the coding sequences of unidentified human genes. XI. The complete sequences of 100 new CDNA clones from brain which code for large proteins in vitro. DNA Res. 5:277-86.

Nagase, T., R. Kikuno, M. Nakayama, M. Hirosawa, et al. 2000. Prediction of the coding sequences of unidentified human genes. XVIII. The complete sequences of 100 new cDNA clones from brain which code for large proteins in vitro. DNA Res. 7:273-81.

Nagase, T., N. Seki, A. Tanaka, K. Ishikawa, et al. 1995. Prediction of the coding sequences of unidentified human genes. IV. The coding sequences of 40 new genes (KIAA0121-KIAA0160) deduced by analysis of cDNA clones from human cell line KG-I. DNA Res. 2:167-74, 199-210.

Nakamura, H., T. Sudo, H. Tsuiki, H. Miyake, et al. 1998. Identification of a novel human homolog of the Drosophila dig, P-dlg, specifically expressed in the gland tissues and interacting with p55. FEBS Lett. 433:63-7.

Nakazawa, H., D. English, P. L. Randell, K. Nakazawa, et al. 1994. UV and skin cancer: specific p53 gene mutation in normal skin as a biologically relevant exposure measurement. Proc Natl Acad Sci USA. 91:360-4.

Neumann, E., M. Schaefer-Ridder, Y. Wang, and P. H. Hofschneider. 1982. Gene transfer into mouse lyoma cells by electroporation in high electric fields. EMBO J. 1:841-845.

Nomoto, S., Y. Tatematsu, T. Takahashi, and H. Osada. 1999. Cloning and characterization of the alternative promoter regions of the human LIMK2 gene responsible for alternative transcripts with tissue-specific expression. Gene. 236:259-71.

Okamoto, M., and T. C. Sudhof. 1997. Mints, Munc 18-interacting proteins in synaptic vesicle exocytosis. J Biol Chem. 272:31459-64.

Okano, H., J. Aruga, T. Nakagawa, C. Shiota, et al. 1991. Myelin basic protein gene and the function of antisense RNA in its repression in myclin-deficient mutant mouse. J Neurochem. 56:560-7.

Okano, I., J. Hiraoka, H. Otera, K. Nunoue, et al. 1995. Identification and characterization of a novel family of serine/threonine kinases containing two N-terminal LIM motifs. J Biol Chem. 270:31321-30.

O'Reilly, D. R., L. K. Miller, and V. A. Luckow. 1992. Baculovirus expression vectors. W. H. Freeman and Company, New York.

Orita, M., H. Iwahana, H. Kanazawa, K. Hayashi, et al. 1989. Detection of polymorphisms of human DNA by gel electrophoresis as single-strand conformation polymorphisms. Proc Natl Acad Sci USA. 86:2766-70.

Osada, H., K. Hasada, J. Inazawa, K. Uchida, et al. 1996. Subcellular localization and protein interaction of the human LIMK2 gene expressing alternative transcripts with tissue-specific regulation. Biochem Biophys Res Commun. 229:582-9.

Osborne, L. R., D. Martindale, S. W. Scherer, X. M. Shi, et al. 1996. Identification of genes from a 500-kb region at 7ql 1.23 that is commonly deleted in Williams syndrome patients. Genomics. 36:328-36.

Oschkinat, H. 1999. A new type of PDZ domain recognition. Nat Struct Biol. 6:408-10.

Ou-Lee, T. M., R. Turgeon, and R. Wu. 1986. Uptake and expression of a foreign gene linked to either a plant virus or Drosophila promoter in protoplasts of rice, wheat and sorghum. Proc. Natl. Acad. Sci. USA. 83:6815-6819.

Palmer, T. D., R. A. Hock, W. R. A. osborne, and A. D. Miller. 1987. Efficient retrovirus-mediated transfer and expression of a human adenosine deaminase gene in diploid skin fibroblasts from an adenosie-deficient human. Proc. Natl. Acad. Sci. USA. 84:1055-1059.

Palzkill, T., W. Huang, and G. M. Weinstock. 1998. Mapping protein-ligand interactions using whole genome phage display libraries. Gene. 221:79-83.

WO89/10134. Chimeric peptides for neuropeptide delivery through the blood-brain barrier. 1989.

Patel, B. K., C. L. Keck, R. S. O'Leary, N. C. Popescu, et al. 1998. Localization of the human stat6 gene to chromosome 12q13.3-q14.1, a region implicated in multiple solid tumors. Genomics. 52:192-200.

Pear, W., G. Nolan, M. Scott, and D. Baltimore. 1993. Production of high-titer helper-free retroviruses by transient transfection. Proc. Natl. Acad. Sci. USA. 90:8392-8396.

Pearce, K. H., Jr., B. J. Potts, L. G. Presta, L. N. Bald, et al. 1997. Mutational analysis of thrombopoietin for identification of receptor and neutralizing antibody sites. J Biol Chem. 272:20595-602.

Perry-O'Keefe, H., X. W. Yao, J. M. Coull, M. Fuchs, et al. 1996. Peptide nucleic acid pre-gel hybridization: an alternative to southern hybridization. Proc Natl Acad Sci USA. 93:14670-5.

Petersen, K. H., D. K. Jensen, M. Egholm, O. Buchardt, et al. 1976. A PNA-DNA linker synthesis of N-((4,4′-dimethoxytrityloxy)ehtyl)-N-(thymin-1-ylacetyl)glycine. Biorganic and Medicianl Chemistry Letters. 5:1119-1124.

Pizzuti, A., F. Amati, G. Calabrese, A. Mari, et al. 1996a. cDNA characterization and chromosomal mapping of two human homologues of the Drosophila dishevelled polarity gene. Hum Mol Genet. 5:953-8.

Pizzuti, A., G. Novelli, A. Mari, A. Ratti, et al. 1996b. Human homologue sequences to the Drosophila dishevelled segment-polarity gene are deleted in the DiGeorge syndrome. Am J Hum Genet. 58:722-9.

Potter, H. 1988. Electroporation in biology: Methods, applications, and instrumentation. Analytical Biochemistry. 174:361-373.

Potter, H., L. Weir, and P. Leder. 1984. Enhancer-dependent expression of human kappa immunoglobulin genes introduced into mouse pre-B Iymphocytes by electroporation. Proc. Natl. Acad. Sci. USA. 81:7161-7165.

Poulat, F., P. S. Barbara, M. Desclozeaux, S. Soullier, et al. 1997. The human testis determining factor SRY binds a nuclear factor containing PDZ protein interaction domains. J Biol Chem. 272:7167-72.

Prasad, R., Y. Gu, H. Alder, T. Nakamura, et al. 1993. Cloning of the ALL-I fusion partner, the AF-6 gene, involved in acute myeloid leukemias with the t(6;11) chromosome translocation. Cancer Res. 53:5624-8.

Presta, L. G. 1992. Antibody engineering. Curr Opin Biotechnol. 3:394-8.

Prosser, J. 1993. Detecting single-base mutations. Trends Biotechnol. 11:238-46.

Raghuram, V., D. D. Mak, and J. K. Foskett. 2001. Regulation of cystic fibrosis transmembrane conductance regulator single-channel gating by bivalent PDZ-domain-mediated interaction. Proc Natl Acad Sci USA. 98:1300-5.

Rassoulzadegan, M., B. Binetruy, and F. Cuzin. 1982. High frequency of gene transfer after fusion between bacteria and eukaryotic cells. Nature. 295:257.

Reczek, D., M. Berryman, and A. Bretscher. 1997. Identification of EBP50: A PDZ-containing phosphoprotein that associates with members of the ezrin-radixin-moesin family. J Cell Biol. 139:169-79.

Reisfeld, R. A., and S. Sell. 1985. Monoclonal antibodies and cancer therapy: Proceedings of the Roche-UCLA symposium held in Park City, Utah, Jan. 26-Feb. 2, 1985. Alan R. Liss, New York. 609 pp.

Ren, Z., and L. W. Black. 1998. Phage T4 SOC and HOC display of biologically active, full-length proteins on the viral capsid. Gene. 215:439-44.

Ren, Z. J., G. K. Lewis, P. T. Wingfield, E. G. Locke, et al. 1996. Phage display of intact domains at high copy number: a system based on SOC, the small outer capsid protein of bacteriophage T4. Protein Sci. 5:1833-43.

Ren, Z. -J. e. a. 1997. CAN. 127:215644.

Reynaud, C., S. Fabre, and P. Jalinot. 2000. The PDZ protein TIP-1 interacts with the Rho effector rhotekin and is involved in Rho signaling to the serum response element. J Biol Chem. 275:33962-8.

Rhodes, C. A., D. A. Pierce, I. J. Mettler, D. Mascarenhas, et al. 1988. Genetically transformed maize plants from protoplasts. Science. 240:204-207.

Riechmann, L., M. Clark, H. Waldmann, and G. Winter. 1988. Reshaping human antibodies for therapy. Nature. 332:323-7.

Rose, J. K., L. Buonocore, and M. Whitt. 1991. A new cationic liposome reagent mediating nearly quantitative transfection of animal cells. BioTechniques. 10:520-525.

Rossi, J. J. 1994. Practical ribozymes. Making ribozymes work in cells. Curr Biol. 4:469-71.

Rossiter, B. J., and C. T. Caskey. 1990. Molecular scanning methods of mutation detection. J Biol Chem. 265:12753-6.

Rousset, R., S. Fabre, C. Desbois, F. Bantignies, et al. 1998. The C-terminus of the HTLV-1 Tax oncoprotein mediates interaction with the PDZ domain of cellular proteins. Oncogene. 16:643-54.

Ruff, P., A. H. Chishti, E. Grimm, D. Bischoff, et al. 1999. Exon skipping truncates the PDZ domain of human erythroid p55 in a patient with chronic myeloid leukemia in acute megakaryoblastic blast crisis. Leuk Res. 23:247-50.

Saha, V., D. M. Lillington, A. N. Shelling, T. Chaplin, et al. 1995. AF6 gene on chromosome band 6q27 maps distal to the minimal region of deletion in epithelial ovarian cancer. Genes Chromosomes Cancer. 14:220-2.

Saiki, R. K., T. L. Bugawan, G. T. Horn, K. B. Mullis, et al. 1986. Analysis of enzymatically amplified beta-globin and HLA-DQ alpha DNA with allele-specific oligonucleotide probes. Nature. 324:163-6.

Saiki, R. K., P. S. Walsh, C. H. Levenson, and H. A. Erlich. 1989. Genetic analysis of amplified DNA with immobilized sequence-specific oligonucleotide probes. Proc Natl Acad Sci USA. 86:6230-4.

Saito, S., M. Matsushima, S. Shirahama, T. Minaguchi, et al. 1998. Complete genomic structure DNA polymorphisms, and alternative splicing of the human AF-6 gene. DNA Res. 5:115-20.

Saleeba, J. A., and R. G. Cotton. 1993. Chemical cleavage of mismatch to detect mutations. Methods Enzymol. 217:286-95.

Sambrook, J. 1989. Molecular cloning: a laboratory manual. Cold Spring Harbor Laboratory, Cold Spring Harbor.

Sandri-Goldin, R. M., A. L. Goldin, J. C. Glorioso, and M. Levine. 1981. High-frequency transfer of cloned herpes simjplex virus type I sequences to mammalian cells by protoplast fusion. Mol. Cell. Biol. 1:7453-752.

Sanger, F., S. Nicklen, and A. R. Coulson. 1977. DNA sequencing with chain-terminating inhibitors. Proc Natl Acad Sci USA. 74:5463-7.

Saunders, J. A., B. F. Matthews, and P. D. Miller. 1989. Plant gene transfer using electrofusion and electroporation. In Electroporation and electrofusion in cell biology. E. Neumann, A. E. Sowers, and C. A. Jordan, editors. Plenum Press, New York. 343-354.

Scanlan, M. J., Y. T. Chen, B. Williamson, A. O. Gure, et al. 1998. Characterization of human colon cancer antigens recognized by autologous antibodies. Int J Cancer. 76:652-8.

Schade, R., C. Staak, C. Hendriksen, M. Erhard, et al. 1996. The production of avian (egg yold) antibodies: IgY. The report and recommendations of ECVAM workshop. Alternatives to Laboratory Animals (ATLA). 24:925-934.

Schaffner, W. 1980. Direct transfer of cloned genes from bacteria to mammalian cells. Proc. Natl. Acad. Sci. USA. 77:2163.

Schiaffino, M. V., M. T. Bassi, E. I. Rugarli, A. Renieri, et al. 1995. Cloning of a human homologue of the Xenopus laevis APX gene from the ocular albinism type 1 critical region. Hum Mol Genet. 4:373-82.

Schook, L. B. 1987. Monoclonal antibody production techniques and applications. Marcel Dekker, Inc., New York. 336 pp.

Scott, J. K., and G. P. Smith. 1990. Searching for peptide ligands with an epitope library. Science. 249:386-90.

Selden, R. F., K. Burke-Howie, M. E. Rowe, H. M. Goodman, et al. 1986. Human growth hormone as a reporter gene in regulation studies employing transient gene expression. Molecular and Cellular Biololgy. 6:3173-3179.

Semenov, M. V., and M. Snyder. 1997. Human dishevelled genes constitute a DHR-containing multigene family. Genomics. 42:302-10.

Shalaby, M. R., H. M. Shepard, L. Presta, M. L. Rodrigues, et al. 1992. Development of humanized bispecific antibodies reactive with cytotoxic lymphocytes and tumor cells overexpressing the HER2 protooncogene. J Exp Med. 175:217-25.

Shigekawa, K., and W. J. Dower. 1988. Electroporation of eukaryotes and prokaryotes: A general approach to the introduction of macomolecules into cells. BioTechniques. 6:742-751.

Shillito, R. 1999. Methods of genetic transformations: Electroporation and polyethylene glycol treatment. In Molecular improvement of cereal crop. I. Vasil, editor. Kluwer, Dordrecht, The Netherlands. 9-20.

Shiratsuchi, T., M. Futamura, K. Oda, H. Nishimori, et al. 1998. Cloning and characterization of BAI-associated protein 1: a PDZ domain-containing protein that interacts with BAI1. Biochem Biophys Res Commun. 247:597-604.

Shopes, B. 1992. A genetically engineered human IgG mutant with enhanced cytolytic activity. J Immunol. 148:2918-22.

Sidhu, S. S., H. B. Lowman, B. C. Cunningham, and J. A. Wells. 2000. Phage display for selection of novel binding peptides. Methods Enzymol. 328:333-63.

Simonsen, C. C., and A. D. Levinson. 1983. Isolation and expression of an altered mouse dihydrofolate reductase CDNA. Proc. Natl. Acad. Sci. USA. 80:2495-2499.

Sirotkin, H., H. O'Donnell, R. DasGupta, S. Halford, et al. 1997. Identification of a new human catenin gene family member (ARVCF) from the region deleted in velo-cardio-facial syndrome. Genomics. 41:75-83.

Smit, J. J., C. A. Mol, L. van Deemter, E. Wagenaar, et al. 1995. Characterization of the promoter region of the human MDR3 P-glycoprotein gene. Biochim Biophys Acta. 1261:44-56.

Smith, F. D., G. S. Oxford, and S. L. Milgram. 1999. Association of the D2 dopamine receptor third cytoplasmic loop with spinophilin, a protein phosphatase-1-interacting protein. J Biol Chem. 274:19894-900.

Smith, G. P. 1991. Surface presentation of protein epitopes using bacteriophage expression systems. Curr Opin Biotechnol. 2:668-73.

Smith, G. P., and J. K. Scott. 1993. Libraries of peptides and proteins displayed on filamentous phage. Methods Enzymol. 217:228-57.

Smith, S. A., P. Holik, J. Stevens, S. Mazoyer, et al. 1996. Isolation of a gene (DLG3) encoding a second member of the discs-large family on chromosome 17q 12-q21. Genomics. 31:145-50.

Snow, B. E., R. A. Hall, A. M. Krumins, G. M. Brothers, et al. 1998. GTPase activating specificity of RGS 12 and binding specificity of an alternatively spliced PDZ (PSD-95/Dlg/ZO-1) domain. J Biol Chem. 273:17749-55.

Songyang, Z., A. S. Fanning, C. Fu, J. Xu, et al 1997. Recognition of unique carboxyl-terminal motifs by distinct PDZ domains. Science. 275:73-7.

Southern, P. J., and P. Berg. 1982. Transformation of mammalian cells to antibiotic resistanced with a bacterial gene under control of the SV40 early region promoter. J. Mol. Appl. Gen. 1:327-341.

Sreekrishna, K., R. H. Potenz, J. A. Cruze, W. R. McCombie, et al. 1988. High level expression of heterologous proteins in methylotrophic yeast Pichia pastoris. J Basic Microbiol. 28:265-78.

Stathakis, D. G., K. B. Hoover, Z. You, and P. J. Bryant. 1997. Human postsynaptic density-95 (PSD95): location of the gene (DLG4) and possible function in normeural as well as in neural tissues. Genomics. 44:71-82.

Stathakis, D. G., D. Lee, and P. J. Bryant. 1998. Fine-scale physical map of the 11q21 region surrounding the human DLG2 locus, the gene encoding Chapsyn-110. Genomics. 54:186-8.

Stein, C. A., and J. S. Cohen. 1988. Oligodeoxynucleotides as inhibitors of gene expression: a review. Cancer Res. 48:2659-68.

Stevenson, G. T., A. Pindar, and C. J. Slade. 1989. A chimeric antibody with dual Fe regions (bisFabFc) prepared by manipulations at the IgG hinge. Anticancer Drug Des. 3:219-30.

Stricker, N. L., K. S. Christopherson, B. A. Yi, P. J. Schatz, et al. 1997. PDZ domain of neuronal nitric oxide synthase recognizes novel C-terminal peptide sequences. Nat Biotechnol. 15:336-42.

Suresh, M. R., A. C. Cuello, and C. Milstein. 1986. Bispecific monoclonal antibodies from hybrid hybridomas. Methods Enzymol. 121:210-28.

Tanahashi, H., and T. Tabira. 1999a. Genomic organization of the human X11 L2 gene (APBA3), a third member of the XlI protein family interacting with Alzheimer's beta-amyloid precursor protein. Neuroreport. 10:2575-8.

Tanahashi, H., and T. Tabira. 1999b. X11 L2, a new member of the XlI protein family, interacts with Alzheimer's beta-amyloid precursor protein. Biochem Biophys Res Commun. 255:663-7.

Tassabehji, M., K. Metcalfe, W. D. Fergusson, M. J. Carette, et al. 1996. LIM-kinase deleted in Williams syndrome. Nat Genet. 13:272-3.

Tejedor, F. J., A. Bokhari, O. Rogero, M. Gorczyca, et al. 1997. Essential role for dlg in synaptic clustering of Shaker K+ channels in vivo. J Neurosci. 17:152-9.

Therrien, M., A. M. Wong, E. Kwan, and G. M. Rubin. 1999. Functional analysis of CNK in RAS signaling. Proc Natl Acad Sci USA. 96:13259-63.

Therrien, M., A. M. Wong, and G. M. Rubin. 1998. CNK, a RAF-binding multidomain protein required for RAS signaling. Cell. 95:343-53.

Thompson, J. A., and e. al. 1995. Maize transformation utilizing silicon carbide whiskers: A review. Euphytica. 85:75-80.

Tilburn, J., C. Scazzocchio, G. G. Taylor, J. H. Zabicky-Zissman, et al. 1983. Transformation by integration in Aspergillus nidulans. Gene. 26:205-21.

Tochio, H., Q. Zhang, P. Mandal, M. Li, et al. 1999. Solution structure of the extended neuronal nitric oxide synthase PDZ domain complexed with an associated peptide. Nat Struct Biol. 6:417-21.

Tomita, S., T. Fujita, Y. Kirino, and T. Suzuki. 2000. PDZ domain-dependent suppression of NF-kappaB/p65-induced Abeta42 production by a neuron-specific X11-like protein. J Biol Chem. 275:13056-60.

Tomita, S., T. Ozaki, H. Taru, S. Oguchi, et al. 1999. Interaction of a neuron-specific protein containing PDZ domains with Alzheimer's amyloid precursor protein. J Biol Chem. 274:2243-54.

Touchman, J. W., Y. Anikster, N. L. Dietrich, V. V. Maduro, et al. 2000. The genomic region encompassing the nephropathic cystinosis gene (CTNS): complete sequencing of a 200-kb segment and discovery of a novel gene within the common cystinosis-causing deletion. Genome Res. 10:165-73.

Touraev, A., and e. al. 1997. Plant male germ line transformation. Plant J. 12:949-956.

Traunecker, A., F. Oliveri, and K. Karjalainen. 1991. Myeloma based expression system for production of large mammalian proteins. Trends Biotechnol. 9:109-13.

Trick, H. N., and e. al. 1997. Recent advances in soybean transformation. Plant Tissue Cult. Biotechnol. 3:9-26.

Tsunoda, S., J. Sierralta, Y. Sun, R. Bodner, et al. 1997. A multivalent PDZ-domain protein assembles signalling complexes in a G-protein-coupled cascade. Nature. 388:243-9.

Tsunoda, S., J. Sierralta, and C. S. Zuker. 1998. Specificity in signaling pathways: assembly into multimolecular signaling complexes. Curr Opin Genet Dev. 8:419-22.

Tuerk, C., and L. Gold. 1990. Systematic evolution of ligands by exponential enrichment: RNA ligands to bacteriophage T4 DNA polymerase. Science. 249:505-10.

U.S. Pat. No. 4,904,582. Novel amphiphilic nucleic acid conjugates. 1988.

Turner, D. L., E. Y. Snyder, and C. L. Cepko. 1990. Lineage-independent determinationh of cell type in the embryonic mouse retina. Neuron. 4:833-845.

Tutt, A., G. T. Stevenson, and M. J. Glennie. 1991. Trispecific F(ab′)3 derivatives that use cooperative signaling via the TCR/CD3 complex and CD2 to activate and redirect resting cytotoxic T cells. J Immunol. 147:60-9.

Ueki, N., N. Seki, K. Yano, Y. Masuho, et al. 1999. Isolation, tissue expression, and chromosomal assignment of a human LIM protein gene, showing homology to rat enigma homologue (ENH). J Hum Genet. 44:256-60.

Ullmer, C., K. Schmuck, A. Figge, and H. Lubbert. 1998. Cloning and characterization of MUPPI, a novel PDZ domain protein. FEBS Lett. 424:63-8.

Van der Bliek, A. M., F. Baas, T. Ten Houte de Lange, P. M. Kooiman, et al 1987. The human mdr3 gene encodes a novel P-glycoprotein homologue and gives rise to alternatively spliced mRNAs in liver. Embo J. 6:3325-31.

van der Bliek, A. M., P. M. Kooiman, C. Schneider, and P. Borst. 1988. Sequence of mdr3 cDNA encoding a human P-glycoprotein. Gene. 71:401-11.

van der Krol, A. R., J. N. Mol, and A. R. Stuitje. 1988a. Modulation of eukaryotic gene expression by complementary RNA or DNA sequences. Biotechniques. 6:958-76.

van der Krol, A. R., J. N. Mol, and A. R. Stuitje. 1988b. Modulation of eukaryotic gene expression by complementary RNA or DNA sequences. Biotechniques. 6:958-76.

Verhoeyen, M., C. Milstein, and G. Winter. 1988. Reshaping human antibodies: grafting an antilysozyme activity. Science. 239:1534-6.

Vitetta, E. S., R. J. Fulton, R. D. May, M. Till, et al. 1987. Redesigning nature's poisons to create anti-tumor reagents. Science. 238:1098-104.

Von Kap-Herr, C., G. Kandala, S. S. Mann, T. C. Hart, et al. 2000. Assignment of PDZ domain-containing protein GIPC gene (C 19orf3) to human chromosome band 19p13.1 by in situ hybridization and radiation hybrid mapping. Cytogenet Cell Genet. 89:234-5.

Watanabe, T. K., A. Saito, M. Suzuki, T. Fujiwara, et al. 1998. cDNA cloning and characterization of a human proteasomal modulator subunit, p27 (PSMD9). Genomics. 50:241-50.

Wells, J. A., and H. B. Lowman. 1992. Rapid evolution of peptide and protein binding properties in vitro. Curr Opin Biotechnol. 3:355-62.

Wells, J. A., M. Vasser, and D. B. Powers. 1985. Cassette mutagenesis: an efficient method for generation of multiple mutations at defined sites. Gene. 34:315-23.

White, K. E., J. Biber, H. Murer, and M. J. Econs. 1998. A PDZ domain-containing protein with homology to Diphor-1 maps to human chromosome 1q21. Ann Hum Genet. 62:287-90.

Whitt, M. A., L. Buonocore, J. K. Rose, V. Ciccarone, et al. 1990. TransfectACE reagent promotes transient transfection frequencies greater than 90%. Focus. 13:8-12.

Wigler, M., A. Pellicer, S. Silversttein, and R. Axel. 1978. Biochemical transfer of single-copy eucaryotic genes using total cellular DNA as donor. Cell. 14:725.

Williams, D. A., I. R. Lemischka, D. G. Nathan, and R. C. Mulligan. 1984. Introduction of a new genetic material into pluripotent haematopoietic stem cells of the mouse. Nature. 310:476-480.

Willott, E., M. S. Balda, A. S. Fanning, B. Jameson, et al. 1993. The tight junction protein ZO-1 is homologous to the Drosophila discs-large tumor suppressor protein of septate junctions. Proc Natl Acad Sci USA. 90:7834-8.

Willott, E., M. S. Balda, M. Heintzelman, B. Jameson, et al. 1992. Localization and differential expression of two isoforms of the tight junction protein ZO-1. Am J Physiol. 262:C 1119-24.

Wolff, E. A., G. J. Schreiber, W. L. Cosand, and H. V. Raff. 1993. Monoclonal antibody homodimers: enhanced antitumor activity in nude mice. Cancer Res. 53:2560-5.

Wong, T. K., and E. Neumann. 1982. Electric field mediated gene transfer. Biochemical and Biophysical Research Communications. 107:584-587.

Wu, Y., D. Dowbenko, S. Spencer, R. Laura, et al. 2000. Interaction of the tumor suppressor PTEN/MMAC with a PDZ domain of MAGI3, a novel membrane-associated guanylate kinase. J Biol Chem. 275:21477-85.

Wyborski, D. L., L. C. DuCoeur, and J. M. Short. 1996. Parameters affecting the use of the lac repressor system in eukaryotic cells and transgenic animals. Environ Mol Mutagen. 28:447-58.

Wyborski, D. L., and J. M. Short. 1991. Analysis of inducers of the E. coli lac repressor system in mammalian cells and whole animals. Nucleic Acids Res. 19:4647-53.

Yang, Q., and N. K. Tonks. 1991. Isolation of a cDNA clone encoding a human protein-tyrosine phosphatase with homology to the cytoskeletal-associated proteins band 4.1, ezrin, and talin. Proc Natl Acad Sci USA. 88:5949-53.

Yelton, M. M., J. E. Hamer, and W. E. Timberlake. 1984. Transformation of Aspergillus nidulans by using a trpc plasmid. Proc Natl Acad Sci USA. 81:1470-4.

Yun, C. H., S. Oh, M. Zizak, D. Steplock, et al. 1997. cAMP-mediated inhibition of the epithelial brush border Na+/H+ exchanger, NHE3, requires an associated regulatory protein. Proc Natl Acad Sci USA. 94:3010-5.

Zhou, G., and e. al. 1983. Introduction of exogenous DNA into cotton embryos. Methods Enzymol. 101:433-481.

Zhu, Z. 1997. CAN. 33:534.

Zoller, M. J., and M. Smith. 1987. Oligonucleotide-directed mutagenesis: a simple method using two oligonucleotide primers and a single-stranded DNA template. Methods Enzymol. 154:329-50.

Zon, G. 1988. Oligonucleotide analogues as potential chemotherapeutic agents. Pharm Res. 5:539-49.

Zuckermann, R. N., E. J. Martin, D. C. Spellmeyer, G. B. Stauber, et al 1994. Discovery of nanomolar ligands for 7-transmembrane G-protein-coupled receptors from a diverse N-(substituted)glycine peptoid library. J Med Chem. 37:2678-85.

Zumbrunn, J., and B. Trueb. 1996. Primary structure of a putative serine protease specific for IGF-binding proteins. FEBS Lett. 398:187-92.

Zumbrunn, J., and B. Trueb. 1997. Localization of the gene for a serine protease with IGF-binding domain (PRSS11) to human chromosome 10q25.3-q26.2. Genomics. 45:461-2.

1. A fusion protein comprising at least a portion of a phage coat protein bonded through the carboxyl-terminus thereof, optionally through a peptide linker, to a PDZ domain binding peptide, where the peptide contains 3-20 amino acid residues.

2. The fusion protein of claim 1, wherein the phage is a filamentous phage.

3. The fusion protein of claim 2, wherein the coat protein is a g8 protein.

4. The fusion protein of claim 1, wherein the PDZ domain binding peptide contains 3-20 amino acid residues.

5. The fusion protein of any of claims 1-4, wherein the phage coat protein comprises the mature phage coat protein.

6. A fusion gene encoding the fusion protein of any one of claims 1-5.

7. A vector comprising the fusion gene of claim 6.

8. A virus particle comprising the vector of claim 7.

9. A library of fusion proteins of any of claims 1-5, wherein the fusion proteins in the library comprise a plurality of PDZ domain binding peptides.

10. A library of vectors of claim 7, wherein the fusion genes encode fusion proteins comprising a plurality of PDZ domain binding peptides.

11. A library of virus particles of claim 8, wherein the fusion genes encode fusion proteins comprising a plurality of PDZ domain binding peptides.

12. A method for producing a PDZ domain binding peptide library comprising: expressing in recombinant host cells a library of variant fusion proteins of claim 9 to form a library of recombinant phage particles displaying the plurality of PDZ binding peptides on the surface thereof.

13. A method for selecting PDZ domain binding peptides comprising:(a) expressing in recombinant host cells a library of variant fusion proteins of claim 9 to form a library of recombinant phage particles displaying the plurality of PDZ binding peptides on the surface thereof; (b) contacting the recombinant phage particles with a target containing a PDZ domain so that at least a portion of the phage particles bind to the target; and (c) separating phage particles that bind to the target from those that do not bind.

14. The method of claim 13, wherein the phage particles contain fusion genes encoding the fusion proteins, further comprising sequencing at least a portion of the fusion gene of a selected phage particle to determine the amino acid sequence of a PDZ domain binding peptide, and optionally, synthesizing the PDZ domain binding peptide.

15. A method for identifying PDZ domain binding protein, comprising:(a) selecting PDZ domain binding peptides using the method of claim 13 to obtain phage particles containing fusion genes encoding the selected PDZ domain binding peptides, and sequencing a portion of the fusion genes to identify the amino acid sequence of at least one of the selected PDZ domain binding peptides; (b) comparing the PDZ domain binding peptide sequence with the carboxyl-terminal amino acid sequence of a group of proteins, and selecting an intracellular protein having a carboxyl-terminal sequence which is identical to or similar to the PDZ domain binding peptide sequence.

16. The method of claim 15, wherein the carboxyl-terminal sequence of the selected intracellular protein is identical to or differs at 1, 2 or 3 positions from the PDZ domain binding peptide sequence.

17. The method of claim 15, further comprising comparing the binding to a PDZ domain, of a selected PDZ domain binding peptide and of a selected intracellular protein or carboxyl-terminal sequence thereof.

18. An assay for a PDZ domain binding compound, comprising: contacting a PDZ domain containing polypeptide with a candidate PDZ domain binding compound, and detecting binding of the polypeptide and compound.

19. A host cell containing the vector of claim 7.

20. An isolated polypeptide comprising a carboxy terminal amino acid sequence having the sequence of a member selected from the group consisting of SEQ ID NOs:1-181, 209-213, 241-601 and 709-714.

21. The polypeptide of claim 20, consisting essentially of a member selected from the group consisting of SEQ ID NOs:1-181, 209-213, 241-601 and 709-714.

22. The polypeptide of claim 20, consisting of a member selected from the group consisting of SEQ ID NOs: 1-181, 209-213, 241-601 and 709-714.

23. A polypeptide that binds to the same epitope as the polypeptide of any of claims 20-22.

24. A polypeptide that competes for binding to a PDZ domain with the polypeptide of any of claims 20-23.

25. A polynucleotide encoding the polypeptide of any of claims 20-24.

26. A method of inhibiting a polypeptide-polypeptide interaction, comprising: contacting a mixture comprising a first and a second polypeptide with an inhibitor of interaction between a PDZ domain and its ligand, wherein the first polypeptide comprises said PDZ domain and the second polypeptide comprises said ligand.

27. The method of claim 26, wherein the first polypeptide is a fusion polypeptide which comprises a PDZ domain and the second polypeptide comprises a ligand of said PDZ domain, and the first polypeptide is attached to a substrate (such as a solid support).

28. The method of claim 26, wherein the first polypeptide is a fusion polypeptide which comprises a PDZ domain and the second polypeptide comprises a ligand of said PDZ domain, and the second polypeptide is attached to the substrate.

29. A method of screening for a substance that modulates interaction between a PDZ domain polypeptide and a molecule known to bind to the PDZ domain of said polypeptide comprising:
(a) contacting a sample containing said polypeptide and molecule with a candidate substance;
(b) determining amount of binding of said molecule to said polypeptide in the presence of said candidate substance;
(c) comparing the amount of binding of step (b) with amount of binding of said molecule to said polypeptide under similar conditions in the absence of said candidate substance;
whereby a difference in amount of binding as determined in (c) indicates that said candidate substance is a substance that modulates said interaction.

30. A method of screening for a substance that inhibits binding of a PDZ domain polypeptide to a molecule known to bind to the PDZ domain of said polypeptide comprising:
(a) contacting a sample containing said polypeptide and molecule with a candidate substance;
(b) determining amount of binding said molecule to said polypeptide in the presence of the candidate substance;
(c) comparing the amount of binding of step (b) with amount of binding of said molecule to said polypeptide under similar conditions in the absence of the candidate substance;
whereby a decrease in amount of binding of the polypeptide and said molecule in the presence of the candidate substance compared to the amount of binding in the absence of said candidate substance as determined in (c) indicates that said candidate substance is a substance that inhibits binding of the PDZ domain polypeptide to the molecule known to bind to the PDZ domain of said polypeptide.

31. A method of screening for a substance that increases binding of a PDZ domain polypeptide to a molecule known to bind to the PDZ domain of said polypeptide comprising:
(a) contacting a sample containing said polypeptide and molecule with a candidate substance;
(b) determining amount of binding said molecule to said polypeptide in the presence of the candidate substance;
(c) comparing the amount of binding of step (b) with amount of binding of said molecule to said polypeptide under similar conditions in the absence of the candidate substance;
whereby an increase in amount of binding of the polypeptide and said molecule in the presence of the candidate substance compared to the amount of binding in the absence of said candidate substance as determined in (c) indicates that said candidate substance is a substance that increases binding of the PDZ domain polypeptide to the molecule known to bind to the PDZ domain of said polypeptide.

32. A method comprising administering a substance to a subject with a condition associated with abnormal binding interaction of a PDZ domain polypeptide and a ligand, wherein said substance is a modulator of said binding interaction.

33. The method of claim 29 or 32, wherein the PDZ domain polypeptide comprises PDZ domain of ERBIN and the molecule known to bind to the polypeptide is δ-catenin, ARVCF or p0071.

34. The method of claim 29 or 32, wherein the PDZ domain polypeptide comprises PDZ domain of DENSIN and the molecule known to bind to the polypeptide is ARVCF, p0071 or δ-catENIN.

35. The method of claim 29 or 32, wherein the PDZ domain polypeptide comprises PDZI and/or 3 of SCRIBBLE and the molecule known to bind to the polypeptide is Z02 (tight junction protein 2), KV1.5, GPR87, ACTININ, p-CATENIN or CD34.

36. The method of claim 29 or 32, wherein the PDZ domain polypeptide comprises PDZ2 domain of SCRIBBLE and the molecule known to bind to the polypeptide is 6-CATENIN, ARVCF or p0071.

37. The method of claim 29 or 32, wherein the PDZ domain polypeptide comprises PDZ7 domain of MUPP and the molecule known to bind to the polypeptide is HTR2B, PDGFRb, δ-catenin, SGK or SSTR3.

38. The method of claim 29 or 32, wherein the PDZ domain polypeptide comprises PDZ6 domain of human INADL and the molecule known to bind to the polypeptide is HTR2B, PDGFRb, δ-catENIN, SGK or SSTR3.

39. The method of claim 29 or 32, wherein the PDZ domain polypeptide comprises PDZ domain of human ZO 1 and the molecule known to bind to the polypeptide is CLAUDIN-17, CLAUDIN-1, CLAUDIN-3, CLAUDIN-7, CLAUDIN-9, CLAUDIN-18, PDGFRA, PDGFRB, δ-catENIN, ARVCF or SGK.

40. The method of claim 29 or 32, wherein the PDZ domain polypeptide comprises PDZ domain of AF6 (MLLT4) and the molecule known to bind to the polypeptide is FYCO1, BLTR2, TM7SF3, OR10C1, CNTNAP2, NECTIN3, SH3D5 or UTROPHIN.

41. The method of claim 29-32, wherein the PDZ domain comprises PDZ3 domain of MUPP and the molecule known to bind to the polypeptide is drosophila NUMB homolog, TGFBR1, IGFBP7 or CD3611.

42. The method of claim 29 or 32, wherein the PDZ domain polypeptide comprises PDZ3 domain of MAGI 1 and the molecule known to bind to the polypeptide is SDOLF, PLEKHA1, PEPP2, MUC12, SLIT1, PARK2, HTR2A or PITPNB.

43. The method of claim 29 or 32, wherein the PDZ domain polypeptide comprises PDZ3 domain of MAGI3 and the molecule known to bind to the polypeptide is JAMI, JAM2, LLT1, PTTG3, CD83 antigen, DELTA-LIKE homolog (Drosophila), TNFRSF18, RGS20, TM4SF6, PARK2, GPR10 or IL2RB.

44. The method of claim 29 or 32, wherein the PDZ domain polypeptide comprises PDZ3 domain of INADL and the molecule known to bind to the polypeptide is BLTR2, JAM1, JAM2, KV8.1, PTTG3, CNTNAP2, NRXN1, NRXN2, NRXN3, TNFRSF18, PTTG1, PARK2, GABRG2, CNTFR, CCR2, GABRG3 or GABRP.

45. The method of claim 29 or 32, wherein the PDZ domain polypeptide comprises PDZ2 of huINADL and the molecule known to bind to the polypeptide is PIWI1, ortholog of mouse PIWI-LIKE HOMOLOG 1, NRXN1, NRXN2, PPP2CA or PPP2CB.

46. The method of claim 29 or 32, wherein the PDZ domain polypeptide comprises PDZ3 domain of huPARD3 and the molecule known to bind to the polypeptide is HRK, DOC1, PIWI or PPP1R3D.

47. The method of claim 29 or 32, wherein the PDZ domain polypeptide comprises PDZ domain of SNTA1 and the molecule known to bind to the polypeptide is MRGX2, NLGN1, NLGN3, SEEKI, CLAUDIN-17, GPR56, SSTR5, SCTR, GRM1, GRM2, GRM3 or GRM5.

48. The method of claim 29 or 32, wherein is the PDZ domain polypeptide comprises PDZ0 of MAGI3 and the molecule known to bind to the polypeptide is LANO, SSTR3, NRCAM, GPR19, GNG5 or HTR2B.

49. The method of claim 29 or 32, wherein the PDZ domain polypeptide comprises PDZ 13 domain of MUPP and the molecule known to bind to the polypeptide is NLGN3, NLGN1, CLAUDIN-16, GPR56, ENIGMA, FZD9, SSTR5, VCAM1 or GPRK6.

50. The method of claim 29 or 32, wherein the PDZ domain polypeptide comprises PDZ2 domain of MAGI3 and the molecule known to bind to the polypeptide is PTEN/MMAC.
